SEQUENCE LISTING (181 sequences)

SEQ ID NO: 1 - protein, 231 aa, Homo sapiens
  REGION 1..231: note = misc_feature - Human gamma 9 TCR subunit
AGHLEQPQISSTKTLSKTARLECVVSGITISATSVYWYRERPGEVIQFLVSISYDGTVRKESGIPSGKFEVDRIPETSTSTLTIHNVEKQDIATYYCALWEAQQELGKKIKVFGPGTKLIITDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIP

SEQ ID NO: 2 - protein, 115 aa, synthetic construct
  REGION 1..115: note = Synthetic: 1D7 VHH
QVQLVQSGGGLVQPGGSLRLSCVASRGKISQYRMGWYRQAPGKQRELVAHISNTGDAAEYVDSVKGRFTISRDSTKNAVYLQMNSLKPEDTAVYKCNSVEDILAWGQGTQVTVKP

SEQ ID NO: 3 - protein, 10 aa, synthetic construct
  REGION 1..10: note = Synthetic: CDR1 of 1D7 and humanized 1D7 (hu1D7) v1, v2, v9-v28, v30-v39, v57-v102, v112, v113, v124, v125, v139, v158 and v158-1-v158-10
RGKISQYRMG

SEQ ID NO: 4 - protein, 10 aa, synthetic construct
  REGION 1..10: note = Synthetic: CDR2 of 1D7 and hu1D7 v1-v10, v18-v29, v31-v39, v57-v102, v112, v113, v124, v125, v139, v158 and v158-1, v158-2, v158-4-v158-10
HISNTGDAAE

SEQ ID NO: 5 - protein, 6 aa, synthetic construct
  REGION 1..6: note = Synthetic: CDR3 of 1D7 and humanized 1D7 v1-v39 v57-v102, and v158
VEDILA

SEQ ID NO: 6 - protein, 130 aa, synthetic construct
  REGION 1..130: note = Synthetic: 5C8 VHH
EVQLVESGGGLVQAGGSLRLSCAASGRPFSNYAMGWFRQAPGKEREFVAAISWSGGSTSYADSVKGRFTISRDNAKNTVYLQMNSPKPEDTAIYYCAAQFSGADYGFGRLGIRGYEYDYWGQGTQVTVKP

SEQ ID NO: 7 - protein, 123 aa, synthetic construct
  REGION 1..123: note = Synthetic: 6C4 VHH
EVQLVESGGGLVQAGGSLRLSCAVSVRTFSDYRMGWFRQAPGKEREFVSTISWSGGLTYYADSVKGRFTISRDNSKNTLYLQMNSLKPEDTAVYYCAAGGGYAGGTYYHPEEWGQGTQVTVKP

SEQ ID NO: 8 - protein, 117 aa, synthetic construct
  REGION 1..117: note = Synthetic: 401A9v1 (non-targeted) VHH
EVQLVESGGGEVQPGGSLRLSCAASGSINSINVMEWYRQAPGKERDLVAGITSDGDTNYAESVKGRFTISRDNAKNTLYLQMSSLRAEDTAVYYCRARDWGSLTDYWGQGTLVTVKP

SEQ ID NO: 9 - protein, 361 aa, synthetic construct
  REGION 1..361: note = Synthetic: Fc region 2 IgG1 xELL knob-IL-2-T3G-H16A-P65R-D84S-C125S
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGSGGSAPGSSSTKKTQLQLEALLLDLQMILNGINNYKNPKLTRMLTFKFYMPKKATELKHLQCLEEELKRLEEVLNLAQSKNFHLRPRSLISNINVIVLELKGSETTFMCEYADETATIVEFLNRWITFSQSIISTLT

SEQ ID NO: 10 - protein, 358 aa, synthetic construct
  REGION 1..358: note = Synthetic: 5C8 VHH-Fc region xELL H435R, T366S, L368A, Y407V hole
EVQLVESGGGLVQAGGSLRLSCAASGRPFSNYAMGWFRQAPGKEREFVAAISWSGGSTSYADSVKGRFTISRDNAKNTVYLQMNSPKPEDTAIYYCAAQFSGADYGFGRLGIRGYEYDYWGQGTQVTVKPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 11 - protein, 495 aa, synthetic construct
  REGION 1..495: note = Synthetic: 5C8 VHH-Fc region 2 IgG1 xELL knob-IL-2-T3A-H16A-E61R-P65R-D84Y-C125S
EVQLVESGGGLVQAGGSLRLSCAASGRPFSNYAMGWFRQAPGKEREFVAAISWSGGSTSYADSVKGRFTISRDNAKNTVYLQMNSPKPEDTAIYYCAAQFSGADYGFGRLGIRGYEYDYWGQGTQVTVKPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGSGGSAPASSSTKKTQLQLEALLLDLQMILNGINNYKNPKLTRMLTFKFYMPKKATELKHLQCLERELKRLEEVLNLAQSKNFHLRPRYLISNINVIVLELKGSETTFMCEYADETATIVEFLNRWITFSQSIISTLT

SEQ ID NO: 12 - protein, 488 aa, synthetic construct
  REGION 1..488: note = Synthetic: 6C4 VHH-Fc region 2 IgG1 xELL knob-IL-2-T3A-H16A-E61R-P65R-D84Y-C125S
EVQLVESGGGLVQAGGSLRLSCAVSVRTFSDYRMGWFRQAPGKEREFVSTISWSGGLTYYADSVKGRFTISRDNSKNTLYLQMNSLKPEDTAVYYCAAGGGYAGGTYYHPEEWGQGTQVTVKPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGSGGSAPASSSTKKTQLQLEALLLDLQMILNGINNYKNPKLTRMLTFKFYMPKKATELKHLQCLERELKRLEEVLNLAQSKNFHLRPRYLISNINVIVLELKGSETTFMCEYADETATIVEFLNRWITFSQSIISTLT

SEQ ID NO: 13 - protein, 351 aa, synthetic construct
  REGION 1..351: note = Synthetic: 6C4 VHH-Fc region xELL H435R, T366S, L368A, Y407V hole
EVQLVESGGGLVQAGGSLRLSCAVSVRTFSDYRMGWFRQAPGKEREFVSTISWSGGLTYYADSVKGRFTISRDNSKNTLYLQMNSLKPEDTAVYYCAAGGGYAGGTYYHPEEWGQGTQVTVKPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 14 - protein, 345 aa, synthetic construct
  REGION 1..345: note = Synthetic: 401A9v1-VHH Fc region xELL H435R, T366S, L368A, Y407V hole
EVQLVESGGGEVQPGGSLRLSCAASGSINSINVMEWYRQAPGKERDLVAGITSDGDTNYAESVKGRFTISRDNAKNTLYLQMSSLRAEDTAVYYCRARDWGSLTDYWGQGTLVTVKPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 15 - protein, 482 aa, synthetic construct
  REGION 1..482: note = Synthetic: 401A9v1 VHH-Fc region 2 IgG1 xELL knob-IL-2-T3A-H16A-E61R-P65R-D84Y-C125S
EVQLVESGGGEVQPGGSLRLSCAASGSINSINVMEWYRQAPGKERDLVAGITSDGDTNYAESVKGRFTISRDNAKNTLYLQMSSLRAEDTAVYYCRARDWGSLTDYWGQGTLVTVKPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGSGGSAPASSSTKKTQLQLEALLLDLQMILNGINNYKNPKLTRMLTFKFYMPKKATELKHLQCLERELKRLEEVLNLAQSKNFHLRPRYLISNINVIVLELKGSETTFMCEYADETATIVEFLNRWITFSQSIISTLT

SEQ ID NO: 16 - protein, 480 aa, synthetic construct
  REGION 1..480: note = Synthetic: 1D7 VHH-Fc region 2 IgG1 xELL knob-IL-2-T3A-H16A-E61R-P65R-D84Y-C125S
QVQLVQSGGGLVQPGGSLRLSCVASRGKISQYRMGWYRQAPGKQRELVAHISNTGDAAEYVDSVKGRFTISRDSTKNAVYLQMNSLKPEDTAVYKCNSVEDILAWGQGTQVTVKPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGSGGSAPASSSTKKTQLQLEALLLDLQMILNGINNYKNPKLTRMLTFKFYMPKKATELKHLQCLERELKRLEEVLNLAQSKNFHLRPRYLISNINVIVLELKGSETTFMCEYADETATIVEFLNRWITFSQSIISTLT

SEQ ID NO: 17 - protein, 115 aa, synthetic construct
  REGION 1..115: note = Synthetic: hu1D7v1 VHH
EVQLLESGGGEVQPGGSLRLSCVASRGKISQYRMGWYRQAPGKGLEWVAHISNTGDAAEYVDSVKGRFTISRDSTKNAVYLQMSSLRAEDTAVYKCNSVEDILAWGQGTLVTVKP

SEQ ID NO: 18 - protein, 115 aa, synthetic construct
  REGION 1..115: note = Synthetic: hu1D7v2 VHH
EVQLLESGGGEVQPGGSLRLSCAASRGKISQYRMGWYRQAPGKGLEWVAHISNTGDAAEYVDSVKGRFTISRDSTKNAVYLQMSSLRAEDTAVYKCNSVEDILAWGQGTLVTVKP

SEQ ID NO: 19 - protein, 115 aa, synthetic construct
  REGION 1..115: note = Synthetic: hu1D7v3 VHH
EVQLLESGGGEVQPGGSLRLSCVASGGKISQYRMGWYRQAPGKGLEWVAHISNTGDAAEYVDSVKGRFTISRDSTKNAVYLQMSSLRAEDTAVYKCNSVEDILAWGQGTLVTVKP

SEQ ID NO: 20 - protein, 115 aa, synthetic construct
  REGION 1..115: note = Synthetic: hu1D7v4 VHH
EVQLLESGGGEVQPGGSLRLSCVASRFKISQYRMGWYRQAPGKGLEWVAHISNTGDAAEYVDSVKGRFTISRDSTKNAVYLQMSSLRAEDTAVYKCNSVEDILAWGQGTLVTVKP

SEQ ID NO: 21 - protein, 115 aa, synthetic construct
  REGION 1..115: note = Synthetic: hu1D7v5 VHH
EVQLLESGGGEVQPGGSLRLSCVASRGTISQYRMGWYRQAPGKGLEWVAHISNTGDAAEYVDSVKGRFTISRDSTKNAVYLQMSSLRAEDTAVYKCNSVEDILAWGQGTLVTVKP

SEQ ID NO: 22 - protein, 115 aa, synthetic construct
  REGION 1..115: note = Synthetic: hu1D7v6 VHH
EVQLLESGGGEVQPGGSLRLSCVASRGKFSQYRMGWYRQAPGKGLEWVAHISNTGDAAEYVDSVKGRFTISRDSTKNAVYLQMSSLRAEDTAVYKCNSVEDILAWGQGTLVTVKP

SEQ ID NO: 23 - protein, 115 aa, synthetic construct
  REGION 1..115: note = Synthetic: hu1D7v7 VHH
EVQLLESGGGEVQPGGSLRLSCVASRGKISSYRMGWYRQAPGKGLEWVAHISNTGDAAEYVDSVKGRFTISRDSTKNAVYLQMSSLRAEDTAVYKCNSVEDILAWGQGTLVTVKP

SEQ ID NO: 24 - protein, 115 aa, synthetic construct
  REGION 1..115: note = Synthetic: hu1D7v8 VHH
EVQLLESGGGEVQPGGSLRLSCVASRGKISQYAMGWYRQAPGKGLEWVAHISNTGDAAEYVDSVKGRFTISRDSTKNAVYLQMSSLRAEDTAVYKCNSVEDILAWGQGTLVTVKP

SEQ ID NO: 25 - protein, 115 aa, synthetic construct
  REGION 1..115: note = Synthetic: hu1D7v9 VHH
EVQLLESGGGEVQPGGSLRLSCVASRGKISQYRMGWYRQAPGKELEWVAHISNTGDAAEYVDSVKGRFTISRDSTKNAVYLQMSSLRAEDTAVYKCNSVEDILAWGQGTLVTVKP

SEQ ID NO: 26 - protein, 115 aa, synthetic construct
  REGION 1..115: note = Synthetic: hu1D7v10 VHH
EVQLLESGGGEVQPGGSLRLSCVASRGKISQYRMGWYRQAPGKGLEWVSHISNTGDAAEYVDSVKGRFTISRDSTKNAVYLQMSSLRAEDTAVYKCNSVEDILAWGQGTLVTVKP

SEQ ID NO: 27 - protein, 115 aa, synthetic construct
  REGION 1..115: note = Synthetic: hu1D7v11 VHH
EVQLLESGGGEVQPGGSLRLSCVASRGKISQYRMGWYRQAPGKGLEWVAAISNTGDAAEYVDSVKGRFTISRDSTKNAVYLQMSSLRAEDTAVYKCNSVEDILAWGQGTLVTVKP

SEQ ID NO: 28 - protein, 115 aa, synthetic construct
  REGION 1..115: note = Synthetic: hu1D7v12 VHH
EVQLLESGGGEVQPGGSLRLSCVASRGKISQYRMGWYRQAPGKGLEWVAHISGTGDAAEYVDSVKGRFTISRDSTKNAVYLQMSSLRAEDTAVYKCNSVEDILAWGQGTLVTVKP

SEQ ID NO: 29 - protein, 115 aa, synthetic construct
  REGION 1..115: note = Synthetic: hu1D7v13 VHH
EVQLLESGGGEVQPGGSLRLSCVASRGKISQYRMGWYRQAPGKGLEWVAHISNSGDAAEYVDSVKGRFTISRDSTKNAVYLQMSSLRAEDTAVYKCNSVEDILAWGQGTLVTVKP

SEQ ID NO: 30 - protein, 115 aa, synthetic construct
  REGION 1..115: note = Synthetic: hu1D7v14 VHH
EVQLLESGGGEVQPGGSLRLSCVASRGKISQYRMGWYRQAPGKGLEWVAHISNTGGAAEYVDSVKGRFTISRDSTKNAVYLQMSSLRAEDTAVYKCNSVEDILAWGQGTLVTVKP

SEQ ID NO: 31 - protein, 115 aa, synthetic construct
  REGION 1..115: note = Synthetic: hu1D7v15 VHH
EVQLLESGGGEVQPGGSLRLSCVASRGKISQYRMGWYRQAPGKGLEWVAHISNTGDSAEYVDSVKGRFTISRDSTKNAVYLQMSSLRAEDTAVYKCNSVEDILAWGQGTLVTVKP

SEQ ID NO: 32 - protein, 227 aa, Homo sapiens
  REGION 1..227: note = misc_feature - human IgG1 Fc region
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 224 aa, Homo sapiens
  REGION 1..224: note = misc_feature - human IgG1 Fc xELL
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 227 aa, synthetic construct
  REGION 1..227: note = Synthetic: Fc region M252Y and M428V (YV) S354C T366W knob
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVVHEALHNHYTQKSLSLSPGK

SEQ ID NO: 35 - protein, 227 aa, synthetic construct
  REGION 1..227: note = Synthetic: Fc region M252Y, M428V, H435R (YVR) T366S, L368A, Y407V hole
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVVHEALHNRYTQKSLSLSPGK

SEQ ID NO: 36 - protein, 224 aa, synthetic construct
  REGION 1..224: note = Synthetic: Fc region xELL H435R
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRYTQKSLSLSPGK

SEQ ID NO: 37 - protein, 224 aa, synthetic construct
  REGION 1..224: note = Synthetic: Fc region xELL M252Y and M428V (YV)
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVVHEALHNHYTQKSLSLSPGK

SEQ ID NO: 38 - protein, 224 aa, synthetic construct
  REGION 1..224: note = Synthetic: Fc region xELL M252Y and M428L (YL)
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 39 - protein, 224 aa, synthetic construct
  REGION 1..224: note = Synthetic: Fc region xELL M252Y, M428L, H435R (YLR)
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNRYTQKSLSLSPGK

SEQ ID NO: 40 - protein, 224 aa, synthetic construct
  REGION 1..224: note = Synthetic: Fc region xELL M252Y, M428V, H435R (YVR)
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVVHEALHNRYTQKSLSLSPGK

SEQ ID NO: 41 - protein, 224 aa, synthetic construct
  REGION 1..224: note = Synthetic: Fc region xELL S354C T366W knob
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 224 aa, synthetic construct
  REGION 1..224: note = Synthetic: Fc region xELL H435R S354C T366W knob
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNRYTQKSLSLSPGK

SEQ ID NO: 43 - protein, 224 aa, synthetic construct
  REGION 1..224: note = Synthetic: Fc region xELL M252Y and M428V (YV) S354C T366W knob
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVVHEALHNHYTQKSLSLSPGK

SEQ ID NO: 44 - protein, 224 aa, synthetic construct
  REGION 1..224: note = Synthetic: Fc region xELL M252Y and M428L (YL) S354C T366W knob
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHNHYTQKSLSLSPGK

SEQ ID NO: 45 - protein, 224 aa, synthetic construct
  REGION 1..224: note = Synthetic: Fc region xELL M252Y, M428L, H435R (YLR) S354C T366W knob
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHNRYTQKSLSLSPGK

SEQ ID NO: 46 - protein, 224 aa, synthetic construct
  REGION 1..224: note = Synthetic: Fc region xELL M252Y, M428V, H435R (YVR) S354C T366W knob
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVVHEALHNRYTQKSLSLSPGK

SEQ ID NO: 47 - protein, 224 aa, synthetic construct
  REGION 1..224: note = Synthetic: Fc region xELL T366S, L368A, Y407V hole
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 48 - protein, 224 aa, synthetic construct
  REGION 1..224: note = Synthetic: Fc region xELL H435R, T366S, L368A, Y407V hole
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 49 - protein, 224 aa, synthetic construct
  REGION 1..224: note = Synthetic: Fc region xELL M252Y and M428V (YV) T366S, L368A, Y407V hole
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVVHEALHNHYTQKSLSLSPGK

SEQ ID NO: 50 - protein, 224 aa, synthetic construct
  REGION 1..224: note = Synthetic: Fc region xELL M252Y and M428L (YL) T366S, L368A, Y407V hole
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHNHYTQKSLSLSPGK

SEQ ID NO: 51 - protein, 224 aa, synthetic construct
  REGION 1..224: note = Synthetic: Fc region xELL M252Y, M428L, H435R (YLR) T366S, L368A, Y407V hole
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHNRYTQKSLSLSPGK

SEQ ID NO: 52 - protein, 224 aa, synthetic construct
  REGION 1..224: note = Synthetic: Fc region xELL M252Y, M428V, H435R (YVR) T366S, L368A, Y407V hole
DKTHTCPPCPAPGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVVHEALHNRYTQKSLSLSPGK

SEQ ID NO: 53 - protein, 227 aa, synthetic construct
  REGION 1..227: note = Synthetic: Fc region H435R
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRYTQKSLSLSPGK

SEQ ID NO: 54 - protein, 227 aa, synthetic construct
  REGION 1..227: note = Synthetic: Fc region M252Y and M428V (YV)
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVVHEALHNHYTQKSLSLSPGK

SEQ ID NO: 55 - protein, 227 aa, synthetic construct
  REGION 1..227: note = Synthetic: Fc region M252Y and M428L (YL)
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 56 - protein, 227 aa, synthetic construct
  REGION 1..227: note = Synthetic: Fc region M252Y, M428L, H435R (YLR)
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHNRYTQKSLSLSPGK

SEQ ID NO: 57 - protein, 227 aa, synthetic construct
  REGION 1..227: note = Synthetic: Fc region M252Y, M428V, H435R (YVR)
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVVHEALHNRYTQKSLSLSPGK

SEQ ID NO: 58 - protein, 227 aa, synthetic construct
  REGION 1..227: note = Synthetic: Fc region S354C T366W knob
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 227 aa, synthetic construct
  REGION 1..227: note = Synthetic: Fc region H435R S354C T366W knob
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNRYTQKSLSLSPGK

SEQ ID NO: 60 - protein, 227 aa, synthetic construct
  REGION 1..227: note = Synthetic: Fc region M252Y and M428L (YL) S354C T366W knob
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHNHYTQKSLSLSPGK

SEQ ID NO: 61 - protein, 227 aa, synthetic construct
  REGION 1..227: note = Synthetic: Fc region M252Y, M428L, H435R (YLR) S354C T366W knob
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHNRYTQKSLSLSPGK

SEQ ID NO: 62 - protein, 227 aa, synthetic construct
  REGION 1..227: note = Synthetic: Fc region M252Y, M428V, H435R (YVR) S354C T366W knob
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVVHEALHNRYTQKSLSLSPGK

SEQ ID NO: 63 - protein, 227 aa, synthetic construct
  REGION 1..227: note = Synthetic: Fc region T366S, L368A, Y407V hole
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 64 - protein, 227 aa, synthetic construct
  REGION 1..227: note = Synthetic: Fc region H435R, T366S, L368A, Y407V hole
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 65 - protein, 227 aa, synthetic construct
  REGION 1..227: note = Synthetic: Fc region M252Y and M428V (YV) T366S, L368A, Y407V hole
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVVHEALHNHYTQKSLSLSPGK

SEQ ID NO: 66 - protein, 227 aa, synthetic construct
  REGION 1..227: note = Synthetic: Fc region M252Y and M428L (YL) T366S, L368A, Y407V hole
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHNHYTQKSLSLSPGK

SEQ ID NO: 67 - protein, 227 aa, synthetic construct
  REGION 1..227: note = Synthetic: Fc region M252Y, M428L, H435R (YLR) T366S, L368A, Y407V hole
DKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHNRYTQKSLSLSPGK

SEQ ID NO: 68 - protein, 223 aa, synthetic construct
  REGION 1..223: note = Synthetic: Fc region xELL P329G, H435R, T366S, L368A, Y407V hole (lacking C-terminal lysine)
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRYTQKSLSLSPG

SEQ ID NO: 69 - protein, 223 aa, synthetic construct
  REGION 1..223: note = Synthetic: Fc region xELL P329G knob (lacking C-terminal lysine)
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 70 - protein, 223 aa, synthetic construct
  REGION 1..223: note = Synthetic: Fc region 2 IgG1 xELL knob (lacking C-terminal lysine)
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 71 - protein, 133 aa, Homo sapiens [sequence omitted]

SEQ ID NO: 72 - protein, 115 aa, synthetic construct
  REGION 1..115: note = Synthetic: hu1D7v16 VHH
EVQLLESGGGEVQPGGSLRLSCVASRGKISQYRMGWYRQAPGKGLEWVAHISNTGDATEYVDSVKGRFTISRDSTKNAVYLQMSSLRAEDTAVYKCNSVEDILAWGQGTLVTVKP

SEQ ID NO: 73 - protein, 115 aa, synthetic construct
  REGION 1..115: note = Synthetic: hu1D7v17 VHH
EVQLLESGGGEVQPGGSLRLSCVASRGKISQYRMGWYRQAPGKGLEWVAHISNTGDAAYYVDSVKGRFTISRDSTKNAVYLQMSSLRAEDTAVYKCNSVEDILAWGQGTLVTVKP

SEQ ID NO: 74 - protein, 115 aa, synthetic construct
  REGION 1..115: note = Synthetic: hu1D7v18 VHH
EVQLLESGGGEVQPGGSLRLSCVASRGKISQYRMGWYRQAPGKGLEWVAHISNTGDAAEYAESVKGRFTISRDSTKNAVYLQMSSLRAEDTAVYKCNSVEDILAWGQGTLVTVKP

SEQ ID NO: 75 - protein, 115 aa, synthetic construct
  REGION 1..115: note = Synthetic: hu1D7v19 VHH
EVQLLESGGGEVQPGGSLRLSCVASRGKISQYRMGWYRQAPGKGLEWVAHISNTGDAAEYVDSVKDRFTISRDSTKNAVYLQMSSLRAEDTAVYKCNSVEDILAWGQGTLVTVKP

SEQ ID NO: 76 - protein, 115 aa, synthetic construct
  REGION 1..115: note = Synthetic: hu1D7v20 VHH
EVQLLESGGGEVQPGGSLRLSCVASRGKISQYRMGWYRQAPGKGLEWVAHISNTGDAAEYVDSVKGRFTISRDNTKNAVYLQMSSLRAEDTAVYKCNSVEDILAWGQGTLVTVKP

SEQ ID NO: 77 - protein, 115 aa, synthetic construct
  REGION 1..115: note = Synthetic: hu1D7v21 VHH
EVQLLESGGGEVQPGGSLRLSCVASRGKISQYRMGWYRQAPGKGLEWVAHISNTGDAAEYVDSVKGRFTISRDSAKNAVYLQMSSLRAEDTAVYKCNSVEDILAWGQGTLVTVKP

SEQ ID NO: 78 - protein, 133 aa, synthetic construct
  REGION 1..133: note = Synthetic: IL-2-T3A-H16A-E61R-P65R-D84Y-C125S (IL-2_X)
APASSSTKKTQLQLEALLLDLQMILNGINNYKNPKLTRMLTFKFYMPKKATELKHLQCLERELKRLEEVLNLAQSKNFHLRPRYLISNINVIVLELKGSETTFMCEYADETATIVEFLNRWITFSQSIISTLT

SEQ ID NO: 79 - protein, 133 aa, synthetic construct
  REGION 1..133: note = Synthetic: IL-2-T3A-H16A-P65R-D84S-C125S (IL-2_Z)
APASSSTKKTQLQLEALLLDLQMILNGINNYKNPKLTRMLTFKFYMPKKATELKHLQCLEEELKRLEEVLNLAQSKNFHLRPRSLISNINVIVLELKGSETTFMCEYADETATIVEFLNRWITFSQSIISTLT

SEQ ID NO: 80 - protein, 115 aa, synthetic construct
  REGION 1..115: note = Synthetic: hu1D7v22 VHH
EVQLLESGGGEVQPGGSLRLSCVASRGKISQYRMGWYRQAPGKGLEWVAHISNTGDAAEYVDSVKGRFTISRDSTKNTVYLQMSSLRAEDTAVYKCNSVEDILAWGQGTLVTVKP

SEQ ID NO: 81 - protein, 115 aa, synthetic construct
  REGION 1..115: note = Synthetic: hu1D7v23 VHH
EVQLLESGGGEVQPGGSLRLSCVASRGKISQYRMGWYRQAPGKGLEWVAHISNTGDAAEYVDSVKGRFTISRDSTKNALYLQMSSLRAEDTAVYKCNSVEDILAWGQGTLVTVKP

SEQ ID NO: 82 - protein, 115 aa, synthetic construct
  REGION 1..115: note = Synthetic: hu1D7v24 VHH
EVQLLESGGGEVQPGGSLRLSCVASRGKISQYRMGWYRQAPGKGLEWVAHISNTGDAAEYVDSVKGRFTISRDSTKNAVYLQMSSLRAEDTAVYKCNAVEDILAWGQGTLVTVKP

SEQ ID NO: 83 - protein, 115 aa, synthetic construct
  REGION 1..115: note = Synthetic: hu1D7v25 VHH
EVQLLESGGGEVQPGGSLRLSCVASRGKISQYRMGWYRQAPGKGLEWVAHISNTGDAAEYVDSVKGRFTISRDSTKNAVYLQMSSLRAEDTAVYKCASVEDILAWGQGTLVTVKP

SEQ ID NO: 84 - protein, 115 aa, synthetic construct
  REGION 1..115: note = Synthetic: hu1D7v26 VHH
EVQLLESGGGEVQPGGSLRLSCVASRGKISQYRMGWYRQAPGKGLEWVAHISNTGDAAEYVDSVKGRFTISRDSTKNAVYLQMSSLRAEDTAVYYCNSVEDILAWGQGTLVTVKP

SEQ ID NO: 85 - protein, 115 aa, synthetic construct; identical to SEQ ID NO: 25
  REGION 1..115: note = Synthetic: hu1D7v27 VHH

SEQ ID NO: 86 - protein, 115 aa, synthetic construct
  REGION 1..115: note = Synthetic: hu1D7v28 VHH
EVQLLESGGGEVQPGGSLRLSCAASRGKISQYRMGWYRQAPGKELEWVAHISNTGDAAEYVDSVKGRFTISRDSTKNAVYLQMSSLRAEDTAVYKCNSVEDILAWGQGTLVTVKP

SEQ ID NO: 87 - protein, 115 aa, synthetic construct
  REGION 1..115: note = Synthetic: hu1D7v29 VHH
EVQLLESGGGEVQPGGSLRLSCVASRGKISSYRMGWYRQAPGKELEWVAHISNTGDAAEYVDSVKGRFTISRDSTKNAVYLQMSSLRAEDTAVYKCNSVEDILAWGQGTLVTVKP

SEQ ID NO: 88 - protein, 115 aa, synthetic construct
  REGION 1..115: note = Synthetic: hu1D7v30 VHH
EVQLLESGGGEVQPGGSLRLSCVASRGKISQYRMGWYRQAPGKELEWVAHISNTGDAAYYVDSVKGRFTISRDSTKNAVYLQMSSLRAEDTAVYKCNSVEDILAWGQGTLVTVKP

SEQ ID NO: 89 - protein, 115 aa, synthetic construct
  REGION 1..115: note = Synthetic: hu1D7v31 VHH
EVQLLESGGGEVQPGGSLRLSCVASRGKISQYRMGWYRQAPGKELEWVAHISNTGDAAEYVAPVKGRFTISRDSTKNAVYLQMSSLRAEDTAVYKCNSVEDILAWGQGTLVTVKP

SEQ ID NO: 90 - protein, 115 aa, synthetic construct
  REGION 1..115: note = Synthetic: hu1D7v32 VHH
EVQLLESGGGEVQPGGSLRLSCVASRGKISQYRMGWYRQAPGKELEWVAHISNTGDAAEYVESVKGRFTISRDSTKNAVYLQMSSLRAEDTAVYKCNSVEDILAWGQGTLVTVKP

SEQ ID NO: 91 - protein, 115 aa, synthetic construct
  REGION 1..115: note = Synthetic: hu1D7v33 VHH
EVQLLESGGGEVQPGGSLRLSCVASRGKISQYRMGWYRQAPGKELEWVAHISNTGDAAEYVDSVKDRFTISRDSTKNAVYLQMSSLRAEDTAVYKCNSVEDILAWGQGTLVTVKP

SEQ ID NO: 92 - protein, 115 aa, synthetic construct
  REGION 1..115: note = Synthetic: hu1D7v34 VHH
EVQLLESGGGEVQPGGSLRLSCVASRGKISQYRMGWYRQAPGKELEWVAHISNTGDAAEYVDSVKGRFTISRDNAKNAVYLQMSSLRAEDTAVYKCNSVEDILAWGQGTLVTVKP

SEQ ID NO: 93 - protein, 115 aa, synthetic construct
  REGION 1..115: note = Synthetic: hu1D7v35 VHH
EVQLLESGGGEVQPGGSLRLSCVASRGKISQYRMGWYRQAPGKELEWVAHISNTGDAAEYVDSVKGRFTISRDSTKNALYLQMSSLRAEDTAVYKCNSVEDILAWGQGTLVTVKP

SEQ ID NO: 94 - protein, 115 aa, synthetic construct
  REGION 1..115: note = Synthetic: hu1D7v36 VHH
EVQLLESGGGEVQPGGSLRLSCVASRGKISQYRMGWYRQAPGKELEWVAHISNTGDAAEYVDSVKGRFTISRDSTKNAVYLQMSSLRAEDTAVYYCNSVEDILAWGQGTLVTVKP

SEQ ID NO: 95 - protein, 115 aa, synthetic construct
  REGION 1..115: note = Synthetic: hu1D7v37 VHH
EVQLLESGGGEVQPGGSLRLSCVASRGKISQYRMGWYRQAPGKELEWVAHISNTGDAAEYVDSVKGRFTISRDSTKNAVYLQMSSLRAEDTAVYKCNTVEDILAWGQGTLVTVKP

SEQ ID NO: 96 - protein, 115 aa, synthetic construct
  REGION 1..115: note = Synthetic: hu1D7v38 VHH
EVQLLESGGGEVQPGGSLRLSCVASRGKISQYRMGWYRQAPGKELEWVAHISNTGDAAEYVDSVKGRFTISRDSTKNAVYLQMSSLRAEDTAVYKCNVVEDILAWGQGTLVTVKP

SEQ ID NO: 97 - protein, 115 aa, synthetic construct
  REGION 1..115: note = Synthetic: hu1D7v39 VHH
EVQLLESGGGEVQPGGSLRLSCVASRGKISQYRMGWYRQAPGKELEWVAHISNTGDAAEYVDSVKGRFTISRDSTKNAVYLQMSSLRAEDTAVYKCNSVEDILAWGQGTLVTVRP

SEQ ID NO: 98 - protein, 115 aa, synthetic construct
  REGION 1..115: note = Synthetic: hu1D7v57 VHH
EVQLLESGGGEVQPGGSLRLSCVASRGKISQYRMGWYRQAPGKERELVAHISNTGDAAEYVDSVKGRFTISRDSTKNALYLQMSSLRAEDTAVYKCNSVEDILAWGQGTLVTVRP

SEQ ID NO: 99 - protein, 115 aa, synthetic construct
  REGION 1..115: note = Synthetic: hu1D7v58 VHH
EVQLLESGGGEVQPGGSLRLSCVASRGKISQYRMGWYRQAPGKERELVAHISNTGDAAEYVDSVKDRFTISRDSTKNALYLQMSSLRAEDTAVYKCNSVEDILAWGQGTLVTVRP

SEQ ID NO: 100 - protein, 115 aa, synthetic construct
  REGION 1..115: note = Synthetic: hu1D7v59 VHH
EVQLLESGGGEVQPGGSLRLSCVASRGKISQYRMGWYRQAPGKERELVAHISNTGDAAEYVAPVKDRFTISRDSTKNALYLQMSSLRAEDTAVYKCNSVEDILAWGQGTLVTVRP

SEQ ID NO: 101 - protein, 115 aa, synthetic construct
  REGION 1..115: note = Synthetic: hu1D7v60 VHH
EVQLLESGGGEVQPGGSLRLSCVASRGKISQYRMGWYRQAPGKERELVAHISNTGDAAEYVDSVKDRFTISRDNAKNALYLQMSSLRAEDTAVYKCNSVEDILAWGQGTLVTVRP

SEQ ID NO: 102 - protein, 115 aa, synthetic construct
  REGION 1..115: note = Synthetic: hu1D7v61 VHH
EVQLLESGGGEVQPGGSLRLSCVASRGKISQYRMGWYRQAPGKERELVAHISNTGDAAEYVDSVKDRFTISRDSTKNALYLQMSSLRAEDTAVYKCNTVEDILAWGQGTLVTVRP

SEQ ID NO: 103 - protein, 115 aa, synthetic construct
  REGION 1..115: note = Synthetic: hu1D7v62 VHH
EVQLLESGGGEVQPGGSLRLSCVASRGKISQYRMGWYRQAPGKERELVAHISNTGDAAEYVAPVKDRFTISRDNAKNALYLQMSSLRAEDTAVYKCNTVEDILAWGQGTLVTVRP

SEQ ID NO: 104 - protein, 115 aa, synthetic construct
  REGION 1..115: note = Synthetic: hu1D7v63 VHH
EVQLLESGGGEVQPGGSLRLSCAASRGKISQYRMGWYRQAPGKERELVAHISNTGDAAEYVAPVKDRFTISRDNAKNALYLQMSSLRAEDTAVYKCNTVEDILAWGQGTLVTVRP

SEQ ID NO: 105 - protein, 115 aa, synthetic construct
  REGION 1..115: note = Synthetic: hu1D7v64 VHH
EVQLLESGGGEVQPGGSLRLSCVASRGKISQYRMGWYRQAPGKERELVAHISNTGDAAEYVESVKDRFTISRDSTKNALYLQMSSLRAEDTAVYKCNSVEDILAWGQGTLVTVRP

SEQ ID NO: 106 - protein, 115 aa, synthetic construct
  REGION 1..115: note = Synthetic: hu1D7v65 VHH
EVQLLESGGGEVQPGGSLRLSCVASRGKISQYRMGWYRQAPGKERELVAHISNTGDAAEYVDAVKDRFTISRDSTKNALYLQMSSLRAEDTAVYKCNSVEDILAWGQGTLVTVRP

SEQ ID NO: 107 - protein, 115 aa, synthetic construct
  REGION 1..115: note = Synthetic: hu1D7v66 VHH
EVQLLESGGGEVQPGGSLRLSCVASRGKISQYRMGWYRQAPGKERELVAHISNTGDAAEYVDTVKDRFTISRDSTKNALYLQMSSLRAEDTAVYKCNSVEDILAWGQGTLVTVRP

SEQ ID NO: 108 - protein, 115 aa, synthetic construct
  REGION 1..115: note = Synthetic: hu1D7v67 VHH
EVQLLESGGGEVQPGGSLRLSCVASRGKISQYRMGWYRQAPGKERELVAHISNTGDAAEYVDSVKDRFTISRDSTKNALYLQMSSLRAEDTAVYKCQSVEDILAWGQGTLVTVRP

SEQ ID NO: 109 - protein, 115 aa, synthetic construct
  REGION 1..115: note = Synthetic: hu1D7v68 VHH
EVQLLESGGGEVQPGGSLRLSCVASRGKISQYRMGWYRQAPGKERELVAHISNTGDAAEYVDSVKDRFTISRDSTKNALYLQMSSLRAEDTAVYKCSSVEDILAWGQGTLVTVRP

SEQ ID NO: 110 - protein, 115 aa, synthetic construct
  REGION 1..115: note = Synthetic: hu1D7v69 VHH
EVQLLESGGGEVQPGGSLRLSCVASRGKISQYRMGWYRQAPGKERELVAHISNTGDAAEYVDSVKDRFTISRDSTKNALYLQMSSLRAEDTAVYKCTSVEDILAWGQGTLVTVRP

SEQ ID NO: 111 - protein, 115 aa, synthetic construct
  REGION 1..115: note = Synthetic: hu1D7v70 VHH
EVQLLESGGGEVQPGGSLRLSCVASRGKISQYRMGWYRQAPGKERELVAHISNTGDAAEYVDSVKDRFTISRDSTKNALYLQMSSLRAEDTAVYKCESVEDILAWGQGTLVTVRP

SEQ ID NO: 112 - protein, 115 aa, synthetic construct
  REGION 1..115: note = Synthetic: hu1D7v71 VHH
EVQLLESGGGEVQPGGSLRLSCVASRGKISQYRMGWYRQAPGKERELVAHISNTGDAAEYVDVVKDRFTISRDSTKNALYLQMSSLRAEDTAVYKCNSVEDILAWGQGTLVTVRP

SEQ ID NO: 113 - protein, 115 aa, synthetic construct
  REGION 1..115: note = Synthetic: hu1D7v72 VHH
EVQLLESGGGEVQPGGSLRLSCVASRGKISQYRMGWYRQAPGKERELVAHISNTGDAAEYVDLVKDRFTISRDSTKNALYLQMSSLRAEDTAVYKCNSVEDILAWGQGTLVTVRP

SEQ ID NO: 114 - protein, 115 aa, synthetic construct
  REGION 1..115: note = Synthetic: hu1D7v73 VHH
EVQLLESGGGEVQPGGSLRLSCVASRGKISQYRMGWYRQAPGKERELVAHISNTGDAAEYVDIVKDRFTISRDSTKNALYLQMSSLRAEDTAVYKCNSVEDILAWGQGTLVTVRP

SEQ ID NO: 115 - protein, 115 aa, synthetic construct
  REGION 1..115: note = Synthetic: hu1D7v74 VHH
EVQLLESGGGEVQPGGSLRLSCVASRGKISQYRMGWYRQAPGKERELVAHISNTGDAAEYVASVKDRFTISRDSTKNALYLQMSSLRAEDTAVYKCNSVEDILAWGQGTLVTVRP

SEQ ID NO: 116 - protein, 115 aa, synthetic construct
  REGION 1..115: note = Synthetic: hu1D7v75 VHH
EVQLLESGGGEVQPGGSLRLSCVASRGKISQYRMGWYRQAPGKERELVAHISNTGDAAEYVGSVKDRFTISRDSTKNALYLQMSSLRAEDTAVYKCNSVEDILAWGQGTLVTVRP

SEQ ID NO: 117 - protein, 115 aa, synthetic construct
  REGION 1..115: note = Synthetic: hu1D7v76 VHH
EVQLLESGGGEVQPGGSLRLSCVASRGKISQYRMGWYRQAPGKERELVAHISNTGDAAEYVVSVKDRFTISRDSTKNALYLQMSSLRAEDTAVYKCNSVEDILAWGQGTLVTVRP

SEQ ID NO: 118 - protein, 115 aa, synthetic construct
  REGION 1..115: note = Synthetic: hu1D7v77 VHH
EVQLLESGGGEVQPGGSLRLSCVASRGKISQYRMGWYRQAPGKERELVAHISNTGDAAEYVDSVKDRFTISRDSTKNALYLQMSSLRAEDTAVYKCYSVEDILAWGQGTLVTVRP

SEQ ID NO: 119 - protein, 115 aa, synthetic construct
  REGION 1..115: note = Synthetic: hu1D7v78 VHH
EVQLLESGGGEVQPGGSLRLSCVASRGKISQYRMGWYRQAPGKERELVAHISNTGDAAEYVDSVKDRFTISRDSTKNALYLQMSSLRAEDTAVYKCNAVEDILAWGQGTLVTVRP

SEQ ID NO: 120 - protein, 115 aa, synthetic construct
  REGION 1..115: note = Synthetic: hu1D7v79 VHH
EVQLLESGGGEVQPGGSLRLSCVASRGKISQYRMGWYRQAPGKERELVAHISNTGDAAEYVDSVKDRFTISRDSTKNALYLQMSSLRAEDTAVYKCNLVEDILAWGQGTLVTVRP

SEQ ID NO: 121 - protein, 115 aa, synthetic construct
  REGION 1..115: note = Synthetic: hu1D7v80 VHH
EVQLLESGGGEVQPGGSLRLSCVASRGKISQYRMGWYRQAPGKERELVAHISNTGDAAEYVDSVKDRFTISRDSTKNALYLQMSSLRAEDTAVYKCNVVEDILAWGQGTLVTVRP

SEQ ID NO: 122 - protein, 115 aa, synthetic construct
  REGION 1..115: note = Synthetic: hu1D7v81 VHH
EVQLLESGGGEVQPGGSLRLSCVASRGKISQYRMGWYRQAPGKERELVAHISNTGDAAEYVDSVKDRFTISRDSTKNALYLQMSSLRAEDTAVYKCASVEDILAWGQGTLVTVRP

SEQ ID NO: 123 - protein, 115 aa, synthetic construct; identical to SEQ ID NO: 105
  REGION 1..115: note = Synthetic: hu1D7v82 VHH

SEQ ID NO: 124 - protein, 115 aa, synthetic construct
  REGION 1..115: note = Synthetic: hu1D7v83 VHH
EVQLLESGGGEVQPGGSLRLSCVASRGKISQYRMGWYRQAPGKERELVAHISNTGDAAEYVDGVKDRFTISRDSTKNALYLQMSSLRAEDTAVYKCNSVEDILAWGQGTLVTVRP

SEQ ID NO: 125 - protein, 115 aa, synthetic construct
  REGION 1..115: note = Synthetic: hu1D7v84 VHH
EVQLLESGGGEVQPGGSLRLSCVASRGKISQYRMGWYRQAPGKERELVAHISNTGDAAEYVEGVKDRFTISRDSTKNALYLQMSSLRAEDTAVYKCNSVEDILAWGQGTLVTVRP

SEQ ID NO: 126 - protein, 115 aa, synthetic construct
  REGION 1..115: note = Synthetic: hu1D7v85 VHH
EVQLLESGGGEVQPGGSLRLSCVASRGKISQYRMGWYRQAPGKERELVAHISNTGDAAEYVSGVKDRFTISRDSTKNALYLQMSSLRAEDTAVYKCNSVEDILAWGQGTLVTVRP

SEQ ID NO: 127 - protein, 115 aa, synthetic construct
  REGION 1..115: note = Synthetic: hu1D7v86 VHH
EVQLLESGGGEVQPGGSLRLSCVASRGKISQYRMGWYRQAPGKERELVAHISNTGDAAEYVGGVKDRFTISRDSTKNALYLQMSSLRAEDTAVYKCNSVEDILAWGQGTLVTVRP

SEQ ID NO: 128 - protein, 115 aa, synthetic construct
  REGION 1..115: note = Synthetic: hu1D7v87 VHH
EVQLLESGGGEVQPGGSLRLSCVASRGKISQYRMGWYRQAPGKERELVAHISNTGDAAEYVQSVKDRFTISRDSTKNALYLQMSSLRAEDTAVYKCNSVEDILAWGQGTLVTVRP

SEQ ID NO: 129 - protein, 115 aa, synthetic construct
  REGION 1..115: note = Synthetic: hu1D7v88 VHH
EVQLLESGGGEVQPGGSLRLSCVASRGKISQYRMGWYRQAPGKERELVAHISNTGDAAEYVQGVKDRFTISRDSTKNALYLQMSSLRAEDTAVYKCNSVEDILAWGQGTLVTVRP

SEQ ID NO: 130 - protein, 115 aa, synthetic construct
  REGION 1..115: note = Synthetic: hu1D7v89 VHH
EVQLLESGGGEVQPGGSLRLSCVASRGKISQYRMGWYRQAPGKERELVAHISNTGDAAEYVDSVKDRFTISRDSTKNALYLQMSSLRAEDTAVYKCQGVEDILAWGQGTLVTVRP

SEQ ID NO: 131 - protein, 115 aa, synthetic construct
  REGION 1..115: note = Synthetic: hu1D7v90 VHH
EVQLLESGGGEVQPGGSLRLSCVASRGKISQYRMGWYRQAPGKERELVAHISNTGDAAEYVDSVKDRFTISRDSTKNALYLQMSSLRAEDTAVYKCSGVEDILAWGQGTLVTVRP

SEQ ID NO: 132 - protein, 115 aa, synthetic construct
  REGION 1..115: note = Synthetic: hu1D7v91 VHH
EVQLLESGGGEVQPGGSLRLSCVASRGKISQYRMGWYRQAPGKERELVAHISNTGDAAEYVDSVKDRFTISRDSTKNALYLQMSSLRAEDTAVYKCGGVEDILAWGQGTLVTVRP

SEQ ID NO: 133 - protein, 115 aa, synthetic construct
  REGION 1..115: note = Synthetic: hu1D7v92 VHH
EVQLLESGGGEVQPGGSLRLSCVASRGKISQYRMGWYRQAPGKERELVAHISNTGDAAEYVDSVKDRFTISRDSTKNALYLQMSSLRAEDTAVYKCQNVEDILAWGQGTLVTVRP

SEQ ID NO: 134 - protein, 115 aa, synthetic construct
  REGION 1..115: note = Synthetic: hu1D7v93 VHH
EVQLLESGGGEVQPGGSLRLSCVASRGKISQYRMGWYRQAPGKERELVAHISNTGDAAEYVDSVKDRFTISRDSTKNALYLQMSSLRAEDTAVYKCNQVEDILAWGQGTLVTVRP

SEQ ID NO: 135 - protein, 115 aa, synthetic construct
  REGION 1..115: note = Synthetic: hu1D7v94 VHH
EVQLLESGGGEVQPGGSLRLSCVASRGKISQYRMGWYRQAPGKERELVAHISNTGDAAEYVDSVKDRFTISRDSTKNALYLQMSSLRAEDTAVYKCSNVEDILAWGQGTLVTVRP

SEQ ID NO: 136 - protein, 115 aa, synthetic construct; identical to SEQ ID NO: 109
  REGION 1..115: note = Synthetic: hu1D7v95 VHH

SEQ ID NO: 137 - protein, 115 aa, synthetic construct
  REGION 1..115: note = Synthetic: hu1D7v96 VHH
EVQLLESGGGEVQPGGSLRLSCVASRGKISQYRMGWYRQAPGKERELVAHISNTGDAAEYVYSVKDRFTISRDSTKNALYLQMSSLRAEDTAVYKCNSVEDILAWGQGTLVTVRP

SEQ ID NO: 138 - protein, 115 aa, synthetic construct; identical to SEQ ID NO: 118
  REGION 1..115: note = Synthetic: hu1D7v97 VHH

SEQ ID NO: 139 - protein, 115 aa, synthetic construct
  REGION 1..115: note = Synthetic: hu1D7v98 VHH
EVQLLESGGGEVQPGGSLRLSCVASRGKISQYRMGWYRQAPGKERELVAHISNTGDAAEYVLSVKDRFTISRDSTKNALYLQMSSLRAEDTAVYKCNSVEDILAWGQGTLVTVRP

SEQ ID NO: 140 - protein, 115 aa, synthetic construct
  REGION 1..115: note = Synthetic: hu1D7v99 VHH
EVQLLESGGGEVQPGGSLRLSCVASRGKISQYRMGWYRQAPGKERELVAHISNTGDAAEYVSSVKDRFTISRDSTKNALYLQMSSLRAEDTAVYKCNSVEDILAWGQGTLVTVRP

SEQ ID NO: 141 - protein, 115 aa, synthetic construct
  REGION 1..115: note = Synthetic: hu1D7v100 VHH
EVQLLESGGGEVQPGGSLRLSCVASRGKISQYRMGWYRQAPGKERELVAHISNTGDAAEYVDSVKDRFTISRDSTKNALYLQMSSLRAEDTAVYYCNSVEDILAWGQGTLVTVRP

SEQ ID NO: 142 - protein, 115 aa, synthetic construct
  REGION 1..115: note = Synthetic: hu1D7v101 VHH
EVQLLESGGGEVQPGGSLRLSCVASRGKISQYRMGWYRQAPGKERELVAHISNTGDAAEYVDSVKDRFTISRDSTKNALYLQMSSLRAEDTAVYKCAAVEDILAWGQGTLVTVRP

SEQ ID NO: 143 - protein, 115 aa, synthetic construct
  REGION 1..115: note = Synthetic: hu1D7v102 VHH
EVQLLESGGGEVQPGGSLRLSCVASRGKISQYRMGWYRQAPGKELEWVAHISNTGDAAEYVDSVKDRFTISRDSTKNALYLQMSSLRAEDTAVYKCNTVEDILAWGQGTLVTVRP

SEQ ID NO: 144 - protein, 10 aa, synthetic construct
  REGION 1..10: note = Synthetic: CDR1 of hu1D7v3
GGKISQYRMG

SEQ ID NO: 145 - protein, 10 aa, synthetic construct
  REGION 1..10: note = Synthetic: CDR1 of hu1D7v4
RFKISQYRMG

SEQ ID NO: 146 - protein, 10 aa, synthetic construct
  REGION 1..10: note = Synthetic: CDR1 of hu1D7v5
RGTISQYRMG

SEQ ID NO: 147 - protein, 10 aa, synthetic construct
  REGION 1..10: note = Synthetic: CDR1 of hu1D7v6
RGKFSQYRMG

SEQ ID NO: 148 - protein, 10 aa, synthetic construct
  REGION 1..10: note = Synthetic: CDR1 of hu1D7v7 and 1D7v29
RGKISSYRMG

SEQ ID NO: 149 - protein, 10 aa, synthetic construct
  REGION 1..10: note = Synthetic: CDR1 of hu1D7v8
RGKISQYAMG

SEQ ID NO: 150 - protein, 10 aa, synthetic construct
  REGION 1..10: note = Synthetic: CDR2 of hu1D7v11, and v158-3
AISNTGDAAE

SEQ ID NO: 151 - protein, 10 aa, synthetic construct
  REGION 1..10: note = Synthetic: CDR2 of hu1D7v12
HISGTGDAAE

SEQ ID NO: 152 - protein, 10 aa, synthetic construct
  REGION 1..10: note = Synthetic: CDR2 of hu1D7v13
HISNSGDAAE

SEQ ID NO: 153 - protein, 10 aa, synthetic construct
  REGION 1..10: note = Synthetic: CDR2 of hu1D7v14
HISNTGGAAE

SEQ ID NO: 154 - protein, 10 aa, synthetic construct
  REGION 1..10: note = Synthetic: CDR2 of hu1D7v15
HISNTGDSAE

SEQ ID NO: 155 - protein, 10 aa, synthetic construct
  REGION 1..10: note = Synthetic: CDR2 of hu1D7v16
HISNTGDATE

SEQ ID NO: 156 - protein, 10 aa, synthetic construct
  REGION 1..10: note = Synthetic: CDR2 of hu1D7v17 and 1D7v30
HISNTGDAAY

SEQ ID NO: 157 - protein, 133 aa, synthetic construct
  REGION 1..133: note = Synthetic: IL-2-T3G-H16A-P65R-D84S-C125S (IL-2_Y)
APGSSSTKKTQLQLEALLLDLQMILNGINNYKNPKLTRMLTFKFYMPKKATELKHLQCLEEELKRLEEVLNLAQSKNFHLRPRSLISNINVIVLELKGSETTFMCEYADETATIVEFLNRWITFSQSIISTLT

SEQ ID NO: 158 - protein, 115 aa, synthetic construct
  REGION 1..115: note = Synthetic: hu1D7v158 VHH
EVQLLESGGGEVQPGGSLRLSCVASRGKISQYRMGWYRQAPGKQRELVAHISNTGDAAEYVAPVKGRFTISRDNAKNALYLQMNSLRAEDTAVYKCNTVEDILAWGQGTLVTVEP

SEQ ID NO: 159 - protein, 115 aa, synthetic construct
  REGION 1..115: note = Synthetic: hu1D7v158-1 VHH
EVQLLESGGGEVQPGGSLRLSCVASRGKISQYRMGWVRQAPGKQRELVAHISNTGDAAEYVAPVKGRFTISRDNAKNALYLQMNSLRAEDTAVYKCNTVEDILAWGQGTLVTVEP

SEQ ID NO: 160 - protein, 482 aa, synthetic construct
  REGION 1..482: note = Synthetic: OprI18v13 VHH-Fc region 2 IgG1 xELL knob-IL-2-T3G-H16A-P65R-D84S-C125S
EVQLVESGGGEVQPGGSLRLSCAASETIFRFNTMAWYRQAPGKRRELVGYITWAGRTGYAESVEGRFTISRDNAKNTVYLQMSSLRAEDTAVYYCNKHGSSFTQDYWGQGTLVTVKPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGSGGSAPGSSSTKKTQLQLEALLLDLQMILNGINNYKNPKLTRMLTFKFYMPKKATELKHLQCLEEELKRLEEVLNLAQSKNFHLRPRSLISNINVIVLELKGSETTFMCEYADETATIVEFLNRWITFSQSIISTLT

SEQ ID NO: 161 - protein, 345 aa, synthetic construct
  REGION 1..345: note = Synthetic: OprI18v13 VHH-Fc region xELL H435R, T366S, L368A, Y407V hole
EVQLVESGGGEVQPGGSLRLSCAASETIFRFNTMAWYRQAPGKRRELVGYITWAGRTGYAESVEGRFTISRDNAKNTVYLQMSSLRAEDTAVYYCNKHGSSFTQDYWGQGTLVTVKPGGGGDKTHTCPPCPAPGGPSVFLFPPKPKDTLMRSRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 162 - protein, 494 aa, synthetic construct
  REGION 1..494: note = Synthetic: 5T4-hz16G10v11 VHH-Fc region 2 IgG1 xELL-knob-IL2-T3G-H16A-P65R-D84S-C125S
EVQLVESGGGEVQPGGSLRLSCAASGRPFSSYAMGWFRQAPGKERETVAAVSRNAGSSYYAESVKGRFTISRDNAKNTVYLQMSSLRAEDTAVYYCAARSAAYSRSSETYTEKHDYTYWGQGTLVTVKPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGSGGSAPGSSSTKKTQLQLEALLLDLQMILNGINNYKNPKLTRMLTFKFYMPKKATELKHLQCLEEELKRLEEVLNLAQSKNFHLRPRSLISNINVIVLELKGSETTFMCEYADETATIVEFLNRWITFSQSIISTLT

SEQ ID NO: 163 - protein, 358 aa, synthetic construct
  REGION 1..358: note = Synthetic: 5D7 VHH-Fc region xELL H435R, T366S, L368A, Y407V hole
EVQLVESGGGLVQAGGSLRLSCIASGRTFSNYAMGWFRQAPGKEREFVAAISWSGGMTDHADSVKGRFTISRDNAKNTVYLQMNSLKPEDTAVYYCAAAFAGDIPYGSSWYGDPTTYDYWGQGTQVTVKPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 164 - protein, 480 aa, synthetic construct
  REGION 1..480: note = Synthetic: 1D7v158 VHH Fc region 2 IgG1 xELL knob-IL-2-T3A-H16A-E61R-P65R-D84Y-C125S
EVQLLESGGGEVQPGGSLRLSCVASRGKISQYRMGWYRQAPGKQRELVAHISNTGDAAEYVAPVKGRFTISRDNAKNALYLQMNSLRAEDTAVYKCNTVEDILAWGQGTLVTVEPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGSGGSAPASSSTKKTQLQLEALLLDLQMILNGINNYKNPKLTRMLTFKFYMPKKATELKHLQCLERELKRLEEVLNLAQSKNFHLRPRYLISNINVIVLELKGSETTFMCEYADETATIVEFLNRWITFSQSIISTLT

SEQ ID NO: 165 - protein, 117 aa, synthetic construct
  REGION 1..117: note = Synthetic: OprI18v13 (non-targeted) VHH
EVQLVESGGGEVQPGGSLRLSCAASETIFRFNTMAWYRQAPGKRRELVGYITWAGRTGYAESVEGRFTISRDNAKNTVYLQMSSLRAEDTAVYYCNKHGSSFTQDYWGQGTLVTVKP

SEQ ID NO: 166 - protein, 115 aa, synthetic construct
  REGION 1..115: note = Synthetic: hu1D7v158-2 VHH
EVQLLESGGGEVQPGGSLRLSCVASRGKISQYRMGWYRQAPGKQRELVSHISNTGDAAEYVAPVKGRFTISRDNAKNALYLQMNSLRAEDTAVYKCNTVEDILAWGQGTLVTVEP

SEQ ID NO: 167 - protein, 115 aa, synthetic construct
  REGION 1..115: note = Synthetic: hu1D7v158-3 VHH
EVQLLESGGGEVQPGGSLRLSCVASRGKISQYRMGWYRQAPGKQRELVAAISNTGDAAEYVAPVKGRFTISRDNAKNALYLQMNSLRAEDTAVYKCNTVEDILAWGQGTLVTVEP

SEQ ID NO: 168 - protein, 115 aa, synthetic construct
  REGION 1..115: note = Synthetic: hu1D7v158-4 VHH
EVQLLESGGGEVQPGGSLRLSCVASRGKISQYRMGWYRQAPGKQRELVAHISNTGDAAEYAAPVKGRFTISRDNAKNALYLQMNSLRAEDTAVYKCNTVEDILAWGQGTLVTVEP

SEQ ID NO: 169 - protein, 115 aa, synthetic construct
  REGION 1..115: note = Synthetic: hu1D7v158-5 VHH
EVQLLESGGGEVQPGGSLRLSCVASRGKISQYRMGWYRQAPGKQRELVAHISNTGDAAEYVEPVKGRFTISRDNAKNALYLQMNSLRAEDTAVYKCNTVEDILAWGQGTLVTVEP

SEQ ID NO: 170 - protein, 115 aa, synthetic construct
  REGION 1..115: note = Synthetic: hu1D7v158-6 VHH
EVQLLESGGGEVQPGGSLRLSCVASRGKISQYRMGWYRQAPGKQRELVAHISNTGDAAEYVASVKGRFTISRDNAKNALYLQMNSLRAEDTAVYKCNTVEDILAWGQGTLVTVEP

SEQ ID NO: 171 - protein, 115 aa, synthetic construct
  REGION 1..115: note = Synthetic: hu1D7v158-7 VHH
EVQLLESGGGEVQPGGSLRLSCVASRGKISQYRMGWYRQAPGKQRELVAHISNTGDAAEYVAPVKGRFTISRDNAKNTLYLQMNSLRAEDTAVYKCNTVEDILAWGQGTLVTVEP

SEQ ID NO: 172 - protein, 115 aa, synthetic construct
  REGION 1..115: note = Synthetic: hu1D7v158-8 VHH
EVQLLESGGGEVQPGGSLRLSCVASRGKISQYRMGWYRQAPGKQRELVAHISNTGDAAEYAESVKGRFTISRDNAKNALYLQMNSLRAEDTAVYKCNTVEDILAWGQGTLVTVEP

SEQ ID NO: 173 - protein, 115 aa, synthetic construct
  REGION 1..115: note = Synthetic: hu1D7v158-9 VHH
EVQLLESGGGEVQPGGSLRLSCVASRGKISQYRMGWYRQAPGKEREFVAHISNTGDAAEYVAPVKGRFTISRDNAKNALYLQMNSLRAEDTAVYKCNTVEDILAWGQGTLVTVEP

SEQ ID NO: 174 - protein, 115 aa, synthetic construct
  REGION 1..115: note = Synthetic: hu1D7v158-10 VHH
EVQLLESGGGEVQPGGSLRLSCVASRGKISQYRMGWYRQAPGKQRELVAHISNTGDAAEYVAPVKGRFTISRDNAKNALYLQMNSLRAEDTAVYYCNTVEDILAWGQGTLVTVEP

SEQ ID NO: 175 - protein, 115 aa, synthetic construct; identical to SEQ ID NO: 112
  REGION 1..115: note = Synthetic: 1D7v112 VHH

SEQ ID NO: 176 - protein, 115 aa, synthetic construct; identical to SEQ ID NO: 113
  REGION 1..115: note = Synthetic: 1D7v113 VHH

SEQ ID NO: 177 - protein, 115 aa, synthetic construct; identical to SEQ ID NO: 124
  REGION 1..115: note = Synthetic: 1D7v124 VHH

SEQ ID NO: 178 - protein, 115 aa, synthetic construct; identical to SEQ ID NO: 125
  REGION 1..115: note = Synthetic: 1D7v125 VHH

SEQ ID NO: 179 - protein, 115 aa, synthetic construct; identical to SEQ ID NO: 139
  REGION 1..115: note = Synthetic: 1D7v139 VHH

SEQ ID NO: 180 - protein, 115 aa, synthetic construct
  REGION 1..115: note = Synthetic: Consensus humanized 1D7 (c1D7)
  VARIANT 23: note = X is V or A
  VARIANT 26: note = X is R or G
  VARIANT 28: note = X is K or T
  VARIANT 29: note = X is I or F
  VARIANT 44: note = X is Q, G or E
  VARIANT 45: note = X is R or L
  VARIANT 47: note = X is L, W or F
  VARIANT 49: note = X is A or S
  VARIANT 50: note = X is H or A
  VARIANT 54: note = X is T or S
  VARIANT 56: note = X is D or G
  VARIANT 57: note = X is A or S
  VARIANT 58: note = X is A or T
  VARIANT 59: note = X is E or Y
  VARIANT 61: note = X is V or A
  VARIANT 62: note = X is D, E, A, G, V, S, Y, L or Q
  VARIANT 63: note = X is S, P, T, A, V, L, I, or G
  VARIANT 66: note = X is G or D
  VARIANT 74: note = X is S or N
  VARIANT 75: note = X is T or A
  VARIANT 78: note = X is A or T
  VARIANT 79: note = X is V or L
  VARIANT 84: note = X is N or S
  VARIANT 95: note = X is K or Y
  VARIANT 97: note = X is N, S, E, Y, A, S, G, Q
  VARIANT 98: note = X is S, T, A, L, V, N or G
  VARIANT 114: note = X is K, R, E, or D
EVQLLESGGGEVQPGGSLRLSCXASXGXXSQYRMGWYRQAPGKXXEXVXXISNXGXXXXYXXXVKXRFTISRDXXKNXXYLQMXSLRAEDTAVYXCXXVEDILAWGQGTLVTVXP

SEQ ID NO: 181 - protein, 212 aa, Homo sapiens
  REGION 1..212: note = misc_feature - human IgG1 Fc-NNT
GGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVNLTCLVKGFYPSDIAVEWESNGQPENNYKTTPPVLDSDGSFFLNSTLTVDKSRWQQGNVFSCSVMHEALHNHYTQKSLSLSPGK

(19) United States
(12) Patent Application Publication
(10) Pub. No.: US 2025/0109203 A1
(43) Pub. Date: Apr. 3, 2025

(54) Gamma Delta T-Cell-Targeted Modified IL-2 Polypeptides and Uses Thereof

(71) Applicant: Inhibrx Biosciences, Inc., La Jolla, CA (US)

(72) Inventors: Bryan R. Becklund, San Diego, CA (US); Kyle S. Jones, San Marcos, CA (US); Kaitlyn N. Robinson, La Jolla, CA (US); Andrew M. Eckles, La Jolla, CA (US); John C. Timmer, San Diego, CA (US); Brendan P. Eckelman, Encinitas, CA (US)

(73) Assignee: Inhibrx Biosciences, Inc., La Jolla, CA (US)

(21) Appl. No.: 18/726,350

(22) PCT Filed: Jan. 4, 2023
(86) PCT No.: PCT/US2023/060073

§ 371 Date: Jul. 2, 2024

Related U.S. Application Data
(60) Provisional application No. 63/417,910, filed on Oct. 20, 2022.
(60) Provisional application No. 63/296,770, filed on Jan. 5, 2022.

(51) Int. Cl. C07K 16/28; A61K 38/20; A61K 39/00; A61K 39/395; C07K 14/55

(52) U.S. Cl. CPC C07K 16/2809; A61K 38/2013; A61K 39/39558; C07K 14/55; A61K 2039/505; C07K 2317/31; C07K 2317/92; C07K 2319/33

(57) ABSTRACT

Provided herein are polypeptides comprising at least one VHH domain that binds γδ TCR and a modified IL-2. Uses of the polypeptides are also provided.

### CROSS-REFERENCE TO RELATED APPLICATIONS

This application claims the benefit of priority of U.S. Provisional Application No. 63/296,770, filed Jan. 5, 2022, and U.S. Provisional Application No. 63/417,910, filed Oct. 20, 2022; each of which is incorporated by reference herein in its entirety for any purpose.

### FIELD

The present invention relates to γδ T-cell-targeted modified IL-2 polypeptides, and methods of using such polypeptides to modulate the biological activity of γδ T-cells. Such methods include, but are not limited to, methods of treating cancer. In some embodiments, the γδ T-cell-targeted modified IL-2 polypeptides are fusion polypeptides comprising a γδ T-cell-binding polypeptide and a modified IL-2.

### BACKGROUND

Activation of T cells is controlled by various molecules, including the cytokine interleukin-2 (IL-2), which is synthesized and secreted by the activated T cell itself. Discovered as a T cell growth factor, IL-2 is a pleiotropic cytokine that modulates the proliferation and cytolytic activity of γδ T cells. IL-2 binds to a high affinity receptor composed of three subunits (IL-2α, IL-2β, and π) on the T cell surface. Signaling through the IL-2 receptor complex triggers the T cell to progress through cell division, driving clonal expansion of the activated T cell. IL-2 has potent effects even at low concentration, therefore its production is precisely controlled and requires both the signal delivered through the TCR/co-receptor complex and a co-stimulatory signal delivered through CD28.

Therefore, targeting IL-2 to γδ T cells may increase the potency and selectivity of the cytotoxic γδ T cell response, and there exists a need for γδ T-cell-targeted modified IL-2 polypeptides.

### SUMMARY

Provided herein are γδ T-cell-targeted modified IL-2 polypeptides and complexes, and methods of using γδ T-cell-targeted modified IL-2 polypeptides and complexes to treat, for example, cancer. In some embodiments, a γδ T-cell-targeted modified IL-2 polypeptide or complex comprises one or more additional binding domains and/or cytokine sequences. Certain embodiments are listed below.

Embodiment 1. A polypeptide comprising at least one VHH domain that binds γδ TCR and a modified IL-2, wherein at least one VHH domain that binds γδ TCR comprises a CDR1 comprising the amino acid sequence of SEQ ID NO: 3, 144, 146, 147, or 148; a CDR2 comprising the amino acid sequence of SEQ ID NO: 4, 150, 151, 152, 153, 154, 155, or 156; and a CDR3 comprising the amino acid sequence of SEQ ID NO: 5, and wherein the modified IL-2 comprises T3A or T3G, H16A, P65R, C125S, and D84S or D84Y mutations relative to a wild type human IL-2 comprising the amino acid sequence of SEQ ID NO: 71.
Embodiment 2. The polypeptide of embodiment 1, wherein the CDR1 comprises the amino acid sequence of SEQ ID NO: 3, the CDR2 comprises the amino acid sequence of SEQ ID NO: 4, and the CDR3 comprises the amino acid sequence of SEQ ID NO: 5.
Embodiment 3. The polypeptide of embodiment 1 or 2, wherein the modified IL-2 comprises T3A, H16A, P65R, C125S, and D84S mutations.
Embodiment 4. The polypeptide of embodiment 1 or 2, wherein the modified IL-2 comprises T3A, H16A, E61R, P65R, C125S, and D84Y mutations.
Embodiment 5. The polypeptide of any one of embodiments 1-4, wherein the modified IL-2 comprises an amino acid sequence that is at least 90%, at least 95%, at least 97%, at least 98%, at least 99%, or 100% identical to the amino acid sequence of SEQ ID NO: 78 or 79 or 157.
Embodiment 6. The polypeptide of any one of embodiments 1, 2, or 4, wherein the modified IL-2 comprises the amino acid sequence of SEQ ID NO: 78.
Embodiment 7. The polypeptide of any one of embodiments 1-6, wherein at least one VHH domain, or each VHH domain, is humanized.
Embodiment 8. The polypeptide of any one of embodiments 1-7, wherein at least one VHH domain comprises SEQ ID NO: 180, wherein X₁, X₂, X₄, X₅, X₆, X₇, X₈, X₉, X₁₀, X₁₁, X₁₂, X₁₃, X₁₄, X₁₅, X₁₆, X₁₇, X₁₈, X₁₉, X₂₀, X₂₁, X₂₂, X₂₃, X₂₄, X₂₅, X₂₆, and X₂₇ are independently selected, and wherein:

| | | |
|---|---|---|
| X₁ is V or A; | X₁₁ is D or G; | X₂₀ is T or A; |
| X₂ is R or G; | X₁₂ is A or S; | X₂₁ is A or T; |
| X₃ is K or T; | X₁₃ is A or T; | X₂₂ is V or L; |
| X₄ is I or F; | X₁₄ is E or Y; | X₂₃ is N or S; |
| X₅ is Q, G or E; | X₁₅ is V or A; | X₂₄ is K or Y; |
| X₆ is R or L; | X₁₆ is D, E, A, G, V, | X₂₅ is N, S, E, Y, A, S, |
| X₇ is L, W or F; | S, Y, L or Q; | G, Q |
| X₈ is A or S; | X₁₇ is S, P, T, A, V, | X₂₆ is S, T, A, L, V, N |
| X₉ is H or A; | L, I, or G; | or G; and |
| X₁₀ is T or S; | X₁₈ is G or D; | X₂₇ is K, R, E, or D. |
| | X₁₉ is S or N; | |

Embodiment 9. The polypeptide of any one of embodiments 1-7, wherein at least one VHH domain comprises SEQ ID NO: 180, wherein X₃ is K, X₁, X₂, X₄, X₅, X₆, X₇, X₈, X₉, X₁₀, X₁₁, X₁₂, X₁₃, X₁₄, X₁₅, X₁₆, X₁₇, X₁₈, X₁₉, X₂₀, X₂₁, X₂₂, X₂₃, X₂₄, X₂₅, X₂₆ and X₂₇ are independently selected, and wherein:

| | | |
|---|---|---|
| X₁ is V or A; | X₁₁ is D or G; | X₂₀ is T or A; |
| X₂ is R or G; | X₁₂ is A or S; | X₂₁ is A or T; |
| X₄ is I or F; | X₁₃ is A or T; | X₂₂ is V or L; |
| X₅ is Q, G or E; | X₁₄ is E or Y; | X₂₃ is N or S; |
| X₆ is R or L; | X₁₅ is V or A; | X₂₄ is K or Y; |
| X₇ is L, W or F; | X₁₆ is D, E, A, L or Q; | X₂₅ is N or S; |
| X₈ is A or S; | X₁₇ is S, P, T, V, L or G; | X₂₆ is S, T or G; and |
| X₉ is H or A; | X₁₈ is Gor D; | X₂₇ is K, R, E, or D. |
| X₁₀ is T or S; | X₁₉ is S or N; | |

Embodiment 10. The polypeptide of any one of embodiments 1-7, wherein at least one VHH domain comprises SEQ ID NO: 180, wherein X₂ is R; X₂₅ is N; and X₁, X₃ is K, X₄, X₅, X₆, X₇, X₈, X₉, X₁₀, X₁₁, X₁₂, X₁₃, X₁₄, X₁₅, X₁₆, X₁₇, X₁₈, X₁₉, X₂₀, X₂₁, X₂₂, X₂₃, X₂₄, X₂₆, and X₂₇ are independently selected, and wherein:

| | | |
|---|---|---|
| X₁ is V or A; | X₁₁ is D or G; | X₁₉ is S or N; |
| X₄ is I or F; | X₁₂ is A or S; | X₂₀ is T or A; |
| X₅ is Q, G or E; | X₁₃ is A or T; | X₂₁ is A or T; |
| X₆ is R or L; | X₁₄ is E or Y; | X₂₂ is V or L; |
| X₇ is L, W or F; | X₁₅ is V or A; | X₂₃ is N or S; |
| X₈ is A or S; | X₁₆ is D, E, A or Q; | X₂₄ is K or Y; |
| X₉ is H or A; | X₁₇ is S, P or G; | X₂₆ is S or T; and |
| X₁₀ is T or S; | X₁₈ is Gor D; | X₂₇ is K, R, E, or D. |

Embodiment 11. The polypeptide of any one of embodiments 1-7, wherein at least one VHH domain comprises SEQ ID NO: 180, wherein X₂ is R; X₃ is K, X₄ is I; X₉ is H; X₂₅ is N; and X₁, X₅, X₆, X₇, X₈, X₁₀, X₁₁, X₁₂, X₁₃, X₁₄, X₁₅, X₁₆, X₁₇, X₁₈, X₁₉, X₂₀, X₂₁, X₂₂, X₂₃, X₂₄, X₂₆, and X₂₇ are independently selected, and wherein:

| | | |
|---|---|---|
| X₁ is V or A; | X₁₃ is A or T; | X₂₀ is T or A; |
| X₅ is Q, G or E; | X₁₄ is E or Y; | X₂₁ is A or T; |
| X₆ is R or L; | X₁₅ is V or A; | X₂₂ is V or L; |
| X₇ is L, W or F; | X₁₆ is D, E, or A; | X₂₃ is N or S; |
| X₈ is A or S; | X₁₇ is S, or P; | X₂₄ is K or Y; |
| X₁₀ is T or S; | X₁₈ is G or D; | X₂₆ is S or T; and |
| X₁₁ is D or G; | X₁₉ is S or N; | X₂₇ is K, R, E, or D. |
| X₁₂ is A or S; | | |

Embodiment 12. The polypeptide of any one of embodiments 1-7, wherein at least one VHH domain comprises SEQ ID NO: 180, wherein X₁ is V; X₂ is R; X₃ is K, X₄ is I; X₉ is H; X₁₀ is T; X₁₁ is D; X₁₂ is A; X₁₃ is A; X₁₄ is E; X₂₅ is N; and X₅, X₆, X₇, X₈, X₁₅, X₁₆, X₁₇, X₁₈, X₁₉, X₂₀, X₂₁, X₂₂, X₂₃, X₂₄, X₂₆, and X₂₇ are independently selected, and wherein:

| | | |
|---|---|---|
| X₅ is Q, G or E; | X₁₇ is S, or P; | X₂₂ is V or L; |
| X₆ is R or L; | X₁₈ is Gor D; | X₂₃ is N or S; |
| X₇ is L, or W; | X₁₉ is S or N; | X₂₄ is K or Y; |
| X₈ is A or S; | X₂₀ is T or A; | X₂₆ is S or T; and |
| X₁₅ is V or A; | X₂₁ is A or T; | X₂₇ is K, R, E, or D. |
| X₁₆ is D, E, or A; | | |

Embodiment 13. The polypeptide of any one of embodiments 1-12, wherein at least one VHH domain comprises an amino acid sequence at least 85%, at least 90%, at least 95%, or at least 99% identical to an amino acid sequence selected from SEQ ID NOs: 2, 17-19, 21-23, 25-31, 72-77, 80, 81, 84-86, 88-93, 95, 97-107, 109, 111-129, 131-133, 135-137, 139-143, 158-159, and 166-179.
Embodiment 14. The polypeptide of any one of embodiments 1-13, wherein at least one VHH domain comprises an amino acid sequence selected from SEQ ID NO: 17-19, 21-23, 25-31, 72-77, 80, 81, 84-86, 88-93, 95, 97-107, 109, 111-129, 131-133, 135-137, 139-143, 158-159, and 166-179.
Embodiment 15. The polypeptide of any one of embodiments 1-14, wherein at least one VHH domain comprises the amino acid sequence of SEQ ID NO: 99, 143, or 158.
Embodiment 16. The polypeptide of any one of embodiments 1-15, comprising two VHH domains.
Embodiment 17. The polypeptide of any one of embodiments 1-15, comprising three VHH domains.
Embodiment 18. The polypeptide of any one of embodiments 1-15, wherein the polypeptide comprises at least one antigen-binding domain that binds an antigen other than γδ TCR.
Embodiment 19. The polypeptide of embodiment 18, wherein the at least one antigen-binding domain is a VHH domain.
Embodiment 20. The polypeptide of embodiment 18, or embodiment 19, wherein the polypeptide comprises at least one VHH domain that binds human serum albumin.
Embodiment 21. The polypeptide of embodiment 20, wherein the polypeptide comprises one VHH domain that binds γδ TCR, one VHH domain that binds human serum albumin, and the modified IL-2.
Embodiment 22. The polypeptide of embodiment 20 or embodiment 21, wherein the modified IL-2 is fused to the C-terminus of the VHH domain that binds human serum albumin.
Embodiment 23. The polypeptide of any one of embodiments 1-22, wherein the polypeptide does not comprise an Fc region.
Embodiment 24. The polypeptide of any one of embodiments 1-22, wherein the polypeptide comprises an Fc region.
Embodiment 25. The polypeptide of embodiment 24, wherein the Fc region comprises an amino acid sequence selected from SEQ ID NOs: 32-70, optionally wherein the Fc region lacks the C-terminal lysine residue.
Embodiment 26. The polypeptide of embodiment 24 or embodiment 24, wherein the modified IL-2 is fused to the C-terminus of the Fc region.
Embodiment 27. The polypeptide of any one of embodiments 1-15, and 24-26, wherein the polypeptide comprises one VHH domain that binds γδ TCR, an Fc region, and a modified IL-2.
Embodiment 28. The polypeptide of any one of embodiments 1-27, wherein the polypeptide comprises at least one antigen-binding domain that binds an antigen other than γδ TCR, optionally wherein at least one antigen-binding domain that binds an antigen other than γδ TCR binds a tumor cell antigen.
Embodiment 29. The polypeptide of embodiment 28, comprising at least one antigen-binding domain that binds Lag3, TGFBR1, TGFBR2, Fas, TNFR2, 1-92-LFA-3, 5T4, Alpha-4 integrin, Alpha-V integrin, alpha4beta1 integrin, alpha4beta7 integrin, AGR2, Anti-Lewis-Y, Apelin J receptor, APRIL, B7-H3, B7-H4, B7-H6, BAFF, BCMA, BTLA, C5 complement, C-242, CA9, CA19-9, (Lewis a), Carbonic anhydrase 9, CD2, CD3, CD6, CD9, CD11 a, CD19, CD20, CD22, CD24, CD25, CD27, CD28, CD30, CD33, CD38, CD39, CD40, CD40L, CD41, CD44, CD44v6, CD47, CD51, CD52, CD56, CD64, CD70, CD71, CD73, CD74, CD80, CD81, CD86, CD95, CD117, CD123, CD125, CD132, (IL-2RG), CD133, CD137, CD138, CD166, CD172A, CD248, CDH6, CEACAM5 (CEA), CEACAM6 (NCA-90), CLAUDIN-3, CLAUDIN-4, cMet, Collagen, Cripto, CSFR, CSFR-1, CTLA4, CTGF, CXCL10, CXCL13, CXCR1, CXCR2, CXCR4, CYR61, DL44, DLK1, DLL3, DLL4, DPP-4, DSG1, EDA, EDB, EGFR, EGFRviii, Endothelin B receptor (ETBR), ENPP3, EpCAM, EPHA2, EPHB2, ERBB3, F protein of RSV, FAP, FcRH5, FGF-2, FGF8, FGFR1, FGFR2, FGFR3, FGFR4, FLT-3, Folate receptor alpha (FRα), GAL3ST1, G-CSF, G-CSFR, GD2, GITR, GLUT1, GLUT4, GM-CSF, GM-CSFR, GP IIb/IIIa receptors, Gp130, GPIIB/IIIA, GPNMB, GPRC5D, GRP78, HAVCAR1, HER2/neu, HER3, HER4, HGF, hGH, HVEM, Hyaluronidase, ICOS, IFNalpha, IFNbeta, IFNgamma, IgE, IgE Receptor (FceRI), IGF, IGF1R, IL1B, IL1R, IL2, IL11, IL12, IL12p40, IL-12R, IL-12Rbetal, IL13, IL13R, IL15, IL17, IL18, IL21, IL23, IL23R, IL27/IL27R (wsx1), IL29, IL-31R, IL31/IL31R, IL2R, IL4, IL4R, IL6, IL6R, Insulin Receptor, Jagged Ligands, Jagged 1, Jagged 2, KISS1-R, LAG-3, LIF-R, Lewis X, LIGHT, LRP4, LRRC26, Ly6G6D, LyPD1, MCSP, Mesothelin, MICA, MICB, MRP4, MUC1, Mucin-16 (MUC16, CA-125), Na/K ATPase, NGF, Nicastrin, Notch Receptors, Notch 1, Notch 2, Notch 3, Notch 4, NOV, OSM-R, OX-40, PAR2, PDGF-AA, PDGF-BB, PDGFRalpha, PDGFRbeta, PD-1, PD-L1, PD-L2, Phosphatidyl-serine, P1GF, PSCA, PSMA, PSGR, RAAG12, RAGE, SLC44A4, Sphingosine 1 Phosphate, STEAP1, STEAP2, TAG-72, TAPA1, TEM-8, TGFbeta, TIGIT, TIM-3, TLR2, TLR4, TLR6, TLR7, TLR8, TLR9, TMEM31, TNFalpha, TNFR, TNFRS12A, TRAIL-R1, TRAIL-R2, Transferrin, Transferrin receptor, TRK-A, TRK-B, TROP-2 uPAR, VAP1, VCAM-1, VEGF, VEGF-A, VEGF-B, VEGF-C, VEGF-D, VEGFR1, VEGFR2, VEGFR3, VISTA, WISP-1, WISP-2, or WISP-3.
Embodiment 30. The polypeptide of embodiment 28 or 29, wherein at least one antigen-binding domain that binds an antigen other than γδ TCR is a VHH domain.
Embodiment 31. The polypeptide of embodiment 30, where each antigen-binding domain that binds an antigen other than γδ TCR is a VHH domain.
Embodiment 32. The polypeptide of any one of embodiments 27-29, wherein at least one antigen-binding domain that binds an antigen other than γδ TCR comprises a heavy chain variable region and a light chain variable region.
Embodiment 33. The polypeptide of embodiment 32, wherein each antigen-binding domain that binds an antigen other than γδ TCR comprises a heavy chain variable region and a light chain variable region.
Embodiment 34. A complex comprising a first polypeptide and a second polypeptide, wherein the first polypeptide is the polypeptide of any one of embodiments 24-33, wherein the first polypeptide comprises a first Fc region, and wherein the second polypeptide comprises a second Fc region, wherein the first and second Fc regions are the same or different.
Embodiment 35. A complex comprising a first polypeptide and a second polypeptide, wherein the first polypeptide comprises at least one VHH domain that binds γδ TCR and a first Fc region and the second polypeptide comprises a second Fc region and a modified IL-2, wherein at least one VHH domain that binds γδ TCR comprises a CDR1 comprising the amino acid sequence of SEQ ID NO: 3, 144, 146, 147, or 148; a CDR2 comprising the amino acid sequence of SEQ ID NO: 4, 150, 151, 152, 153, 154, 155, or 156; and a CDR3 comprising the amino acid sequence of SEQ ID NO: 5, and wherein the modified IL-2 comprises T3A or T3G, H16A, P65R, C125S, and D84S or D84Y mutations relative to a wild type human IL-2 comprising the amino acid sequence of SEQ ID NO: 71.
Embodiment 36. The complex of embodiment 34, wherein the CDR1 comprises the amino acid sequence of SEQ ID NO: 3, the CDR2 comprises the amino acid sequence of SEQ ID NO: 4, and the CDR3 comprises the amino acid sequence of SEQ ID NO: 5.
Embodiment 37. The complex of embodiment 34 or embodiment 35, wherein the modified IL-2 comprises T3A, H16A, P65R, C125S, and D84S mutations.
Embodiment 38. The complex of embodiment 34 or embodiment 35, wherein the modified IL-2 comprises T3A, H16A, E61R, P65R, C125S, and D84Y mutations.
Embodiment 39. The complex of any one of embodiments 29-32, wherein the modified IL-2 comprises an amino acid sequence that is at least 90%, at least 95%, at least 97%, at least 98%, at least 99%, or 100% identical to the amino acid sequence of SEQ ID NO: 78 or 79 or 157.
Embodiment 40. The complex of any one of embodiments 34, 35, and 37, wherein the modified IL-2 comprises the amino acid sequence of SEQ ID NO: 78.
Embodiment 41. The complex of any one of embodiments 34-39, wherein at least one VHH domain, or each VHH domain, is humanized.
Embodiment 42. The complex of any one of embodiment 35-41, wherein at least one VHH domain comprises SEQ ID NO: 180, wherein X₁, X₂, X₄, X₅, X₆, X₇, X₈, X₉, X₁₀, X₁₁, X₁₂, X₁₃, X₁₄, X₁₅, X₁₆, X₁₇, X₁₈, X₁₉, X₂₀, X₂₁, X₂₂, X₂₃, X₂₄, X₂₅, X₂₆, and X₂₇ are independently selected, and wherein:

| | | |
|---|---|---|
| X₁ is V or A; | X₁₁ is D or G; | X₂₀ is T or A; |
| X₂ is R or G; | X₁₂ is A or S; | X₂₁ is A or T; |
| X₃ is K or T; | X₁₃ is A or T; | X₂₂ is V or L; |
| X₄ is I or F; | X₁₄ is E or Y; | X₂₃ is N or S; |
| X₅ is Q, G or E; | X₁₅ is V or A; | X₂₄ is K or Y; |
| X₆ is R or L; | X₁₆ is D, E, A, G, | X₂₅ is N, S, E, Y, A, S, |
| X₇ is L, W or F; | V, S, Y, L or Q; | G, Q; |
| X₈ is A or S; | X₁₇ is S, P, T, A, | X₂₆ is S, T, A, L, V, N |
| X₉ is H or A; | V, L, I, or G; | or G; and |
| X₁₀ is T or S; | X₁₈ is G or D; | X₂₇ is K, R, E, or D. |
| | X₁₉ is S or N: | |

Embodiment 43. The complex of any one of embodiment 35-41, wherein at least one VHH domain comprises SEQ ID NO: 180, wherein X₃ is K, X₁, X₂, X₄, X₅, X₆, X₇, X₈, X₉, X₁₀, X₁₁, X₁₂, X₁₃, X₁₄, X₁₅, X₁₆, X₁₇, X₁₈, X₁₉, X₂₀, X₂₁, X₂₂, X₂₃, X₂₄, X₂₅, X₂₆ and X₂₇ are independently selected, and wherein:

| | | |
|---|---|---|
| X₁ is V or A; | X₁₁ is D or G; | X₂₀ is T or A; |
| X₂ is R or G; | X₁₂ is A or S; | X₂₁ is A or T; |
| X₄ is I or F; | X₁₃ is A or T; | X₂₂ is V or L; |
| X₅ is Q, G or E; | X₁₄ is E or Y; | X₂₃ is N or S; |
| X₆ is R or L; | X₁₅ is V or A; | X₂₄ is K or Y; |
| X₇ is L, W or F; | X₁₆ is D, E, A, L or Q; | X₂₅ is N or S; |
| X₈ is A or S; | X₁₇ is S, P, T, V, L or G; | X₂₆ is S, T or G; and |
| X₉ is H or A; | X₁₈ is G or D; | X₂₇ is K, R, E, or D. |
| X₁₀ is T or S; | X₁₉ is S or N; | |

Embodiment 44. The complex of any one of embodiment 35-41, wherein at least one VHH domain comprises SEQ ID NO: 180, wherein X₂ is R; X₂₅ is N; and X₁, X₃ is K, X₄, X₅, X₆, X₇, X₈, X₉, X₁₀, X₁₁, X₁₂, X₁₃, X₁₄, X₁₅, X₁₆, X₁₇, X₁₈, X₁₉, X₂₀, X₂₁, X₂₂, X₂₃, X₂₄, X₂₆, and X₂₇ are independently selected, and wherein:

| | | |
|---|---|---|
| X₁ is V or A; | X₁₁ is D or G; | X₁₉ is S or N; |
| X₄ is I or F; | X₁₂ is A or S; | X₂₀ is T or A; |
| X₅ is Q, G or E; | X₁₃ is A or T; | X₂₁ is A or T; |
| X₆ is R or L; | X₁₄ is E or Y; | X₂₂ is V or L; |
| X₇ is L, W or F; | X₁₅ is V or A; | X₂₃ is N or S; |
| X₈ is A or S; | X₁₆ is D, E, A or Q; | X₂₄ is K or Y; |
| X₉ is H or A; | X₁₇ is S, P or G; | X₂₆ is S or T; and |
| X₁₀ is T or S; | X₁₈ is G or D; | X₂₇ is K, R, E, or D. |

Embodiment 45. The complex of any one of embodiment 35-41, wherein at least one VHH domain comprises SEQ ID NO: 180, wherein X₃ is K, X₄ is I; X₉ is H; X₂₅ is N; and X₁, X₅, X₆, X₇, X₈, X₁₀, X₁₁, X₁₂, X₁₃, X₁₄, X₁₅, X₁₆, X₁₇, X₁₈, X₁₉, X₂₀, X₂₁, X₂₂, X₂₃, X₂₄, X₂₆, and X₂₇ are independently selected, and wherein:

| | | |
|---|---|---|
| X₁ is V or A; | X₁₃ is A or T; | X₂₀ is T or A; |
| X₅ is Q, G or E; | X₁₄ is E or Y; | X₂₁ is A or T; |
| X₆ is R or L; | X₁₅ is V or A; | X₂₂ is V or L; |
| X₇ is L, W or F; | X₁₆ is D, E, or A; | X₂₃ is N or S; |
| X₈ is A or S; | X₁₇ is S, or P; | X₂₄ is K or Y; |
| X₁₀ is T or S; | X₁₈ is G or D; | X₂₆ is S or T; and |
| X₁₁ is D or G; | X₁₉ is S or N; | X₂₇ is K, R, E, or D. |
| X₁₂ is A or S; | | |

Embodiment 46. The complex of any one of embodiment 35-41, wherein at least one VHH domain comprises SEQ ID NO: 180, wherein X₁ is V; X₂ is R; X₃ is K, X₄ is I; X₉ is H; X₁₀ is T; X₁₁ is D; X₁₂ is A; X₁₃ is A; X₁₄ is E; X₂₅ is N; and X₅, X₆, X₇, X₈, X₁₅, X₁₆, X₁₇, X₁₈, X₁₉, X₂₀, X₂₁, X₂₂, X₂₃, X₂₄, X₂₆, and X₂₇ are independently selected, and wherein:

| | | |
|---|---|---|
| X₅ is Q, G or E; | X₁₇ is S, or P; | X₂₂ is V or L; |
| X₆ is R or L; | X₁₈ is Gor D; | X₂₃ is N or S; |
| X₇ is L, or W; | X₁₉ is S or N; | X₂₄ is K or Y; |
| X₈ is A or S: | X₂₀ is T or A; | X₂₆ is S or T; and |
| X₁₅ is V or A; | X₂₁ is A or T; | X₂₇ is K, R, E, or D. |
| X₁₆ is D, E, or A; | | |

Embodiment 47. The complex of any one of embodiment 35-46, wherein at least one VHH domain comprises an amino acid sequence at least 85%, at least 90%, at least 95%, or at least 99% identical to an amino acid sequence selected from SEQ ID NOs: 2, 17-19, 21-23, 25-31, 72-77, 80, 81, 84-86, 88-93, 95, 97-107, 109, 111-129, 131-133, 135-137, 139-143, 158-159, and 166-179.
Embodiment 48. The complex of any one of embodiments 35-46, wherein at least one VHH domain comprises an amino acid sequence selected from SEQ ID NO: 17-19, 21-23, 25-31, 72-77, 80, 81, 84-86, 88-93, 95, 97-107, 109, 111-129, 131-133, 135-137, 139-143, 158-159, and 166-179.
Embodiment 49. The complex of any one of embodiments 35-48, wherein at least one VHH domain comprises the amino acid sequence of SEQ ID NO: 99 143, or 158.
Embodiment 50. The complex of any one of embodiments 34-49, wherein the second polypeptide comprises at least one antigen-binding domain.
Embodiment 51. The complex of embodiment 50, wherein the second polypeptide comprises at least one antigen-binding domain that binds an antigen other than γδ TCR, optionally wherein if the antigen-binding domain that binds an antigen other than a γδ TCR comprises a heavy chain variable region and a light chain variable region, then the heavy chain variable region is fused to a heavy chain constant region comprising the second Fc region.
Embodiment 52. The complex of embodiment 51, wherein at least one antigen-binding domain that binds an antigen other than γδ TCR binds a tumor cell antigen.
Embodiment 53. The complex of embodiment 51 or 52, wherein at least one antigen-binding domain that binds an antigen other than γδ TCR binds Lag3, TGFBR1, TGFBR2, Fas, TNFR2, 1-92-LFA-3, 5T4, Alpha-4 integrin, Alpha-V integrin, alpha4beta1 integrin, alpha4beta7 integrin, AGR2, Anti-Lewis-Y, Apelin J receptor, APRIL, B7-H3, B7-H4, B7-H6, BAFF, BCMA, BTLA, C5 complement, C-242, CA9, CA19-9, (Lewis a), Carbonic anhydrase 9, CD2, CD3, CD6, CD9, CD11a, CD19, CD20, CD22, CD24, CD25, CD27, CD28, CD30, CD33, CD38, CD39, CD40, CD40L, CD41, CD44, CD44v6, CD47, CD51, CD52, CD56, CD64, CD70, CD71, CD73, CD74, CD80, CD81, CD86, CD95, CD117, CD123, CD125, CD132, (IL-2RG), CD133, CD137, CD138, CD166, CD172A, CD248, CDH6, CEACAM5 (CEA), CEACAM6 (NCA-90), CLAUDIN-3, CLAUDIN-4, cMet, Collagen, Cripto, CSFR, CSFR-1, CTLA4, CTGF, CXCL10, CXCL13, CXCR1, CXCR2, CXCR4, CYR61, DL44, DLK1, DLL3, DLL4, DPP-4, DSG1, EDA, EDB, EGFR, EGFRviii, Endothelin B receptor (ETBR), ENPP3, EpCAM, EPHA2, EPHB2, ERBB3, F protein of RSV, FAP, FcRH5, FGF-2, FGF8, FGFR1, FGFR2, FGFR3, FGFR4, FLT-3, Folate receptor alpha (FRα), GAL3ST1, G-CSF, G-CSFR, GD2, GITR, GLUT1, GLUT4, GM-CSF, GM-CSFR, GP IIb/IIIa receptors, Gp130, GPIIB/IIIA, GPNMB, GPRC5D, GRP78, HAVCAR1, HER2/neu, HER3, HER4, HGF, hGH, HVEM, Hyaluronidase, ICOS, IFNalpha, IFNbeta, IFNgamma, IgE, IgE Receptor (FceRI), IGF, IGF1R, IL1B, IL1R, IL2, IL11, IL12, IL12p40, IL-12R, IL-12Rbetal, IL13, IL13R, IL15, IL17, IL18, IL21, IL23, IL23R, IL27/IL27R (wsx1), IL29, IL-31R, IL31/IL31R, IL2R, IL4, IL4R, IL6, IL6R, Insulin Receptor, Jagged Ligands, Jagged 1, Jagged 2, KISS1-R, LAG-3, LIF-R, Lewis X, LIGHT, LRP4, LRRC26, Ly6G6D, LyPD1, MCSP, Mesothelin, MICA, MICB, MRP4, MUC1, Mucin-16 (MUC16, CA-125), Na/K ATPase, NGF, Nicastrin, Notch Receptors, Notch 1, Notch 2, Notch 3, Notch 4, NOV, OSM-R, OX-40, PAR2, PDGF-AA, PDGF-BB, PDGFRalpha, PDGFRbeta, PD-1, PD-L1, PD-L2, Phosphatidyl-serine, P1GF, PSCA, PSMA, PSGR, RAAG12, RAGE, SLC44A4, Sphingosine 1 Phosphate, STEAP1, STEAP2, TAG-72, TAPA1, TEM-8, TGFbeta, TIGIT, TIM-3, TLR2, TLR4, TLR6, TLR7, TLR8, TLR9, TMEM31, TNFalpha, TNFR, TNFRS12A, TRAIL-R1, TRAIL-R2, Transferrin, Transferrin receptor, TRK-A, TRK-B, TROP-2 uPAR, VAP1, VCAM-1, VEGF, VEGF-A, VEGF-B, VEGF-C, VEGF-D, VEGFR1, VEGFR2, VEGFR3, VISTA, WISP-1, WISP-2, or WISP-3.
Embodiment 54. The complex of any one of embodiments 50-53, wherein at least one antigen-binding domain of the second polypeptide is a VHH domain.
Embodiment 55. The complex of embodiment 54, wherein the second polypeptide comprises at least one VHH domain that binds γδ TCR.
Embodiment 56. The complex of embodiment 55, wherein the second polypeptide comprises at least one VHH domain that binds γδ TCR comprising a CDR1 comprising the amino acid sequence of SEQ ID NO: 3, 144, 146, 147, or 148; a CDR2 comprising the amino acid sequence of SEQ ID NO: 4, 150, 151, 152, 153, 154, 155, or 156; and a CDR3 comprising the amino acid sequence of SEQ ID NO: 5.
Embodiment 57. The complex of embodiment 55 or 56, wherein the second polypeptide comprises at least one VHH domain that binds γδ TCR comprising a CDR1 comprising the amino acid sequence of SEQ ID NO: 3; a CDR2 comprising the amino acid sequence of SEQ ID NO: 4; and a CDR3 comprising the amino acid sequence of SEQ ID NO: 5.
Embodiment 58. The complex of any one of embodiments 55-57, wherein at least one VHH domain, or each VHH domain, of the second polypeptide is humanized.
Embodiment 59. The complex of any one of embodiments 55-58, wherein at least one VHH domain comprises SEQ ID NO: 180, wherein X₁, X₂, X₄, X₅, X₆, X₇, X₈, X₉, X₁₀, X₁₁, X₁₂, X₁₃, X₁₄, X₁₅, X₁₆, X₁₇, X₁₈, X₁₉, X₂₀, X₂₁, X₂₂, X₂₃, X₂₄, X₂₅, X₂₆, and X₂₇ are independently selected, and wherein:

| | | |
|---|---|---|
| X₁ is V or A; | X₁₁ is D or G; | X₂₀ is T or A; |
| X₂ is R or G; | X₁₂ is A or S; | X₂₁ is A or T; |
| X₃ is K or T; | X₁₃ is A or T; | X₂₂ is V or L; |
| X₄ is I or F; | X₁₄ is E or Y; | X₂₃ is N or S; |
| X₅ is Q, G or E; | X₁₅ is V or A; | X₂₄ is K or Y; |
| X₆ is R or L; | X₁₆ is D, E, A, G, | X₂₅ is N, S, E, Y, A, S, |
| X₇ is L, W or F; | V, S, Y, L or Q; | G, Q; |
| X₈ is A or S; | X₁₇ is S, P, T, A, | X₂₆ is S, T, A, L, V, N |
| X₉ is H or A; | V, L, I, or G; | or G; and |
| X₁₀ is T or S; | X₁₈ is G or D; | X₂₇ is K, R, E, or D. |
| | X₁₉ is S or N; | |

Embodiment 60. The complex of any one of embodiments 55-58, wherein at least one VHH domain comprises SEQ ID NO: 180, wherein X₃ is K, X₁, X₂, X₄, X₅, X₆, X₇, X₈, X₉, X₁₀, X₁₁, X₁₂, X₁₃, X₁₄, X₁₅, X₁₆, X₁₇, X₁₈, X₁₉, X₂₀, X₂₁, X₂₂, X₂₃, X₂₄, X₂₅, X₂₆ and X₂₇ are independently selected, and wherein:

| | | |
|---|---|---|
| X₁ is V or A; | X₁₁ is D or G; | X₂₀ is T or A; |
| X₂ is R or G; | X₁₂ is A or S; | X₂₁ is A or T; |
| X₄ is I or F; | X₁₃ is A or T: | X₂₂ is V or L; |
| X₅ is Q, G or E; | X₁₄ is E or Y; | X₂₃ is N or S; |
| X₆ is R or L; | X₁₅ is V or A; | X₂₄ is K or Y; |
| X₇ is L, W or F; | X₁₆ is D, E, A, L or Q; | X₂₅ is N or S; |
| X₈ is A or S; | X₁₇ is S, P, T, V, L or G; | X₂₆ is S, T or G; and |
| X₉ is H or A; | X₁₈ is Gor D; | X₂₇ is K, R, E, or D. |
| X₁₀ is T or S; | X₁₉ is S or N; | |

Embodiment 61. The complex of any one of embodiments 55-58, wherein at least one VHH domain comprises SEQ ID NO: 180, wherein X₂ is R; X₂₅ is N; and X₁, X₃ is K, X₄, X₅, X₆, X₇, X₈, X₉, X₁₀, X₁₁, X₁₂, X₁₃, X₁₄, X₁₅, X₁₆, X₁₇, X₁₈, X₁₉, X₂₀, X₂₁, X₂₂, X₂₃, X₂₄, X₂₆, and X₂₇ are independently selected, and wherein:

| | | |
|---|---|---|
| X₁ is V or A; | X₁₁ is D or G; | X₁₉ is S or N; |
| X₄ is I or F; | X₁₂ is A or S; | X₂₀ is T or A; |
| X₅ is Q, G or E; | X₁₃ is A or T; | X₂₁ is A or T; |
| X₆ is R or L; | X₁₄ is E or Y; | X₂₂ is V or L; |
| X₇ is L, W or F; | X₁₅ is V or A; | X₂₃ is N or S; |
| X₈ is A or S; | X₁₆ is D, E, A or Q; | X₂₄ is K or Y; |
| X₉ is H or A; | X₁₇ is S, P or G; | X₂₆ is S or T; and |
| X₁₀ is T or S; | X₁₈ is G or D; | X₂₇ is K, R, E, or D. |

Embodiment 62. The complex of any one of embodiments 55-58, wherein at least one VHH domain comprises SEQ ID NO: 180, wherein X₃ is K, X₄ is I; X₉ is H; X₂₅ is N; and X₁, X₅, X₆, X₇, X₈, X₁₀, X₁₁, X₁₂, X₁₃, X₁₄, X₁₅, X₁₆, X₁₇, X₁₈, X₁₉, X₂₀, X₂₁, X₂₂, X₂₃, X₂₄, X₂₆, and X₂₇ are independently selected, and wherein:

| | | |
|---|---|---|
| X₁ is V or A; | X₁₃ is A or T; | X₂₀ is T or A; |
| X₅ is Q, G or E; | X₁₄ is E or Y; | X₂₁ is A or T; |
| X₆ is R or L; | X₁₅ is V or A; | X₂₂ is V or L; |
| X₇ is L, W or F; | X₁₆ is D, E, or A; | X₂₃ is N or S; |
| X₈ is A or S; | X₁₇ is S, or P; | X₂₄ is K or Y; |
| X₁₀ is T or S; | X₁₈ is G or D; | X₂₆ is S or T; and |
| X₁₁ is D or G; | X₁₉ is S or N; | X₂₇ is K, R, E, or D. |
| X₁₂ is A or S; | | |

Embodiment 63. The complex of any one of embodiments 55-58, wherein at least one VHH domain comprises SEQ ID NO: 180, wherein X₁ is V; X₂ is R; X₃ is K, X₄ is I; X₉ is H; X₁₀ is T; X₁₁ is D; X₁₂ is A; X₁₃ is A; X₁₄ is E; X₂₅ is N; and X₅, X₆, X₇, X₈, X₁₅, X₁₆, X₁₇, X₁₈, X₁₉, X₂₀, X₂₁, X₂₂, X₂₃, X₂₄, X₂₆, and X₂₇ are independently selected, and wherein: X₅ is Q, G or E; X₁₇ is S, or P; X₂₂ is V or L;

| | | |
|---|---|---|
| X₅ is Q, G or E; | X₁₇ is S, or P; | X₂₂ is V or L; |
| X₆ is R or L; | X₁₈ is G or D; | X₂₃ is N or S; |
| X₇ is L, or W; | X₁₉ is S or N; | X₂₄ is K or Y; |
| X₈ is A or S; | X₂₀ is T or A; | X₂₆ is S or T; and |
| X₁₅ is V or A; | X₂₁ is A or T; | X₂₇ is K, R, E, or D. |
| X₁₆ is D, E, or A; | | |

Embodiment 64. The complex of any one of embodiments 55-63, wherein at least one VHH domain of the second polypeptide comprises an amino acid sequence at least 85%, 90%, 95%, or at least 99% identical to an amino acid sequence selected from SEQ ID NOs: 2, 17-19, 21-23, 25-31, 72-77, 80, 81, 84-86, 88-93, 95, 97-107, 109, 111-129, 131-133, 135-137, 139-143, 158-159, and 166-179.
Embodiment 65. The complex of any one of embodiments 55-64, wherein at least one VHH domain of the second polypeptide comprises an amino acid sequence selected from SEQ ID NO: 17-19, 21-23, 25-31, 72-77, 80, 81, 84-86, 88-93, 95, 97-107, 109, 111-129, 131-133, 135-137, 139-143, 158-159, and 166-179.
Embodiment 66. The complex of any one of embodiments 55-65, wherein at least one VHH domain of the second polypeptide comprises the amino acid sequence of SEQ ID NO: 99, 143, or 158.
Embodiment 67. The complex of any one of embodiments 55-66, wherein the second polypeptide comprises one VHH domain that binds γδ TCR.
Embodiment 68. The complex of any one of embodiments 34-67, wherein the first Fc region comprises at least one knob mutation and the second Fc region comprises at least one hole mutation; or wherein the first Fc region comprises at least one hole mutation and the second Fc region comprises at least one knob mutation.
Embodiment 69. The complex of embodiment 68, wherein the first or second Fc region comprises a T366W mutation and the other of the first or second Fc region comprises T366S, L368A, and Y407V mutations.
Embodiment 70. The complex of embodiment 69, wherein the Fc region comprising the T366S, L368A, and Y407V mutations further comprises a H435R or H435K mutation.
Embodiment 71. The complex of any one of embodiments 34-70, wherein the complex forms under physiological conditions.
Embodiment 72. The polypeptide or complex of any one of embodiments 1-71, wherein the γδ TCR is human γδ TCR.
Embodiment 73. The polypeptide or complex of any one of embodiments 1-72, wherein the VHH domain binds to a human γδ TCR comprising human gamma9 and human delta2.
Embodiment 74. A pharmaceutical composition comprising the polypeptide or complex of any one of embodiments 1-73, and a pharmaceutically acceptable carrier.
Embodiment 75. An isolated nucleic acid that encodes the polypeptide or complex of any one of embodiments 1-73.
Embodiment 76. A vector comprising the nucleic acid of embodiment 75.
Embodiment 77. A host cell comprising the nucleic acid of embodiment 75 or the vector of embodiment 76.
Embodiment 78. A host cell that expresses the polypeptide or complex of any one of embodiments 1-73.
Embodiment 79. A method of producing the polypeptide or complex of any one of embodiments 1-73, comprising incubating the host cell of embodiment 77 or embodiment 78 under conditions suitable for expression of the polypeptide or complex.
Embodiment 80. The method of embodiment 79, further comprising isolating the polypeptide or complex.
Embodiment 81. A method of increasing γδ T cell proliferation comprising contacting T cells with the polypeptide or complex of any one of embodiments 1-73.
Embodiment 82. The method of embodiment 81, wherein the T cells are in vitro.
Embodiment 83. The method of embodiment 81, wherein the T cells are in vivo.
Embodiment 84. A method of treating cancer comprising administering to a subject with cancer a pharmaceutically effective amount of the polypeptide or complex of any one of embodiments 1-73, or the pharmaceutical composition of embodiment 74.
Embodiment 85. The method of embodiment 83, wherein the cancer is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer; gastrointestinal cancer; glioblastoma; hepatic carcinoma; hepatoma; intra-epithelial neoplasm; kidney or renal cancer; larynx cancer; liver cancer; lung cancer; small-cell lung cancer; non-small cell lung cancer; adenocarcinoma of the lung; squamous carcinoma of the lung; melanoma; myeloma; neuroblastoma; oral cavity cancer; ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma; Hodgkin's lymphoma; non-Hodgkin's lymphoma; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; chronic lymphocytic leukemia (CLL); acute lymphoblastic leukemia (ALL); Hairy cell leukemia; and chronic myeloblastic leukemia.
Embodiment 86. The method of embodiment 84 or 85, further comprising administering an additional therapeutic agent.
Embodiment 87. The method of embodiment 86, wherein the additional therapeutic agent is an anti-cancer agent.
Embodiment 88. The method of embodiment 87, wherein the anti-cancer agent is selected from a chemotherapeutic agent, an anti-cancer biologic, radiation therapy, CAR-T therapy, and an oncolytic virus.
Embodiment 89. The method of embodiment 88, wherein the additional therapeutic agent is an anti-cancer biologic.
Embodiment 90. The method of embodiment 89, wherein the anti-cancer biologic is an agent that inhibits PD-1 and/or PD-L1.
Embodiment 91. The method of embodiment 89, wherein the anti-cancer biologic is an agent that inhibits VISTA, gpNMB, B7H3, B7H4, HHLA2, CTLA4, or TIGIT.
Embodiment 92. The method of any one of embodiments 89-91, wherein the anti-cancer agent is an antibody.
Embodiment 93. The method of embodiment 87, wherein the anti-cancer biologic is a cytokine.
Embodiment 94. The method of embodiment 88, wherein the anti-cancer agent is CAR-T therapy.
Embodiment 95. The method of embodiment 88, wherein the anti-cancer agent is an oncolytic virus.
Embodiment 96. The method of any one of embodiments 84-95, further comprising tumor resection and/or radiation therapy.

### BRIEF DESCRIPTION OF THE FIGURES

FIGS. 1A, 1C, 1E, and 1G show the median fluorescence intensity of intracellular phosphorylated STAT5 staining in γδ T cells, NK cells, and αβ T cells after treatment with monovalent or bivalent single domain antibodies specific for the γδ TCR linked to affinity reduced IL-2 variants IL-2_X or IL-2_Y as assessed by flow cytometry. FIGS. 1B, ID, 1F, and 1H show the percent of cells with phosphorylated STAT5 staining on γδ T cells, NK cells, and αβ T cells after treatment with monovalent or bivalent single domain antibodies specific for the γδ TCR linked to an affinity reduced IL-2 variants IL-2_X or IL-2_Y as assessed by flow cytometry.

FIG. 2A shows the percentage of γδ T cells that are proliferating in response to treatment with a γδ TCR targeted low affinity IL-2_X (cx11005) or a non-targeted low affinity IL-2_X (cx9452). FIG. 2B shows the percentage of all cells that are γδ T cells after treatment with the above test articles. FIG. 2C shows the percentage of αβ T cells that are proliferating after treatment, and FIG. 2D shows the percentage of all cells that are αβ T cells after treatment.

FIG. 3A shows the median fluorescence intensity of intracellular phosphorylated STAT5 in Vδ2′ γδ T cells and αβ T cells after treatment with a monovalent single domain antibody specific for the γδ TCR linked to an affinity reduced IL-2 variant IL-2_X (cx11026) as assessed by flow cytometry. FIG. 3B shows the percent of cells with phosphorylated STAT5 staining in Vδ2′ γδ T cells and αβ T cells after treatment with a monovalent single domain antibody specific for the γδ TCR linked to an affinity reduced IL-2 variant IL-2_X as assessed by flow cytometry.

FIG. 4A shows the percentage of Vδ2⁺ γδ T cells that proliferated in response to treatment with a γδ TCR targeted low affinity IL-2_X (cx11026) or a non-targeted low affinity IL-2_X (cx9452) as determined by flow cytometric analysis of CellTrace Violet dilution. FIG. 4B shows the percentage of total CD3⁺ T cells that are Vδ2⁺ γδ T cells after treatment.

FIG. 5A-5F show the activity of bispecific monovalent anti-γδ TCR targeted low affinity IL-2_X polypeptides. FIG. 5A shows the percentage of Vδ2⁻ γδ T cells that proliferated as determined by dilution of CellTrace violet upon treatment with an alternative format (1D7×HSA×IL-2_X, cx11486) monovalent bispecific antibody targeting the γδ TCR in combination with an anti-human serum albumin VHH and low affinity IL-2_X. FIG. 5B shows the percentage of Vδ2⁺ γδ T amongst the total CD3⁺ T cell population after treatment with cx11486. FIG. 5C-5F show the median fluorescence intensity (5C-5D) and the percent of Vδ2⁺ γδ T cells (5E-5F) with intracellular phosphorylated STAT5 after treatment with the monovalent 1D7×HSA×IL-2_X or 1D7×IL-2_X molecules in the absence (5C and 5E) or presence (5D and 5F) of 50 mg/ml HSA as assessed by flow cytometry.

FIG. 6A, 6B, 6C, 6D, 6E, 6F, 6G, 6H, 6I, 6J, 6K show binding of the γδ TCR-binding VHH, 1D7, and humanized variants thereof formatted as monovalent VHH-hIgG1-Fc fusion proteins, as assessed by flow cytometry on expanded human Vδ2+ γδ T cells.

FIG. 7A shows target cell killing curves (plotted as % of dead target cells) and 7B shows the live cell count after treatment with Vγ9Vδ2 T cells expanded with a γδTCR-targeted IL-2 molecule (1D7×IL-2_X) at increasing effector:target cell ratios as determined in a FACS based killing assay.

FIG. 8A-8B show the target cell killing curves (plotted as overlap of caspase3/7-green and cyto-ID red over time) of THP-1 (8A, left), HT29 (8A, right), Daudi (8B, top left), NCI-H460 (8B, top right), MM1 S (8B, bottom left), and A375 (8B, bottom right) cells after treatment with Vγ9Vδ2 T cells expanded with a γδ TCR-targeted IL-2 molecule (1D7×IL-2_X).

FIG. 9A-9D show the proliferation (9A-9B) and improved killing activity (9C-9D) of Vδ2+ cells in cancer patient PMBCs (9A, 9C) and DTCs (9B, 9D) after one week incubation in the presence or absence of the monovalent γδTCR-targeted IL-2 molecule 1D7×IL-2_X.

FIG. 10A-10F show the target cell killing curves (plotted as overlap of caspase3/7-green and cyto-ID red) for 5T4 expressing T47D target cells incubated with zoledronate expanded γδ T cells (10A-10B), enriched γδ T cells (10C-10D) or enriched αβ T cells (10E-10F) and treated with increasing concentrations of the bispecific molecules: 5T4×5C8×IL-2_Y or 5T4×5D7×IL-2_Y; the monovalent/monospecific molecules: 5C8×IL-2_Y or 5D7×IL-2_Y; or the control non-targeted IL-2_Y molecule. Plots for cells after 3.5 hours (10A, 10C, and 10E), and after 12 hours (10B, 10D, and 10F) are shown.

FIG. 11A-11C show the serum concentration and in vivo γδ TCR-targeted IL-2 enhancement of expansion of γδT cells. FIG. 11A plots the serum concentration of γδTCR-targeted IL-2_X (drug exposure) 24 hours after administration of a single 0.1, 1.0, or 10 mg/kg dose of the monovalent γδTCR-targeted IL-2_X (cx12168). FIG. 11B plots the total Vγ9Vδ2 T cell count for each treatment group and vehicle control at day 10 (9 days after dosing). FIG. 11C plots the fold expansion relative to the vehicle control group.

FIG. 12A shows the binding curves of monovalent HSA polypeptides comprising the parental (1D7×HSA×IL-2_X) or new optimized humanized 1D7v158 (1D7v158×HSA×IL-2_X) VHH domains. FIGS. 12B and 12C show the median fluorescence intensity (12B) and the percent of Vδ2⁺ γδ T cells (12C) with intracellular phosphorylated STAT5 after treatment with 1D7×HSA×IL-2_X or 1D7v158×HSA×IL-2_X as assessed by flow cytometry.

FIG. 13A-13F show the cross-reactivity with cynomolgus γδ TCR. FIG. 13A shows show binding of the γδ TCR-binding VHH, 1D7, and the humanized variant 1D7v9 formatted as bivalent VHH-hIgG1-xELL Fc fusion proteins, as assessed by flow cytometry on cynomolgus Vγ9+γδ T cells. FIG. 13B shows the percent of cells with phosphorylated STAT5 staining on cynomolgus monkey γδ T cells and αβ T cells from three donors after treatment with monovalent γδ TCR linked to an affinity reduced IL-2 variant (cx11026) as assessed by flow cytometry. FIGS. 13C and 13E show the percentage of cynomolgus monkey γδ T cells that are proliferating in response to treatment with a monovalent γδ TCR targeted low affinity IL-2_X (cx11026: 1D7×IL-2_X) or a non-targeted low affinity IL-2_X (cx9452: UT×IL-2_X) from two different cynomolgus monkey donors. FIGS. 13D and 13F show the percentage of all cells that are γδ T cells after treatment with the above test articles in these donors.

FIG. 14A-14C show sequence alignments of the parental anti-γδ TCR VHH 1D7 (SEQ ID NO: 2) with humanized 1D7 variants having a binding affinity (KD) of 100 nM as determined by flow cytometry (see, Table 2). FIG. 14D shows the sequence align of the parental anti-γδ TCR VHH 1D7 (SEQ ID NO: 2) with a consensus sequence (SEQ ID NO: 180) representing the aligned humanized 1D7 variants.

FIG. 15 shows five nonlimiting exemplary formats of γδ T-cell targeted modified IL-2 polypeptides. The first format shows an exemplary bivalent anti-γδ T cell construct with a single modified IL-2 polypeptide, which is a complex comprising a first polypeptide comprising an anti-γδ TCR VHH domain and an Fc region and a second polypeptide comprising an anti-γδ TCR VHH domain, an Fc region, and a modified IL-2 polypeptide. It is contemplated that one of the anti-γδ TCR VHH domains in such a structure could be replaced with one or more anti-antigen VHH domains (having specificity for an antigen other than γδ TCR), see e.g., the third format described below. The second format shows an exemplary monovalent anti-γδ T cell construct with a single modified IL-2 polypeptide, which is a complex comprising a first polypeptide comprising an anti-γδ TCR VHH domain, an Fc region, and a modified IL-2 polypeptide, and a second polypeptide that comprises an Fc region. The third format shows an exemplary bispecific, monovalent anti-γδ T cell×antigen construct with a single modified IL-2 polypeptide, which is a complex comprising a first polypeptide comprising an anti-γδ TCR VHH domain and an Fc region and a second polypeptide comprising an anti-antigen VHH domain (having specificity for an antigen other than γδ TCR), an Fc region, and a modified IL-2 polypeptide. The fourth format shows an exemplary monovalent anti-γδ T cell construct, which comprises an anti-γδ TCR VHH, an anti-HSA VHH, and a modified IL-2 polypeptide. The fifth format shows an exemplary monovalent anti-γδ T cell construct, which comprises an anti-antigen VHH domain, and anti-γδ TCR VHH, an anti-HSA VHH, and a modified IL-2 polypeptide. The order of tandem VHH domains may be changed. Similarly, for molecules comprising two polypeptide chains, VHH domains may be located on either chain.

### DETAILED DESCRIPTION

Embodiments provided herein relate to γδ T-cell-targeted modified IL-2 polypeptides and their use in various methods of treating, for example, cancer.

### Definitions and Various Embodiments

The section headings used herein are for organizational purposes only and are not to be construed as limiting the subject matter described.

All references cited herein, including patent applications, patent publications, and Genbank Accession numbers are herein incorporated by reference, as if each individual reference were specifically and individually indicated to be incorporated by reference in its entirety.

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3rd. edition (2001) Cold Spring Harbor Laboratory Press, Cold Spring Harbor, N.Y. CURRENT PROTOCOLS IN MOLECULAR BIOLOGY (F. M. Ausubel, et al. eds., (2003)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 1993); and updated versions thereof.

Unless otherwise defined, scientific and technical terms used in connection with the present disclosure shall have the meanings that are commonly understood by those of ordinary skill in the art. Further, unless otherwise required by context or expressly indicated, singular terms shall include pluralities and plural terms shall include the singular. For any conflict in definitions between various sources or references, the definition provided herein will control.

In general, the numbering of the residues in an immunoglobulin heavy chain is that of the EU index as in Kabat et al., Sequences of Proteins of Immunological Interest, 5th Ed. Public Health Service, National Institutes of Health, Bethesda, Md. (1991). The “EU index as in Kabat” refers to the residue numbering of the human IgG1 EU antibody.

It is understood that embodiments of the invention described herein include “consisting” and/or “consisting essentially of” embodiments. As used herein, the singular form “a”, “an”, and “the” includes plural references unless indicated otherwise. Use of the term “or” herein is not meant to imply that alternatives are mutually exclusive.

In this application, the use of “or” means “and/or” unless expressly stated or understood by one skilled in the art. In the context of a multiple dependent claim, the use of “or” refers back to more than one preceding independent or dependent claim.

The phrase “reference sample”, “reference cell”, or “reference tissue”, denote a sample with at least one known characteristic that can be used as a comparison to a sample with at least one unknown characteristic. In some embodiments, a reference sample can be used as a positive or negative indicator. A reference sample can be used to establish a level of protein and/or mRNA that is present in, for example, healthy tissue, in contrast to a level of protein and/or mRNA present in the sample with unknown characteristics. In some embodiments, the reference sample comes from the same subject, but is from a different part of the subject than that being tested. In some embodiments, the reference sample is from a tissue area surrounding or adjacent to the cancer. In some embodiments, the reference sample is not from the subject being tested, but is a sample from a subject known to have, or not to have, a disorder in question. In some embodiments, the reference sample is from the same subject, but from a point in time before the subject developed cancer. In some embodiments, the reference sample is from a benign cancer sample, from the same or a different subject. When a negative reference sample is used for comparison, the level of expression or amount of the molecule in question in the negative reference sample will indicate a level at which one of skill in the art will appreciate, given the present disclosure, that there is no and/or a low level of the molecule. When a positive reference sample is used for comparison, the level of expression or amount of the molecule in question in the positive reference sample will indicate a level at which one of skill in the art will appreciate, given the present disclosure, that there is a level of the molecule.

The terms “benefit”, “clinical benefit”, “responsiveness”, and “therapeutic responsiveness” as used herein in the context of benefiting from or responding to administration of a therapeutic agent, can be measured by assessing various endpoints, e.g., inhibition, to some extent, of disease progression, including slowing down and complete arrest; reduction in the number of disease episodes and/or symptoms; reduction in lesion size; inhibition (that is, reduction, slowing down or complete stopping) of disease cell infiltration into adjacent peripheral organs and/or tissues; inhibition (that is, reduction, slowing down or complete stopping) of disease spread; relief, to some extent, of one or more symptoms associated with the disorder; increase in the length of disease-free presentation following treatment, for example, progression-free survival; increased overall survival; higher response rate; and/or decreased mortality at a given point of time following treatment. A subject or cancer that is “non-responsive” or “fails to respond” is one that has failed to meet the above noted qualifications to be “responsive”.

The terms “nucleic acid molecule”, “nucleic acid” and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides comprised in the nucleic acid molecule or polynucleotide.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification. In some embodiments, a polypeptide is a “complex” of a first polypeptide and a second polypeptide.

“IL-2” or “Interleukin-2” as used herein refers to any native, mature IL-2 that results from processing of an IL-2 precursor in a cell. The term includes IL-2 from any vertebrate source, including mammals such as primates (e.g., humans and cynomolgus or rhesus monkeys) and rodents (e.g., mice and rats), unless otherwise indicated. The term also includes naturally-occurring variants of IL-2, such as splice variants or allelic variants. A nonlimiting exemplary human IL-2 amino acid sequence is shown, e.g., in GenBank Accession No. NP_000577.2. See SEQ ID NO. 71 (mature form).

“Modified IL-2” as used herein refers to a polypeptide that differs from a wild type IL-2 amino acid sequence by a substitution at at least one amino acid position.

The term “IL-2 activity” or “biological activity” of IL-2, as used herein, includes any biological effect or at least one of the biologically relevant functions of IL-2. In some embodiments, IL-2 activity includes the ability of IL-2 to induce T cell proliferation and/or activate natural killer (NK) cells. Nonlimiting exemplary IL-2 activities include increasing STAT5 phosphorylation and increasing proliferation of γδ T cells.

The term “specifically binds” to an antigen or epitope is a term that is well understood in the art, and methods to determine such specific binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. A single-domain antibody (sdAb) or VHH-containing polypeptide “specifically binds” or “preferentially binds” to a target if it binds with greater affinity, avidity, more readily, and/or with greater duration than it binds to other substances. For example, a sdAb or VHH-containing polypeptide that specifically or preferentially binds to a γδ T cell is a sdAb or VHH-containing polypeptide that binds this epitope with greater affinity, avidity, more readily, and/or with greater duration than it binds to other T cells or non-T cells. It is also understood by reading this definition that; for example, a sdAb or VHH-containing polypeptide that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding. “Specificity” refers to the ability of a binding protein to selectively bind an antigen.

The terms “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. In some embodiments, by “reduce” or “inhibit” is meant the ability to cause an overall decrease of 10% or greater. In some embodiments, by “reduce” or “inhibit” is meant the ability to cause an overall decrease of 50% or greater. In some embodiments, by “reduce” or “inhibit” is meant the ability to cause an overall decrease of 75%, 85%, 90%, 95%, or greater. In some embodiments, the amount noted above is inhibited or decreased over a period of time, relative to a control over the same period of time.

As used herein, the term “direct inhibition” and similar terms refers to an inhibition profile in which increasing antibody concentrations result in increasing inhibition. In some embodiments, after a certain concentration, maximal inhibition is reached and the inhibition profile plateaus. Maximal inhibition need not be 100% inhibition, but may be at least 50%, 55%, 60%, 65%, 70%, 75%, 80%, 85%, or 90%.

As used herein, the term “epitope” refers to a site on a target molecule (for example, an antigen, such as a protein, nucleic acid, carbohydrate or lipid) to which an antigen-binding molecule (for example, a sdAb or VHH-containing polypeptide) binds. Epitopes often include a chemically active surface grouping of molecules such as amino acids, polypeptides or sugar side chains and have specific three-dimensional structural characteristics as well as specific charge characteristics. Epitopes can be formed both from contiguous and/or juxtaposed noncontiguous residues (for example, amino acids, nucleotides, sugars, lipid moiety) of the target molecule. Epitopes formed from contiguous residues (for example, amino acids, nucleotides, sugars, lipid moiety) typically are retained on exposure to denaturing solvents whereas epitopes formed by tertiary folding typically are lost on treatment with denaturing solvents. An epitope may include but is not limited to at least 3, at least 5 or 8-10 residues (for example, amino acids or nucleotides). In some embodiments, an epitope is less than 20 residues (for example, amino acids or nucleotides) in length, less than 15 residues or less than 12 residues. Two antibodies may bind the same epitope within an antigen if they exhibit competitive binding for the antigen. In some embodiments, an epitope can be identified by a certain minimal distance to a CDR residue on the antigen-binding molecule. In some embodiments, an epitope can be identified by the above distance, and further limited to those residues involved in a bond (for example, a hydrogen bond) between a residue of the antigen-binding molecule and an antigen residue. An epitope can be identified by various scans as well, for example an alanine or arginine scan can indicate one or more residues that the antigen-binding molecule can interact with. Unless explicitly denoted, a set of residues as an epitope does not exclude other residues from being part of the epitope for a particular antigen-binding molecule. Rather, the presence of such a set designates a minimal series (or set of species) of epitopes. Thus, in some embodiments, a set of residues identified as an epitope designates a minimal epitope of relevance for the antigen, rather than an exclusive list of residues for an epitope on an antigen.

A “nonlinear epitope” or “conformational epitope” comprises noncontiguous polypeptides, amino acids and/or sugars within the antigenic protein to which an antigen-binding molecule specific to the epitope binds. In some embodiments, at least one of the residues will be noncontiguous with the other noted residues of the epitope; however, one or more of the residues can also be contiguous with the other residues.

A “linear epitope” comprises contiguous polypeptides, amino acids and/or sugars within the antigenic protein to which an antigen-binding molecule specific to the epitope binds. It is noted that, in some embodiments, not every one of the residues within the linear epitope need be directly bound (or involved in a bond) by the antigen-binding molecule. In some embodiments, linear epitopes can be from immunizations with a peptide that effectively consisted of the sequence of the linear epitope, or from structural sections of a protein that are relatively isolated from the remainder of the protein (such that the antigen-binding molecule can interact, at least primarily), just with that sequence section.

The term “antibody” is used in the broadest sense and encompass various polypeptides that comprise antibody-like antigen-binding domains, including but not limited to conventional antibodies (typically comprising at least one heavy chain and at least one light chain), single-domain antibodies (sdAbs, comprising at least one VHH domain and an Fc region), VHH-containing polypeptides (polypeptides comprising at least one VHH domain), and fragments of any of the foregoing so long as they exhibit the desired antigen-binding activity. In some embodiments, an antibody comprises a dimerization domain. Such dimerization domains include, but are not limited to, heavy chain constant domains (comprising CH1, hinge, CH2, and CH3, where CH1 typically pairs with a light chain constant domain, CL, while the hinge mediates dimerization) and Fc regions (comprising hinge, CH2, and CH3, where the hinge mediates dimerization).

The term antibody also includes, but is not limited to, chimeric antibodies, humanized antibodies, and antibodies of various species such as camelid (including llama), shark, mouse, human, cynomolgus monkey, etc.

The term “antigen-binding domain” as used herein refers to a portion of an antibody sufficient to bind antigen. In some embodiments, an antigen binding domain of a conventional antibody comprises three heavy chain CDRs and three light chain CDRs. Thus, in some embodiments, an antigen binding domain comprises a heavy chain variable region comprising CDR1-FR2-CDR2-FR3-CDR3, and any portions of FR1 and/or FR4 required to maintain binding to antigen, and a light chain variable region comprising CDR1-FR2-CDR2-FR3-CDR3, and any portions of FR1 and/or FR4 required to maintain binding to antigen. In some embodiments, an antigen-binding domain of an sdAb or VHH-containing polypeptide comprises three CDRs of a VHH domain. Thus, in some embodiments, an antigen binding domain of an sdAb or VHH-containing polypeptide comprises a VHH domain comprising CDR1-FR2-CDR2-FR3-CDR3, and any portions of FR1 and/or FR4 required to maintain binding to antigen.

The term “VHH” or “VHH domain” or “VHH antigen-binding domain” as used herein refers to the antigen-binding portion of a single-domain antibody, such as a camelid antibody or shark antibody. In some embodiments, a VHH comprises three CDRs and four framework regions, designated FR1, CDR1, FR2, CDR2, FR3, CDR3, and FR4. In some embodiments, a VHH may be truncated at the N-terminus or C-terminus such that it comprises only a partial FR1 and/or FR4, or lacks one or both of those framework regions, so long as the VHH substantially maintains antigen binding and specificity.

The terms “single domain antibody” and “sdAb” are used interchangeably herein to refer to an antibody comprising at least one monomeric domain, such as a VHH domain, without a light chain, and an Fc region. In some embodiments, an sdAb is a dimer of two polypeptides wherein each polypeptide comprises at least one VHH domain and an Fc region. As used herein, the terms “single domain antibody” and “sdAb” encompass polypeptides that comprise multiple VHH domains, such as a polypeptide having the structure VHH₁-VHH₂-Fc or VHH₁-VHH₂-VHH₃-Fc, wherein VHH₁, VHH₂, and VHH₃ may be the same or different.

The term “VHH-containing polypeptide” refers to a polypeptide that comprises at least one VHH domain. In some embodiments, a VHH polypeptide comprises two, three, or four or more VHH domains, wherein each VHH domain may be the same or different. In some embodiments, a VHH-containing polypeptide comprises an Fc region. In some such embodiments, the VHH-containing polypeptide may be referred to as an sdAb. Further, in some such embodiments, the VHH polypeptide may form a dimer. Nonlimiting structures of VHH-containing polypeptides, which are also sdAbs, include VHH₁-Fc, VHH₁-VHH₂-Fc, and VHH₁-VHH₂-VHH₃-Fc, wherein VHH₁, VHH₂, and VHH₃ may be the same or different. In some embodiments of such structures, one VHH may be connected to another VHH by a linker, or one VHH may be connected to the Fc by a linker. In some such embodiments, the linker comprises 1-20 amino acids, preferably 1-20 amino acids predominantly composed of glycine and, optionally, serine. In some embodiments, when a VHH-containing polypeptide comprises an Fc, it forms a dimer. Thus, the structure VHH₁-VHH₂-Fc, if it forms a dimer, is considered to be tetravalent (i.e., the dimer has four VHH domains). Similarly, the structure VHH₁-VHH₂-VHH₃-Fc, if it forms a dimer, is considered to be hexavalent (i.e., the dimer has six VHH domains).

The term “monoclonal antibody” refers to an antibody (including an sdAb or VHH-containing polypeptide) of a substantially homogeneous population of antibodies, that is, the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. Thus, a sample of monoclonal antibodies can bind to the same epitope on the antigen. The modifier “monoclonal” indicates the character of the antibody as being obtained from a substantially homogeneous population of antibodies, and is not to be construed as requiring production of the antibody by any particular method. For example, the monoclonal antibodies may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example.

The term “CDR” denotes a complementarity determining region as defined by at least one manner of identification to one of skill in the art. In some embodiments, CDRs can be defined in accordance with any of the Chothia numbering schemes, the Kabat numbering scheme, a combination of Kabat and Chothia, the AbM definition, and/or the contact definition. A VHH comprises three CDRs, designated CDR1, CDR2, and CDR3.

The term “heavy chain constant region” as used herein refers to a region comprising at least three heavy chain constant domains, CH1, hinge, CH2, and CH3. Of course, non-function-altering deletions and alterations within the domains are encompassed within the scope of the term “heavy chain constant region,” unless designated otherwise. Nonlimiting exemplary heavy chain constant regions include γ, δ, and α. Nonlimiting exemplary heavy chain constant regions also include ε and μ. Each heavy constant region corresponds to an antibody isotype. For example, an antibody comprising a γ constant region is an IgG antibody, an antibody comprising a δ constant region is an IgD antibody, and an antibody comprising an α constant region is an IgA antibody. Further, an antibody comprising a μ constant region is an IgM antibody, and an antibody comprising an F constant region is an IgE antibody. Certain isotypes can be further subdivided into subclasses. For example, IgG antibodies include, but are not limited to, IgG1 (comprising a γ₁ constant region), IgG2 (comprising a γ₂ constant region), IgG3 (comprising a γ₃ constant region), and IgG4 (comprising a γ₄ constant region) antibodies; IgA antibodies include, but are not limited to, IgA1 (comprising an α₁ constant region) and IgA2 (comprising an α₂ constant region) antibodies; and IgM antibodies include, but are not limited to, IgM1 and IgM2.

A “Fc region” as used herein refers to a portion of a heavy chain constant region comprising CH2 and CH3. In some embodiments, an Fc region comprises a hinge, CH2, and CH3. In various embodiments, when an Fc region comprises a hinge, the hinge mediates dimerization between two Fc-containing polypeptides. An Fc region may be of any antibody heavy chain constant region isotype discussed herein. In some embodiments, an Fc region is an IgG1, IgG2, IgG3, or IgG4.

An “acceptor human framework” as used herein is a framework comprising the amino acid sequence of a heavy chain variable domain (VH) framework derived from a human immunoglobulin framework or a human consensus framework, as discussed herein. An acceptor human framework derived from a human immunoglobulin framework or a human consensus framework can comprise the same amino acid sequence thereof, or it can contain amino acid sequence changes. In some embodiments, the number of amino acid changes are fewer than 10, or fewer than 9, or fewer than 8, or fewer than 7, or fewer than 6, or fewer than 5, or fewer than 4, or fewer than 3, across all of the human frameworks in a single antigen binding domain, such as a VHH.

“Affinity” refers to the strength of the sum total of noncovalent interactions between a single binding site of a molecule (for example, an antibody, such as an sdAb, or VHH-containing polypeptide) and its binding partner (for example, an antigen). The affinity or the apparent affinity of a molecule X for its partner Y can generally be represented by the dissociation constant (K_{D}) or the K_{D-apparent}, respectively. Affinity can be measured by common methods known in the art (such as, for example, ELISA K_{D}, KinExA, flow cytometry, and/or surface plasmon resonance devices), including those described herein. Such methods include, but are not limited to, methods involving BIAcore®, Octet®, or flow cytometry.

The term “K_{D}”, as used herein, refers to the equilibrium dissociation constant of an antigen-binding molecule/antigen interaction. When the term “K_{D}” is used herein, it includes K_{D} and K_{D-apparent}.

In some embodiments, the K_{D} of the antigen-binding molecule is measured by flow cytometry using an antigen-expressing cell line and fitting the mean fluorescence measured at each antibody concentration to a non-linear one-site binding equation (Prism Software graphpad). In some such embodiments, the K_{D} is K_{D-apparent}.

The term “biological activity” refers to any one or more biological properties of a molecule (whether present naturally as found in vivo, or provided or enabled by recombinant means). Biological properties include, but are not limited to, binding a ligand, inducing or increasing cell proliferation (such as T cell proliferation), and inducing or increasing expression of cytokines.

An “agonist” or “activating” antibody is one that increases and/or activates a biological activity of the target antigen. In some embodiments, the agonist antibody binds to an antigen and increases its biologically activity by at least about 20%, 40%, 60%, 80%, 85% or more.

An “antagonist”, a “blocking” or “neutralizing” antibody is one that inhibits, decreases and/or inactivates a biological activity of the target antigen. In some embodiments, the neutralizing antibody binds to an antigen and reduces its biologically activity by at least about 20%, 40%, 60%, 80%, 85% 90%, 95%, 99% or more.

An “affinity matured” sdAb or VHH-containing polypeptide refers to a sdAb or VHH-containing polypeptide with one or more alterations in one or more CDRs compared to a parent sdAb or VHH-containing polypeptide that does not possess such alterations, such alterations resulting in an improvement in the affinity of the sdAb or VHH-containing polypeptide for antigen.

A “humanized VHH” as used herein refers to a VHH in which one or more framework regions have been substantially replaced with human framework regions. In some instances, certain framework region (FR) residues of the human immunoglobulin are replaced by corresponding non-human residues. Furthermore, the humanized VHH can comprise residues that are found neither in the original VHH nor in the human framework sequences, but are included to further refine and optimize sdAb VHH-containing polypeptide performance. In some embodiments, a humanized sdAb or VHH-containing polypeptide comprises a human Fc region. As will be appreciated, a humanized sequence can be identified by its primary sequence and does not necessarily denote the process by which the antibody was created.

An “effector-positive Fc region” possesses an “effector function” of a native sequence Fc region. Exemplary “effector functions” include Fc receptor binding; C1q binding and complement dependent cytotoxicity (CDC); Fc receptor binding; antibody-dependent cell-mediated cytotoxicity (ADCC); phagocytosis; down regulation of cell surface receptors (for example B-cell receptor); and B-cell activation, etc. Such effector functions generally require the Fc region to be combined with a binding domain (for example, an antibody variable domain) and can be assessed using various assays.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification. In some embodiments, a “variant Fc region” comprises an amino acid sequence which differs from that of a native sequence Fc region by virtue of at least one amino acid modification, yet retains at least one effector function of the native sequence Fc region. In some embodiments, the variant Fc region has at least one amino acid substitution compared to a native sequence Fc region or to the Fc region of a parent polypeptide, for example, from about one to about ten amino acid substitutions, and preferably, from about one to about five amino acid substitutions in a native sequence Fc region or in the Fc region of the parent polypeptide. In some embodiments, the variant Fc region herein will possess at least about 80% sequence identity with a native sequence Fc region and/or with an Fc region of a parent polypeptide, at least about 90% sequence identity therewith, at least about 95%, at least about 96%, at least about 97%, at least about 98%, or at least about 99% sequence identity therewith.

“Fc receptor” or “FcR” describes a receptor that binds to the Fc region of an antibody. In some embodiments, an FcγR is a native human FcR. In some embodiments, an FcR is one which binds an IgG antibody (a gamma receptor) and includes receptors of the FcγRI, FcγRII, and FcγRIII subclasses, including allelic variants and alternatively spliced forms of those receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain Inhibiting receptor FcγRIIB contains an immunoreceptor tyrosine-based inhibition motif (ITIM) in its cytoplasmic domain. (See, for example, Daeron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. For example, the term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, for example, Ghetie and Ward, Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.).

The term “substantially similar” or “substantially the same,” as used herein, denotes a sufficiently high degree of similarity between two or more numeric values such that one of skill in the art would consider the difference between the two or more values to be of little or no biological and/or statistical significance within the context of the biological characteristic measured by said value. In some embodiments the two or more substantially similar values differ by no more than about any one of 5%, 10%, 15%, 20%, 25%, or 50%.

A polypeptide “variant” means a biologically active polypeptide having at least about 80% amino acid sequence identity with the native sequence polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, or deleted, at the N- or C-terminus of the polypeptide. In some embodiments, a variant will have at least about 80% amino acid sequence identity. In some embodiments, a variant will have at least about 90% amino acid sequence identity. In some embodiments, a variant will have at least about 95% amino acid sequence identity with the native sequence polypeptide.

As used herein, “percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

An amino acid substitution may include but are not limited to the replacement of one amino acid in a polypeptide with another amino acid. Exemplary substitutions are shown in Table 1. Amino acid substitutions may be introduced into an antibody of interest and the products screened for a desired activity, for example, retained/improved antigen binding, decreased immunogenicity, or improved ADCC or CDC.

TABLE 1

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term “vector” is used to describe a polynucleotide that can be engineered to contain a cloned polynucleotide or polynucleotides that can be propagated in a host cell. A vector can include one or more of the following elements: an origin of replication, one or more regulatory sequences (such as, for example, promoters and/or enhancers) that regulate the expression of the polypeptide of interest, and/or one or more selectable marker genes (such as, for example, antibiotic resistance genes and genes that can be used in colorimetric assays, for example, 0-galactosidase). The term “expression vector” refers to a vector that is used to express a polypeptide of interest in a host cell.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Nonlimiting exemplary mammalian cells include, but are not limited to, NSO cells, PER.C6® cells (Crucell), and 293 and CHO cells, and their derivatives, such as 293-6E, CHO-DG44, CHO-K1, CHO-S, and CHO-DS cells. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) a provided herein.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated”.

The terms “individual” and “subject” are used interchangeably herein to refer to an animal; for example, a mammal. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. In some examples, an “individual” or “subject” refers to an individual or subject in need of treatment for a disease or disorder. In some embodiments, the subject to receive the treatment can be a patient, designating the fact that the subject has been identified as having a disorder of relevance to the treatment, or being at adequate risk of contracting the disorder.

A “disease” or “disorder” as used herein refers to a condition where treatment is needed and/or desired.

The term “tumor cell”, “cancer cell”, “cancer”, “tumor”, and/or “neoplasm”, unless otherwise designated, are used herein interchangeably and refer to a cell (or cells) exhibiting an uncontrolled growth and/or abnormal increased cell survival and/or inhibition of apoptosis which interferes with the normal functioning of bodily organs and systems. Included in this definition are benign and malignant cancers, polyps, hyperplasia, as well as dormant tumors or micrometastases.

The terms “cancer” and “tumor” encompass solid and hematological/lymphatic cancers and also encompass malignant, pre-malignant, and benign growth, such as dysplasia. Exemplary cancers include, but are not limited to: basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

The term “non-tumor cell” as used herein refers to a normal cells or tissue. Exemplary non-tumor cells include, but are not limited to: T-cells, B-cells, natural killer (NK) cells, natural killer T (NKT) cells, dendritic cells, monocytes, macrophages, epithelial cells, fibroblasts, hepatocytes, interstitial kidney cells, fibroblast-like synoviocytes, osteoblasts, and cells located in the breast, skeletal muscle, pancreas, stomach, ovary, small intestines, placenta, uterus, testis, kidney, lung, heart, brain, liver, prostate, colon, lymphoid organs, bone, and bone-derived mesenchymal stem cells. The term “a cell or tissue located in the periphery” as used herein refers to non-tumor cells not located near tumor cells and/or within the tumor microenvironment.

The term “cells or tissue within the tumor microenvironment” as used herein refers to the cells, molecules, extracellular matrix and/or blood vessels that surround and/or feed a tumor cell. Exemplary cells or tissue within the tumor microenvironment include, but are not limited to: tumor vasculature; tumor-infiltrating lymphocytes; fibroblast reticular cells; endothelial progenitor cells (EPC); cancer-associated fibroblasts; pericytes; other stromal cells; components of the extracellular matrix (ECM); dendritic cells; antigen presenting cells; T-cells; regulatory T-cells (Treg cells); macrophages; neutrophils; myeloid-derived suppressor cells (MDSCs) and other immune cells located proximal to a tumor. Methods for identifying tumor cells, and/or cells/tissues located within the tumor microenvironment are well known in the art, as described herein, below.

In some embodiments, an “increase” or “decrease” refers to a statistically significant increase or decrease, respectively. As will be clear to the skilled person, “modulating” can also involve effecting a change (which can either be an increase or a decrease) in affinity, avidity, specificity and/or selectivity of a target or antigen, for one or more of its ligands, binding partners, partners for association into a homomultimeric or heteromultimeric form, or substrates; effecting a change (which can either be an increase or a decrease) in the sensitivity of the target or antigen for one or more conditions in the medium or surroundings in which the target or antigen is present (such as pH, ion strength, the presence of co-factors, etc.); and/or cellular proliferation or cytokine production, compared to the same conditions but without the presence of a test agent. This can be determined in any suitable manner and/or using any suitable assay known per se or described herein, depending on the target involved.

As used herein, “an immune response” is meant to encompass cellular and/or humoral immune responses that are sufficient to inhibit or prevent onset or ameliorate the symptoms of disease (for example, cancer or cancer metastasis). “An immune response” can encompass aspects of both the innate and adaptive immune systems.

As used herein, “treatment” is an approach for obtaining beneficial or desired clinical results. “Treatment” as used herein, covers any administration or application of a therapeutic for disease in a mammal, including a human. For purposes of this disclosure, beneficial or desired clinical results include, but are not limited to, any one or more of: alleviation of one or more symptoms, diminishment of extent of disease, preventing or delaying spread (for example, metastasis, for example metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In-line with the above, the term treatment does not require one-hundred percent removal of all aspects of the disorder.

“Ameliorating” means a lessening or improvement of one or more symptoms as compared to not administering a therapeutic agent. “Ameliorating” also includes shortening or reduction in duration of a symptom.

The term “anti-cancer agent” is used herein in its broadest sense to refer to agents that are used in the treatment of one or more cancers. Exemplary classes of such agents in include, but are not limited to, chemotherapeutic agents, anti-cancer biologics (such as cytokines, receptor extracellular sdomain-Fc fusions, and antibodies), radiation therapy, CAR-T therapy, therapeutic oligonucleotides (such as antisense oligonucleotides and siRNAs) and oncolytic viruses.

The term “biological sample” means a quantity of a substance from a living thing or formerly living thing. Such substances include, but are not limited to, blood, (for example, whole blood), plasma, serum, urine, amniotic fluid, synovial fluid, endothelial cells, leukocytes, monocytes, other cells, organs, tissues, bone marrow, lymph nodes and spleen.

The term “control” or “reference” refers to a composition known to not contain an analyte (“negative control”) or to contain an analyte (“positive control”). A positive control can comprise a known concentration of analyte.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. Unless otherwise specified, the terms “reduce”, “inhibit”, or “prevent” do not denote or require complete prevention over all time, but just over the time period being measured.

A “therapeutically effective amount” of a substance/molecule, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. A therapeutically effective amount may be delivered in one or more administrations. A therapeutically effective amount refers to an amount effective, at dosages and for periods of time necessary, to achieve the desired therapeutic and/or prophylactic result.

The terms “pharmaceutical formulation” and “pharmaceutical composition” are used interchangeably and refer to a preparation which is in such form as to permit the biological activity of the active ingredient(s) to be effective, and which contains no additional components which are unacceptably toxic to a subject to which the formulation would be administered. Such formulations may be sterile.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and are compatible with other ingredients of the formulation. The pharmaceutically acceptable carrier is appropriate for the formulation employed.

Administration “in combination with” one or more further therapeutic agents includes simultaneous (concurrent) and sequential administration in any order.

The term “concurrently” is used herein to refer to administration of two or more therapeutic agents, where at least part of the administration overlaps in time, or where the administration of one therapeutic agent falls within a short period of time relative to administration of the other therapeutic agent, or wherein the therapeutic effects of both agents overlap for at least a period of time.

The term “sequentially” is used herein to refer to administration of two or more therapeutic agents that does not overlap in time, or wherein the therapeutic effects of the agents do not overlap.

As used herein, “in conjunction with” refers to administration of one treatment modality in addition to another treatment modality. As such, “in conjunction with” refers to administration of one treatment modality before, during, or after administration of the other treatment modality to the individual.

The term “package insert” is used to refer to instructions customarily included in commercial packages of therapeutic products, that contain information about the indications, usage, dosage, administration, combination therapy, contraindications and/or warnings concerning the use of such therapeutic products.

An “article of manufacture” is any manufacture (for example, a package or container) or kit comprising at least one reagent, for example, a medicament for treatment of a disease or disorder (for example, cancer), or a probe for specifically detecting a biomarker described herein. In some embodiments, the manufacture or kit is promoted, distributed, or sold as a unit for performing the methods described herein.

The terms “label” and “detectable label” mean a moiety attached, for example, to an antibody or antigen to render a reaction (for example, binding) between the members of the specific binding pair, detectable. The labeled member of the specific binding pair is referred to as “detectably labeled.” Thus, the term “labeled binding protein” refers to a protein with a label incorporated that provides for the identification of the binding protein. In some embodiments, the label is a detectable marker that can produce a signal that is detectable by visual or instrumental means, for example, incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Examples of labels for polypeptides include, but are not limited to, the following: radioisotopes or radionuclides (for example, ³H, ¹⁴C, ³⁵S, ⁹⁰Y ⁹⁹Tc, ¹¹¹In, ¹²⁵I, ¹³¹I, ¹⁷⁷Lu, ¹⁶⁶Ho, or ¹⁵³Sm); chromogens, fluorescent labels (for example, FITC, rhodamine, lanthanide phosphors), enzymatic labels (for example, horseradish peroxidase, luciferase, alkaline phosphatase); chemiluminescent markers; biotinyl groups; predetermined polypeptide epitopes recognized by a secondary reporter (for example, leucine zipper pair sequences, binding sites for secondary antibodies, metal binding domains, epitope tags); and magnetic agents, such as gadolinium chelates. Representative examples of labels commonly employed for immunoassays include moieties that produce light, for example, acridinium compounds, and moieties that produce fluorescence, for example, fluorescein. In this regard, the moiety itself may not be detectably labeled but may become detectable upon reaction with yet another moiety.

### Exemplary γδ T Cell-Targeted Modified IL-2 Polypeptides

γδ T cell-targeted modified IL-2 polypeptides are provided herein. In various embodiments, the γδ T cell-targeted modified IL-2 polypeptides comprise at least one VHH domain that binds γδ T cells and a modified IL-2. In some embodiments, a γδ T cell-targeted modified IL-2 polypeptide provided herein comprises one, two, three, four, five, six, seven, or eight VHH domains that bind γδ T cells. In some embodiments, a γδ T cell-targeted modified IL-2 polypeptide provided herein comprises one, two, three, or four VHH domains that bind γδ T cells.

In some embodiments, a γδ T cell-targeted modified IL-2 polypeptide comprises at least one VHH domain that binds γδ T cells, an Fc region, and a modified IL-2. In some embodiments, a γδ T cell-targeted modified IL-2 polypeptide provided herein comprises one, two, three, or four VHH domains that bind γδ T cells, an Fc region, and a modified IL-2. In some embodiments, an Fc region mediates dimerization of the γδ T cell-targeted modified IL-2 polypeptide at physiological conditions such that a dimer is formed that doubles the number of γδ T cell binding sites. For example, a γδ T cell-targeted modified IL-2 polypeptide comprising three VHH domains that bind γδ T cells, an Fc region, and a modified IL-2 is trivalent as a monomer, but at physiological conditions, the Fc region may mediate dimerization, such that the γδ T cell-targeted modified IL-2 polypeptide exists as a hexavalent dimer under such conditions.

In some embodiments, a γδ T cell-targeted modified IL-2 polypeptide is a complex of a first polypeptide comprising a first VHH domain that binds γδ T cells, a first Fc domain, and a modified IL-2 polypeptide; and a second polypeptide comprising a second VHH domain that binds γδ T cells and a second Fc domain. In some embodiments, a γδ T cell-targeted modified IL-2 polypeptide is a complex of a first polypeptide comprising a first VHH domain that binds γδ T cells, a first Fc domain, and a modified IL-2 polypeptide; and a second polypeptide comprising an antigen-binding domain that binds an antigen other than γδ T cells and a second Fc domain. In some embodiments, a γδ T cell-targeted modified IL-2 polypeptide is a complex of a first polypeptide comprising an antigen-binding domain that an antigen other than γδ T cells, a first Fc domain, and a modified IL-2 polypeptide; and a second polypeptide comprising a second VHH domain that binds γδ T cells and a second Fc domain. In some embodiments, a γδ T cell-targeted modified IL-2 polypeptide is a complex of a first polypeptide comprising a first VHH domain that binds γδ T cells, a first Fc domain; and a second polypeptide comprising a second Fc domain and a modified IL-2 polypeptide. In some embodiments, a γδ T cell-targeted modified IL-2 polypeptide is a complex of a first polypeptide comprising a first VHH domain that binds γδ T cells, a first Fc domain and a modified IL-2 polypeptide; and a second polypeptide comprising a second Fc domain. In some such embodiments, the first or second Fc domain comprises “knob” mutation(s) and the other Fc domain comprises “hole” mutation(s). Thus, in some embodiments, a γδ T cell-targeted modified IL-2 polypeptide is a complex of a first polypeptide and a second polypeptide. In some such embodiments, the complex comprises two γδ T cell-binding VHH domains and one modified IL-2 polypeptide. In some such embodiments, the complex comprises one γδ T cell-binding VHH domain, one antigen-binding domain that binds an antigen other than γδ TCR and one modified IL-2 polypeptide. In some embodiments, a γδ T cell-targeted modified IL-2 polypeptide is a complex of a first polypeptide comprising a first VHH domain that binds γδ T cells and a first Fc domain; and a second polypeptide comprising a second Fc domain, a modified IL-2 polypeptide, and optionally, a second VHH domain or an antigen-binding domain that binds an antigen other than γδ TCR. In some such embodiments, the first or second Fc domain comprises “knob” mutation(s) and the other Fc domain comprises “hole” mutation(s). Thus, in some embodiments, a γδ T cell-targeted modified IL-2 complex comprises a γδ T cell-binding VHH domain and a modified IL-2 polypeptide within separate polypeptides of the complex.

In some embodiments, the γδ T cell-targeted modified IL-2 polypeptides provided herein comprise a γδ T cell-binding VHH and an antigen-binding domain that binds an antigen other than γδ T cells. In some such embodiments, the antigen is Lag3, CTLA4, TGFBR1, TGFBR2, Fas, TNFR2, PD1, PDL1, or TIM3. In some embodiments, the antigen is 1-92-LFA-3, 5T4, Alpha-4 integrin, Alpha-V integrin, alpha4beta1 integrin, alpha4beta7 integrin, AGR2, Anti-Lewis-Y, Apelin J receptor, APRIL, B7-H3, B7-H4, B7-H6, BAFF, BCMA, BTLA, C5 complement, C-242, CA9, CA19-9, (Lewis a), Carbonic anhydrase 9, CD2, CD3, CD6, CD9, CD11a, CD19, CD20, CD22, CD24, CD25, CD27, CD28, CD30, CD33, CD38, CD39, CD40, CD40L, CD41, CD44, CD44v6, CD47, CD51, CD52, CD56, CD64, CD70, CD71, CD73, CD74, CD80, CD81, CD86, CD95, CD117, CD123, CD125, CD132, (IL-2RG), CD133, CD137, CD138, CD166, CD172A, CD248, CDH6, CEACAM5 (CEA), CEACAM6 (NCA-90), CLAUDIN-3, CLAUDIN-4, cMet, Collagen, Cripto, CSFR, CSFR-1, CTLA4, CTGF, CXCL10, CXCL13, CXCR1, CXCR2, CXCR4, CYR61, DL44, DLK1, DLL3, DLL4, DPP-4, DSG1, EDA, EDB, EGFR, EGFRviii, Endothelin B receptor (ETBR), ENPP3, EpCAM, EPHA2, EPHB2, ERBB3, F protein of RSV, FAP, FcRH5, FGF-2, FGF8, FGFR1, FGFR2, FGFR3, FGFR4, FLT-3, Folate receptor alpha (FRα), GAL3ST1, G-CSF, G-CSFR, GD2, GITR, GLUT1, GLUT4, GM-CSF, GM-CSFR, GP IIb/IIIa receptors, Gp130, GPIIB/IIIA, GPNMB, GPRC5D, GRP78, HAVCAR1, HER2/neu, HER3, HER4, HGF, hGH, HVEM, Hyaluronidase, ICOS, IFNalpha, IFNbeta, IFNgamma, IgE, IgE Receptor (FceRI), IGF, IGF1R, IL1B, IL1R, IL2, IL11, IL12, IL12p40, IL-12R, IL-12Rbetal, IL13, IL13R, IL15, IL17, IL18, IL21, IL23, IL23R, IL27/IL27R (wsx1), IL29, IL-31R, IL31/IL31R, IL2R, IL4, IL4R, IL6, IL6R, Insulin Receptor, Jagged Ligands, Jagged 1, Jagged 2, KISS1-R, LAG-3, LIF-R, Lewis X, LIGHT, LRP4, LRRC26, Ly6G6D, LyPD1, MCSP, Mesothelin, MICA, MICB, MRP4, MUC1, Mucin-16 (MUC16, CA-125), Na/K ATPase, NGF, Nicastrin, Notch Receptors, Notch 1, Notch 2, Notch 3, Notch 4, NOV, OSM-R, OX-40, PAR2, PDGF-AA, PDGF-BB, PDGFRalpha, PDGFRbeta, PD-1, PD-L1, PD-L2, Phosphatidyl-serine, P1GF, PSCA, PSMA, PSGR, RAAG12, RAGE, SLC44A4, Sphingosine 1 Phosphate, STEAP1, STEAP2, TAG-72, TAPA1, TEM-8, TGFbeta, TIGIT, TIM-3, TLR2, TLR4, TLR6, TLR7, TLR8, TLR9, TMEM31, TNFalpha, TNFR, TNFRS12A, TRAIL-R1, TRAIL-R2, Transferrin, Transferrin receptor, TRK-A, TRK-B, TROP-2 uPAR, VAP1, VCAM-1, VEGF, VEGF-A, VEGF-B, VEGF-C, VEGF-D, VEGFR1, VEGFR2, VEGFR3, VISTA, WISP-1, WISP-2, or WISP-3. In some embodiments, the γδ T cell-targeted modified IL-2 polypeptide comprises a γδ T cell-binding VHH and an antigen-binding domain that binds a tumor cell antigen. In some such embodiments, the γδ T-cell-targeted modified IL-2 polypeptides comprising an antigen binding domain that binds a tumor cell antigen increase γδ T-cell mediated killing of tumor cells expressing the antigen.

Exemplary VHH Domains that Bind γδ T Cells

In various embodiments, a VHH domain that binds γδ TCR comprises a CDR1 sequence selected from SEQ ID NOs: 3, 144, 145, 146, 147, 148, and 149; a CDR2 sequence selected from SEQ ID NOs: 4, 150, 151, 152, 153, 154, 155, and 156; and a CDR3 sequence of SEQ ID NO: 5. In various embodiments, a VHH domain that binds γδ TCR comprises a CDR1, a CDR2, and a CDR3, respectively, comprising the amino acid sequences of SEQ ID NOs: 3, 4, and 5; 144, 4, and 5; 145, 4, and 5; 146, 4, and 5; 147, 4, and 5; 148, 4, and 5; 149, 4, and 5; 3, 150, and 5;3, 151, and 5;3, 152, and 5;3, 153, and 5;3, 154, and 5;3, 155, and 5; or 3, 156, and 5. In various embodiments, a VHH domain that binds γδ TCR comprises a CDR1 sequence selected from SEQ ID NOs: 3, 144, 146, 147, and 148; a CDR2 sequence selected from SEQ ID NOs: 4, 150, 151, 152, 153, 154, 155, and 156; and a CDR3 sequence of SEQ ID NO: 5. In various embodiments, a VHH domain that binds γδ TCR comprises a CDR1, a CDR2, and a CDR3, respectively comprising the amino acid sequences of SEQ ID NOs: 3, 4, and 5; 144, 4, and 5; 146, 4, and 5; 147, 4, and 5; 148, 4, and 5; 3, 150, and 5; 3, 151, and 5; 3, 152, and 5; 3, 153, and 5; 3, 154, and 5; 3, 155, and 5; or 3, 156, and 5. In various embodiments, a VHH domain that binds γδ TCR comprises a CDR1 sequence of SEQ ID NO: 3; a CDR2 sequence of SEQ ID NO: 4; and a CDR3 sequence 3 of SEQ ID NO: 5. In various embodiments, the VHH domain is humanized.

In some embodiments, a VHH domain that binds γδ TCR comprises SEQ ID NO: 180, wherein X₁, X₂, X₄, X₅, X₆, X₇, X₈, X₉, X₁₀, X₁₁, X₁₂, X₁₃, X₁₄, X₁₅, X₁₆, X₁₇, X₁₈, X₁₉, X₂₀, X₂₁, X₂₂, X₂₃, X₂₄, X₂₅, X₂₆, and X₂₇ are independently selected, and wherein:

| | | |
|---|---|---|
| X₁ is V or A; | X₁₁ is D or G; | X₂₀ is T or A; |
| X₂ is R or G; | X₁₂ is A or S; | X₂₁ is A or T; |
| X₃ is K or T; | X₁₃ is A or T; | X₂₂ is V or L; |
| X₄ is I or F; | X₁₄ is E or Y; | X₂₃ is N or S; |
| X₅ is Q, G or E; | X₁₅ is V or A; | X₂₄ is K or Y; |
| X₆ is R or L; | X₁₆ is D, E, A, G, V, S, Y, L | X₂₅ is N, S, E, Y, A, S, |
| X₇ is L, W or F; | or Q; | G, Q; |
| X₈ is A or S; | X₁₇ is S, P, T, A, V, L, I, or | X₂₆ is S, T, A, L, V, N |
| X₉ is H or A; | G; | or G; and |
| X₁₀ is T or S; | X₁₈ is G or D; | X₂₇ is K, R, E, or D. |
| | X₁₉ is S or N; | |

In some embodiments, a VHH domain that binds γδ TCR comprises SEQ ID NO: 180, wherein X₃ is K, and X₁, X₂, X₄, X₅, X₆, X₇, X₈, X₉, X₁₀, X₁₁, X₁₂, X₁₃, X₁₄, X₁₅, X₁₆, X₁₇, X₁₈, X₁₉, X₂₀, X₂₁, X₂₂, X₂₃, X₂₄, X₂₅, X₂₆, and X₂₇ are independently selected, and wherein:

| | | |
|---|---|---|
| X₁ is V or A; | X₁₁ is D or G; | X₂₀ is T or A; |
| X₂ is R or G; | X₁₂ is A or S; | X₂₁ is A or T; |
| X₄ is I or F; | X₁₃ is A or T; | X₂₂ is V or L; |
| X₅ is Q, G or E; | X₁₄ is E or Y; | X₂₃ is N or S; |
| X₆ is R or L; | X₁₅ is V or A; | X₂₄ is K or Y; |
| X₇ is L, W or F; | X₁₆ is D, E, A, L or Q; | X₂₅ is N or S; |
| X₈ is A or S; | X₁₇ is S, P, T, V, L or G; | X₂₆ is S, T or G; and |
| X₉ is H or A; | X₁₈ is G or D; | X₂₇ is K, R, E, or D. |
| X₁₀ is T or S; | X₁₉ is S or N; | |

In some embodiments, a VHH domain that binds γδ TCR comprises SEQ ID NO: 180, wherein X₂ is R; X₂₅ is N; and X₁, X₃ is K, X₄, X₅, X₆, X₇, X₈, X₉, X₁₀, X₁₁, X₁₂, X₁₃, X₁₄, X₁₅, X₁₆, X₁₇, X₁₈, X₁₉, X₂₀, X₂₁, X₂₂, X₂₃, X₂₄, X₂₆, and X₂₇ are independently selected, and wherein:

| | | |
|---|---|---|
| X₁ is V or A; | X₁₁ is D or G; | X₁₉ is S or N; |
| X₄ is I or F; | X₁₂ is A or S; | X₂₀ is T or A; |
| X₅ is Q, G or E; | X₁₃ is A or T; | X₂₁ is A or T; |
| X₆ is R or L; | X₁₄ is E or Y; | X₂₂ is V or L; |
| X₇ is L, W or F; | X₁₅ is V or A; | X₂₃ is N or S; |
| X₈ is A or S; | X₁₆ is D, E, A or Q; | X₂₄ is K or Y; |
| X₉ is H or A; | X₁₇ is S, P or G; | X₂₆ is S or T; and |
| X₁₀ is T or S; | X₁₈ is Gor D; | X₂₇ is K, R, E, or D. |

In some embodiments, a VHH domain that binds γδ TCR comprises SEQ ID NO: 180, wherein X₂ is R; X₃ is K, X₄ is I; X₉ is H; X₂₅ is N; and X₁, X₅, X₆, X₇, X₈, X₁₀, X₁₁, X₁₂, X₁₃, X₁₄, X₁₅, X₁₆, X₁₇, X₁₈, X₁₉, X₂₀, X₂₁, X₂₂, X₂₃, X₂₄, X₂₆, and X₂₇ are independently selected, and wherein:

| | | |
|---|---|---|
| X₁ is V or A; | X₁₃ is A or T; | X₂₀ is T or A; |
| X₅ is Q, G or E; | X₁₄ is E or Y; | X₂₁ is A or T; |
| X₆ is R or L; | X₁₅ is V or A; | X₂₂ is V or L; |
| X₇ is L, W or F; | X₁₆ is D, E, or A; | X₂₃ is N or S; |
| X₈ is A or S; | X₁₇ is S, or P; | X₂₄ is K or Y; |
| X₁₀ is T or S; | X₁₈ is Gor D; | X₂₆ is S or T; and |
| X₁₁ is D or G; | X₁₉ is S or N; | X₂₇ is K, R, E, or D. |
| X₁₂ is A or S; | | |

In some embodiments, a VHH domain that binds γδ TCR comprises SEQ ID NO: 180, wherein X₁ is V; X₂ is R; X₃ is K, X₄ is I; X₉ is H; X₁₀ is T; X₁ is D; X₁₂ is A; X₁₃ is A; X₁₄ is E; X₂₅ is N; and X₅, X₆, X₇, X₈, X₁₅, X₁₆, X₁₇, X₁₈, X₁₉, X₂₀, X₂₁, X₂₂, X₂₃, X₂₄, X₂₆, and X₂₇ are independently selected, and wherein:

| | | |
|---|---|---|
| X₅ is Q, G or E; | X₁₇ is S, or P; | X₂₂ is V or L; |
| X₆ is R or L; | X₁₈ is Gor D; | X₂₃ is N or S; |
| X₇ is L, or W; | X₁₉ is S or N; | X₂₄ is K or Y; |
| X₈ is A or S; | X₂₀ is T or A; | X₂₆ is S or T; and |
| X₁₅ is V or A; | X₂₁ is A or T; | X₂₇ is K, R, E, or D. |
| X₁₆ is D, E, or A; | | |

In some embodiments, a VHH domain that binds γδ TCR comprises an amino acid sequence that is at least 85%, at least 90%, at least 95%, at least 96%, at least 97%, at least 98%, at least 99% identical to an amino acid sequence selected from SEQ ID NOs: 2, 17-31, 72-77, 80-143, 158-159, and 166-179. In some embodiments, a VHH domain that binds γδ TCR comprises an amino acid sequence selected from SEQ ID NOs: 2, 17-31, 72-77, 80-143, 158-159, and 166-179, wherein position 114 of the VHH domain has been substituted with a lysine (K) an aspartate (D), a glutamate (E), or an arginine (R). In some embodiments, a VHH domain that binds γδ TCR comprises an amino acid sequence that is at least 85%, at least 90%, at least 95%, at least 96%, at least 97%, at least 98%, at least 99% identical to an amino acid sequence selected from SEQ ID NOs: 2, 17-19, 21-23, 25-31, 72-77, 80, 81, 84-86, 88-93, 95, 97-107, 109, 111-129, 131-133, 135-137, 139-143, 158-159, and 166-179. In some embodiments, a VHH domain that binds γδ TCR comprises an amino acid sequence selected from SEQ ID NOs: 2, 17-19, 21-23, 25-31, 72-77, 80, 81, 84-86, 88-93, 95, 97-107, 109, 111-129, 131-133, 135-137, 139-143, 158-159, and 166-179, wherein position 114 of the VHH domain has been substituted with a lysine (K) an aspartate (D), a glutamate (E), or an arginine (R). In some embodiments, a VHH domain that binds γδ TCR comprises an amino acid sequence selected from SEQ ID NOs: 2, 17-31, 72-77, 80-143, 158-159, and 166-179. In some embodiments, a VHH domain that binds γδ TCR comprises an amino acid sequence selected from SEQ ID NOs: 2, 17-19, 21-23, 25-31, 72-77, 80, 81, 84-86, 88-93, 95, 97-107, 109, 111-129, 131-133, 135-137, 139-143, 158-159, and 166-179.

In some embodiments, a VHH domain that binds γδ TCR comprises a CDR1 sequence of SEQ ID NO: 3, a CDR2 sequence of SEQ ID NO: 4, and a CDR3 of SEQ ID NO: 5. In some embodiments, the VHH domain comprises an amino acid sequence at least 85%, 90%, 95%, or at least 99% identical SEQ ID NO: 99, 143, or 158. In some embodiments, the VHH domain comprises an amino acid sequence selected from SEQ ID NO: 99, 143, and 158 wherein position 114 of the VHH domain has been substituted with a lysine (K) an aspartate (D), a glutamate (E), or an arginine (R). In some embodiments, the VHH domain comprises the amino acid sequence of SEQ ID NO: 99. In some embodiments, the VHH domain comprises the amino acid sequence of SEQ ID NO: 143. In some embodiments, the VHH domain comprises the amino acid sequence of SEQ ID NO: 158.

In various embodiments, a γδ T cell-binding-binding polypeptide comprises one, two, three, or four VHH domains that bind γδ T cells.

In some embodiments, a VHH domain that binds γδ T-cells may be humanized. Humanized antibodies (such as sdAbs or VHH-containing polypeptides) are useful as therapeutic molecules because humanized antibodies reduce or eliminate the human immune response to non-human antibodies, which can result in an immune response to an antibody therapeutic, and decreased effectiveness of the therapeutic. Generally, a humanized antibody comprises one or more variable domains in which CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (for example, the antibody from which the CDR residues are derived), for example, to restore or improve antibody specificity or affinity.

Humanized antibodies and methods of making them are reviewed, for example, in Almagro and Fransson, (2008) Front. Biosci. 13: 1619-1633, and are further described, for example, in Riechmann et al., (1988) Nature 332:323-329; Queen et al., (1989) Proc. Natl Acad. Sci. USA 86: 10029-10033; U.S. Pat. No. 5,821,337, 7,527,791, 6,982,321, and 7,087,409; Kashmiri et al., (2005) Methods 36:25-34; Padlan, (1991) Mol. Immunol. 28:489-498 (describing “resurfacing”); Dall'Acqua et al., (2005) Methods 36:43-60 (describing “FR shuffling”); and Osbourn et al., (2005) Methods 36:61-68 and Klimka et al., (2000) Br. J. Cancer, 83:252-260 (describing the “guided selection” approach to FR shuffling).

Human framework regions that can be used for humanization include but are not limited to: framework regions selected using the “best-fit” method (see, for example, Sims et al. (1993) J. Immunol. 151:2296); framework regions derived from the consensus sequence of human antibodies of a particular subgroup of heavy chain variable regions (see, for example, Carter et al. (1992) Proc. Natl. Acad. Sci. USA, 89:4285; and Presta et al. (1993) J. Immunol, 151:2623); human mature (somatically mutated) framework regions or human germline framework regions (see, for example, Almagro and Fransson, (2008) Front. Biosci. 13:1619-1633); and framework regions derived from screening FR libraries (see, for example, Baca et al., (1997) J. Biol. Chem. 272: 10678-10684 and Rosok et al., (1996) J. Biol. Chem. 271:22611-22618). Typically, the FR regions of a VHH are replaced with human FR regions to make a humanized VHH. In some embodiments, certain FR residues of the human FR are replaced in order to improve one or more properties of the humanized VHH. VHH domains with such replaced residues are still referred to herein as “humanized.”

### Exemplary Modified IL-2 Polypeptides

γδ T-cell-targeted modified IL-2 polypeptides comprising a γδ T-cell-binding VHH and a modified IL-2 are provided herein. In some such embodiments, the γδ T-cell-targeted modified IL-2 polypeptide comprises a γδ T-cell-binding VHH, an Fc region, and a modified IL-2. In some embodiments, the modified IL-2 comprises at least one amino acid substitution that reduces the affinity of the modified IL-2 for an IL-2 receptor compared to a wild type IL-2. In various embodiments, the polypeptide comprising a modified IL-2 provided herein is an agonist of an IL-2R. In some embodiments, the modified IL-2 is a modified human IL-2, and the IL-2R is a human IL-2R. In some embodiments, the modified IL-2 binds a human IL-2R with an affinity at least 2-fold, 3-fold, 4-fold, 5-fold, 6-fold, 7-fold, 8-fold, 9-fold, at least 10-fold, at least 20-fold, at least 30-fold, at least 50-fold, or at least 100-fold lower than the affinity of human wild type IL-2 for the IL-2R.

In various embodiments, the modified IL-2 comprises at least one substitution at at least one amino acid position selected from T3, H16, E61, P65, D84, and C125. In some embodiments, the modified IL-2 comprises substitutions at amino acid positions T3, H16, E61, P65, D84, and C125. In some embodiments, the modified IL-2 comprises substitutions at amino acid positions T3, H16, P65, D84, and C125. In some embodiments, the modified IL-2 comprises substitutions T3A, H16A, E61R, P65R, D84Y, and C125S. In some embodiments, the modified IL-2 comprises substitutions T3A, H16A, P65R, D84S, and C125S. In some embodiments, the modified IL-2 comprises an amino acid sequence at least 90%, 91%, 92%, 93%, 94%, 95%, 96%, 97%, 98%, or 99% identical to SEQ ID NO: 78 or 79 or 157. In some such embodiments, the modified IL-2 comprises the amino acid sequence of SEQ ID NO: 78. In some such embodiments, the modified IL-2 comprises the amino acid sequence of SEQ ID NO: 79. In some such embodiments, the modified IL-2 comprises the amino acid sequence of SEQ ID NO: 157.

In some embodiments, a γδ T-cell-targeted modified IL-2 polypeptide comprises at least one VHH domain that binds γδ TCR, at least one VHH domain that binds an antigen other than γδ TCR, and a modified IL-2. In some such embodiments, at least one VHH domain of the polypeptide binds human serum albumin. In some embodiments, the γδ T-cell-targeted modified IL-2 polypeptide comprises one VHH domain that binds γδ TCR, one VHH domain that binds human serum albumin, and a modified IL-2. In some embodiments, the γδ T-cell-targeted modified IL-2 polypeptide comprises at least one VHH domain that binds γδ TCR, at least one VHH domain that binds human serum albumin, at least one VHH domain that binds an antigen other than γδ TCR or human serum albumin, and a modified IL-2. In some embodiments, the γδ T-cell-targeted modified IL-2 polypeptide comprises one VHH domain that binds γδ TCR, one VHH domain that binds human serum albumin, one VHH domain that binds an antigen other than γδ TCR or human serum albumin, and a modified IL-2. In some such embodiments, the antigen other than γδ TCR or human serum albumin is 1-92-LFA-3, 5T4, Alpha-4 integrin, Alpha-V integrin, alpha4beta1 integrin, alpha4beta7 integrin, AGR2, Anti-Lewis-Y, Apelin J receptor, APRIL, B7-H3, B7-H4, B7-H6, BAFF, BCMA, BTLA, C5 complement, C-242, CA9, CA19-9, (Lewis a), Carbonic anhydrase 9, CD2, CD3, CD6, CD9, CD11a, CD19, CD20, CD22, CD24, CD25, CD27, CD28, CD30, CD33, CD38, CD39, CD40, CD40L, CD41, CD44, CD44v6, CD47, CD51, CD52, CD56, CD64, CD70, CD71, CD73, CD74, CD80, CD81, CD86, CD95, CD117, CD123, CD125, CD132, (IL-2RG), CD133, CD137, CD138, CD166, CD172A, CD248, CDH6, CEACAM5 (CEA), CEACAM6 (NCA-90), CLAUDIN-3, CLAUDIN-4, cMet, Collagen, Cripto, CSFR, CSFR-1, CTLA4, CTGF, CXCL10, CXCL13, CXCR1, CXCR2, CXCR4, CYR61, DL44, DLK1, DLL3, DLL4, DPP-4, DSG1, EDA, EDB, EGFR, EGFRviii, Endothelin B receptor (ETBR), ENPP3, EpCAM, EPHA2, EPHB2, ERBB3, F protein of RSV, FAP, Fas, FcRH5, FGF-2, FGF8, FGFR1, FGFR2, FGFR3, FGFR4, FLT-3, Folate receptor alpha (FRα), GAL3ST1, G-CSF, G-CSFR, GD2, GITR, GLUT1, GLUT4, GM-CSF, GM-CSFR, GP IIb/IIIa receptors, Gp130, GPIIB/IIIA, GPNMB, GPRC5D, GRP78, HAVCAR1, HER2/neu, HER3, HER4, HGF, hGH, HVEM, Hyaluronidase, ICOS, IFNalpha, IFNbeta, IFNgamma, IgE, IgE Receptor (FceRI), IGF, IGF1R, IL1B, IL1R, IL2, IL11, IL12, IL12p40, IL-12R, IL-12Rbetal, IL13, IL13R, IL15, IL17, IL18, IL21, IL23, IL23R, IL27/IL27R (wsx1), IL29, IL-31R, IL31/IL31R, IL2R, IL4, IL4R, IL6, IL6R, Insulin Receptor, Jagged Ligands, Jagged 1, Jagged 2, KISS1-R, LAG-3, LIF-R, Lewis X, LIGHT, LRP4, LRRC26, Ly6G6D, LyPD1, MCSP, Mesothelin, MICA, MICB, MRP4, MUC1, Mucin-16 (MUC16, CA-125), Na/K ATPase, NGF, Nicastrin, Notch Receptors, Notch 1, Notch 2, Notch 3, Notch 4, NOV, OSM-R, OX-40, PAR2, PDGF-AA, PDGF-BB, PDGFRalpha, PDGFRbeta, PD-1, PD-L1, PD-L2, Phosphatidyl-serine, P1GF, PSCA, PSMA, PSGR, RAAG12, RAGE, SLC44A4, Sphingosine 1 Phosphate, STEAP1, STEAP2, TAG-72, TAPA1, TEM-8, TGFbeta, TGFBR1, TGFBR2, TIGIT, TIM-3, TLR2, TLR4, TLR6, TLR7, TLR8, TLR9, TMEM31, TNFalpha, TNFR, TNFRS12A, TRAIL-R1, TRAIL-R2, Transferrin, Transferrin receptor, TRK-A, TRK-B, TROP-2 uPAR, VAP1, VCAM-1, VEGF, VEGF-A, VEGF-B, VEGF-C, VEGF-D, VEGFR1, VEGFR2, VEGFR3, VISTA, WISP-1, WISP-2, or WISP-3. In some embodiments, the polypeptide does not comprise an Fc region. In some embodiments, the VHH domain that binds an antigen other than γδ TCR, e.g., a VHH domain that binds human serum albumin, increases the half-life of the polypeptide relative to a polypeptide comprising a VHH domain that binds γδ TCR and a modified IL-2 and does not comprise a VHH that binds human serum albumin. In some embodiments the modified IL-2 is fused to the C-terminus of the VHH domain that binds human serum albumin.

In some embodiments, the γδ T-cell-targeted modified IL-2 polypeptide comprises a γδ T-cell-binding VHH, an Fc region, and a modified IL-2. In some such embodiments, the Fc region comprises an amino acid sequence selected from SEQ ID NOs: 32-70. In some embodiments, a γδ T cell-targeted modified IL-2 polypeptide includes an Fc region comprising an amino acid sequence selected from SEQ ID NOs: 32-70, wherein the Fc region lacks the C-terminal lysine residue.

In some embodiments, the modified IL-2 is fused to the C-terminus of the Fc region, which is in turn fused to the C-terminus of the γδ T-cell-binding VHH. In some embodiments, the γδ T-cell-binding VHH and Fc region are connected by a linker and the Fc region and the modified IL-2 are connected by a linker. In some embodiments, the Fc region comprises at least one knob or hole mutation. In some such embodiments, the γδ T-cell-targeted modified IL-2 polypeptide is the first polypeptide of a complex that also comprises a second polypeptide that comprises a γδ T-cell-binding domain and a second Fc region. In some embodiments, the γδ T-cell-targeted modified IL-2 polypeptide comprises a VHH comprising the amino acid sequence of SEQ ID NO: 99, 143, or 158 and a modified IL-2 polypeptide comprising the amino acid sequence of SEQ ID NO: 78 or 79 or 157. In some embodiments, the γδ T-cell-targeted modified IL-2 polypeptide comprises a VHH comprising the amino acid sequence of SEQ ID NO: 99 and a modified IL-2 polypeptide comprising the amino acid sequence of SEQ ID NO: 78. In some embodiments, the γδ T-cell-targeted modified IL-2 polypeptide comprises a VHH comprising the amino acid sequence of SEQ ID NO: 143 and a modified IL-2 polypeptide comprising the amino acid sequence of SEQ ID NO: 78. In some embodiments, the γδ T-cell-targeted modified IL-2 polypeptide comprises a VHH comprising the amino acid sequence of SEQ ID NO: 158 and a modified IL-2 polypeptide comprising the amino acid sequence of SEQ ID NO: 78. In some embodiments, the complex comprises a first polypeptide comprising a VHH comprising the amino acid sequence of SEQ ID NO: 99, 143, or 158 and a modified IL-2 polypeptide comprising the amino acid sequence of SEQ ID NO: 78 or 79 or 157, and a second polypeptide comprising a γδ T-cell-binding domain and an Fc region. In some embodiments, the complex comprises a first polypeptide comprising a VHH comprising the amino acid sequence of SEQ ID NO: 99 and a modified IL-2 polypeptide comprising the amino acid sequence of SEQ ID NO: 78, and a second polypeptide comprising a γδ T-cell-binding domain and an Fc region. In some embodiments, the complex comprises a first polypeptide comprising a VHH comprising the amino acid sequence of SEQ ID NO: 143 and a modified IL-2 polypeptide comprising the amino acid sequence of SEQ ID NO: 78, and a second polypeptide comprising a γδ T-cell-binding domain and an Fc region. In some embodiments, the complex comprises a first polypeptide comprising a VHH comprising the amino acid sequence of SEQ ID NO: 158 and a modified IL-2 polypeptide comprising the amino acid sequence of SEQ ID NO: 78, and a second polypeptide comprising a γδ T-cell-binding domain and an Fc region. In some embodiments, the second polypeptide comprises the amino acid sequence of SEQ ID NO: 99, 143, or 158.

In some embodiments, a γδ T-cell-targeted modified IL-2 polypeptide is a complex comprising a first polypeptide and a second polypeptide, wherein the first polypeptide comprises at least one VHH domain that binds γδ TCR and a first Fc region, and the second polypeptide comprises a second Fc region and a modified IL-2. In some such embodiments, at least one VHH domain that binds γδ TCR comprises a CDR1 comprising the amino acid sequence of SEQ ID NO: 3, 144, 146, 147, or 148; a CDR2 comprising the amino acid sequence of SEQ ID NO: 4, 150, 151, 152, 153, 154, 155, or 156; and a CDR3 comprising the amino acid sequence of SEQ ID NO: 5. In some embodiments, at least one VHH domain of the γδ T-cell-targeted modified IL-2 complex comprises the CDR1 sequence of SEQ ID NO: 3, the CDR2 sequence of SEQ ID NO: 4, and the CDR3 sequence of SEQ ID NO: 5.

In some embodiments, at least one VHH domain, or each VHH domain of the γδ T-cell-targeted modified IL-2 complex, is humanized. In some embodiments, at least one VHH domain of the γδ T-cell-targeted modified IL-2 complex comprises SEQ ID NO: 180, wherein X₁, X₂, X₄, X₅, X₆, X₇, X₈, X₉, X₁₀, X₁₁, X₁₂, X₁₃, X₁₄, X₁₅, X₁₆, X₁₇, X₁₈, X₁₉, X₂₀, X₂₁, X₂₂, X₂₃, X₂₄, X₂₅, X₂₆, and X₂₇ are independently selected, and wherein:

| | | |
|---|---|---|
| X₁ is V or A; | X₁₁ is D or G; | X₂₀ is T or A; |
| X₂ is R or G; | X₁₂ is A or S; | X₂₁ is A or T; |
| X₃ is K or T; | X₁₃ is A or T; | X₂₂ is V or L; |
| X₄ is I or F; | X₁₄ is E or Y; | X₂₃ is N or S; |
| X₅ is Q, G or E; | X₁₅ is V or A; | X₂₄ is K or Y; |
| X₆ is R or L; | X₁₆ is D, E, A, G, V, S, Y, L | X₂₅ is N, S, E, Y, A, S, |
| X₇ is L, W or F; | or Q; | G, Q; |
| X₈ is A or S; | X₁₇ is S, P, T, A, V, L, I, or | X₂₆ is S, T, A, L, V, N |
| X₉ is H or A; | G; | or G; and |
| X₁₀ is T or S; | X₁₈ is Gor D; | X₂₇ is K, R, E, or D. |
| | X₁₉ is S or N; | |

In some embodiments, at least one VHH domain of the γδ T-cell-targeted modified IL-2 complex comprises SEQ ID NO: 180, wherein X₃ is K, and X₁, X₂, X₄, X₅, X₆, X₇, X₈, X₉, X₁₀, X₁₁, X₁₂, X₁₃, X₁₄, X₁₅, X₁₆, X₁₇, X₁₈, X₁₉, X₂₀, X₂₁, X₂₂, X₂₃, X₂₄, X₂₅, X₂₆, and X₂₇ are independently selected, and wherein:

| | | |
|---|---|---|
| X₁ is V or A; | X₁₁ is D or G; | X₂₀ is T or A; |
| X₂ is R or G; | X₁₂ is A or S; | X₂₁ is A or T; |
| X₄ is I or F; | X₁₃ is A or T; | X₂₂ is V or L; |
| X₅ is Q, G or E; | X₁₄ is E or Y; | X₂₃ is N or S; |
| X₆ is R or L; | X₁₅ is V or A; | X₂₄ is K or Y; |
| X₇ is L, W or F; | X₁₆ is D, E, A, L or Q; | X₂₅ is N or S; |
| X₈ is A or S; | X₁₇ is S, P, T, V, L or G; | X₂₆ is S, T or G; and |
| X₉ is H or A; | X₁₈ is Gor D; | X₂₇ is K, R, E, or D. |
| X₁₀ is T or S; | X₁₉ is S or N; | |

In some embodiments, at least one VHH domain of the γδ T-cell-targeted modified IL-2 complex comprises SEQ ID NO: 180, wherein X₂ is R; X₂₅ is N; and X₁, X₃ is K, X₄, X₅, X₆, X₇, X₈, X₉, X₁₀, X₁₁, X₁₂, X₁₃, X₁₄, X₁₅, X₁₆, X₁₇, X₁₈, X₁₉, X₂₀, X₂₁, X₂₂, X₂₃, X₂₄, X₂₆, and X₂₇ are independently selected, and wherein:

| | | |
|---|---|---|
| X₁ is V or A; | X₁₁ is D or G; | X₁₉ is S or N; |
| X₄ is I or F; | X₁₂ is A or S; | X₂₀ is T or A; |
| X₅ is Q, G or E; | X₁₃ is A or T; | X₂₁ is A or T; |
| X₆ is R or L; | X₁₄ is E or Y; | X₂₂ is V or L; |
| X₇ is L, W or F; | X₁₅ is V or A; | X₂₃ is N or S; |
| X₈ is A or S; | X₁₆ is D, E, A or Q; | X₂₄ is K or Y; |
| X₉ is H or A; | X₁₇ is S, P or G; | X₂₆ is S or T; and |
| X₁₀ is T or S; | X₁₈ is Gor D; | X₂₇ is K, R, E, or D. |

In some embodiments, at least one VHH domain of the γδ T-cell-targeted modified IL-2 complex comprises SEQ ID NO: 180, wherein X₂ is R; X₃ is K, X₄ is I; X₉ is H; X₂₅ is N; and X₁, X₅, X₆, X₇, X₈, X₁₀, X₁₁, X₁₂, X₁₃, X₁₄, X₁₅, X₁₆, X₁₇, X₁₈, X₁₉, X₂₀, X₂₁, X₂₂, X₂₃, X₂₄, X₂₆, and X₂₇ are independently selected, and wherein:

| | | |
|---|---|---|
| X₁ is V or A; | X₁₃ is A or T; | X₂₀ is T or A; |
| X₅ is Q, G or E; | X₁₄ is E or Y; | X₂₁ is A or T; |
| X₆ is R or L; | X₁₅ is V or A; | X₂₂ is V or L; |
| X₇ is L, W or F; | X₁₆ is D, E, or A; | X₂₃ is N or S; |
| X₈ is A or S; | X₁₇ is S, or P; | X₂₄ is K or Y; |
| X₁₀ is T or S; | X₁₈ is G or D; | X₂₆ is S or T; and |
| X₁₁ is D or G; | X₁₉ is S or N; | X₂₇ is K, R, E, or D. |
| X₁₂ is A or S; | | |

In some embodiments, at least one VHH domain of the γδ T-cell-targeted modified IL-2 complex comprises SEQ ID NO: 180, wherein X₁ is V; X₂ is R; X₃ is K, X₄ is I; X₉ is H; X₁₀ is T; X₁₁ is D; X₁₂ is A; X₁₃ is A; X₁₄ is E; X₂₅ is N; and X₅, X₆, X₇, X₈, X₁₅, X₁₆, X₁₇, X₁₈, X₁₉, X₂₀, X₂₁, X₂₂, X₂₃, X₂₄, X₂₆, and X₂₇ are independently selected, and wherein:

| | | |
|---|---|---|
| X₅ is Q, G or E; | X₁₇ is S, or P; | X₂₂ is V or L; |
| X₆ is R or L; | X₁₈ is G or D; | X₂₃ is N or S; |
| X₇ is L, or W; | X₁₉ is S or N; | X₂₄ is K or Y; |
| X₈ is A or S; | X₂₀ is T or A; | X₂₆ is S or T; and |
| X₁₅ is V or A; | X₂₁ is A or T; | X₂₇ is K, R, E, or D. |
| X₁₆ is D, E, or A; | | |

In some embodiments, at least one VHH domain of the γδ T-cell-targeted modified IL-2 complex comprises an amino acid sequence at least 85%, at least 90%, at least 95%, or at least 99% identical to an amino acid sequence selected from SEQ ID NOs: 2, 17-19, 21-23, 25-31, 72-77, 80, 81, 84-86, 88-93, 95, 97-107, 109, 111-129, 131-133, 135-137, 139-143, 158-159, and 166-179. In some embodiments, a VHH domain that binds γδ TCR comprises an amino acid sequence selected from SEQ ID NOs: 2, 17-19, 21-23, 25-31, 72-77, 80, 81, 84-86, 88-93, 95, 97-107, 109, 111-129, 131-133, 135-137, 139-143, 158-159, and 166-179, wherein position 114 of the VHH domain has been substituted with a lysine (K) an aspartate (D), a glutamate (E), or an arginine (R). In some such embodiments, at least one VHH domain comprises an amino acid sequence selected from SEQ ID NOs: 17-19, 21-23, 25-31, 72-77, 80, 81, 84-86, 88-93, 95, 97-107, 109, 111-129, 131-133, 135-137, 139-143, 158-159, and 166-179. In some embodiments, at least one VHH domain comprises the amino acid sequence of SEQ ID NO: 99, 143, or 158.

In some embodiments, the modified IL-2 of the γδ T-cell-targeted modified IL-2 complex comprises T3A or T3G, H16A, P65R, C125S, and D84S or D84Y mutations relative to a wild type human IL-2. In some embodiments, the modified IL-2 comprises T3A, H16A, P65R, C125S, and D84S mutations. In some embodiments, the modified IL-2 comprises T3A, H16A, E61R, P65R, C125S, and D84Y mutations. In some embodiments, the modified IL-2 comprises an amino acid sequence that is at least 90%, at least 95%, at least 97%, at least 98%, at least 99%, or 100% identical to the amino acid sequence of SEQ ID NO: 78 or 79 or 157. In some embodiments, the modified IL-2 comprises the amino acid sequence of SEQ ID NO: 78.

In some embodiments, the second polypeptide of the γδ T-cell-targeted modified IL-2 complex comprises at least one antigen-binding domain. In some such embodiments, the at least one antigen-binding domain of the second polypeptide is a VHH domain. In some embodiments, the second polypeptide comprises at least one VHH domain that binds γδ TCR.

In any of the embodiments described herein, the modified IL-2 may be a modified human IL-2. In various embodiments, the amino acid positions of the substitutions correspond to the amino acid positions in SEQ ID NO: 71.

In various embodiments, an Fc region included in a γδ T-cell-targeted modified IL-2 polypeptide is a human Fc region, or is derived from a human Fc region. In some embodiments, the Fc region included in a γδ T-cell-targeted modified IL-2 polypeptide is derived from a human Fc region and lacks the C-terminal lysine residue. In some embodiments, the Fc region included in a γδ T-cell-targeted modified IL-2 polypeptide is derived from a human Fc region and comprises the C-terminal lysine residue. In some embodiments, the C-terminal amino acid of the Fc region is an amino acid other than lysine.

In some embodiments, an Fc region included in a γδ T-cell-targeted modified IL-2 polypeptide is derived from a human Fc region, and comprises a three amino acid deletion in the lower hinge corresponding to IgG1 E233, L234, and L235, herein referred to as “Fc xELL.” Fc xELL polypeptides do not engage FcγRs and thus are referred to as “effector silent” or “effector null”, however in some embodiments, xELL Fc regions bind FcRn and therefore have extended half-life and transcytosis associated with FcRn mediated recycling.

In some embodiments, the Fc region included in a γδ T-cell-targeted modified IL-2 polypeptide is derived from a human Fc region and comprises mutations M252Y and M428V, herein referred to as “Fc-YV”. In some embodiments, such mutations enhance binding to FcRn at the acidic pH of the endosome (near 6.5), while losing detectable binding at neutral pH (about 7.2), allowing for enhanced FcRn mediated recycling and extended half-life.

In some embodiments, the Fc region included in a γδ T-cell-targeted modified IL-2 polypeptide is derived from a human Fc region and comprises mutations designed for heterodimerization, herein referred to as “knob” and “hole”. In some embodiments, the “knob” Fc region comprises the mutation T366W. In some embodiments, the “hole” Fc region comprises mutations T366S, L368A, and Y407V. In some embodiments, Fc regions used for heterodimerization comprise additional mutations, such as the mutation S354C on a first member of a heterodimeric Fc pair that forms an asymmetric disulfide with a corresponding mutation Y349C on the second member of a heterodimeric Fc pair. In some embodiments, one member of a heterodimeric Fc pair comprises the modification H435R or H435K to prevent protein A binding while maintaining FcRn binding. In some embodiments, one member of a heterodimeric Fc pair comprises the modification H435R or H435K, while the second member of the heterodimeric Fc pair is not modified at H435. In various embodiments, the hold Fc region comprises the modification H435R or H435K (referred to as “hole-R” in some instances when the modification is H435R), while the knob Fc region does not. In some instances, the hole-R mutation improves purification of the heterodimer over homodimeric hole Fc regions that may be present.

Nonlimiting exemplary Fc regions that may be used in a γδ T-cell-targeted modified IL-2 polypeptide include Fc regions comprising the amino acid sequences of SEQ ID NOs: 32-70. In some embodiments, a γδ T cell-targeted modified IL-2 polypeptide includes an Fc region comprising an amino acid sequence selected from SEQ ID NOs: 32-70, wherein the Fc region lacks the C-terminal lysine residue. In some embodiments, a γδ T cell-targeted modified IL-2 polypeptide includes an Fc region comprising an amino acid sequence selected from SEQ ID NOs: 34, 35, 41-52, 58-70. In some embodiments, a γδ T cell-targeted modified IL-2 polypeptide includes an Fc region comprising an amino acid sequence selected from SEQ ID NOs: 34, 35, 41-52, 58-70, wherein the Fc region lacks the C-terminal lysine residue.

### Exemplary Activities of γδ T-Cell-Targeted Modified IL-2 Polypeptides

In various embodiments, the γδ T-cell-targeted modified IL-2 polypeptides provided herein stimulate γδ T-cells in vitro and/or in vivo. Stimulation or activity of γδ T-cells in vitro and/or in vivo may be determined, in some embodiments, using the methods provided in the Examples herein. In some embodiments, the γδ T-cells stimulating activity of the modified IL-2 is greater and/or more specifically targeted to cytotoxic T-cells when fused to a γδ T-cell-binding VHH than when used alone. In some embodiments, toxicity of IL-2 is reduced by specifically targeting it to γδ T-cells. In some embodiments, the γδ T-cell-targeted modified IL-2 polypeptides provided herein increase T-cell activation and/or proliferation in vitro and/or in vivo. In some embodiments, the γδ T-cell-targeted modified IL-2 polypeptides provided herein stimulate activation and/or proliferation of γδ T-cells in vivo, and accordingly, the γδ T-cell-targeted modified IL-2 polypeptides may be used in a method of treating cancer.

In various embodiments, the γδ T-cell-targeting modified IL-2 polypeptides provided herein are agonists of IL-2R activity. Agonist activity may be determined, in some embodiments, using the methods provided in the Examples herein. In some embodiments, the γδ T-cell-targeting modified IL-2 polypeptides provided herein are agonists of IL-2R activity when targeted to γδ T-cells, but show little or no agonist activity in the absence of targeting.

In some embodiments, the γδ T-cell-targeting modified IL-2 polypeptides provided herein increase proliferation of γδ T-cells in vitro and/or in vivo. In some embodiments, the polypeptide increases γδ T-cell proliferation in the presence of Treg cells. In some such embodiments, the γδ T-cells are activated γδ T-cells. In some embodiments, a γδ T-cell-targeting modified IL-2 polypeptide provided herein increases activated γδ T-cells proliferation in vitro. In some embodiments, the γδ T-cell-targeting modified IL-2 polypeptide increases activated γδ T-cells proliferation by at least 1.5-fold, at least 2-fold, at least 3-fold, or by at least 5-fold relative to γδ T-cell proliferation in the absence of the polypeptide. In some embodiments, the polypeptide increases proliferation of activated γδ T-cells by at least 1.5-fold, at least 2-fold, at least 3-fold, or by at least 5-fold and does not substantially increase the proliferation of resting γδ T-cells, relative to the proliferation observed in the absence of the polypeptide.

The increase in proliferation of activated γδ T-cells may be determined by any method in the art, such as for example, the methods provided in the Examples herein. A nonlimiting exemplary assay is as follows. γδ T-cells may be isolated from one or more healthy human donors. The T-cells are stained with CellTrace Violet (CTV) and contacted with a polypeptide comprising a modified IL-2, and then analyzed by FACS. Loss of CTV staining indicates proliferation. In some embodiments, an increase in γδ T-cell proliferation is determined as an average from a set of experiments or from pooled T-cells, such as by measuring proliferation of γδ T-cells isolated from different healthy human donors. In some embodiments, an increase in γδ T-cell proliferation is determined as an average from experiments carried out using T-cells from at least five or at least ten different healthy donors, or from a pool of T-cells from at least five or at least ten different healthy donors.

In some embodiments, the γδ T-cell-targeting modified IL-2 polypeptides provided herein increase STAT5 phosphorylation (i.e., pSTAT5 expression) in γδ T-cells in vitro and/or in vivo. pSTAT5 expression indicates T-cell activation. In some embodiments, a γδ T-cell-targeting modified IL-2 polypeptide provided herein increases pSTAT5 expression in γδ T-cells in vitro. In some embodiments, the γδ T-cell-targeting modified IL-2 polypeptide increases pSTAT5 expression on γδ T-cells by at least 1.5-fold, at least 2-fold, at least 3-fold, or by at least 5-fold relative to pSTAT5 expression in the absence of the polypeptide. In some embodiments, the polypeptide increases pSTAT5 expression on γδ T-cells in the presence of Treg cells. The increase in pSTAT5 expression in γδ T-cells may be determined by any method in the art, such as for example, the methods provided in the Examples herein.

In some embodiments, the γδ T-cell-targeting modified IL-2 polypeptides provided herein increase the proliferation of activated γδ T-cells which are capable of killing cells from tumor cell lines including but not limited to: Daudi and Raji the Burkitt's B cell lymphoma cells, THP-1 acute monocytic leukemia cells, HT29 colorectal adenocarcinoma cells, Daudi Burkitts lymphoma cells, NCI-H460 lung carcinoma cells, MM1S multiple myeloma cells, A375 malignant melanoma cells, and OVCAR ovarian cancer cells. In some embodiment, the γδ T-cell-targeting modified IL-2 polypeptides provided herein increase the proliferation γδ T-cells present in patient samples from hematological and solid tumor indications including but not limited to ovarian cancer, colorectal cancer (CRC), acute monocytic leukemia, and B cell acute lymphoblastic leukemia (B-ALL). In certain embodiment, proliferated patient γδ T-cells exhibit cancer cell killing activity. The proliferation and cell killing activity of γδ T-cells may be determined by any method in the art, such as for example, the methods provided in the Examples herein.

### Polypeptide Expression and Production

Nucleic acid molecules comprising polynucleotides that encode a γδ T-cell-targeted modified IL-2 polypeptide are provided. In some embodiments, the nucleic acid molecule may also encode a leader sequence that directs secretion of the γδ T-cell-targeted modified IL-2 polypeptide, which leader sequence is typically cleaved such that it is not present in the secreted polypeptide. The leader sequence may be a native heavy chain (or VHH) leader sequence, or may be another heterologous leader sequence.

Nucleic acid molecules can be constructed using recombinant DNA techniques conventional in the art. In some embodiments, a nucleic acid molecule is an expression vector that is suitable for expression in a selected host cell.

Vectors comprising nucleic acids that encode the γδ T-cell-targeted modified IL-2 polypeptides described herein are provided. Such vectors include, but are not limited to, DNA vectors, phage vectors, viral vectors, retroviral vectors, etc. In some embodiments, a vector is selected that is optimized for expression of polypeptides in a desired cell type, such as CHO or CHO-derived cells, or in NSO cells. Exemplary such vectors are described, for example, in Running Deer et al., Biotechnol. Prog. 20:880-889 (2004).

In some embodiments, a γδ T-cell-targeted modified IL-2 polypeptide may be expressed in prokaryotic cells, such as bacterial cells; or in eukaryotic cells, such as fungal cells (such as yeast), plant cells, insect cells, and mammalian cells. Such expression may be carried out, for example, according to procedures known in the art. Exemplary eukaryotic cells that may be used to express polypeptides include, but are not limited to, COS cells, including COS 7 cells; 293 cells, including 293-6E cells; CHO cells, including CHO-S, DG44. Lec13 CHO cells, and FUT8 CHO cells; PER.C6® cells (Crucell); and NSO cells. In some embodiments, the γδ T-cell-targeted modified IL-2 polypeptides may be expressed in yeast. See, e.g., U.S. Publication No. US 2006/0270045 A1. In some embodiments, a particular eukaryotic host cell is selected based on its ability to make desired post-translational modifications to the polypeptide. For example, in some embodiments, CHO cells produce polypeptides that have a higher level of sialylation than the same polypeptide produced in 293 cells.

Introduction of one or more nucleic acids (such as vectors) into a desired host cell may be accomplished by any method, including but not limited to, calcium phosphate transfection, DEAE-dextran mediated transfection, cationic lipid-mediated transfection, electroporation, transduction, infection, etc. Nonlimiting exemplary methods are described, for example, in Sambrook et al., Molecular Cloning, A Laboratory Manual, 3^{rd} ed. Cold Spring Harbor Laboratory Press (2001). Nucleic acids may be transiently or stably transfected in the desired host cells, according to any suitable method.

Host cells comprising any of the nucleic acids or vectors described herein are also provided. In some embodiments, a host cell that expresses a γδ T-cell-targeted modified IL-2 polypeptide described herein is provided. The γδ T-cell-targeted modified IL-2 polypeptides expressed in host cells can be purified by any suitable method. Such methods include, but are not limited to, the use of affinity matrices or hydrophobic interaction chromatography. Suitable affinity ligands include the ROR1 ECD and agents that bind Fc regions. For example, a Protein A, Protein G, Protein A/G, or an antibody affinity column may be used to bind the Fc region and to purify a γδ T-cell-targeted modified IL-2 polypeptide that comprises an Fc region. Hydrophobic interactive chromatography, for example, a butyl or phenyl column, may also be suitable for purifying some polypeptides such as antibodies. Ion exchange chromatography (for example anion exchange chromatography and/or cation exchange chromatography) may also be suitable for purifying some polypeptides such as antibodies. Mixed-mode chromatography (for example reversed phase/anion exchange, reversed phase/cation exchange, hydrophilic interaction/anion exchange, hydrophilic interaction/cation exchange, etc.) may also be suitable for purifying some polypeptides such as antibodies. Many methods of purifying polypeptides are known in the art.

In some embodiments, the γδ T-cell-targeted modified IL-2 polypeptide is produced in a cell-free system. Nonlimiting exemplary cell-free systems are described, for example, in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

In some embodiments, γδ T-cell-targeted modified IL-2 polypeptides prepared by the methods described above are provided. In some embodiments, the γδ T-cell-targeted modified IL-2 polypeptide is prepared in a host cell. In some embodiments, the γδ T-cell-targeted modified IL-2 polypeptide is prepared in a cell-free system. In some embodiments, the γδ T-cell-targeted modified IL-2 polypeptide is purified. In some embodiments, a cell culture media comprising a γδ T-cell-targeted modified IL-2 polypeptide is provided.

In some embodiments, compositions comprising antibodies prepared by the methods described above are provided. In some embodiments, the composition comprises a γδ T-cell-targeted modified IL-2 polypeptide prepared in a host cell. In some embodiments, the composition comprises a γδ T-cell-targeted modified IL-2 polypeptide prepared in a cell-free system. In some embodiments, the composition comprises a purified γδ T-cell-targeted modified IL-2 polypeptide.

### Exemplary Methods of Treating Diseases Using γδ T-Cell-Targeted Modified IL-2 Polypeptides

In some embodiments, methods of treating disease in an individual comprising administering a γδ T-cell-targeted modified IL-2 polypeptide are provided. Such diseases include any disease that would benefit from increased proliferation and activation of γδ T-cells. In some embodiments, increased proliferation or activation of γδ T-cells is induced by a γδ T-cell-targeted modified IL-2 polypeptide. In some embodiments, methods for treating cancer in an individual are provided.

The method comprises administering to the individual an effective amount of a γδ T-cell-targeted modified IL-2 polypeptide provided herein. Such methods of treatment may be in humans or animals. In some embodiments, methods of treating humans are provided. Nonlimiting exemplary cancers that may be treated with γδ T-cell-targeted modified IL-2 polypeptides provided herein include basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer; gastrointestinal cancer; glioblastoma; hepatic carcinoma; hepatoma; intra-epithelial neoplasm; kidney or renal cancer; larynx cancer; liver cancer; lung cancer; small-cell lung cancer; non-small cell lung cancer; adenocarcinoma of the lung; squamous carcinoma of the lung; melanoma; myeloma; neuroblastoma; oral cavity cancer; ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; and vulval cancer; lymphoma; Hodgkin's lymphoma; non-Hodgkin's lymphoma; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; chronic lymphocytic leukemia (CLL); acute lymphoblastic leukemia (ALL); Hairy cell leukemia; and chronic myeloblastic leukemia.

The γδ T-cell-targeted modified IL-2 polypeptides can be administered as needed to subjects. Determination of the frequency of administration can be made by persons skilled in the art, such as an attending physician based on considerations of the condition being treated, age of the subject being treated, severity of the condition being treated, general state of health of the subject being treated and the like. In some embodiments, an effective dose of a γδ T-cell-targeted modified IL-2 polypeptides is administered to a subject one or more times. In some embodiments, an effective dose of a γδ T-cell-targeted modified IL-2 polypeptides is administered to the subject daily, semiweekly, weekly, every two weeks, once a month, etc. An effective dose of a γδ T-cell-targeted modified IL-2 polypeptides is administered to the subject at least once. In some embodiments, the effective dose of a γδ T-cell-targeted modified IL-2 polypeptides may be administered multiple times, including multiple times over the course of at least a month, at least six months, or at least a year.

In some embodiments, pharmaceutical compositions are administered in an amount effective for treating (including prophylaxis of) cancer and/or increasing T-cell proliferation. The therapeutically effective amount is typically dependent on the weight of the subject being treated, his or her physical or health condition, the extensiveness of the condition to be treated, or the age of the subject being treated. In general, antibodies may be administered in an amount in the range of about 0.05 mg/kg body weight to about 100 mg/kg body weight per dose.

In some embodiments, γδ T-cell targeted modified IL-2 polypeptides can be administered in vivo by various routes, including, but not limited to, intravenous, intra-arterial, parenteral, intraperitoneal or subcutaneous. The appropriate formulation and route of administration may be selected according to the intended application.

In some embodiments, a therapeutic treatment using a γδ T-cell-targeted modified IL-2 polypeptide is achieved by increasing T-cell proliferation and/or activation, and/or by bringing γδ T-cells in contact with cancer cells. In some embodiments, increasing T-cell proliferation and/or activation inhibits growth of cancer.

### Pharmaceutical Compositions

In some embodiments, compositions comprising γδ T cell-targeted modified IL-2 polypeptides are provided in formulations with a wide variety of pharmaceutically acceptable carriers (see, for example, Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7^{th} ed., Lippencott Williams and Wilkins (2004); Kibbe et al., Handbook of Pharmaceutical Excipients, 3^{rd} ed., Pharmaceutical Press (2000)). Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available. Non-limiting exemplary carriers include saline, buffered saline, dextrose, water, glycerol, ethanol, and combinations thereof.

In some embodiments, a pharmaceutical composition comprises a γδ T-cell-targeted modified IL-2 polypeptide at a concentration of at least 10 mg/mL.

### Combination Therapy

γδ T-cell-targeted modified IL-2 polypeptides can be administered alone or in combination with other modes of treatment, such as other anti-cancer agents. They can be provided before, substantially contemporaneous with, or after other modes of treatment (i.e., concurrently or sequentially). In some embodiments, the method of treatment described herein can further include administering: radiation therapy, chemotherapy, vaccination, targeted tumor therapy, CAR-T therapy, oncolytic virus therapy, cancer immunotherapy, cytokine therapy, surgical resection, chromatin modification, ablation, cryotherapy, an antisense agent against a tumor target, a siRNA agent against a tumor target, a microRNA agent against a tumor target or an anti-cancer/tumor agent, or a biologic, such as an antibody, cytokine, or receptor extracellular domain-Fc fusion.

In some embodiments, a γδ T-cell-targeted modified IL-2 polypeptide provided herein is given concurrently with a second therapeutic agent, for example, a PD-1 or PD-L1 therapy. Examples of PD-1/PD-L1 therapy include nivolumab (BMS); pidilizumab (CureTech, CT-011), pembrolizumab (Merck); durvalumab (Medimmune/AstraZeneca); atezolizumab (Genentech/Roche); avelumab (Pfizer); AMP-224 (Amplimmune); BMS-936559; AMP-514 (Amplimmune); MDX-1105 (Merck); TSR-042 (Tesaro/AnaptysBio, ANB-011); STI-A1010 (Sorrento Therapeutics); STI-A1110 (Sorrento Therapeutics); and other agents that are directed against programmed death-1 (PD-1) or programmed death ligand 1 (PD-L1).

In some embodiments, a γδ T-cell-targeted modified IL-2 polypeptide provided herein is given concurrently with an immune stimulatory agent, for example, an agonist of a member of the Tumor Necrosis Factor Receptor Super Family (TNFRSF) or a member the B7 family. Nonlimiting examples of immune stimulatory TNFRSF members include OX40, GITR, 41BB, CD27, and HVEM. Nonlimiting examples of B7 family members include CD28 and ICOS. Thus, in some embodiments, a γδ T-cell-targeted modified IL-2 polypeptide provided herein is given concurrently with an agonist, such as an agonist antibody, of OX40, GITR, 41BB, CD27, HVEM, CD28, and/or ICOS.

In some embodiments, a γδ T-cell-targeted modified IL-2 polypeptide provided herein is given concurrently with CAR-T (chimeric antigen receptor T-cell) therapy, oncolytic virus therapy, cytokine therapy, and/or agents that target other checkpoint molecules, such as VISTA, gpNMB, B7H3, B7H4, HHLA2, CTLA4, TIGIT, etc.

### Nonlimiting Exemplary Methods of Diagnosis and Treatment

In some embodiments, the methods described herein are useful for evaluating a subject and/or a specimen from a subject (e.g. a cancer patient). In some embodiments, evaluation is one or more of diagnosis, prognosis, and/or response to treatment.

In some embodiments, the methods described herein comprise evaluating a presence, absence, or level of a protein. In some embodiments, the methods described herein comprise evaluating a presence, absence, or level of expression of a nucleic acid. The compositions described herein may be used for these measurements. For example, in some embodiments, the methods described herein comprise contacting a specimen of the tumor or cells cultured from the tumor with a therapeutic agent as described herein.

In some embodiments, the evaluation may direct treatment (including treatment with the antibodies described herein). In some embodiments, the evaluation may direct the use or withholding of adjuvant therapy after resection. Adjuvant therapy, also called adjuvant care, is treatment that is given in addition to the primary, main or initial treatment. By way of non-limiting example, adjuvant therapy may be an additional treatment usually given after surgery where all detectable disease has been removed, but where there remains a statistical risk of relapse due to occult disease. In some embodiments, the antibodies are used as an adjuvant therapy in the treatment of a cancer. In some embodiments, the antibodies are used as the sole adjuvant therapy in the treatment of a cancer. In some embodiments, the antibodies described herein are withheld as an adjuvant therapy in the treatment of a cancer. For example, if a patient is unlikely to respond to an antibody described herein or will have a minimal response, treatment may not be administered in the interest of quality of life and to avoid unnecessary toxicity from ineffective chemotherapies. In such cases, palliative care may be used.

In some embodiments the molecules are administered as a neoadjuvant therapy prior to resection. In some embodiments, neoadjuvant therapy refers to therapy to shrink and/or downgrade the tumor prior to any surgery. In some embodiments, neoadjuvant therapy means chemotherapy administered to cancer patients prior to surgery. In some embodiments, neoadjuvant therapy means an antibody is administered to cancer patients prior to surgery. Types of cancers for which neoadjuvant chemotherapy is commonly considered include, for example, breast, colorectal, ovarian, cervical, bladder, and lung. In some embodiments, the antibodies are used as a neoadjuvant therapy in the treatment of a cancer. In some embodiments, the use is prior to resection.

In some embodiments, the tumor microenvironment contemplated in the methods described herein is one or more of: tumor vasculature; tumor-infiltrating lymphocytes; fibroblast reticular cells; endothelial progenitor cells (EPC); cancer-associated fibroblasts; pericytes; other stromal cells; components of the extracellular matrix (ECM); dendritic cells; antigen presenting cells; T-cells; regulatory T-cells; macrophages; neutrophils; and other immune cells located proximal to a tumor.

### Kits

Also provided are articles of manufacture and kits that include any of γδ T cell-targeted modified IL-2 polypeptides as described herein, and suitable packaging. In some embodiments, the invention includes a kit with (i) a γδ T cell-targeted modified IL-2 polypeptide, and (ii) instructions for using the kit to administer the γδ T cell-targeted modified IL-2 polypeptide to an individual.

Suitable packaging for compositions described herein are known in the art, and include, for example, vials (e.g., sealed vials), vessels, ampules, bottles, jars, flexible packaging (e.g., sealed Mylar or plastic bags), and the like. These articles of manufacture may further be sterilized and/or sealed. Also provided are unit dosage forms comprising the compositions described herein. These unit dosage forms can be stored in a suitable packaging in single or multiple unit dosages and may also be further sterilized and sealed. Instructions supplied in the kits of the invention are typically written instructions on a label or package insert (e.g., a paper sheet included in the kit), but machine-readable instructions (e.g., instructions carried on a magnetic or optical storage disk) are also acceptable. The instructions relating to the use of the antibodies generally include information as to dosage, dosing schedule, and route of administration for the intended treatment or industrial use. The kit may further comprise a description of selecting an individual suitable or treatment.

The containers may be unit doses, bulk packages (e.g., multi-dose packages) or sub-unit doses. For example, kits may also be provided that contain sufficient dosages of molecules disclosed herein to provide effective treatment for an individual for an extended period, such as about any of a week, 2 weeks, 3 weeks, 4 weeks, 6 weeks, 8 weeks, 3 months, 4 months, 5 months, 6 months, 7 months, 8 months, 9 months, or more. Kits may also include multiple unit doses of molecules and instructions for use and packaged in quantities sufficient for storage and use in pharmacies, for example, hospital pharmacies and compounding pharmacies. In some embodiments, the kit includes a dry (e.g., lyophilized) composition that can be reconstituted, resuspended, or rehydrated to form generally a stable aqueous suspension of antibody.

### EXAMPLES

The examples discussed below are intended to be purely exemplary of the invention and should not be considered to limit the invention in any way. The examples are not intended to represent that the experiments below are all or the only experiments performed. Efforts have been made to ensure accuracy with respect to numbers used (for example, amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: γδTCR Targeted IL-2 Activates the STAT5 Signaling Pathway in γδ T Cells

T-cell activation through IL-2 signaling leads to the phosphorylation of the transcription factor STAT5. PBMCs were isolated from healthy donor leukopaks by lymphoprep density gradient centrifugation. The cells were labeled for 20 minutes at room temperature with the following fluorescently conjugated antibodies: non-competing anti-γδTCR-FITC, anti-CD3-BV785, and anti-CD56-BV421. After washing, 200,000 PBMCs were seeded per well in a 96-well plate. The cells were treated with a titration of the fusion protein comprising either IL-2_X or IL-2_Y fused to the C-terminus of either anti-γδTCR-5C8 or anti-γδTCR-6C4 starting at an initial concentration of 100 nM and titrating across the plate 1:5 in duplicate. The plates were incubated for 20 minutes at 37° C./5% CO₂. The cells were fixed with BD Cytofix/Cytoperm™ (BD Biosciences), permeabilized in 90% ice-cold methanol, and levels of phosphorylated STAT5 (“pSTAT5”) on cells were measured by flow cytometry using a phospho-specific anti-pSTAT5-PE antibody (1:70) and gating on CD3-BV785⁺ γδTCR-FITC⁺ to identify γδ T cells, CD56-BV421⁺ CD3-BV785⁻ to identify NK cells, and CD3-BV785⁺ γδTCR-FITCX⁻ to identify αβ T-cells.

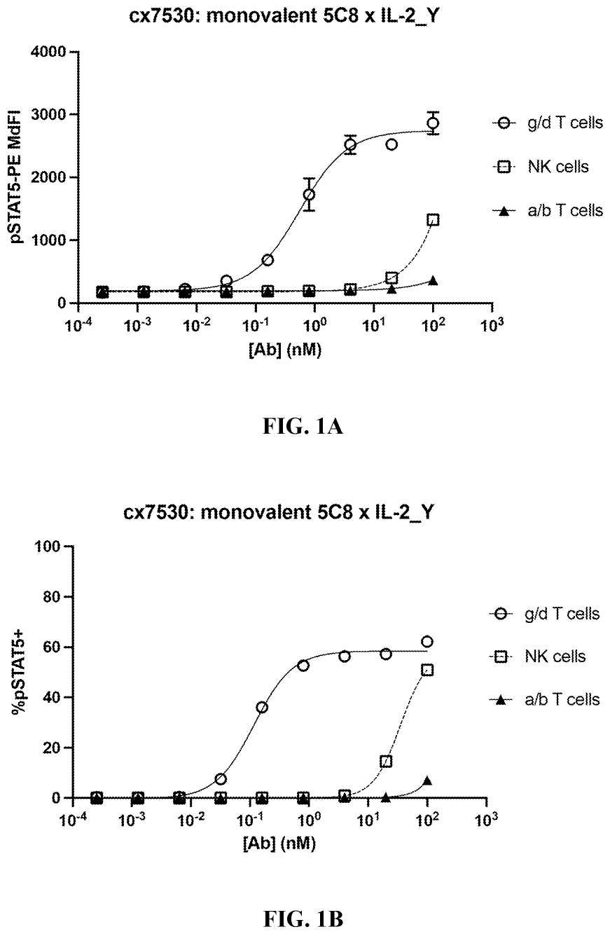

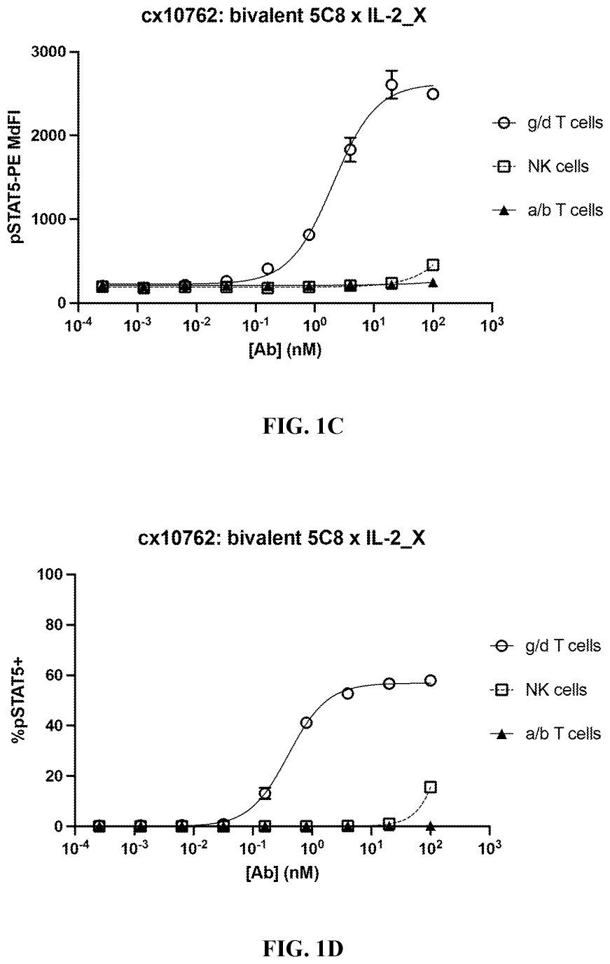

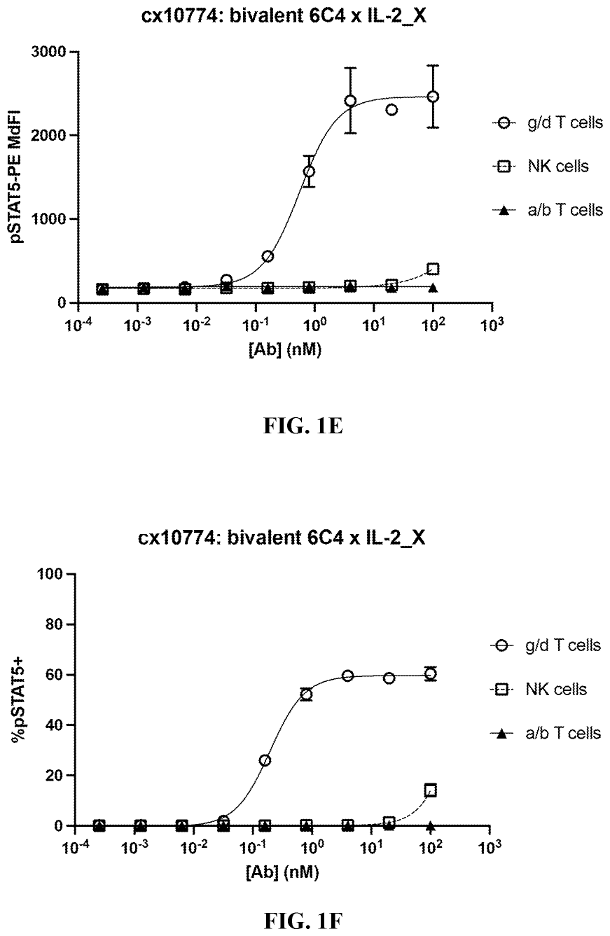

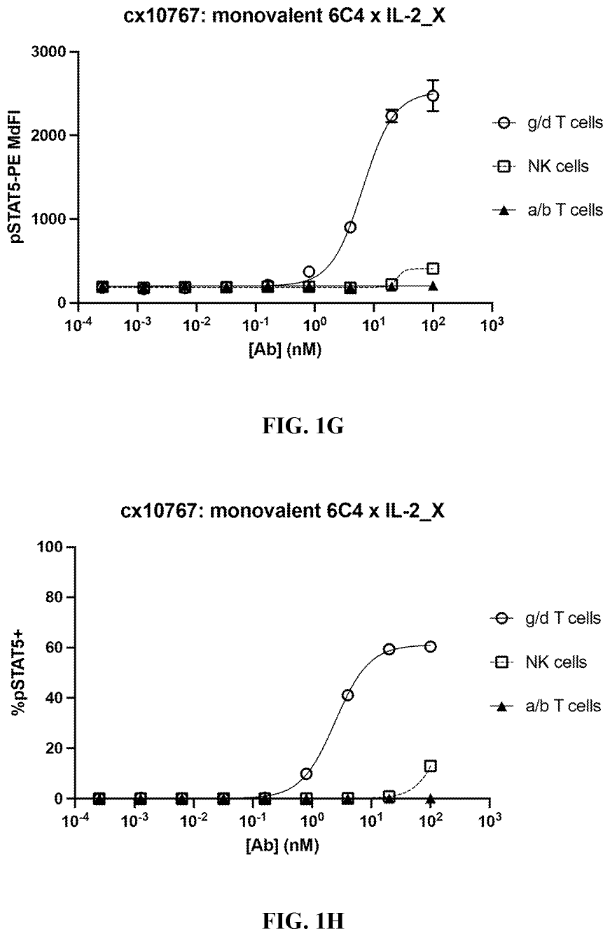

As shown in FIG. 1A-1H, treatment with bivalent or monovalent anti-γδTCR-5C8 or anti-γδTCR-6C4 targeted IL-2_X or IL-2_Y led to a dose-dependent increase in the percentage of pSTAT5⁺ γδ T cells and the pSTAT5 median fluorescent intensity on γδ T-cells. In contrast, there was little pSTAT5 activation on NK or αβ T-cells below 100 nM.

### Example 2: Enhanced Proliferation and Accumulation of γδ T Cells Upon Treatment with γSTCR-Targeted Low Affinity IL-2_X

IL-2 promotes the activation and proliferation of T cell populations. In order to assess the effect of γδTCR-targeted IL-2_X on proliferation, PBMCs were isolated from healthy donor leukopaks using lymphoprep density gradient medium. The cells were labeled with CellTrace Violet proliferative dye for 10 minutes at 37° C. After washing, the cells were resuspended in RPMI+10% FBS and 300,000 cells were added per well in a 96-well plate. The cells were treated with a titration of the fusion protein comprising IL-2_X fused to the C-terminal of either anti-γδTCR-5C8 or a non-targeted control VHH starting at an initial concentration of 100 nM and titrating across the plate 1:5 in duplicate. The plates were incubated at 37° C./5% C02 for 7 days. The cells were labeled with the following fluorescently conjugated antibodies: CD3-BV785, γδTCR-FITC, and the viability dye propidium iodide for 30 minutes at 4° C. The cells were washed and analyzed by flow cytometry.

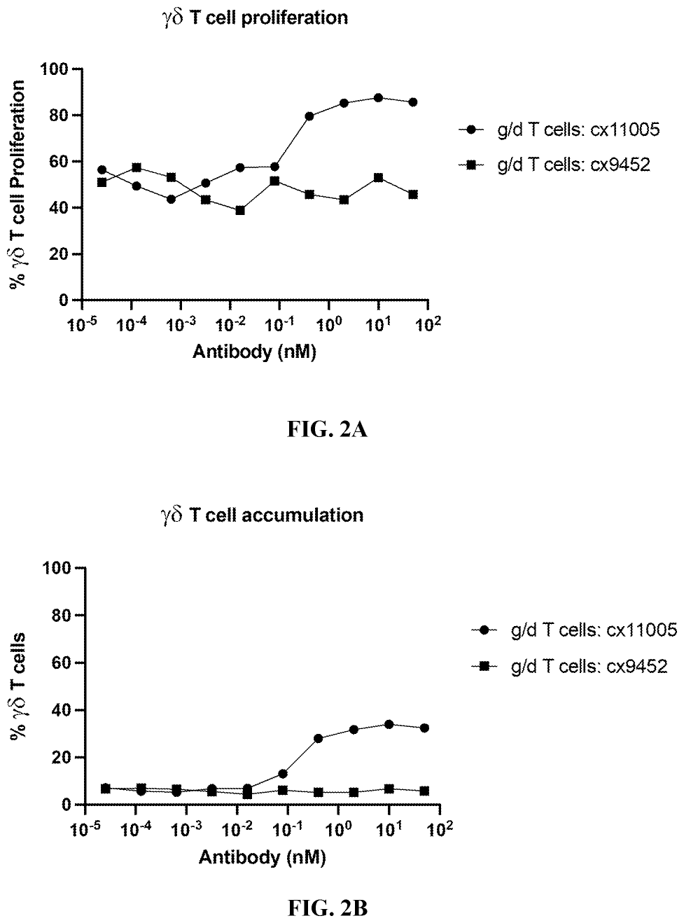

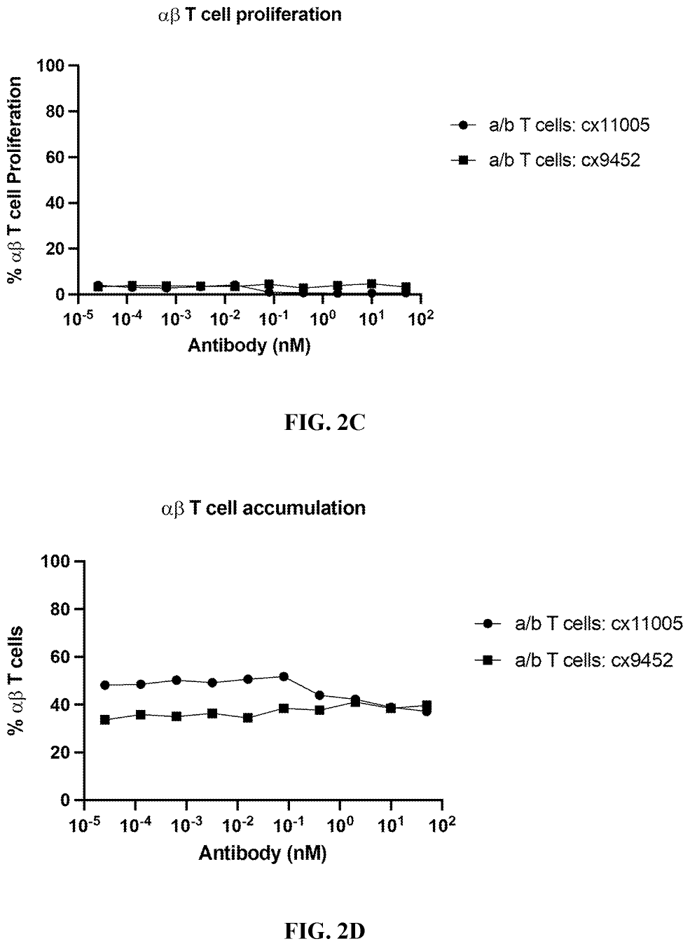

As shown in FIG. 2A-2D, treatment with anti-γδTCR-5C8 targeted IL-2_X led to a dose-dependent increase in the proliferation of γδ T cells. The percentage of γδ T cells among the total CD3⁺ population also increased with anti-γδTCR-5C8 targeted IL-2_X treatment along with a concomitant decrease in αβ T cells. The effect was specific for γδ T cells as the αβ T cell population did not proliferate in response to treatment with anti-γδTCR-5C8 targeted IL-2_X. The non-targeted VHH fused to IL-2_X did not promote the proliferation of either γδ or αβ T cells demonstrating target specificity.

### Example 3: γSTCR Targeted IL-2 Activates the STAT5 Signaling in Vδ2⁺ γδ T Cells

IL-2 signaling leads to the phosphorylation of the transcription factor STAT5 and activation of downstream gene expression. To assess STAT5 phosphorylation PBMCs were isolated from healthy human donor whole blood by lymphoprep density gradient centrifugation. The cells were labeled for 20 minutes at room temperature with the following fluorescently conjugated antibodies: non-competing anti-γδTCR-FITC, anti-CD3-BV785, and anti-Vδ2-BV421. After washing, 400,000 PBMCs were seeded per well in a 96-well plate. The cells were treated with a titration of the fusion protein comprising either IL-2_X fused to the C-terminal of anti-γδTCR-1D7 starting at an initial concentration of 50 nM and titrating across the plate 1:3 in duplicate. The plates were incubated for 20 minutes at 37° C./5% C02. The cells were fixed with BD Cytofix/Cytoperm™ (BD Biosciences), permeabilized in 90% ice-cold BD Phosflow™ Perm Buffer III (BD Biosciences), and levels of phosphorylated STAT5 (“pSTAT5”) on Vδ2⁺ γδ T cells or Vδ2⁻ γδ⁻ alpha beta T cells were measured by flow cytometry using a phospho-specific anti-pSTAT5-PE antibody (1:70).

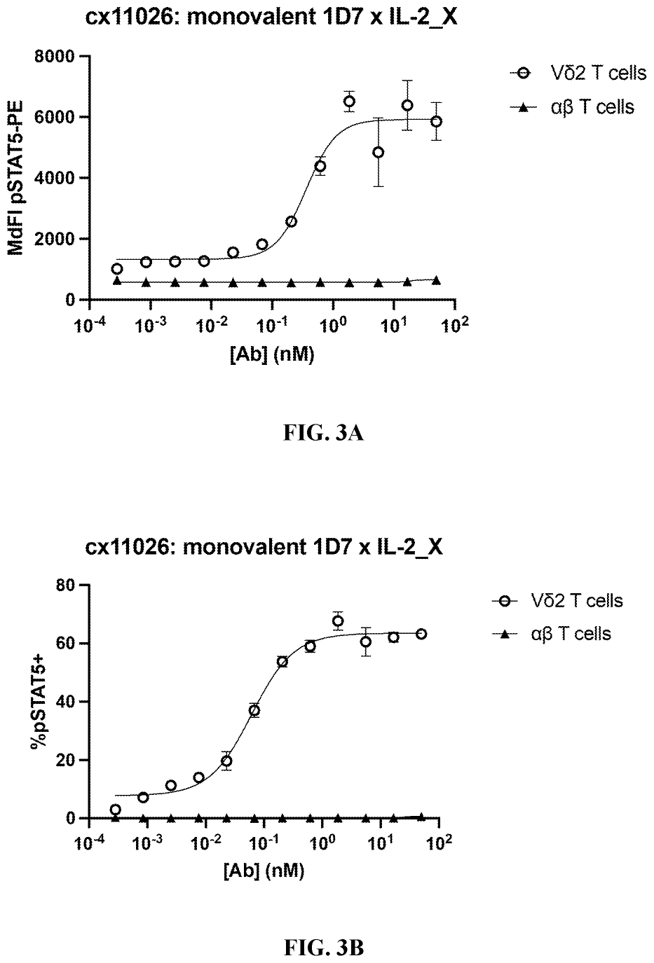

As shown in FIG. 3A-3B, treatment with monovalent anti-γδTCR-1D7 targeted IL-2_X led to a dose-dependent increase in the percentage of pSTAT5+Vδ2+γδ T cells (FIG. 3B) and the pSTAT5 median fluorescent intensity on Vδ2+γδ T cells (FIG. 3A). In contrast, there was no detectable pSTAT5 on αβ T cells at any of the concentrations tested.

### Example 4: Treatment with γSTCR-Targeted Low Affinity IL-2_X Potently Expands Vδ2⁺ γδ T Cells

IL-2 enhances the proliferation and effector function of γδ T cells. To determine the effect of γSTCR-targeted IL-2_X on proliferation, PBMCs were isolated and labeled with CellTrace Violet and added 300,000 per well as described in Example 2. The cells were treated with a titration of the fusion protein comprising IL-2_X fused to the C-terminal of either anti-γδTCR-1D7 or a non-targeted control VHH starting at an initial concentration of 100 nM and titrating across the plate 1:5 in duplicate. The plates were incubated at 37° C./5% C02 for 7 days. The cells were labeled with the following fluorescently conjugated antibodies: CD3-BV785, γSTCR-FITC, Vδ2-PE and the viability dye propidium iodide for 30 minutes at 4° C. The cells were washed and analyzed by flow cytometry.

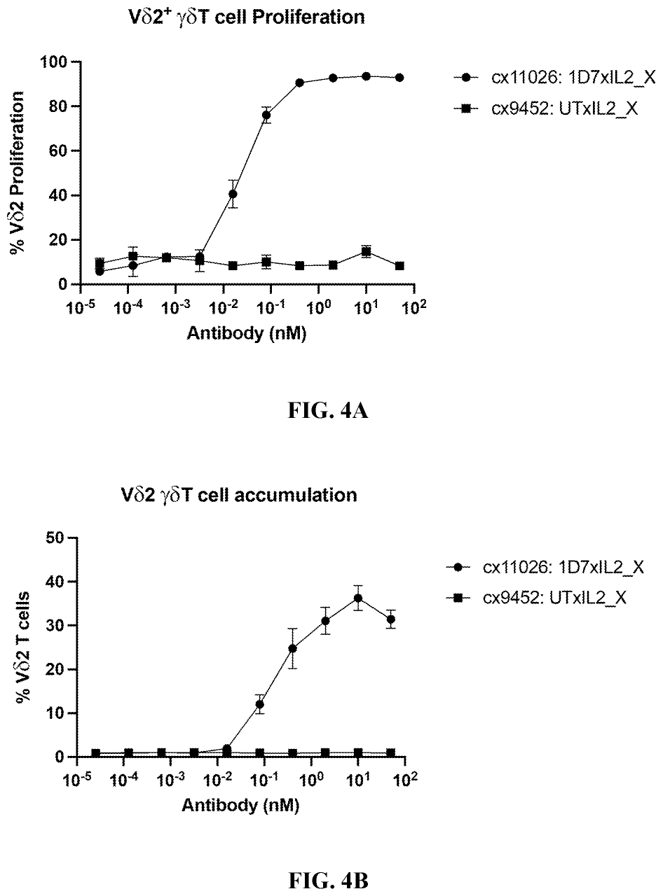

As shown in FIG. 4A-4B, treatment with anti-γδTCR-1D7 (cx11026) targeted IL-2_X led to a dose-dependent increase in the proliferation of Vδ2 γδ T cells (FIG. 4A). Additionally, the percentage of γδ T cells amongst the total CD3⁺ T cell population substantially increased with anti-γδTCR-1D7 targeted IL-2_X treatment (FIG. 4B). The non-targeted VHH (cx9452) fused to IL-2_X did not promote the proliferation of either Vδ2 γδ T cells demonstrating target specificity.

### Example 5: Treatment with a Bispecific Anti-γSTCR/Anti-Human Serum Albumin (HSA) Targeted Low Affinity IL-2_X Enhances Vδ2⁺ γδ T Cell Proliferation

An alternative monovalent format anti-γδTCR targeted IL-2 polypeptide was developed, which comprised an anti-γδTCR VHH domain and an anti-human serum albumin (HSA) VHH domain paired with a low affinity IL-2_X. To test this construct, PBMCs were isolated and labeled with CellTrace Violet and added 300,000 per well as described in Example 2. The cells were treated with a titration of the bispecific fusion protein comprising IL-2_X fused to the C-terminus of anti-HSA VHH, which was fused to the C-terminus of anti-γδTCR-1D7 VHH starting at an initial concentration of 25 nM and titrating across the plate 1:5 in duplicate. After 7 days in culture, the cells were labeled with the following fluorescently conjugated antibodies: CD3-BV785, γδTCR-FITC, Vδ2-PE and the viability dye propidium iodide for 30 minutes at 4° C. The cells were washed and analyzed by flow cytometry. Proliferation of Vδ2⁺ γδ T cells was assessed by CTV dilution of viable CD3+γδ⁺ Vδ2⁺ T cells.

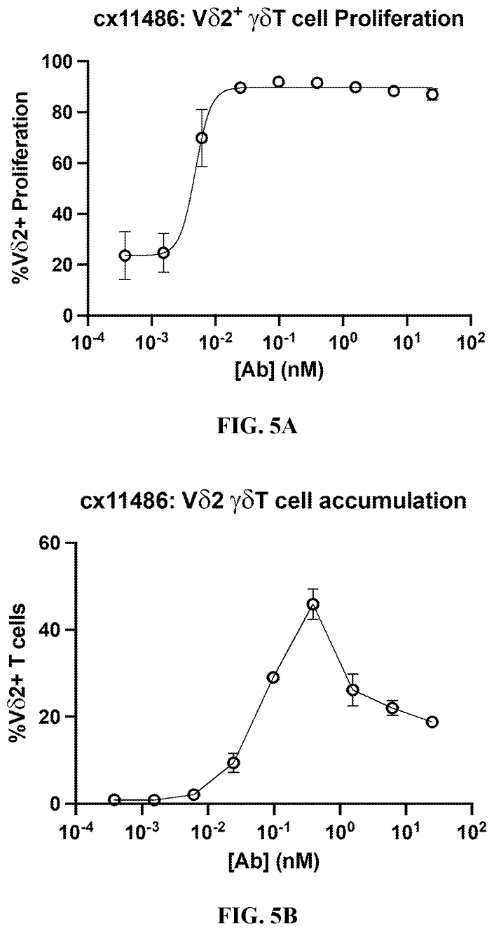

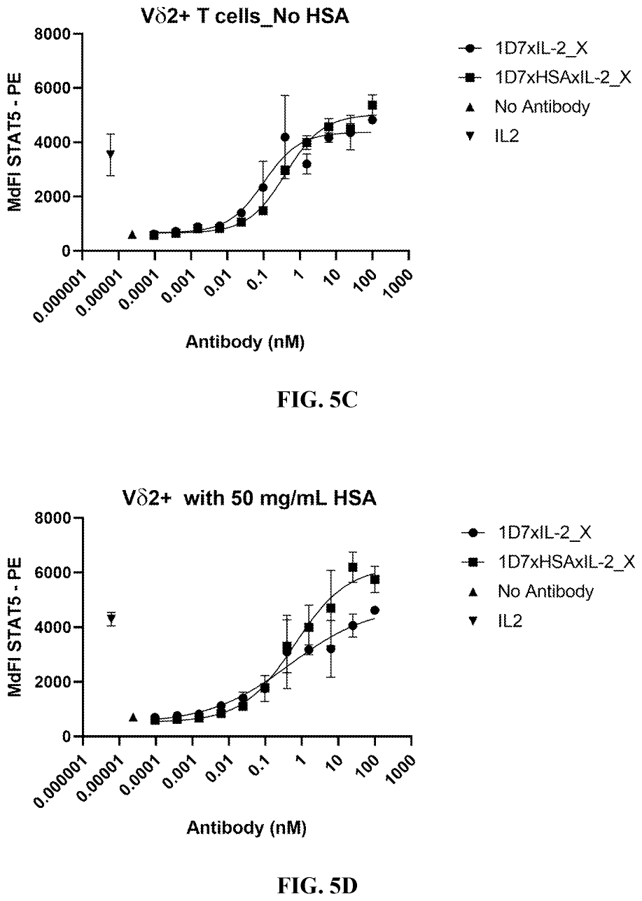

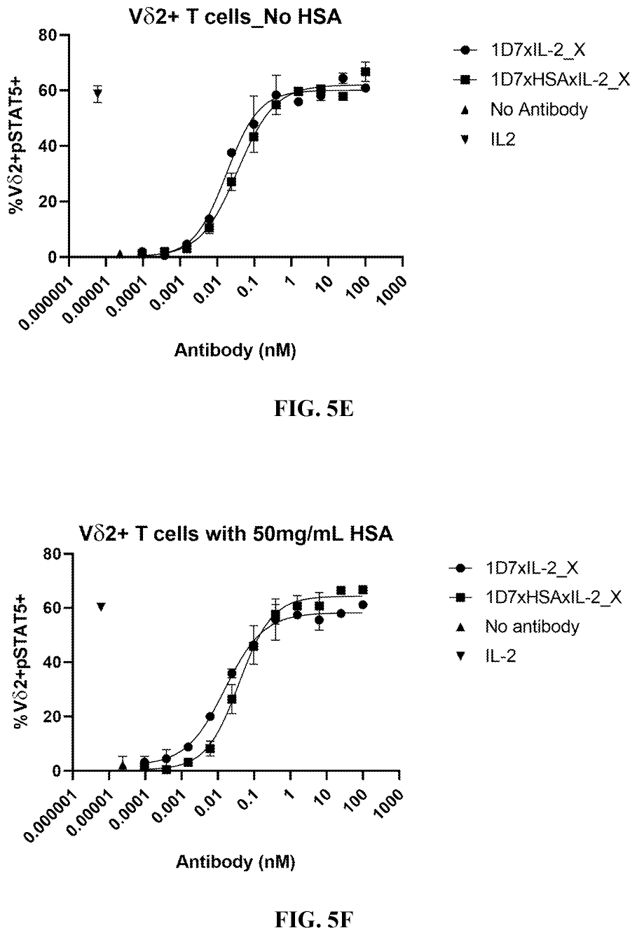

As shown in FIG. 5A-5B, treatment with the alternative format bispecific anti-γδTCR-1D7 VHH×anti-HSA VHH targeted IL-2_X (cx11486) led to a dose-dependent increase in the proliferation (FIG. 5A) and accumulation of Vδ2 γδ T cells (FIG. 5B).

In further studies the monovalent anti-γδTCR-1D7 VHH×anti-HSA VHH targeted IL-2_X (1D7×HSA×IL-2_X; cx11486) and monovalent 1D7×IL-2_X (cx11026) molecules were evaluated in an STAT5 signaling assay in the presence and absence of free HSA. To assess STAT5 phosphorylation frozen PBMCs (previously isolated from donor leukopaks) were thawed and washed twice using CTL thawing reagent. Cells were resuspended at 20×10\hat{ }6 cells/mL in complete assay media and surface stained in PBS for 15 minutes at 4° C. with Zombie Aqua™ (diluted 1:500). The cells were washed once with PBS/2% FBS and labeled with the following fluorescently conjugated antibodies: anti-CD3-BV785, anti-Vγ2-BV421 for 30 minutes at 4 C. After washing 400,000 PBMCs were seeded per well in 100 μL of media in a 96 well U-bottom plate. The cells were treated with a titration of the fusion protein added at 4× the final starting concentration of 100 nM and titrated down 1:4 in assay media, untreated and 50 IU/mL of IL-2 controls were included. The final volume of each well was raised to 200 uL with complete assay media or complete media with 50 mg/mL of HSA final concentration and the cells were incubated for 25 minutes at 37 C/5% CO2. Post-incubation, cells were fixed in 100 uL/well of ice cold BD cytofix buffer for 30 minutes at 4 C, washed with FACS buffer once, and then permeabilized with 100 uL/well of ice cold BD Phosflow Permeabilization buffer III for 30 minutes. Cells were washed 3× with ice cold 1× eBioscience permeabilization buffer. The cells were then stained overnight at 4 C with the pSTAT5-PE antibody diluted in 1× eBioscience permeabilization buffer. The following morning, cells were washed 2× with FACS buffer, resuspended in 70 ul of FACS buffer and levels of phosphorylated STAT5 (“pSTAT5”) on cells were measured by flow cytometry (Novocyte) gating on CD3-BV785⁺ Vγ2-BV421⁺ to identify γδ T cells.

As shown in FIG. 5C-5F titration of both monovalent anti-γδTCR-1D7 targeted IL-2_X formats led to the dose-dependent increase in the pSTAT5 median fluorescent intensity on Vδ2⁺ γδ T cells (FIG. 5C-5D) and the percentage of Vd2+ g/d T cells that bound to the phospho-specific STAT5 antibody (FIG. 5E-5F), indicating that both formats prompt internal IL-2 signaling. The presence of HSA (FIGS. 5D and 5F) did not inhibit the Vd2+ T cell response to IL-2 signaling on either protein.

### Example 6: Development of γδ TCR-Binding VHH Domains

Single domain antibodies targeting human γδ TCR were generated via immunization of llamas with enriched γδ T-cells and a heterodimeric knob-in-hole construct consisting of human gamma9 extracellular domain cloned with a reduced effector knob Fc paired with human delta2 extracellular domain containing a reduced effector hole Fc. Following the development of specific anti-γδ TCR antibody titers, llama peripheral blood mononuclear cells (PBMCs) were isolated from 500 mL of blood from the immunized animal and total mRNA was isolated using the Qiagen RNeasy Maxi Kit and subsequently converted to first strand cDNA using Thermo Superscript IV Reverse Transcriptase and oligo-dT priming. VHH sequences were specifically amplified via PCR using the cDNA as template and cloned into a yeast surface display vector as VHH-Fc-AGA2 fusion proteins. The Fc was a human IgG1 Fc (SEQ ID NO: 32) or, in some cases, a variant IgG1 Fc with reduced effector function (e.g., Fc xELL; SEQ ID NO: 33).

Yeast libraries displaying the VHH-Fc-AGA2 fusion proteins were enriched using recombinant forms of the γδ TCR ECD via magnetic bead isolation followed by fluorescence activated cell sorting (FACS). Sorted yeast were plated out and isolated colonies were picked into 96-well blocks and grown in media that switched the expression from surface displayed VHH-Fc to secretion into the media. Supernatants from the 96-well yeast secretion cultures were applied to 293F cells transiently transfected with γδ TCR (γδ TCR positive) or untransfected 293F cells (γδ TCR negative), washed, treated with fluorophore labelled anti-human IgG1 Fc secondary antibody, and analyzed by 96-well flow cytometry.

Nucleic acid sequences encoding VHHs that bound to γδ TCR positive cells and not to γδ TCR negative cells were cloned in-frame with a human Fc xELL encoding region into mammalian expression vectors, and expressed by transient transfection in HEK293 Freestyle cells (293F cells) or CHO cells using polyethylenimine. Supernatant was collected after 3-7 days, secreted recombinant protein was purified by protein A chromatography, and concentration was calculated from the absorbance at 280 nm and extinction coefficient.

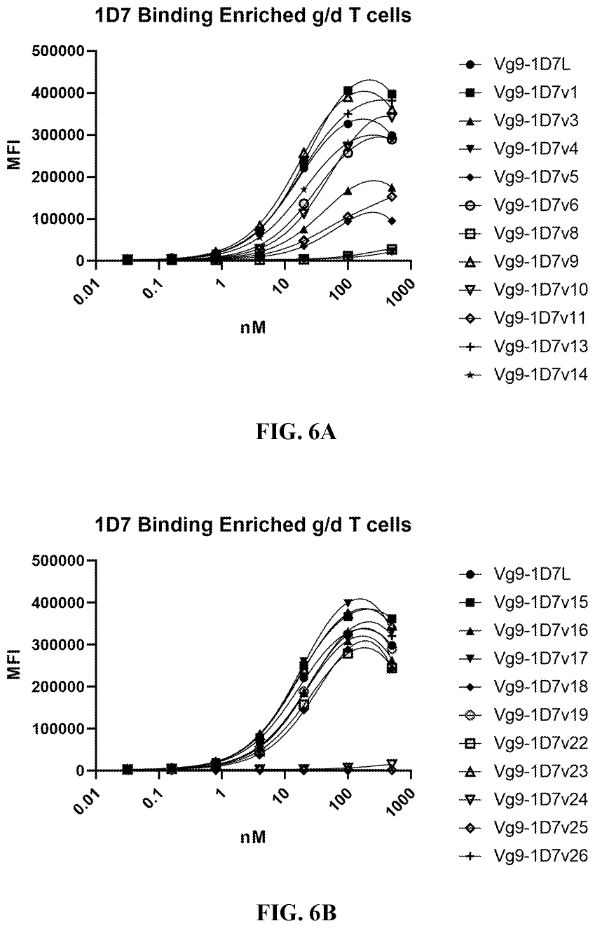

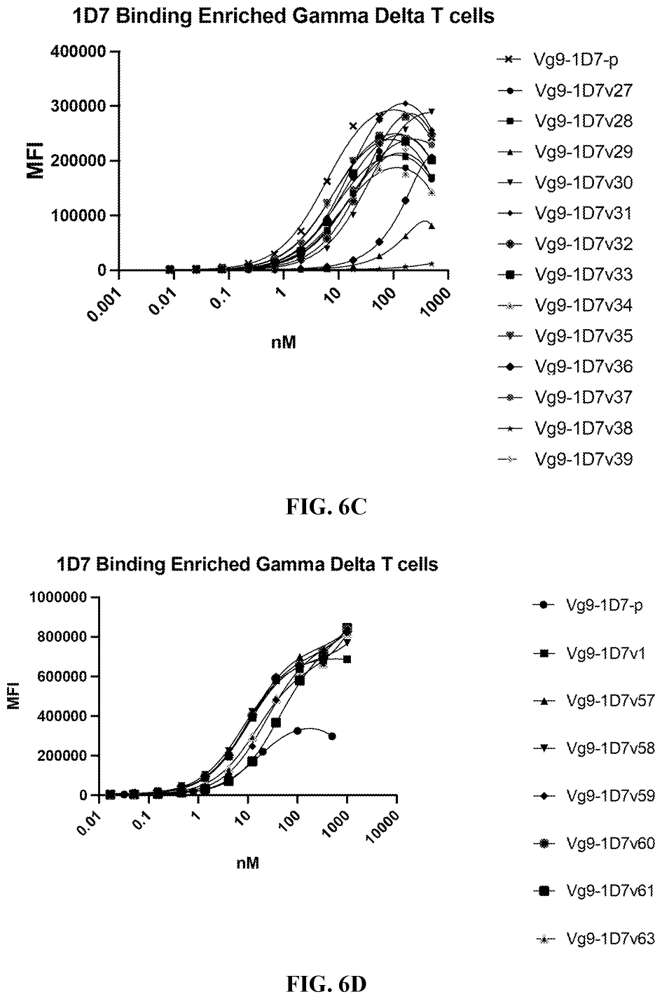

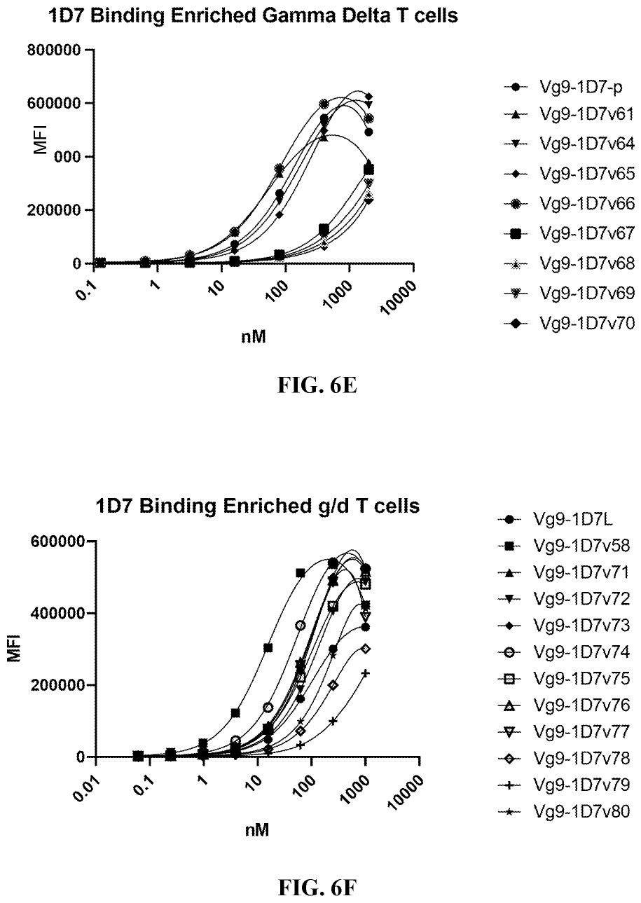

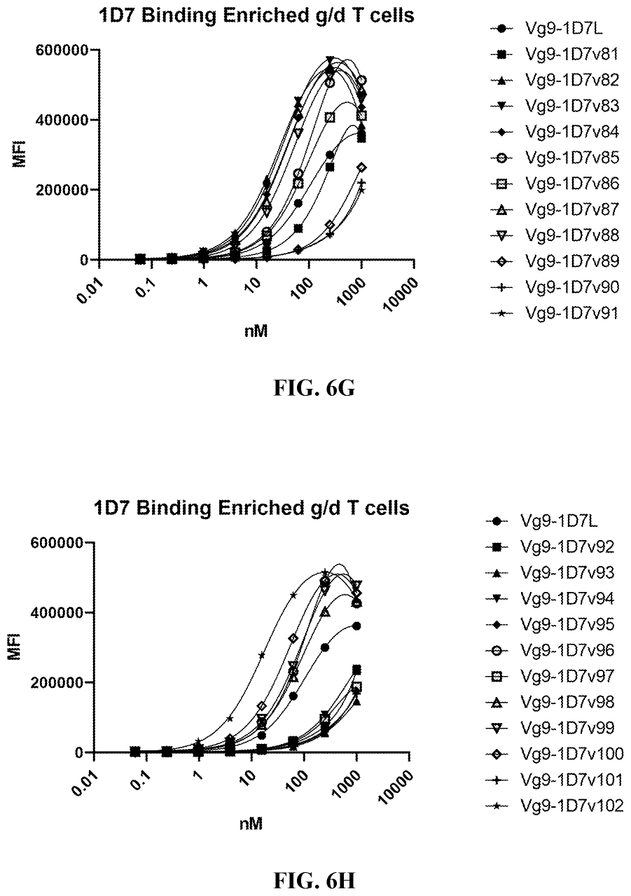

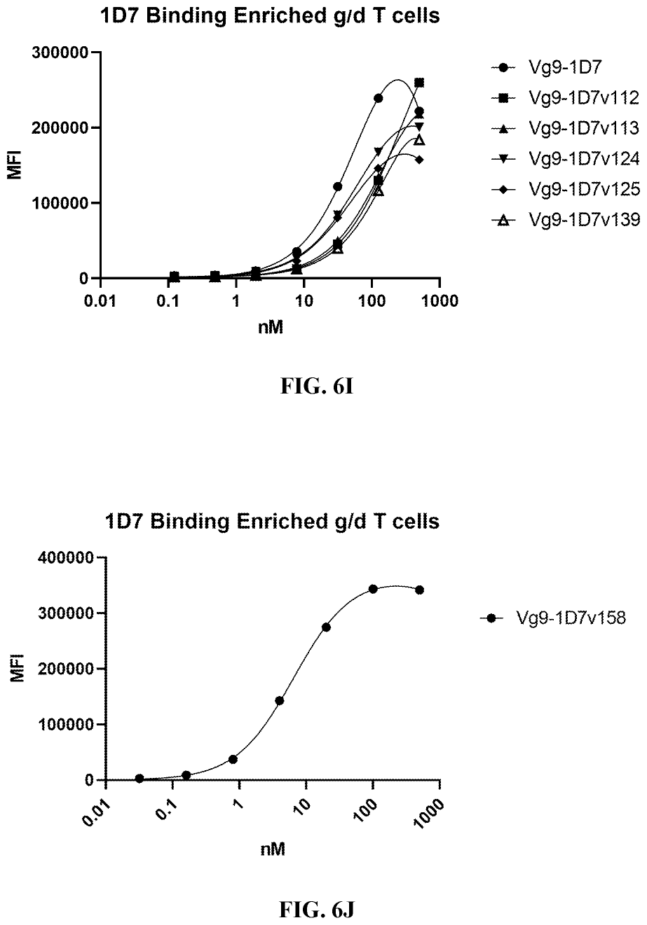

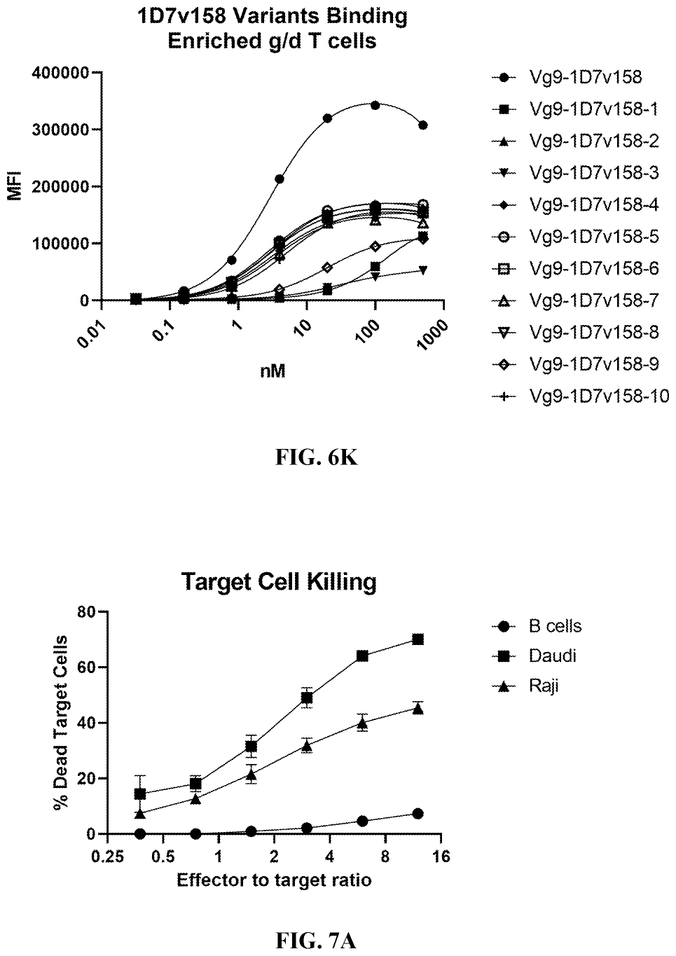

Binding of the γδ TCR-binding VHH, 1D7, and humanized versions thereof formatted as monomeric VHH-hIgG1-Fc fusion proteins using a non-dimerizing human IgG1 Fc variant region lacking a hinge Fc NNT, was assessed by flow cytometry on fresh or frozen expanded human Vδ2+ γδ T cells. Vδ2⁺ γδ T cells were expanded from PBMCs from healthy human donor peripheral blood leukopacks. Freshly isolated PBMCs were resuspended at 1.0×10\hat{ }6 cells/mL in complete RPMI media supplemented with 10% FBS, 5 uM zoledronate, and 500 IU/ml IL-2. On day 3, half of the media was removed, and IL-2 was added at a final concentration of 500 IU/mL. For the remainder of the expansion, the media was changed and IL-2 was added at a final concentration of 500 IU/ml every 2-3 days for a total of two weeks. Purity was assessed by flow cytometry and typically was greater than 80% Vδ2⁺ γδ T cells. Fresh expanded γδ T cells were plated in a 96-well plate at: 30,000 cells per well or thawed expanded γδ T cells were plated in a 96-well plate at: 50,000 cells per well in FACS buffer (PBS, 1% BSA, 0.1% NaN₃, pH 7.4). Untransfected HEK293F cells were used as a γδ TCR-negative control and plated at 30,000 cells per well in a separate plate. Test polypeptides were then diluted to 2× the final concentration of 1000 nM and 3-, 4-, and 5-fold serial dilutions were made. FACS buffer with no polypeptide was used as a secondary antibody-only control. Polypeptide dilutions were added to an equal volume of cells, and assay plates were incubated for 30 minutes at 4° C. After washing twice with 150 μL of FACS buffer per well, the cells were resuspended in FACS buffer with fluorescently-labeled anti-human Fc antibody diluted 1:1000 or 1:2000 to detect binding. Assay plates were incubated at 4° C. for 20 minutes and washed once with 150 μL of FACS buffer per well. Binding to γδ T cells was determined by flow cytometry using an Intllicyt iQue Plus and anti-human A647 median fluorescence intensity was calculated using onboard software. The data was plotted and analyzed using GraphPad Prism analysis software. The binding curves for the initial humanized 1D7 VHH fusion proteins are shown in FIG. 6A-6I. The binding curve for an improved humanized variant 1D7v158 is shown in FIG. 6J, this variant has a more favorable pI profile and has a binding affinity comparable to the parental VHH. Additional humanized variants based on 1D7v158 were also generated and the binding curves of the parental 1D7v158 and the further variants are shown in FIG. 6K.

The affinities (K_{D}) of the 1D7 (“1D7-p”) and humanized versions thereof was determined from the flow cytometry binding data using a nonlinear fit model, and are shown in Table 2.

The amino acid sequences of the 1D7 VHH and humanized versions thereof are provided in the Table of Certain Sequences provided below. It is provided that the amino acid at residue 114 in any of the disclosed 1D7 VHH domains may be a lysine (K) an aspartate (D), a glutamate (E), or an arginine (R). For example, the parental 1D7 VHH (SEQ ID NO:2) comprises a lysine (K) at residue 114, hu1D7v39 VHH (SEQ ID NO:97) comprises an arginine (R) at residue, and hu1D7v158 (SEQ ID NO:158) comprises a glutamate (E) at residue 114. Accordingly, the residue at position 114 may be substituted with a lysine, aspartate, glutamate, or arginine.

TABLE 2

| clone | K_{D} (nM) | clone | K_{D} (nM) | clone | K_{D} (nM) |
|---|---|---|---|---|---|
| 1D7-p | 6.09 | 1D7v35 | 8.40 | 1D7v86 | 126.20 |
| 1D7v1 | 5.23 | 1D7v36 | 1505.00 | 1D7v87 | 44.90 |
| 1D7v2 | | 1D7v37 | 17.45 | 1D7v88 | 61.47 |
| 1D7v3 | 51.15 | 1D7v38 | 20172.00 | 1D7v89 | 1950.00 |
| 1D7v4 | 8858.00 | 1D7v39 | 7.99 | 1D7v90 | 95.56 |
| 1D7v5 | 108.40 | 1D7v57 | 10.49 | 1D7v91 | 154.10 |
| 1D7v6 | 35.14 | 1D7v58 | 8.07 | 1D7v92 | 104.00 |
| 1D7v7 | | 1D7v59 | 23.79 | 1D7v93 | 1087.00 |
| 1D7v8 | 7873.00 | 1D7v60 | 9.67 | 1D7v94 | 752.20 |
| 1D7v9 | 17.93 | 1D7v61 | 41.28 | 1D7v95 | 196.30 |
| 1D7v10 | 60.94 | 1D7v62 | | 1D7v96 | 191.40 |
| 1D7v11 | 42.22 | 1D7v63 | 14.73 | 1D7v97 | 1047.00 |
| 1D7v12 | | 1D7v64 | 195.30 | 1D7v98 | 113.80 |
| 1D7v13 | 18.20 | 1D7v65 | 347.40 | 1D7v99 | 115.90 |
| 1D7v14 | 21.47 | 1D7v66 | 93.27 | 1D7v100 | 61.91 |
| 1D7v15 | 16.42 | 1D7v67 | 1744.00 | 1D7v101 | 270.50 |
| 1D7v16 | 24.63 | 1D7v68 | 722.30 | 1D7v102 | 17.96 |
| 1D7v17 | 20.09 | 1D7v69 | 1014.00 | 1D7v112 | 226.5 |
| 1D7v18 | 42.48 | 1D7v70 | 440.80 | 1D7v113 | 191.8 |
| 1D7v19 | 26.73 | 1D7v71 | 135.40 | 1D7v124 | 72.19 |
| 1D7v20 | | 1D7v72 | 181.90 | 1D7v125 | 59.04 |
| 1D7v21 | | 1D7v73 | 180.20 | 1D7v139 | 340.8 |
| 1D7v22 | 29.14 | 1D7v74 | 60.39 | 1D7v158 | 6.544* |
| 1D7v23 | 18.13 | 1D7v75 | 118.10 | 1D7v158 | 2.962\hat{ } |
| 1D7v24 | 9444.00 | 1D7v76 | 125.10 | 1D7v158-1 | 205.0 |
| 1D7v25 | ~866246 | 1D7v77 | 155.60 | 1D7v158-2 | 3.281 |
| 1D7v26 | 29.04 | 1D7v78 | 481.20 | 1D7v158-3 | 27.97 |
| 1D7v27 | 9.59 | 1D7v79 | 496.80 | 1D7v158-4 | 3.126 |
| 1D7v28 | 15.68 | 1D7v80 | 646.20 | 1D7v158-5 | 2.820 |
| 1D7v29 | 7231.00 | 1D7v81 | 843.40 | 1D7v158-6 | 2.816 |
| 1D7v30 | 38.40 | 1D7v82 | 30.47 | 1D7v158-7 | 3.468 |
| 1D7v31 | 16.69 | 1D7v83 | 36.32 | 1D7v158-8 | 2.930 |
| 1D7v32 | 35.74 | 1D7v84 | 43.63 | 1D7v158-9 | 20.01 |
| 1D7v33 | 12.98 | 1D7v85 | 168.30 | 1D7v158-10 | 4.975 |
| 1D7v34 | 11.66 | | | | |

*determined from data in FIG. 6J
\hat{ }determined from data in FIG. 6K

### Example 7: PBMCs Expanded with γδTCR-Targeted IL-2 Selectively Kill Transformed B Cell Lines while Sparing Normal B Cells

Naturally occurring Vγ9Vδ2 T cells can distinguish healthy cells from transformed cells due to the increased production of stress related ligands that are produced by cancerous cells leading to their elimination. The ability of Vγ9Vδ2 T cells expanded with a γδTCR-targeted TL,-2 molecule (cxl 1026) to selectively kill the Burkitt's B cell lymphoma cell lines Raji and Daudi while sparing normal healthy B cells isolated from an autologous donor was examined in a FACS based killing assay. γδ T-cells were expanded from thawed frozen PBMCs (from a healthy donor leukopak), briefly, resuspended to 1.0×10\hat{ }6 cells/mL, treated with 1 nM of cx11026 and incubated at 37 C/5% CO2 for 10 days, on day 5, cells were spun down, resuspended and redosed with 1 nM of cx11026. On day 10, cells were counted and 200,000 cells were taken for cell surface staining on the Novocyte. Cells were put into FACS tubes, washed once with 1 mL of FACs buffer and stained to assess purity using anti-human CD3-BV785 and anti-human Vδ2-PE in 100 μL of FACS buffer for 30 minutes at 4 C. The expanded cells were resuspended to 2.0×10 \hat{ }6 cell/ml for the killing assay. B cells were isolated, from thawed frozen PBMCs (from a healthy donor leukopak), using a STEMcell EasySep Human B cell isolation kit and resuspended at 1×10\hat{ }6 cells/mL in complete assay media. For the FACS assay Daudi, Raji, and isolated B cells were washed and labeled with CellTrace™ Violet (CTV) for 10 minutes at 37 C. The cells were resuspended in complete RPMI media and 10,000 cells were added in 150 ul of complete RPMI media per well in 96-well plates, and plates incubated for 20 minutes. Expanded Vγ9Vδ2 T cell effector cells were then added at a highest concentration of 12:1 ratio of effector to target cells in 50 uL and titrated down the plate 1:2 in triplicate, the cells were incubated at 37 C/5% CO2 overnight. The following day, the cells were labeled for 30 minutes at 4 C with anti-human CD3-BV785, anti-human-Vd2-PE, the viability marker Propidium Iodide, and apoptosis marker Apotracker-Green. The cells were washed twice in FACS buffer and analyzed by flow cytometry (Novocyte). Target cell killing is shown in FIG. 7A and the live cell count is shown in FIG. 7B.

As shown in FIG. 7A-7B PBMCs expanded with a γδTCR-targeted IL-2 molecule (1D7×IL-2_X) selectively kill Daudi and Raji the Burkitt's B cell lymphoma cells while largely sparing normal healthy B cells.

### Example 8: PBMCs Expanded with γδTCR-Targeted IL-2 Demonstrate Potent Cytotoxicity Across a Broad Panel of Tumor Cell Lines

The ability of PBMCs expanded with a γδTCR-targeted IL-2 molecule (cx11026) to kill target cell lines derived from a variety of hematological and solid tumors was evaluated. Briefly, PBMCs isolated from Leuko 75 were expanded with 1 nM of cx11026 essentially as described in Example 7, after a two-week expansion protocol, the cells were resuspended to 2.0×10\hat{ }6 cell/ml for the killing assay. 1-2×10\hat{ }6 target cells were washed with HBSS, labeled with Cyto-ID red for 6 minutes following the kit instructions, cells were then resuspended in complete RPMI media and seeded in 100 ul/well (10,000 cells). Control wells using untreated PBMCs, or containing only target cells were included. Adherent cells were plated in flat bottom and non-adherent cells were plated on Poly-L coated 96-well plates, plates were incubated for 20 minutes at room temperature, followed by 2 hours at 37 C/5% CO2 prior to addition of effector cells. Effector cells were then added at a 10:1 ratio to the target cells, 100,000/well in 50 μL volume. Caspase 3/7-green was added at a final concentration of 1.25 μM in 50 μL per well. The cells were allowed to settle for 20 minutes at room temperature and cell killing was analyzed using an Incucyte cell imager. After 30 minutes to equilibrate the temperature, target cell killing was assessed every 1.5 hours by overlap of the caspase3/7-green with cyto-ID red. After masks were made for target and dead cells the overlap the data was plotted and analyzed using GraphPad Prism analysis software.

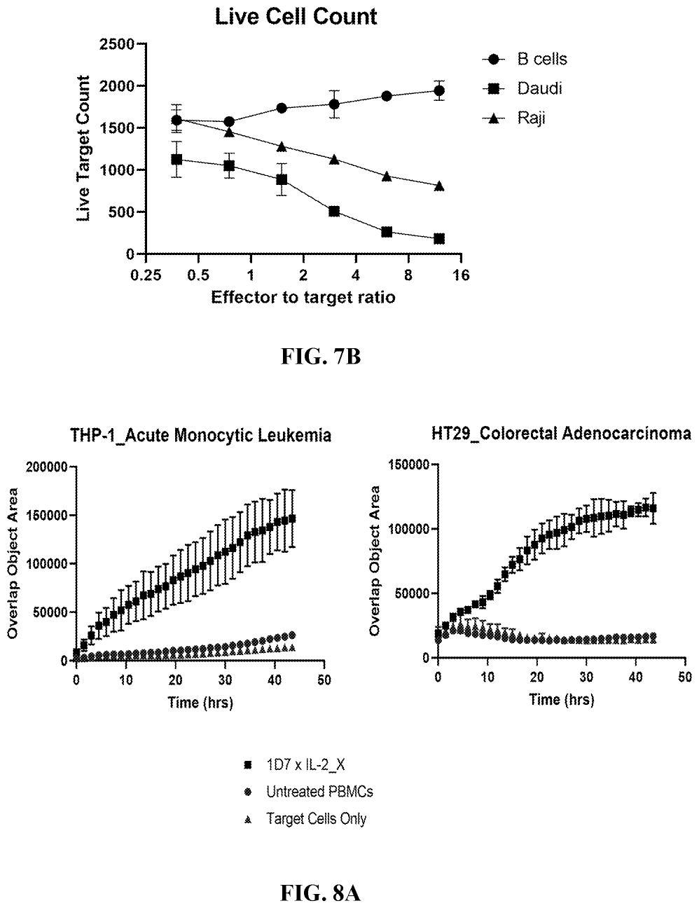

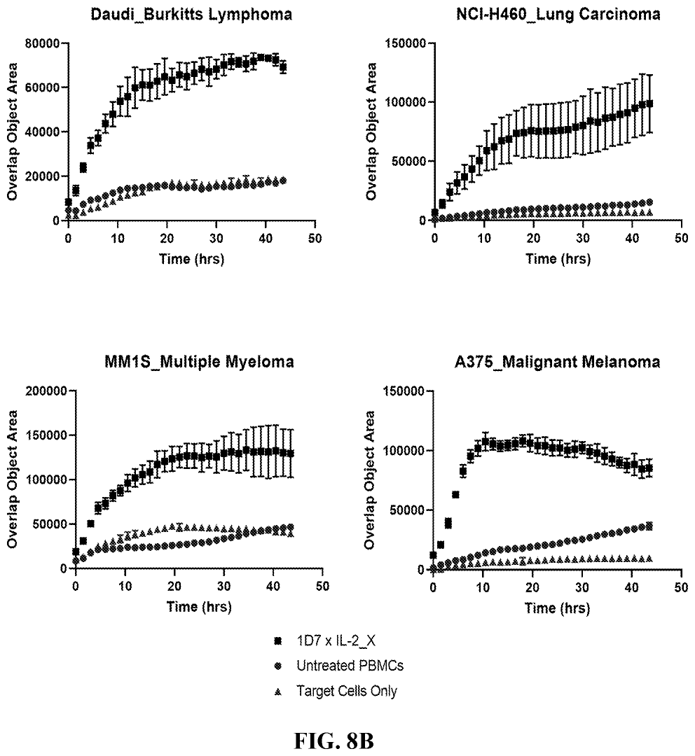

As shown in FIG. 8A-8B treatment of PMBCs with a γδTCR-targeted IL-2 molecule (1D7×IL-2_X) led to the expansion of Vγ9Vδ2 T cells and broad anti-tumor killing activity across a wide variety of cell lines, including THP-1 acute monocytic leukemia, HT29 colorectal adenocarcinoma, Daudi Burkitts lymphoma, NCI-H460 lung carcinoma, MM1S multiple myeloma, and A375 malignant melanoma cell lines.

### Example 9: Expansion and Activity of Vγ9Vδ2 T Cells from Cancer Patient PBMCs and DTCs

The ability of γδTCR-targeted IL-2 to expand the Vγ9Vδ2 T cell population present in patient samples from hematological and solid tumor indications was evaluated using the monovalent γδTCR-targeted IL-2 molecule 1D7×IL-2_X (cx11026). Briefly, cancer patient PBMCs and dissociated tumor cells (DTCs) from several indications including ovarian, colorectal (CRC), acute monocytic leukemia (AML) or B cell acute lymphoblastic leukemia (B-ALL) were thawed and labeled with the proliferative dye CellTrace Violet (CTV) for 10 minutes at 37 C. After washing the cells were resuspended in complete media and 0.5-1.0×10 \hat{ }6 cells were plated per well. The cells were either left untreated or treated with cx11026 at a final concentration of 1 nM and proliferation was assessed one week later by flow cytometry by gating on Vδ2+ T cells and determining CTV dilution.

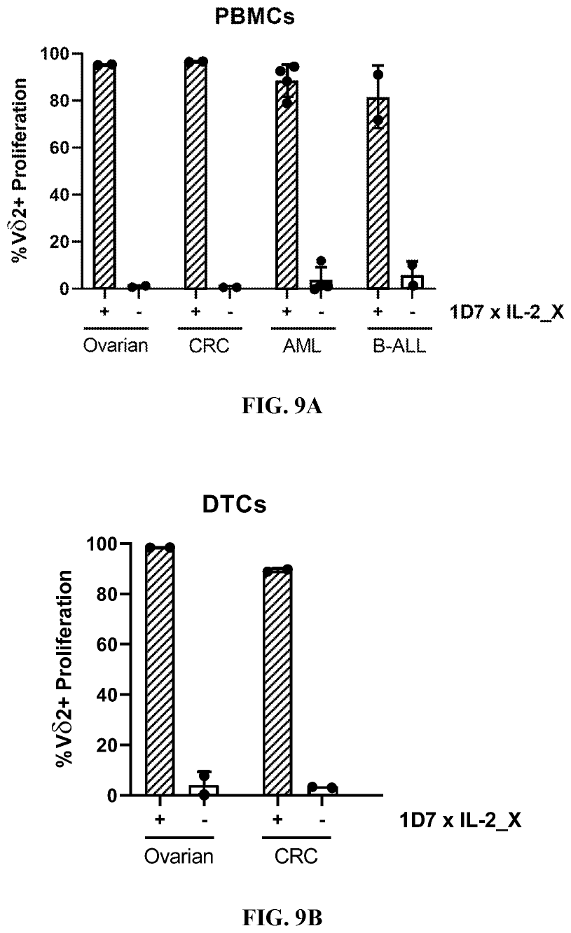

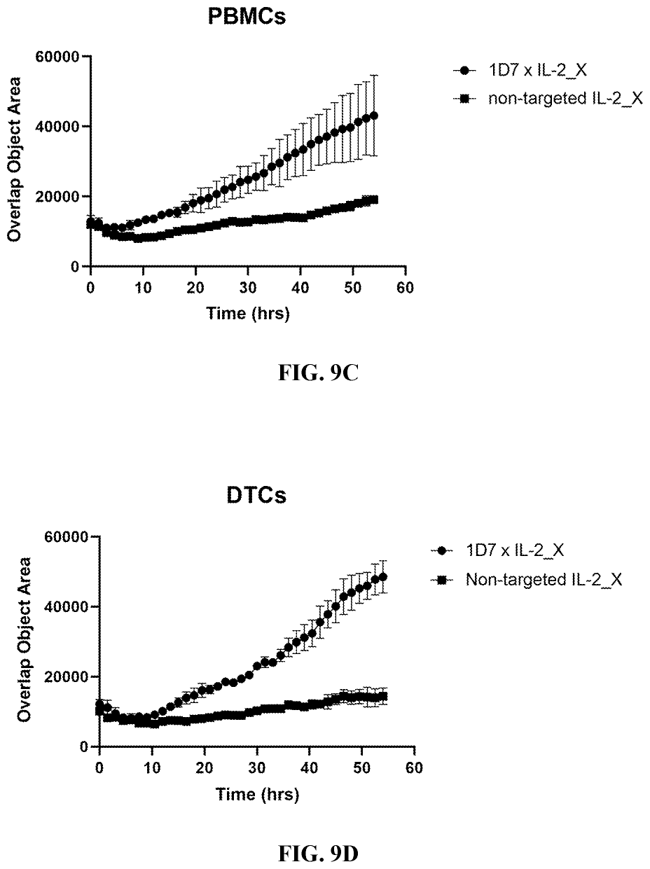

As shown in FIG. 9A-9B Vγ9Vδ2 T cells were present in all the cancer patient samples tested. Moreover, treatment with a monovalent γδTCR-targeted IL-2 molecule (1D7×IL-2_X) led to a dramatic increase in Vγ9Vδ2 T cell proliferation across all tumor indications indicating the cells are functional and respond to treatment with a monovalent γδTCR-targeted IL-2 molecule.

PBMCs or DTCs from an ovarian cancer patient and the matched tumor cell line OVCAR3 were used to determine if γδTCR-targeted IL-2 expanded PBMCs and DTCs from patient samples are capable of mediating tumor cell killing. Briefly, ovarian cancer patient PBMCs and DTCs were thawed, washed and resuspended to 1.0×10\hat{ }6 cells/mL. 2 mL of cells, or 2 million cells per donor, were dosed with 1 nM of either the monovalent γδTCR-targeted IL-2 molecule 1D7×IL-2_X (cx11026) or the non-targeted modified IL-2_X (cx9452) and incubated at 37 C/5% CO2 for 10 days. On day 5, cells were counted using trypan-blue. Half of the cells were spun down resuspended to a final total volume of 2 mL of complete media, and redosed with 1 nM of the same test article. On day 10, cells were counted and 200,000 cells were taken for cell surface staining on the Novocyte. Cells were put into FACS tubes, washed once with 1 mL of FACs buffer and stained to assess purity using anti-human CD3-BV785 and anti-human Vδ2-PE in 100 μL of FACS buffer for 30 minutes at 4 C. The expanded cells were resuspended to 2.0×10 \hat{ }6 cell/ml for the killing assay. OVCAR3 target cells were counted and 1-2×10 \hat{ }6 cells were removed, washed, and labeled with Cyto-ID red for 6 minutes following the kit instructions. The labeled OVCAR3 cells were resuspended in complete RPMI media and seeded in 100 uL/well (10,000 cells). Effector cells were added at a 10:1 ratio of effector to target cells (100,000/well) in 50 uL volume. Caspase 3/7-green was added at a final concentration of 1.25 uM in 50 uL per well. The cells were allowed to settle for 20 minutes at room temperature and cell killing was analyzed using an Incucyte cell imager. After 30 minutes to equilibrate the temperature, target cell killing was assessed every 1.5 hours by overlap of the caspase3/7-green with cyto-ID red. After 4 days, the cells were removed from the cell imager. After masks were made for target and dead cells the overlap data was graphed and analyzed using Prism.

As shown in FIG. (9C-9D) treatment of patient derived PMBCs (FIG. 9C) or DTCs (FIG. 9D) with a γδTCR-targeted IL-2 molecule (1D7×IL-2_X) led to improved killing of OVCAR3 target cells.

### Example 10: Development and Evaluation of Bispecific γδ TCR-Targeted IL-2 Molecules

Bispecific γδTCR-targeted IL-2 molecules with binding domains for γδTCR and a target cell antigen (5T4), were generated comprising an anti-γδTCR (5C8 or 5D7) VHH domain and an anti-5T4 VHH domain linked to a low affinity IL-2_Y (5T4×δγTCR xIL-2_Y molecules cx907land cx9075, respectively), also generated were a non-targeted-IL-2_Y (cx1603), and two monospecific/monovalent γδTCR-targeted IL-2 molecules comprising an anti-γδTCR (5C8 or 5D7) VHH and a VHH for an irrelevant target (monovalent δγTCR×IL-2_Y molecules cx7537 and cx7530, respectively). The Vγ9Vδ2 T cell-mediated killing activity was assessed in a killing assay. Briefly, expanded γδ T cells were prepared essentially as described in Example 6, and enriched γδ and αβ T cells were isolated from PBMCs (from a healthy donor leukopak) using Easysep Stemcell enrichment kits. T47D breast cancer target cells expressing 5T4 were labeled with cytoID red following the kit instructions (Enzo), washed and resuspended in complete RPMI media to a final concentration of 0.1×10\hat{ }6, and 100 μL (10 k) of resuspended cells were plated per well in a 96-well flat bottom moat plate and allowed to settle for 15 minutes at room temperature and then placed in the 37C incubator to adhere for 2 hours. Expanded Vγ9Vδ2 T cells, enriched γδ T cells, or enriched αβ T cells were resuspended in complete RPMI media to 1.0×10 \hat{ }6 cells/ml and added at a 5:1 effector to target ratio to the T47D cells. Caspase 3/7-Green was added at a final concentration of 1.25 M to detect apoptotic cells. The test articles were added to the plates at a final concentration of 30 nM and titrated down the plate 1:6. Target cell killing was analyzed using an Incucyte cell imager and determining the overlap of cyto ID red and caspase 3/7 indicating apoptotic T47D target cells. After masks were made for target and dead cells the overlap data was the data was plotted and analyzed using GraphPad Prism analysis software.

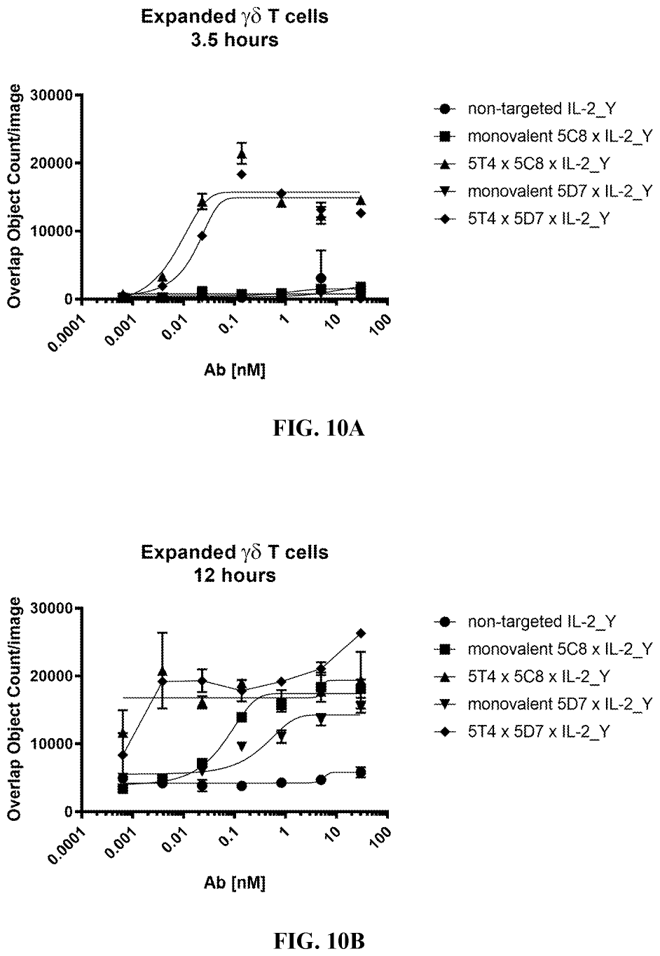

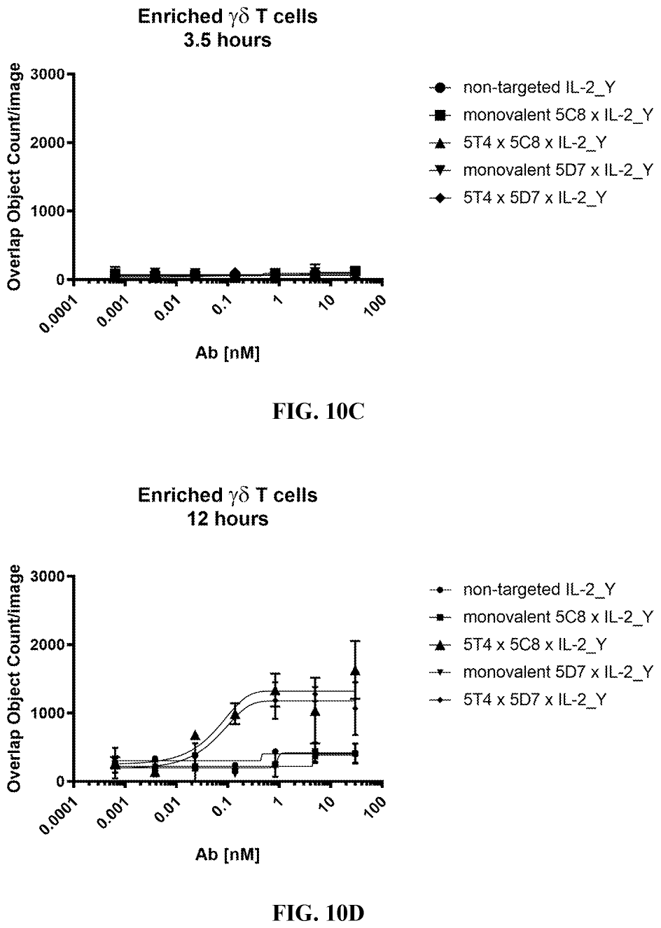

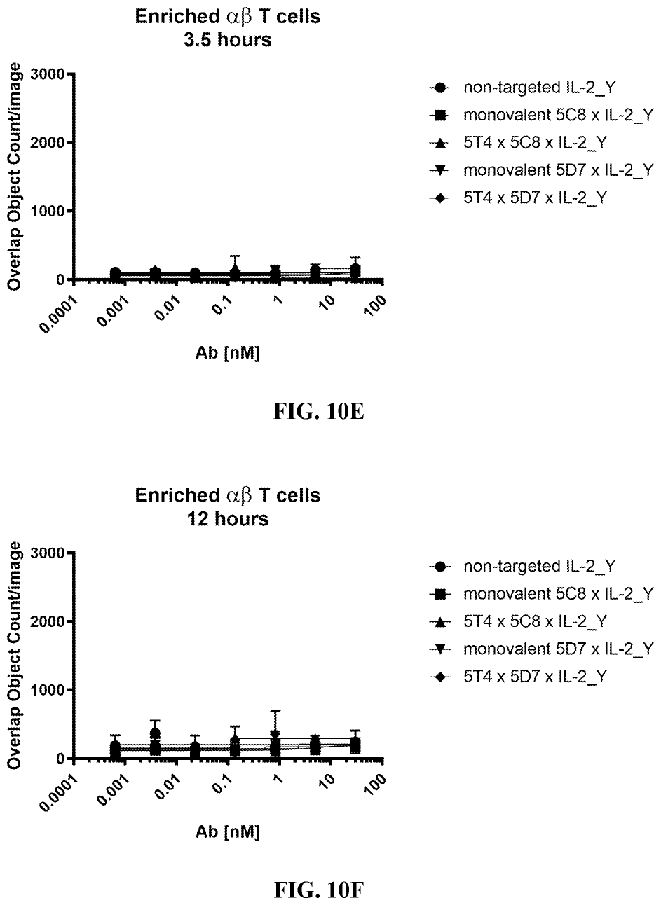

As shown in FIG. 10A-10D the bispecific constructs exhibited higher activity demonstrating that the addition of a target cell binding domain (e.g., 5T4) led to more rapid and potent killing of target cells (e.g., T47D) by both expanded γδ T cells (FIG. 10A-10B) and freshly isolated enriched γδ T cells (FIG. 10C-10D). The expanded γδ T cells exhibited higher activity. As shown in FIG. 10E-10F, enriched αβ T cells did not demonstrate killing activity demonstrating that the killing activity was specific for γδ T cells.

### Example 11: Enhanced In Vivo Expansion of γδ T Cells Upon Treatment with γδTCR-Targeted IL-2

IL-2 enhances the proliferation and effector function of γδ T cells. To determine the effect of γδTCR-targeted IL-2_X on Vγ9Vδ2 T cell proliferation in vivo, human PBMCs were engrafted into immunodeficient NSG mice and the mice were treated with a monovalent γδTCR-targeted IL-2_X comprising anti-γδ VHH domain 1D7v158. Briefly, frozen PBMCs that had been isolated from healthy donor leukopaks were thawed and γδ T cells were purified using the Stemcell Easysep Human γδ T cell isolated kit and the enriched γδ T cells were added back to the PBMCs totaling 10-20% of the total CD3⁺ population (PBMCs/γδ T cells). NSG mice (5 per group) were injected IV with 10×10 \hat{ }6 human PBMCs/γδ T cells in PBS. One day later, the mice were injected IV with a single 10, 1, or 0.1 mg/kg dose of the monovalent γδTCR-targeted IL-2_X (cx12168) or vehicle alone. One day after injection with cx12168 the mice were bled, and the levels of cx12168 in the serum were determined by ELISA. Additionally, the mice were bled 5, 7, 10, 14, 21, 28, and 35 days after the cells were injected. Blood samples were spun down and plasma removed. The red blood cells (RBCs) were lysed for 7 minutes using 1×RBC lysis buffer (Biolegend). The remaining cells were washed with FACS buffer and labeled in 100 μl of FACS buffer containing the following fluorescently labeled anti-human antibodies: γδ TCR-FITC, CD3-BV785, NKG2D-APC, Vγ₂-PE, DNAM1-PE/Cy7, CD45-APC/Cy7. After labeling the cells were washed, resuspended in FACS buffer, and analyzed using the Novocyte Quanteon flow cytometer. The number of Vγ9Vδ2 T cells was determined by gating on human CD45+CD3+ γδ TCR+Vδ2+ T cells.

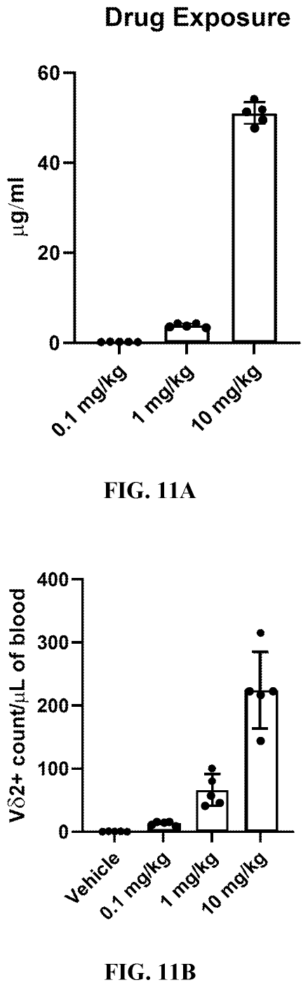

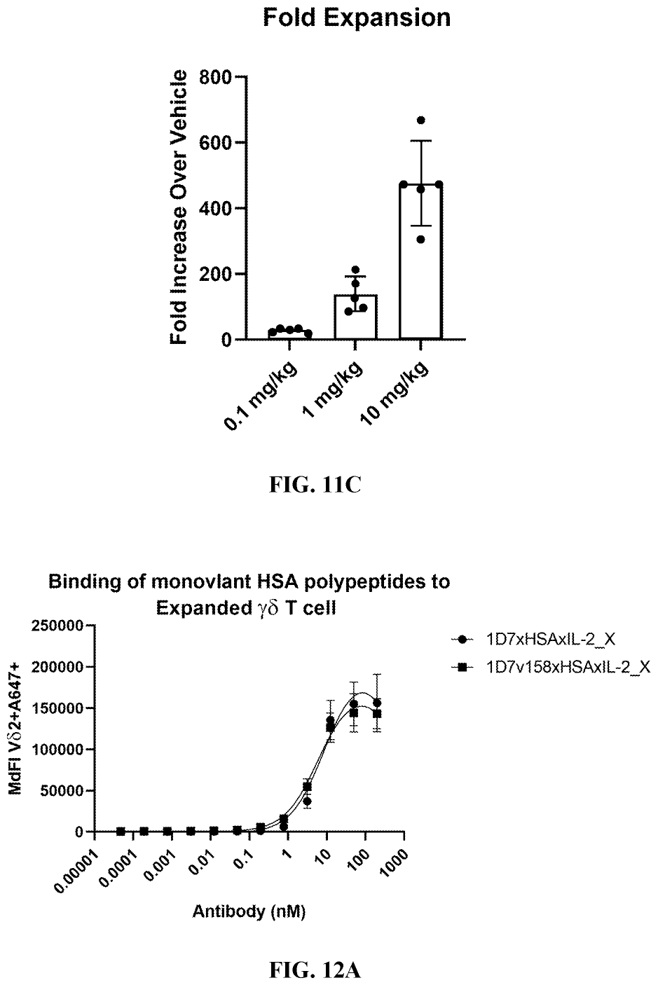

The serum γδTCR-targeted IL-2_X concentration (drug exposure) 24 hours after dosing, and the total Vγ9Vδ2 T cell count and fold expansion relative to vehicle alone at day 10 (9 days after dosing) are shown in FIGS. 11A, 111B, and 11C, respectively. These data show that treatment with monovalent γδTCR-targeted IL-2_X led to an increase in the total Vγ9Vδ2 T cell count compared to the vehicle control group. The increase in Vγ9Vδ2 T cell count correlated with increasing γδTCR-targeted TL-2_X exposure.

### Example 12: Binding and Activity of h1D7v158 Anti-γδTCR VHH Molecules

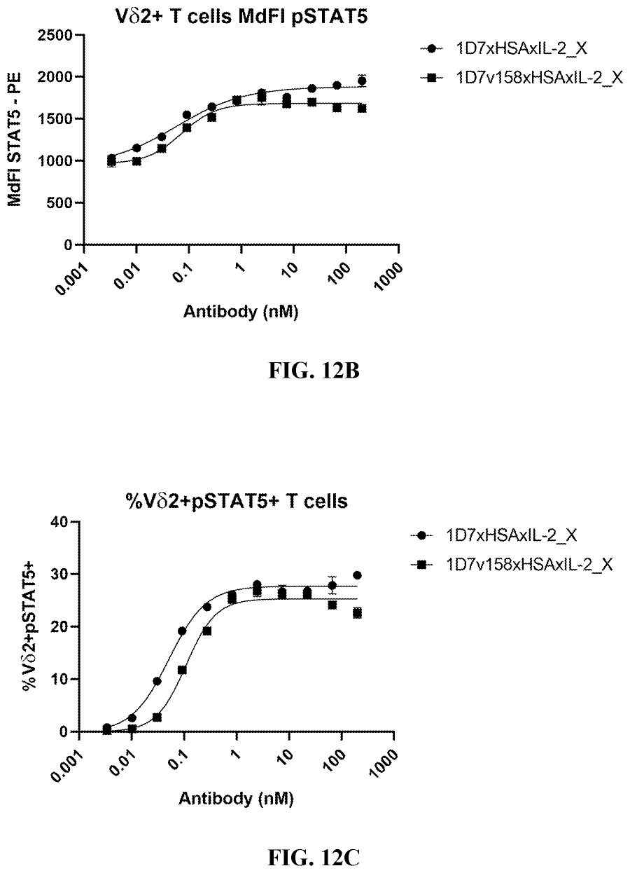

As described above an alternative monovalent format anti-γδTCR targeted IL-2 molecule was developed, comprising an anti-γδTCR VHH domain and an anti-human serum albumin (HSA) VHH. Studies were performed to evaluate the binding and activity of γδTCR-HSA targeted IL-2 molecules comprising the parental 1D7 or the h1D7v158 anti-γδTCR VHHs (1D7×HSA×IL-2_X and 1D7v158×HSA×IL-2_X, respectively). To evaluate binding to γδ T cells frozen zoledronate expanded γδ T cells were thawed and washed twice using 1×CTL thawing reagent in assay media, plated in a 96 well plate 100,000 cells per well, test articles were added in 50 uL to each well at 4× the final starting concentration of 100 nM and titrated 1:4 across. 50 μL of FACS buffer was added for the final total volume of 200 uL, incubated at 4 C for 30 minutes, washed twice, and stained with anti-Vγ2-PE, anti-CD3-BV785, anti-VHH-Alexa647 in FACS buffer for 30 minutes at 4 C. The cells were washed 2× with FACS buffer, resuspended in 70 μL of FACS buffer and binding to γδ T cells was determined by flow cytometry on a Quanteon flow cytometer. The data was plotted and analyzed using GraphPad Prism analysis software. As shown in FIG. 12A the curves for monovalent HSA molecules comprising the parental or new optimized humanized 1D7v158 are similar.

The molecules were also evaluated in an STAT5 signaling assay (in the absence of HSA) essentially as described in Example 5 above. As shown in FIG. 12B-12C titration of the monovalent HSA molecules comprising the parental or new optimized humanized 1D7v158 led to a similar dose-dependent increase in the pSTAT5 median fluorescent intensity on Vδ2⁺ γδ T cells (FIG. 12B) and the percentage of Vd2+ g/d T cells that bound to the phospho-specific STAT5 antibody (FIG. 12C).

### Example 13: The Anti-γδ TCR-Binding VHH 1D7 Binds Cynomolgus Monkey γδ T Cells and Effectively Targets Fusion Molecules

The ability of bivalent molecules comprising an anti-γδ TCR-binding VHH (1D7 or 1D7v9) fused to human IgG1 Fe xELL (SEQ ID NO: 33) to specifically bind to the Vγ₉Vδ2 subset from cynomologus monkey PBMCs was examined. Briefly, expanded γδ T cells from a cynomologus donor were thawed, washed twice using CTL thawing reagent, and resuspended at 0.5×10 \hat{ }6 cells/mL in complete assay media. 200 μL of the resuspended cells, or 100,000 cells/well, were added to a non-sterile 96 well U-bottom plate. Cells were spun down and 100 μL of FACs buffer was added to the wells. Antibodies were added in 100 uL to the U-bottom plates at 2× the final starting concentration of 100 nM and titrated down the plate 1:5 in FACs buffer. For wells without added test articles, 100 uL/well of FACs buffer was added instead. The plates were incubated for 30 minutes at 4 C. Cells were washed twice with FACs buffer and stained with anti-CD3-BV421, anti-Vγ9-FITC, and anti-human Fcγ-A647 in 50 μL of FACS buffer for 30 minutes at 4 C. After incubation, the plates were washed twice with FACS buffer, resuspended in 70 ul of FACS buffer and binding to γδ T cells was determined by flow cytometry on a Novocyte.

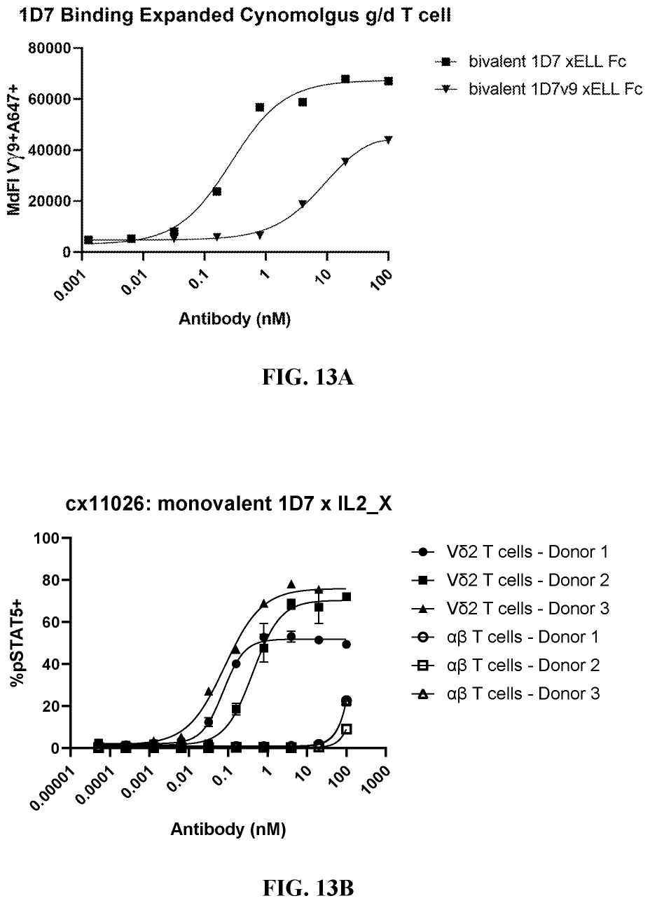

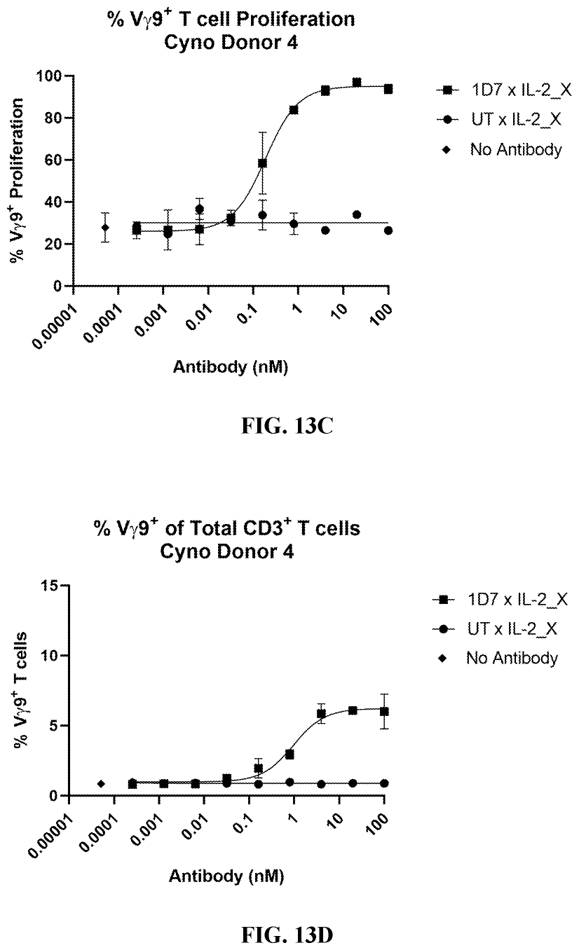

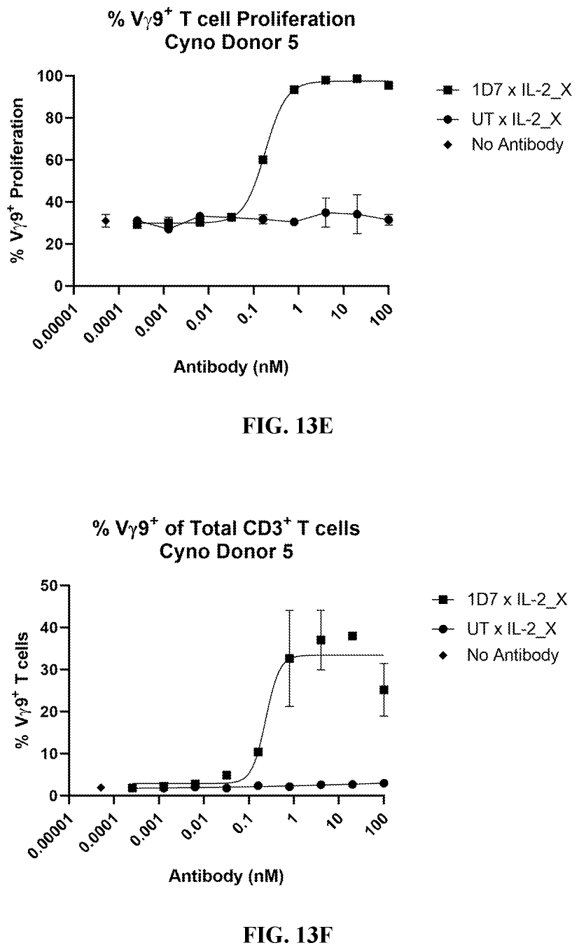

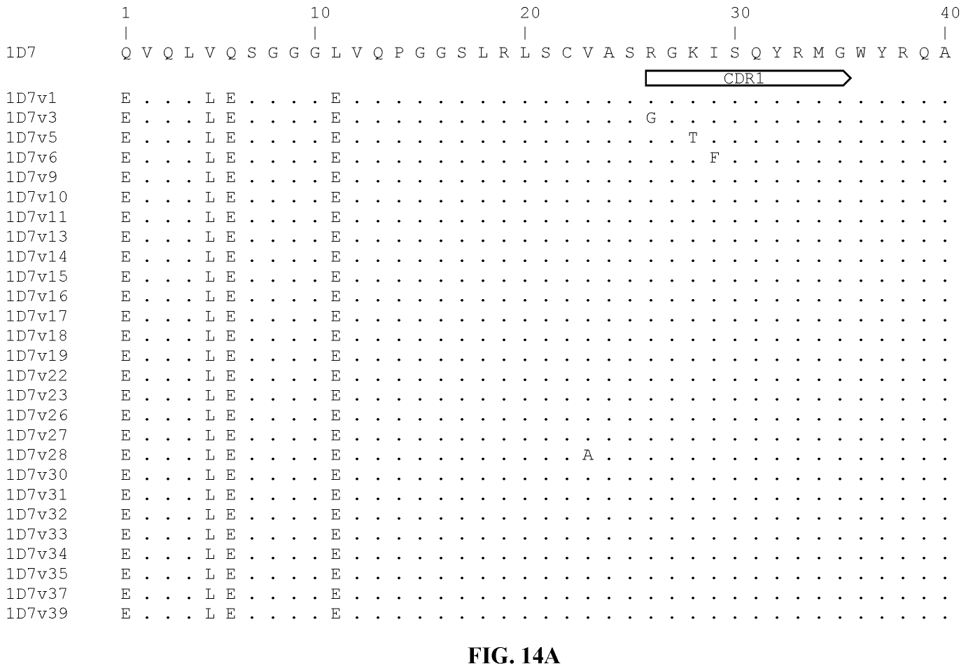

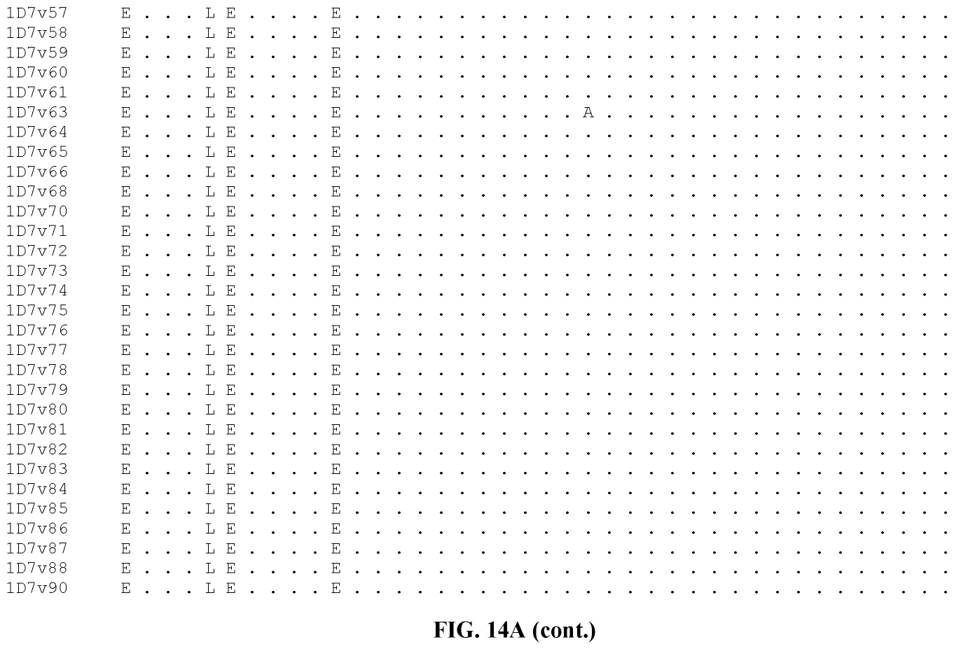

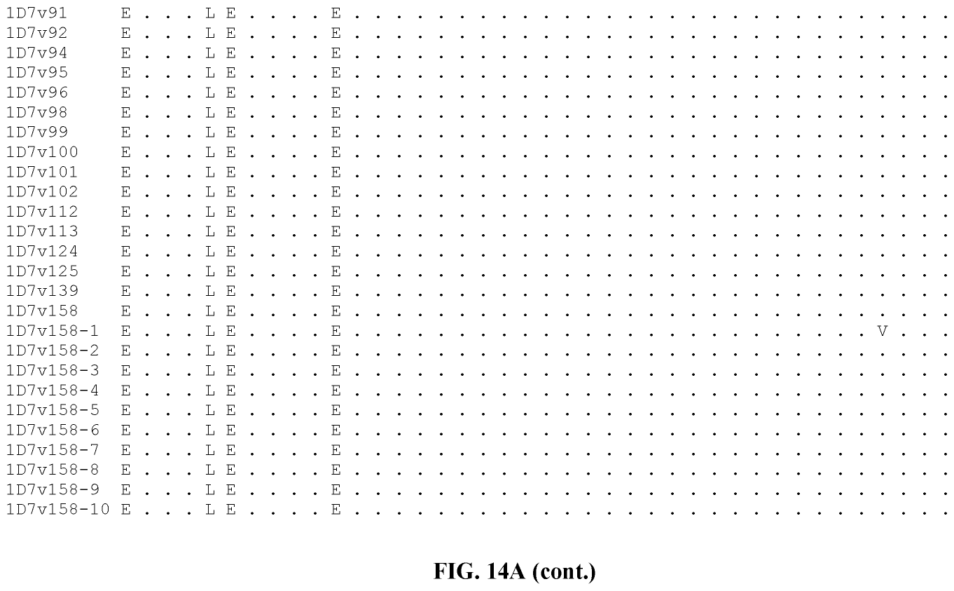

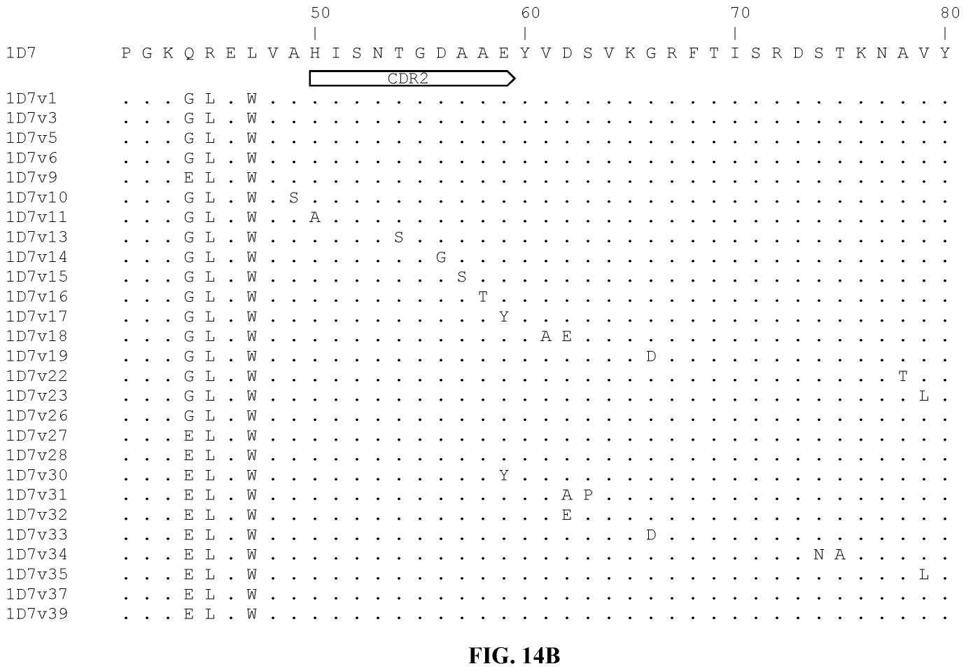

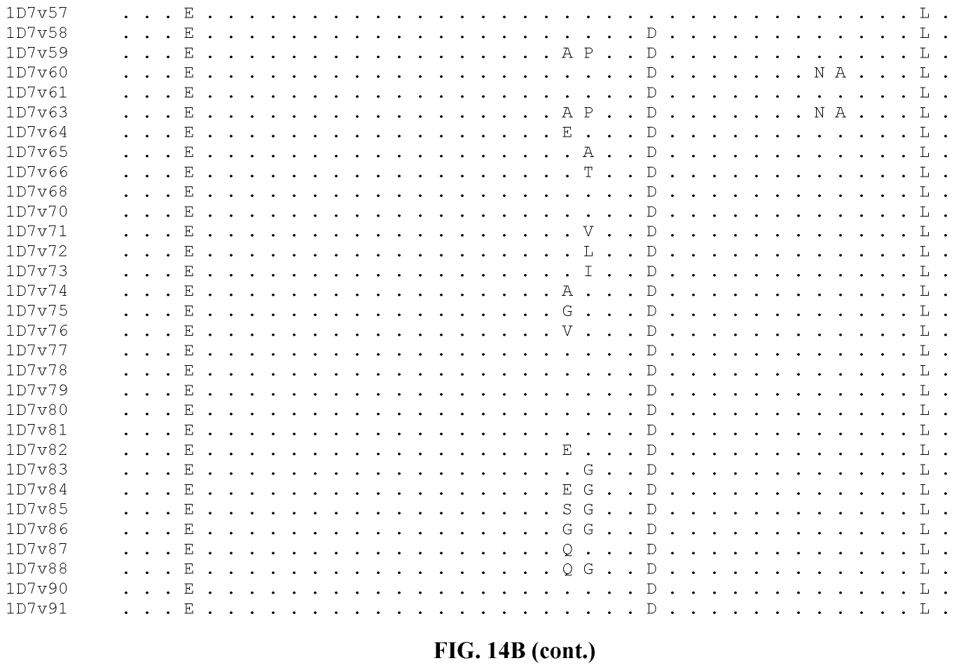

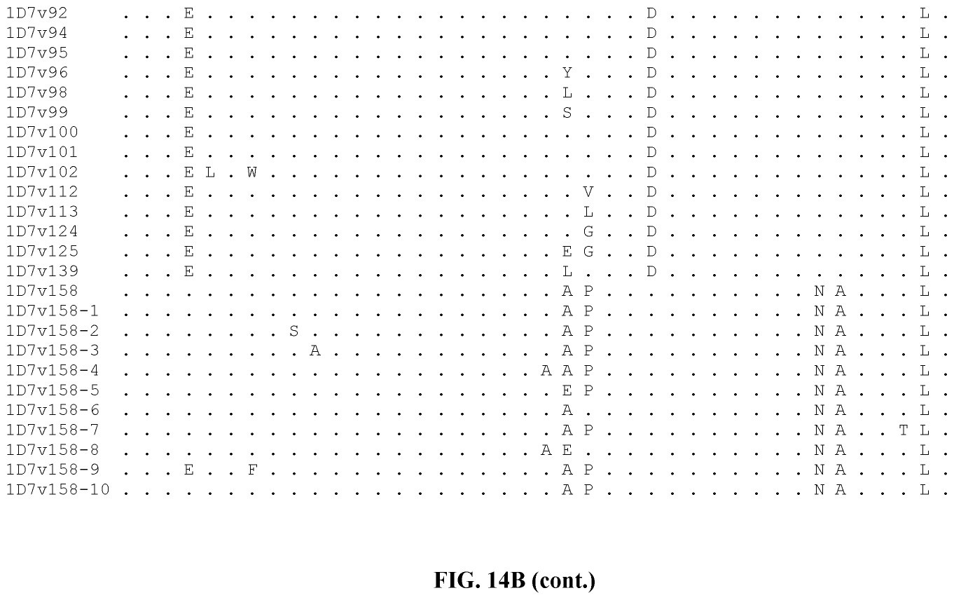

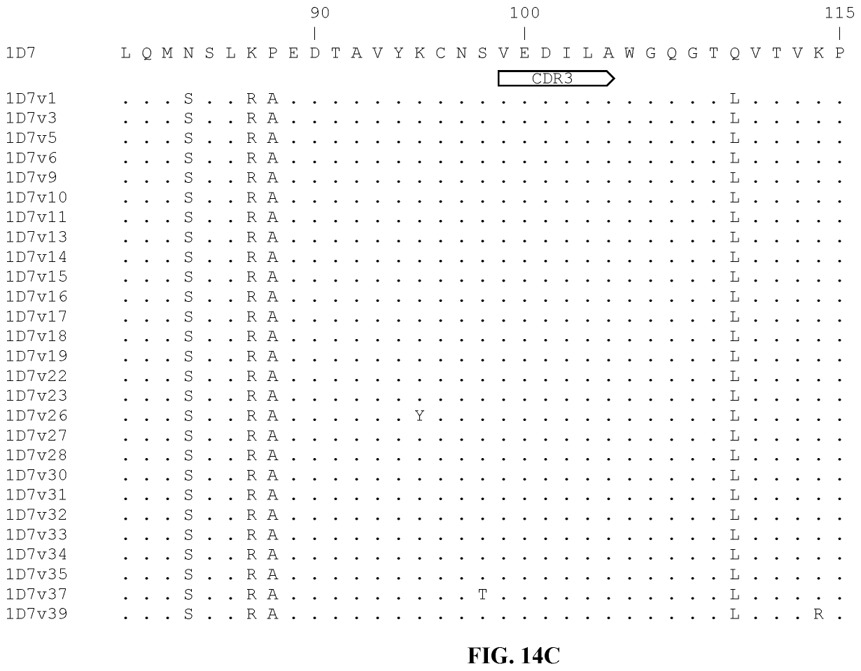

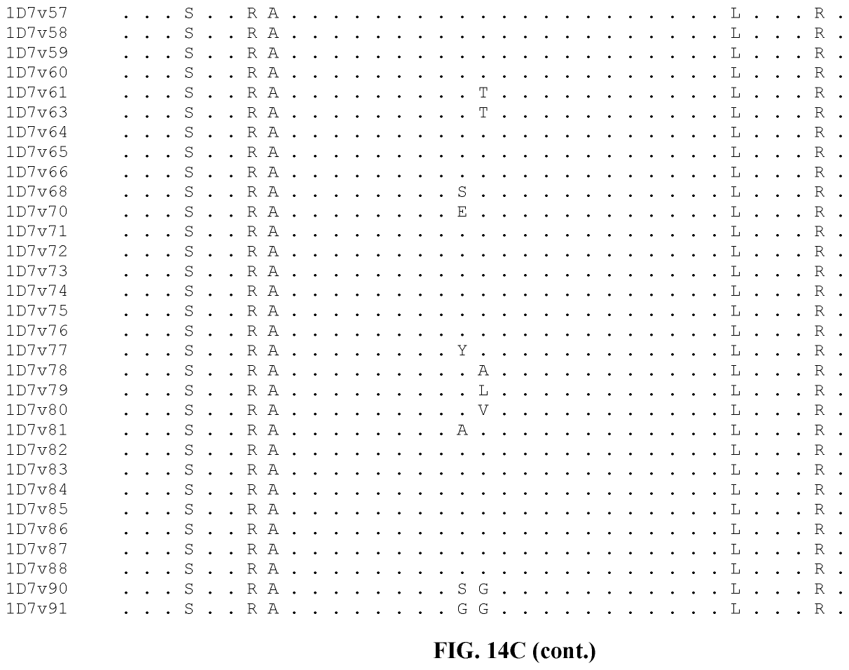

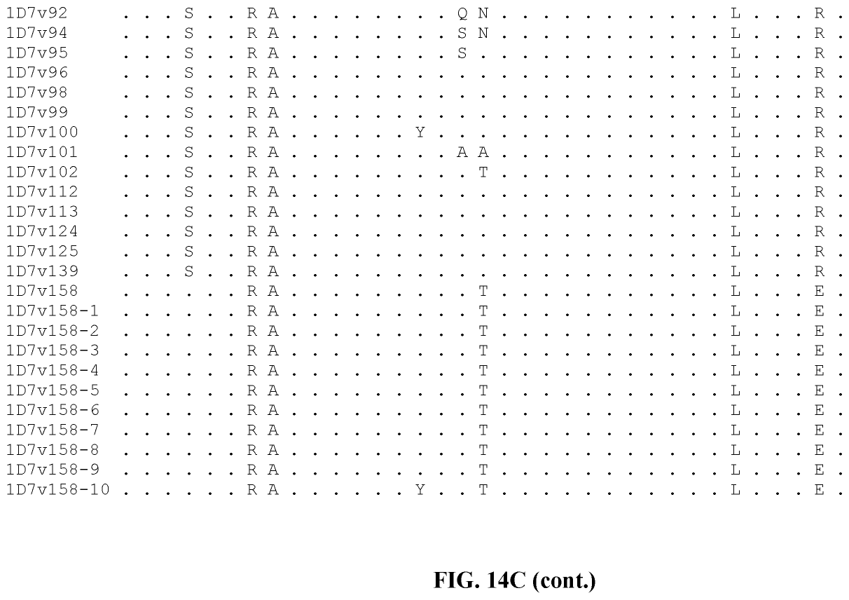

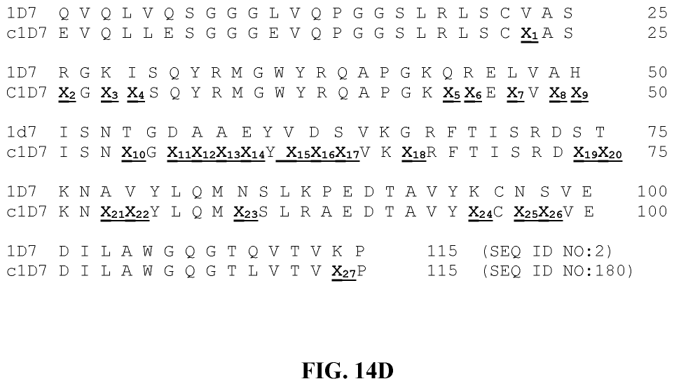

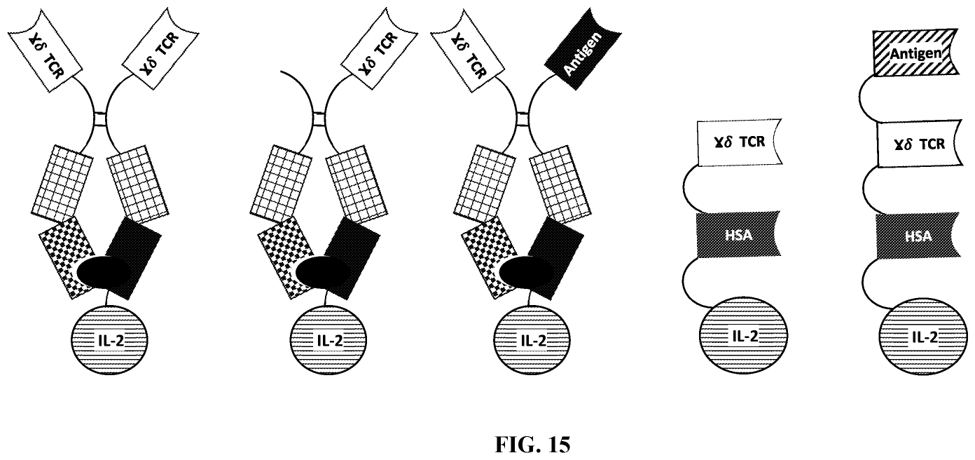

As shown in FIG. 13A, both bivalent molecules bound specifically to the Vγ9+ γδ T cell subset in a dose-dependent manner. The bivalent comprising the 1D7v9 VHH exhibits a lower binding affinity for cynomolgus γδ T cells, a lower affinity for this humanized variant was also observed for human γδ T cells (see Example 6, above).

The ability of a monovalent γδTCR-targeted IL-2_X molecule comprising the anti-γδ TCR-binding VHH 1D7 (cx11026) to enhance IL2-signaling through STAT5 phosphorylation was tested across several cynomolgus monkey PBMC donors. Briefly, PBMCs from three donors were isolated and rested overnight in complete medium. The cells were labeled for 20 minutes at room temperature with the following fluorescently conjugated antibodies: non-competing anti-Vγ9-FITC, anti-CD3-B421. After washing, 400,000 PBMCs were seeded per well in a 96-well plate. The cells were treated with a titration of the fusion protein comprising IL-2_X fused to the C-terminal of anti-γδTCR-1D7 (cx11026) added at 4× the final starting concentration of 100 nM and titrating across the plate 1:5 in assay media (50 uL). The final total volume was 200 uL/well. The plates were incubated for 20 minutes at 37° C./5% CO₂. The cells were fixed in 100 uL/well of BD Fixation/Permeabilization buffer for 45 minutes, then permeabilized with 100 uL/well of BD Permeabilization buffer III for 1 hour and washed 3× with FACS buffer. The cells were then stained overnight at 4 C with 50 uL/well of FACS diluted pSTAT5 antibody. The following day, cells were washed 2× with FACS buffer, resuspended in 70 μL of FACS buffer levels of phosphorylated STAT5 (“pSTAT5”) on Vγ9⁺ γδ T cells or Vγ9⁺ γδ⁻ alpha beta T cells were measured by flow cytometry on a Quanteon flow cytometer.

As shown in FIG. 13B treatment with monovalent anti-γδTCR-1D7 targeted IL-2_X led to a dose-dependent increase in the percentage of pSTAT5⁺ Vγ9⁺ γδ T cells across all 3 cynomolgus monkey PBMC donor samples. There was no detectable pSTAT5 on αβ T cells from any of the donors.

The ability of a monovalent γδTCR-targeted IL-2_X molecule comprising the anti-γδ TCR-binding VHH 1D7 (cx11026) to enhance Vγ9⁺ γδ T cell proliferation was tested across several cynomolgus monkey PBMC donors. Briefly, PBMCs were freshly isolated from fresh cynomolgus blood samples and labeled with CellTrace Violet and added 300,000 per well as described in Example 2. The fusion protein comprising IL-2_X fused to the C-terminal of either anti-γδTCR-1D7 (cx11026) or a non-targeted control VHH (cx9452) were added in 50 uL at 4× the final starting concentration of 100 nM and titrated across 1:5 in assay media. 50 uL of media was added for a final total volume of 200 uL/well and assay plates were incubated at 37 C/5% CO2 for 7 days. The cells were labeled with the following fluorescently conjugated antibodies: CD3-APC, Vγ9-FITC, DNAM1-PE, NKGD-APC/Cy7 and the viability dye propidium iodide for 30 minutes at 4° C. The cells were washed and analyzed by flow cytometry on a Quanteon flow cytometer.

Data from two representative donors is provided in FIG. 13C-13F and show that treatment with anti-γδTCR-1D7 (cx11026) targeted IL-2_X led to a dose-dependent increase in the proliferation of Vγ9 γδ T cells (FIGS. 13C and 13E). Additionally, the percentage of γδ T cells amongst the total CD3⁺ T cell population substantially increased with anti-γδTCR-1D7 targeted IL-2_X treatment (FIGS. 13D and 13F). The non-targeted VHH (cx9452) fused to IL-2_X did not promote the proliferation of Vγ₉ γδ T cells demonstrating target specificity.

Together these studies demonstrate that the human γδ TCR-binding VHH, 1D7 and humanized versions, specifically cross react with cynomolgus γδ T cells and that these VHH domains effectively target fusion molecules (e.g., γδTCR×IL-2_X) to cynomolgus γδ T cells.

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein.

Table of Certain Complexes

| | | SEQ |
|---|---|---|
| | | ID |
| Complex | Polypeptides‡ | NO. |
| cx7530 | OprI18v13 VHH-Fc region 2 IgG1 | 160 |
| monovalent 5C8 VHH- | xELL knob-IL-2-T3G-H16A-P65R- | |
| modified IL-2 | D84S-C125S | |
| (“monovalent 5C8 x IL- | 5C8 VHH-Fc region xELL H435R, | 10 |
| 2_Y”) | T366S, L368A, Y407V hole | |
| cx10762 | 5C8 VHH-Fc region 2 IgG1 xELL | 11 |
| bivalent 5C8 VHH- | knob-IL-2-T3A-H16A-E61R-P65R- | |
| modified IL-2 (“ bivalent | D84Y-C125S | |
| 5C8 x IL-2_X”) | 5C8 VHH-Fc region xELL H435R, | 10 |
| | T366S, L368A, Y407V hole | |
| cx10774 | 6C4 VHH-Fc region 2 IgG1 xELL | 12 |
| bivalent 6C4 VHH- | knob-IL-2-T3A-H16A-E61R-P65R- | |
| modified IL-2 (“bivalent | D84Y-C125S | |
| 6C4 x IL-2_X”) | 6C4 VHH-Fc region xELL H435R, | 13 |
| | T366S, L368A, Y407V hole | |
| cx10767 | 6C4 VHH-Fc region 2 IgG1 xELL | 12 |
| monovalent 6C4- | knob-IL-2-T3A-H16A-E61R-P65R- | |
| modified IL-2 | D84Y-C125S | |
| (“monovalent 6C4 x IL- | 401A9v1 VHH-Fc region xELL H435R, | 14 |
| 2_X”) | T366S, L368A, Y407V hole | |
| cx11005 | 5C8 VHH-Fc region 2 IgG1 xELL | 11 |
| monovalent 5C8 VHH- | knob-IL-2-T3A-H16A-E61R-P65R- | |
| modified IL-2 | D84Y-C125S | |
| | Fc region xELL H435R, T366S, L368A, | 48 |
| | Y407V hole | |
| cx9452 | 401A9v1 VHH-Fc region 2 IgG1 xELL | 15 |
| non-targeted modified | knob-IL-2-T3A-H16A-E61R-P65R- | |
| IL-2 (“non-targeted IL- | D84Y-C125S | |
| 2_X”) | 401A9v1 VHH-Fc region xELL | 14 |
| | H435R, T366S, L368A, Y407V hole | |
| cx11026 | 1D7 VHH-Fc region 2 IgG1 xELL | 16 |
| monovalent 1D7 VHH- | knob-IL-2-T3A-H16A-E61R-P65R- | |
| modified IL-2 | D84Y-C125S | |
| (“monovalent 1D7 x IL- | Fc region xELL H435R, T366S, L368A, | 48 |
| 2_X”) | Y407V hole | |
| cx1603 | OprI18v13 VHH-Fc region 2 IgG1 | 160 |
| non-targeted IL-2_Y | xELL knob-IL-2-T3G-H16A-P65R- | |
| | D84S-C125S | |
| | OprI18v13 VHH-Fc region xELL | 161 |
| | VHH-Fc region xELL H435R, T366S, | |
| | L368A, Y407V hole | |
| cx9071 | 5T4-hz16G10v11 VHH-Fc region 2 | 162 |
| bispecific | IgG1 xELL knob-IL2-T3G-H16A- | |
| (“5T4 x 5C8 x IL-2_Y”) | P65R-D84S-C125S | |
| | 5C8 VHH-Fc region xELL H435R, | 10 |
| | T366S, L368A, Y407V hole | |
| cx7537 | OprI18v13 VHH-Fc region 2 IgG1 | 160 |
| (“monovalent 5D7 x IL- | xELL knob-IL-2-T3G-H16A-P65R- | |
| 2_Y”) | D84S-C125S | |
| | 5D7 VHH-Fc region xELL H435R, | 163 |
| | T366S, L368A, Y407V hole | |
| cx9075 | 5T4-hz16G10v11 VHH-Fc region 2 | 162 |
| bispecific (“5T4 x 5D7 x | IgG1 xELL knob-IL2 | |
| IL-2_Y”) | 5D7 VHH-Fc region xELL H435R, | 163 |
| | T366S, L368A, Y407V hole | |
| cx12168 | 1D7v158 VHH-Fc region 2 IgG1 xELL | 164 |
| (“monovalent 1D7v158 | knob-IL-2-T3A-H16A-E61R-P65R- | |
| targeted IL-2_X”) | D84Y-C125S | |
| | Fc region xELL H435R, T366S, L368A, | 48 |
| | Y407V hole | |

‡OprI18v13 VHH and 401A9v1 VHH domains are used as non-binding control VHH domains

Table of Certain Sequences

| SEQ | | |
|---|---|---|
| ID | | |
| NO | Description | Sequence |
| 1 | Human gamma 9 TCR | AGHLEQPQISSTKTLSKTARLECVVSGITISATSVYWYRERPGE |
| | subunit | VIQFLVSISYDGTVRKESGIPSGKFEVDRIPETSTSTLTIHNVE |
| | | KQDIATYYCALWEAQQELGKKIKVFGPGTKLIITDKQLDADVSP |
| | | KPTIFLPSIAETKLQKAGTYLCLLEKFFPDVIKIHWEEKKSNTI |
| | | LGSQEGNTMKTNDTYMKFSWLTVPEKSLDKEHRCIVRHENNKNG |
| | | VDQEIIFPPIP |
| 2 | 1D7 VHH | QVQLVQSGGGLVQPGGSLRLSCVASRGKISQYRMGWYRQAPGKQ |
| | | RELVAHISNTGDAAEYVDSVKGRFTISRDSTKNAVYLQMNSLKP |
| | | EDTAVYKCNSVEDILAWGQGTQVTVKP |
| 3 | CDR1 of 1D7 and | RGKISQYRMG |
| | humanized 1D7 | |
| | (hu1D7) v1, v2, v9- | |
| | v28, v30-v39, v57- | |
| | v102, v112, v113, | |
| | v124, v125, v139, v158 | |
| | and v158-1-v158-10 | |
| 144 | CDR1 of hu1D7v3 | GGKISQYRMG |
| 145 | CDR1 of hu1D7v4 | RFKISQYRMG |
| 146 | CDR1 of hu1D7v5 | RGTISQYRMG |
| 147 | CDR1 of hu1D7v6 | RGKFSQYRMG |
| 148 | CDR1 of hu1D7v7 and | RGKISSYRMG |
| | 1D7v29 | |
| 149 | CDR1 of hu1D7v8 | RGKISQYAMG |
| 4 | CDR2 of 1D7 and | HISNTGDAAE |
| | hu1D7 v1-v10, v18- | |
| | v29, v31-v39, v57- | |
| | v102, v112, v113, | |
| | v124, v125, v139, v158 | |
| | and v158-1, v158-2, | |
| | v158-4-v158-10 | |
| 150 | CDR2 of hu1D7v11, | AISNTGDAAE |
| | and v158-3 | |
| 151 | CDR2 of hu1D7v12 | HISGTGDAAE |
| 152 | CDR2 of hu1D7v13 | HISNSGDAAE |
| 153 | CDR2 of hu1D7v14 | HISNTGGAAE |
| 154 | CDR2 of hu1D7v15 | HISNTGDSAE |
| 155 | CDR2 of hu1D7v16 | HISNTGDATE |
| 156 | CDR2 of hu1D7v17 | HISNTGDAAY |
| | and 1D7v30 | |
| 5 | CDR3 of 1D7 and | VEDILA |
| | humanized 1D7 v1-v39 | |
| | v57-v102, and v158 | |
| 6 | 5C8 VHH | EVQLVESGGGLVQAGGSLRLSCAASGRPFSNYAMGWFRQAPGKE |
| | | REFVAAISWSGGSTSYADSVKGRFTISRDNAKNTVYLQMNSPKP |
| | | EDTAIYYCAAQFSGADYGFGRLGIRGYEYDYWGQGTQVTVKP |
| 7 | 6C4 VHH | EVQLVESGGGLVQAGGSLRLSCAVSVRTFSDYRMGWFRQAPGKE |
| | | REFVSTISWSGGLTYYADSVKGRFTISRDNSKNTLYLQMNSLKP |
| | | EDTAVYYCAAGGGYAGGTYYHPEEWGQGTQVTVKP |
| 8 | 401A9v1 (non- | EVQLVESGGGEVQPGGSLRLSCAASGSINSINVMEWYRQAPGKE |
| | targeted) VHH | RDLVAGITSDGDTNYAESVKGRFTISRDNAKNTLYLQMSSLRAE |
| | | DTAVYYCRARDWGSLTDYWGQGTLVTVKP |
| 165 | OprI18v13 (non- | EVQLVESGGGEVQPGGSLRLSCAASETIFRFNTMAWYRQAPGKR |
| | targeted) VHH | RELVGYITWAGRTGYAESVEGRFTISRDNAKNTVYLQMSSLRAE |
| | | DTAVYYCNKHGSSFTQDYWGQGTLVTVKP |
| 17 | hu1D7v1 VHH | EVQLLESGGGEVQPGGSLRLSCVASRGKISQYRMGWYRQAPGKG |
| | | LEWVAHISNTGDAAEYVDSVKGRFTISRDSTKNAVYLQMSSLRA |
| | | EDTAVYKCNSVEDILAWGQGTLVTVKP |
| 18 | hu1D7v2 VHH | EVQLLESGGGEVQPGGSLRLSCAASRGKISQYRMGWYRQAPGKG |
| | | LEWVAHISNTGDAAEYVDSVKGRFTISRDSTKNAVYLQMSSLRA |
| | | EDTAVYKCNSVEDILAWGQGTLVTVKP |
| 19 | hu1D7v3 VHH | EVQLLESGGGEVQPGGSLRLSCVASGGKISQYRMGWYRQAPGKG |
| | | LEWVAHISNTGDAAEYVDSVKGRFTISRDSTKNAVYLQMSSLRA |
| | | EDTAVYKCNSVEDILAWGQGTLVTVKP |
| 20 | hu1D7v4 VHH | EVQLLESGGGEVQPGGSLRLSCVASRFKISQYRMGWYRQAPGKG |
| | | LEWVAHISNTGDAAEYVDSVKGRFTISRDSTKNAVYLQMSSLRA |
| | | EDTAVYKCNSVEDILAWGQGTLVTVKP |
| 21 | hu1D7v5 VHH | EVQLLESGGGEVQPGGSLRLSCVASRGTISQYRMGWYRQAPGKG |
| | | LEWVAHISNTGDAAEYVDSVKGRFTISRDSTKNAVYLQMSSLRA |
| | | EDTAVYKCNSVEDILAWGQGTLVTVKP |
| 22 | hu1D7v6 VHH | EVQLLESGGGEVQPGGSLRLSCVASRGKFSQYRMGWYRQAPGKG |
| | | LEWVAHISNTGDAAEYVDSVKGRFTISRDSTKNAVYLQMSSLRA |
| | | EDTAVYKCNSVEDILAWGQGTLVTVKP |
| 23 | hu1D7v7 VHH | EVQLLESGGGEVQPGGSLRLSCVASRGKISSYRMGWYRQAPGKG |
| | | LEWVAHISNTGDAAEYVDSVKGRFTISRDSTKNAVYLQMSSLRA |
| | | EDTAVYKCNSVEDILAWGQGTLVTVKP |
| 24 | hu1D7v8 VHH | EVQLLESGGGEVQPGGSLRLSCVASRGKISQYAMGWYRQAPGKG |
| | | LEWVAHISNTGDAAEYVDSVKGRFTISRDSTKNAVYLQMSSLRA |
| | | EDTAVYKCNSVEDILAWGQGTLVTVKP |
| 25 | hu1D7v9 VHH | EVQLLESGGGEVQPGGSLRLSCVASRGKISQYRMGWYRQAPGKE |
| | | LEWVAHISNTGDAAEYVDSVKGRFTISRDSTKNAVYLQMSSLRA |
| | | EDTAVYKCNSVEDILAWGQGTLVTVKP |
| 26 | hu1D7v10 VHH | EVQLLESGGGEVQPGGSLRLSCVASRGKISQYRMGWYRQAPGKG |
| | | LEWVSHISNTGDAAEYVDSVKGRFTISRDSTKNAVYLQMSSLRA |
| | | EDTAVYKCNSVEDILAWGQGTLVTVKP |
| 27 | hu1D7v11 VHH | EVQLLESGGGEVQPGGSLRLSCVASRGKISQYRMGWYRQAPGKG |
| | | LEWVAAISNTGDAAEYVDSVKGRFTISRDSTKNAVYLQMSSLRA |
| | | EDTAVYKCNSVEDILAWGQGTLVTVKP |
| 28 | hu1D7v12 VHH | EVQLLESGGGEVQPGGSLRLSCVASRGKISQYRMGWYRQAPGKG |
| | | LEWVAHISGTGDAAEYVDSVKGRFTISRDSTKNAVYLQMSSLRA |
| | | EDTAVYKCNSVEDILAWGQGTLVTVKP |
| 29 | hu1D7v13 VHH | EVQLLESGGGEVQPGGSLRLSCVASRGKISQYRMGWYRQAPGKG |
| | | LEWVAHISNSGDAAEYVDSVKGRFTISRDSTKNAVYLQMSSLRA |
| | | EDTAVYKCNSVEDILAWGQGTLVTVKP |
| 30 | hu1D7v14 VHH | EVQLLESGGGEVQPGGSLRLSCVASRGKISQYRMGWYRQAPGKG |
| | | LEWVAHISNTGGAAEYVDSVKGRFTISRDSTKNAVYLQMSSLRA |
| | | EDTAVYKCNSVEDILAWGQGTLVTVKP |
| 31 | hu1D7v15 VHH | EVQLLESGGGEVQPGGSLRLSCVASRGKISQYRMGWYRQAPGKG |
| | | LEWVAHISNTGDSAEYVDSVKGRFTISRDSTKNAVYLQMSSLRA |
| | | EDTAVYKCNSVEDILAWGQGTLVTVKP |
| 72 | hu1D7v16 VHH | EVQLLESGGGEVQPGGSLRLSCVASRGKISQYRMGWYRQAPGKG |
| | | LEWVAHISNTGDATEYVDSVKGRFTISRDSTKNAVYLQMSSLRA |
| | | EDTAVYKCNSVEDILAWGQGTLVTVKP |
| 73 | hu1D7v17 VHH | EVQLLESGGGEVQPGGSLRLSCVASRGKISQYRMGWYRQAPGKG |
| | | LEWVAHISNTGDAAYYVDSVKGRFTISRDSTKNAVYLQMSSLRA |
| | | EDTAVYKCNSVEDILAWGQGTLVTVKP |
| 74 | hu1D7v18 VHH | EVQLLESGGGEVQPGGSLRLSCVASRGKISQYRMGWYRQAPGKG |
| | | LEWVAHISNTGDAAEYAESVKGRFTISRDSTKNAVYLQMSSLRA |
| | | EDTAVYKCNSVEDILAWGQGTLVTVKP |
| 75 | hu1D7v19 VHH | EVQLLESGGGEVQPGGSLRLSCVASRGKISQYRMGWYRQAPGKG |
| | | LEWVAHISNTGDAAEYVDSVKDRFTISRDSTKNAVYLQMSSLRA |
| | | EDTAVYKCNSVEDILAWGQGTLVTVKP |
| 76 | hu1D7v20 VHH | EVQLLESGGGEVQPGGSLRLSCVASRGKISQYRMGWYRQAPGKG |
| | | LEWVAHISNTGDAAEYVDSVKGRFTISRDNTKNAVYLQMSSLRA |
| | | EDTAVYKCNSVEDILAWGQGTLVTVKP |
| 77 | hu1D7v21 VHH | EVQLLESGGGEVQPGGSLRLSCVASRGKISQYRMGWYRQAPGKG |
| | | LEWVAHISNTGDAAEYVDSVKGRFTISRDSAKNAVYLQMSSLRA |
| | | EDTAVYKCNSVEDILAWGQGTLVTVKP |
| 80 | hu1D7v22 VHH | EVQLLESGGGEVQPGGSLRLSCVASRGKISQYRMGWYRQAPGKG |
| | | LEWVAHISNTGDAAEYVDSVKGRFTISRDSTKNTVYLQMSSLRA |
| | | EDTAVYKCNSVEDILAWGQGTLVTVKP |
| 81 | hu1D7v23 VHH | EVQLLESGGGEVQPGGSLRLSCVASRGKISQYRMGWYRQAPGKG |
| | | LEWVAHISNTGDAAEYVDSVKGRFTISRDSTKNALYLQMSSLRA |
| | | EDTAVYKCNSVEDILAWGQGTLVTVKP |
| 82 | hu1D7v24 VHH | EVQLLESGGGEVQPGGSLRLSCVASRGKISQYRMGWYRQAPGKG |
| | | LEWVAHISNTGDAAEYVDSVKGRFTISRDSTKNAVYLQMSSLRA |
| | | EDTAVYKCNAVEDILAWGQGTLVTVKP |
| 83 | hu1D7v25 VHH | EVQLLESGGGEVQPGGSLRLSCVASRGKISQYRMGWYRQAPGKG |
| | | LEWVAHISNTGDAAEYVDSVKGRFTISRDSTKNAVYLQMSSLRA |
| | | EDTAVYKCASVEDILAWGQGTLVTVKP |
| 84 | hu1D7v26 VHH | EVQLLESGGGEVQPGGSLRLSCVASRGKISQYRMGWYRQAPGKG |
| | | LEWVAHISNTGDAAEYVDSVKGRFTISRDSTKNAVYLQMSSLRA |
| | | EDTAVYYCNSVEDILAWGQGTLVTVKP |
| 85 | hu1D7v27 VHH | EVQLLESGGGEVQPGGSLRLSCVASRGKISQYRMGWYRQAPGKE |
| | | LEWVAHISNTGDAAEYVDSVKGRFTISRDSTKNAVYLQMSSLRA |
| | | EDTAVYKCNSVEDILAWGQGTLVTVKP |
| 86 | hu1D7v28 VHH | EVQLLESGGGEVQPGGSLRLSCAASRGKISQYRMGWYRQAPGKE |
| | | LEWVAHISNTGDAAEYVDSVKGRFTISRDSTKNAVYLQMSSLRA |
| | | EDTAVYKCNSVEDILAWGQGTLVTVKP |
| 87 | hu1D7v29 VHH | EVQLLESGGGEVQPGGSLRLSCVASRGKISSYRMGWYRQAPGKE |
| | | LEWVAHISNTGDAAEYVDSVKGRFTISRDSTKNAVYLQMSSLRA |
| | | EDTAVYKCNSVEDILAWGQGTLVTVKP |
| 88 | hu1D7v30 VHH | EVQLLESGGGEVQPGGSLRLSCVASRGKISQYRMGWYRQAPGKE |
| | | LEWVAHISNTGDAAYYVDSVKGRFTISRDSTKNAVYLQMSSLRA |
| | | EDTAVYKCNSVEDILAWGQGTLVTVKP |
| 89 | hu1D7v31 VHH | EVQLLESGGGEVQPGGSLRLSCVASRGKISQYRMGWYRQAPGKE |
| | | LEWVAHISNTGDAAEYVAPVKGRFTISRDSTKNAVYLQMSSLRA |
| | | EDTAVYKCNSVEDILAWGQGTLVTVKP |
| 90 | hu1D7v32 VHH | EVQLLESGGGEVQPGGSLRLSCVASRGKISQYRMGWYRQAPGKE |
| | | LEWVAHISNTGDAAEYVESVKGRFTISRDSTKNAVYLQMSSLRA |
| | | EDTAVYKCNSVEDILAWGQGTLVTVKP |
| 91 | hu1D7v33 VHH | EVQLLESGGGEVQPGGSLRLSCVASRGKISQYRMGWYRQAPGKE |
| | | LEWVAHISNTGDAAEYVDSVKDRFTISRDSTKNAVYLQMSSLRA |
| | | EDTAVYKCNSVEDILAWGQGTLVTVKP |
| 92 | hu1D7v34 VHH | EVQLLESGGGEVQPGGSLRLSCVASRGKISQYRMGWYRQAPGKE |
| | | LEWVAHISNTGDAAEYVDSVKGRFTISRDNAKNAVYLQMSSLRA |
| | | EDTAVYKCNSVEDILAWGQGTLVTVKP |
| 93 | hu1D7v35 VHH | EVQLLESGGGEVQPGGSLRLSCVASRGKISQYRMGWYRQAPGKE |
| | | LEWVAHISNTGDAAEYVDSVKGRFTISRDSTKNALYLQMSSLRA |
| | | EDTAVYKCNSVEDILAWGQGTLVTVKP |
| 94 | hu1D7v36 VHH | EVQLLESGGGEVQPGGSLRLSCVASRGKISQYRMGWYRQAPGKE |
| | | LEWVAHISNTGDAAEYVDSVKGRFTISRDSTKNAVYLQMSSLRA |
| | | EDTAVYYCNSVEDILAWGQGTLVTVKP |
| 95 | hu1D7v37 VHH | EVQLLESGGGEVQPGGSLRLSCVASRGKISQYRMGWYRQAPGKE |
| | | LEWVAHISNTGDAAEYVDSVKGRFTISRDSTKNAVYLQMSSLRA |
| | | EDTAVYKCNTVEDILAWGQGTLVTVKP |
| 96 | hu1D7v38 VHH | EVQLLESGGGEVQPGGSLRLSCVASRGKISQYRMGWYRQAPGKE |
| | | LEWVAHISNTGDAAEYVDSVKGRFTISRDSTKNAVYLQMSSLRA |
| | | EDTAVYKCNVVEDILAWGQGTLVTVKP |
| 97 | hu1D7v39 VHH | EVQLLESGGGEVQPGGSLRLSCVASRGKISQYRMGWYRQAPGKE |
| | | LEWVAHISNTGDAAEYVDSVKGRFTISRDSTKNAVYLQMSSLRA |
| | | EDTAVYKCNSVEDILAWGQGTLVTVRP |
| 98 | hu1D7v57 VHH | EVQLLESGGGEVQPGGSLRLSCVASRGKISQYRMGWYRQAPGKE |
| | | RELVAHISNTGDAAEYVDSVKGRFTISRDSTKNALYLQMSSLRA |
| | | EDTAVYKCNSVEDILAWGQGTLVTVRP |
| 99 | hu1D7v58 VHH | EVQLLESGGGEVQPGGSLRLSCVASRGKISQYRMGWYRQAPGKE |
| | | RELVAHISNTGDAAEYVDSVKDRFTISRDSTKNALYLQMSSLRA |
| | | EDTAVYKCNSVEDILAWGQGTLVTVRP |
| 100 | hu1D7v59 VHH | EVQLLESGGGEVQPGGSLRLSCVASRGKISQYRMGWYRQAPGKE |
| | | RELVAHISNTGDAAEYVAPVKDRFTISRDSTKNALYLQMSSLRA |
| | | EDTAVYKCNSVEDILAWGQGTLVTVRP |
| 101 | hu1D7v60 VHH | EVQLLESGGGEVQPGGSLRLSCVASRGKISQYRMGWYRQAPGKE |
| | | RELVAHISNTGDAAEYVDSVKDRFTISRDNAKNALYLQMSSLRA |
| | | EDTAVYKCNSVEDILAWGQGTLVTVRP |
| 102 | hu1D7v61 VHH | EVQLLESGGGEVQPGGSLRLSCVASRGKISQYRMGWYRQAPGKE |
| | | RELVAHISNTGDAAEYVDSVKDRFTISRDSTKNALYLQMSSLRA |
| | | EDTAVYKCNTVEDILAWGQGTLVTVRP |
| 103 | hu1D7v62 VHH | EVQLLESGGGEVQPGGSLRLSCVASRGKISQYRMGWYRQAPGKE |
| | | RELVAHISNTGDAAEYVAPVKDRFTISRDNAKNALYLQMSSLRA |
| | | EDTAVYKCNTVEDILAWGQGTLVTVRP |
| 104 | hu1D7v63 VHH | EVQLLESGGGEVQPGGSLRLSCAASRGKISQYRMGWYRQAPGKE |
| | | RELVAHISNTGDAAEYVAPVKDRFTISRDNAKNALYLQMSSLRA |
| | | EDTAVYKCNTVEDILAWGQGTLVTVRP |
| 105 | hu1D7v64 VHH | EVQLLESGGGEVQPGGSLRLSCVASRGKISQYRMGWYRQAPGKE |
| | | RELVAHISNTGDAAEYVESVKDRFTISRDSTKNALYLQMSSLRA |
| | | EDTAVYKCNSVEDILAWGQGTLVTVRP |
| 106 | hu1D7v65 VHH | EVQLLESGGGEVQPGGSLRLSCVASRGKISQYRMGWYRQAPGKE |
| | | RELVAHISNTGDAAEYVDAVKDRFTISRDSTKNALYLQMSSLRA |
| | | EDTAVYKCNSVEDILAWGQGTLVTVRP |
| 107 | hu1D7v66 VHH | EVQLLESGGGEVQPGGSLRLSCVASRGKISQYRMGWYRQAPGKE |
| | | RELVAHISNTGDAAEYVDTVKDRFTISRDSTKNALYLQMSSLRA |
| | | EDTAVYKCNSVEDILAWGQGTLVTVRP |
| 108 | hu1D7v67 VHH | EVQLLESGGGEVQPGGSLRLSCVASRGKISQYRMGWYRQAPGKE |
| | | RELVAHISNTGDAAEYVDSVKDRFTISRDSTKNALYLQMSSLRA |
| | | EDTAVYKCQSVEDILAWGQGTLVTVRP |
| 109 | hu1D7v68 VHH | EVQLLESGGGEVQPGGSLRLSCVASRGKISQYRMGWYRQAPGKE |
| | | RELVAHISNTGDAAEYVDSVKDRFTISRDSTKNALYLQMSSLRA |
| | | EDTAVYKCSSVEDILAWGQGTLVTVRP |
| 110 | hu1D7v69 VHH | EVQLLESGGGEVQPGGSLRLSCVASRGKISQYRMGWYRQAPGKE |
| | | RELVAHISNTGDAAEYVDSVKDRFTISRDSTKNALYLQMSSLRA |
| | | EDTAVYKCTSVEDILAWGQGTLVTVRP |
| 111 | hu1D7v70 VHH | EVQLLESGGGEVQPGGSLRLSCVASRGKISQYRMGWYRQAPGKE |
| | | RELVAHISNTGDAAEYVDSVKDRFTISRDSTKNALYLQMSSLRA |
| | | EDTAVYKCESVEDILAWGQGTLVTVRP |
| 112 | hu1D7v71 VHH | EVQLLESGGGEVQPGGSLRLSCVASRGKISQYRMGWYRQAPGKE |
| | | RELVAHISNTGDAAEYVDVVKDRFTISRDSTKNALYLQMSSLRA |
| | | EDTAVYKCNSVEDILAWGQGTLVTVRP |
| 113 | hu1D7v72 VHH | EVQLLESGGGEVQPGGSLRLSCVASRGKISQYRMGWYRQAPGKE |
| | | RELVAHISNTGDAAEYVDLVKDRFTISRDSTKNALYLQMSSLRA |
| | | EDTAVYKCNSVEDILAWGQGTLVTVRP |
| 114 | hu1D7v73 VHH | EVQLLESGGGEVQPGGSLRLSCVASRGKISQYRMGWYRQAPGKE |
| | | RELVAHISNTGDAAEYVDIVKDRFTISRDSTKNALYLQMSSLRA |
| | | EDTAVYKCNSVEDILAWGQGTLVTVRP |
| 115 | hu1D7v74 VHH | EVQLLESGGGEVQPGGSLRLSCVASRGKISQYRMGWYRQAPGKE |
| | | RELVAHISNTGDAAEYVASVKDRFTISRDSTKNALYLQMSSLRA |
| | | EDTAVYKCNSVEDILAWGQGTLVTVRP |
| 116 | hu1D7v75 VHH | EVQLLESGGGEVQPGGSLRLSCVASRGKISQYRMGWYRQAPGKE |
| | | RELVAHISNTGDAAEYVGSVKDRFTISRDSTKNALYLQMSSLRA |
| | | EDTAVYKCNSVEDILAWGQGTLVTVRP |
| 117 | hu1D7v76 VHH | EVQLLESGGGEVQPGGSLRLSCVASRGKISQYRMGWYRQAPGKE |
| | | RELVAHISNTGDAAEYVVSVKDRFTISRDSTKNALYLQMSSLRA |
| | | EDTAVYKCNSVEDILAWGQGTLVTVRP |
| 118 | hu1D7v77 VHH | EVQLLESGGGEVQPGGSLRLSCVASRGKISQYRMGWYRQAPGKE |
| | | RELVAHISNTGDAAEYVDSVKDRFTISRDSTKNALYLQMSSLRA |
| | | EDTAVYKCYSVEDILAWGQGTLVTVRP |
| 119 | hu1D7v78 VHH | EVQLLESGGGEVQPGGSLRLSCVASRGKISQYRMGWYRQAPGKE |
| | | RELVAHISNTGDAAEYVDSVKDRFTISRDSTKNALYLQMSSLRA |
| | | EDTAVYKCNAVEDILAWGQGTLVTVRP |
| 120 | hu1D7v79 VHH | EVQLLESGGGEVQPGGSLRLSCVASRGKISQYRMGWYRQAPGKE |
| | | RELVAHISNTGDAAEYVDSVKDRFTISRDSTKNALYLQMSSLRA |
| | | EDTAVYKCNLVEDILAWGQGTLVTVRP |
| 121 | hu1D7v80 VHH | EVQLLESGGGEVQPGGSLRLSCVASRGKISQYRMGWYRQAPGKE |
| | | RELVAHISNTGDAAEYVDSVKDRFTISRDSTKNALYLQMSSLRA |
| | | EDTAVYKCNVVEDILAWGQGTLVTVRP |
| 122 | hu1D7v81 VHH | EVQLLESGGGEVQPGGSLRLSCVASRGKISQYRMGWYRQAPGKE |
| | | RELVAHISNTGDAAEYVDSVKDRFTISRDSTKNALYLQMSSLRA |
| | | EDTAVYKCASVEDILAWGQGTLVTVRP |
| 123 | hu1D7v82 VHH | EVQLLESGGGEVQPGGSLRLSCVASRGKISQYRMGWYRQAPGKE |
| | | RELVAHISNTGDAAEYVESVKDRFTISRDSTKNALYLQMSSLRA |
| | | EDTAVYKCNSVEDILAWGQGTLVTVRP |
| 124 | hu1D7v83 VHH | EVQLLESGGGEVQPGGSLRLSCVASRGKISQYRMGWYRQAPGKE |
| | | RELVAHISNTGDAAEYVDGVKDRFTISRDSTKNALYLQMSSLRA |
| | | EDTAVYKCNSVEDILAWGQGTLVTVRP |
| 125 | hu1D7v84 VHH | EVQLLESGGGEVQPGGSLRLSCVASRGKISQYRMGWYRQAPGKE |
| | | RELVAHISNTGDAAEYVEGVKDRFTISRDSTKNALYLQMSSLRA |
| | | EDTAVYKCNSVEDILAWGQGTLVTVRP |
| 126 | hu1D7v85 VHH | EVQLLESGGGEVQPGGSLRLSCVASRGKISQYRMGWYRQAPGKE |
| | | RELVAHISNTGDAAEYVSGVKDRFTISRDSTKNALYLQMSSLRA |
| | | EDTAVYKCNSVEDILAWGQGTLVTVRP |
| 127 | hu1D7v86 VHH | EVQLLESGGGEVQPGGSLRLSCVASRGKISQYRMGWYRQAPGKE |
| | | RELVAHISNTGDAAEYVGGVKDRFTISRDSTKNALYLQMSSLRA |
| | | EDTAVYKCNSVEDILAWGQGTLVTVRP |
| 128 | hu1D7v87 VHH | EVQLLESGGGEVQPGGSLRLSCVASRGKISQYRMGWYRQAPGKE |
| | | RELVAHISNTGDAAEYVQSVKDRFTISRDSTKNALYLQMSSLRA |
| | | EDTAVYKCNSVEDILAWGQGTLVTVRP |
| 129 | hu1D7v88 VHH | EVQLLESGGGEVQPGGSLRLSCVASRGKISQYRMGWYRQAPGKE |
| | | RELVAHISNTGDAAEYVQGVKDRFTISRDSTKNALYLQMSSLRA |
| | | EDTAVYKCNSVEDILAWGQGTLVTVRP |
| 130 | hu1D7v89 VHH | EVQLLESGGGEVQPGGSLRLSCVASRGKISQYRMGWYRQAPGKE |
| | | RELVAHISNTGDAAEYVDSVKDRFTISRDSTKNALYLQMSSLRA |
| | | EDTAVYKCQGVEDILAWGQGTLVTVRP |
| 131 | hu1D7v90 VHH | EVQLLESGGGEVQPGGSLRLSCVASRGKISQYRMGWYRQAPGKE |
| | | RELVAHISNTGDAAEYVDSVKDRFTISRDSTKNALYLQMSSLRA |
| | | EDTAVYKCSGVEDILAWGQGTLVTVRP |
| 132 | hu1D7v91 VHH | EVQLLESGGGEVQPGGSLRLSCVASRGKISQYRMGWYRQAPGKE |
| | | RELVAHISNTGDAAEYVDSVKDRFTISRDSTKNALYLQMSSLRA |
| | | EDTAVYKCGGVEDILAWGQGTLVTVRP |
| 133 | hu1D7v92 VHH | EVQLLESGGGEVQPGGSLRLSCVASRGKISQYRMGWYRQAPGKE |
| | | RELVAHISNTGDAAEYVDSVKDRFTISRDSTKNALYLQMSSLRA |
| | | EDTAVYKCQNVEDILAWGQGTLVTVRP |
| 134 | hu1D7v93 VHH | EVQLLESGGGEVQPGGSLRLSCVASRGKISQYRMGWYRQAPGKE |
| | | RELVAHISNTGDAAEYVDSVKDRFTISRDSTKNALYLQMSSLRA |
| | | EDTAVYKCNQVEDILAWGQGTLVTVRP |
| 135 | hu1D7v94 VHH | EVQLLESGGGEVQPGGSLRLSCVASRGKISQYRMGWYRQAPGKE |
| | | RELVAHISNTGDAAEYVDSVKDRFTISRDSTKNALYLQMSSLRA |
| | | EDTAVYKCSNVEDILAWGQGTLVTVRP |
| 136 | hu1D7v95 VHH | EVQLLESGGGEVQPGGSLRLSCVASRGKISQYRMGWYRQAPGKE |
| | | RELVAHISNTGDAAEYVDSVKDRFTISRDSTKNALYLQMSSLRA |
| | | EDTAVYKCSSVEDILAWGQGTLVTVRP |
| 137 | hu1D7v96 VHH | EVQLLESGGGEVQPGGSLRLSCVASRGKISQYRMGWYRQAPGKE |
| | | RELVAHISNTGDAAEYVYSVKDRFTISRDSTKNALYLQMSSLRA |
| | | EDTAVYKCNSVEDILAWGQGTLVTVRP |
| 138 | hu1D7v97 VHH | EVQLLESGGGEVQPGGSLRLSCVASRGKISQYRMGWYRQAPGKE |
| | | RELVAHISNTGDAAEYVDSVKDRFTISRDSTKNALYLQMSSLRA |
| | | EDTAVYKCYSVEDILAWGQGTLVTVRP |
| 139 | hu1D7v98 VHH | EVQLLESGGGEVQPGGSLRLSCVASRGKISQYRMGWYRQAPGKE |
| | | RELVAHISNTGDAAEYVLSVKDRFTISRDSTKNALYLQMSSLRA |
| | | EDTAVYKCNSVEDILAWGQGTLVTVRP |
| 140 | hu1D7v99 VHH | EVQLLESGGGEVQPGGSLRLSCVASRGKISQYRMGWYRQAPGKE |
| | | RELVAHISNTGDAAEYVSSVKDRFTISRDSTKNALYLQMSSLRA |
| | | EDTAVYKCNSVEDILAWGQGTLVTVRP |
| 141 | hu1D7v100 VHH | EVQLLESGGGEVQPGGSLRLSCVASRGKISQYRMGWYRQAPGKE |
| | | RELVAHISNTGDAAEYVDSVKDRFTISRDSTKNALYLQMSSLRA |
| | | EDTAVYYCNSVEDILAWGQGTLVTVRP |
| 142 | hu1D7v101 VHH | EVQLLESGGGEVQPGGSLRLSCVASRGKISQYRMGWYRQAPGKE |
| | | RELVAHISNTGDAAEYVDSVKDRFTISRDSTKNALYLQMSSLRA |
| | | EDTAVYKCAAVEDILAWGQGTLVTVRP |
| 143 | hu1D7v102 VHH | EVQLLESGGGEVQPGGSLRLSCVASRGKISQYRMGWYRQAPGKE |
| | | LEWVAHISNTGDAAEYVDSVKDRFTISRDSTKNALYLQMSSLRA |
| | | EDTAVYKCNTVEDILAWGQGTLVTVRP |
| 175 | 1D7v112 VHH | EVQLLESGGGEVQPGGSLRLSCVASRGKISQYRMGWYRQAPGKE |
| | | RELVAHISNTGDAAEYVDVVKDRFTISRDSTKNALYLQMSSLRA |
| | | EDTAVYKCNSVEDILAWGQGTLVTVRP |
| 176 | 1D7v113 VHH | EVQLLESGGGEVQPGGSLRLSCVASRGKISQYRMGWYRQAPGKE |
| | | RELVAHISNTGDAAEYVDLVKDRFTISRDSTKNALYLQMSSLRA |
| | | EDTAVYKCNSVEDILAWGQGTLVTVRP |
| 177 | 1D7v124 VHH | EVQLLESGGGEVQPGGSLRLSCVASRGKISQYRMGWYRQAPGKE |
| | | RELVAHISNTGDAAEYVDGVKDRFTISRDSTKNALYLQMSSLRA |
| | | EDTAVYKCNSVEDILAWGQGTLVTVRP |
| 178 | 1D7v125 VHH | EVQLLESGGGEVQPGGSLRLSCVASRGKISQYRMGWYRQAPGKE |
| | | RELVAHISNTGDAAEYVEGVKDRFTISRDSTKNALYLQMSSLRA |
| | | EDTAVYKCNSVEDILAWGQGTLVTVRP |
| 179 | 1D7v139 VHH | EVQLLESGGGEVQPGGSLRLSCVASRGKISQYRMGWYRQAPGKE |
| | | RELVAHISNTGDAAEYVLSVKDRFTISRDSTKNALYLQMSSLRA |
| | | EDTAVYKCNSVEDILAWGQGTLVTVRP |
| 158 | hu1D7v158 VHH | EVQLLESGGGEVQPGGSLRLSCVASRGKISQYRMGWYRQAPGKQ |
| | | RELVAHISNTGDAAEYVAPVKGRFTISRDNAKNALYLQMNSLRA |
| | | EDTAVYKCNTVEDILAWGQGTLVTVEP |
| 159 | hu1D7v158-1 VHH | EVQLLESGGGEVQPGGSLRLSCVASRGKISQYRMGWVRQAPGKQ |
| | | RELVAHISNTGDAAEYVAPVKGRFTISRDNAKNALYLQMNSLRA |
| | | EDTAVYKCNTVEDILAWGQGTLVTVEP |
| 166 | hu1D7v158-2 VHH | EVQLLESGGGEVQPGGSLRLSCVASRGKISQYRMGWYRQAPGKQ |
| | | RELVSHISNTGDAAEYVAPVKGRFTISRDNAKNALYLQMNSLRA |
| | | EDTAVYKCNTVEDILAWGQGTLVTVEP |
| 167 | hu1D7v158-3 VHH | EVQLLESGGGEVQPGGSLRLSCVASRGKISQYRMGWYRQAPGKQ |
| | | RELVAAISNTGDAAEYVAPVKGRFTISRDNAKNALYLQMNSLRA |
| | | EDTAVYKCNTVEDILAWGQGTLVTVEP |
| 168 | hu1D7v158-4 VHH | EVQLLESGGGEVQPGGSLRLSCVASRGKISQYRMGWYRQAPGKQ |
| | | RELVAHISNTGDAAEYAAPVKGRFTISRDNAKNALYLQMNSLRA |
| | | EDTAVYKCNTVEDILAWGQGTLVTVEP |
| 169 | hu1D7v158-5 VHH | EVQLLESGGGEVQPGGSLRLSCVASRGKISQYRMGWYRQAPGKQ |
| | | RELVAHISNTGDAAEYVEPVKGRFTISRDNAKNALYLQMNSLRA |
| | | EDTAVYKCNTVEDILAWGQGTLVTVEP |
| 170 | hu1D7v158-6 VHH | EVQLLESGGGEVQPGGSLRLSCVASRGKISQYRMGWYRQAPGKQ |
| | | RELVAHISNTGDAAEYVASVKGRFTISRDNAKNALYLQMNSLRA |
| | | EDTAVYKCNTVEDILAWGQGTLVTVEP |
| 171 | hu1D7v158-7 VHH | EVQLLESGGGEVQPGGSLRLSCVASRGKISQYRMGWYRQAPGKQ |
| | | RELVAHISNTGDAAEYVAPVKGRFTISRDNAKNTLYLQMNSLRA |
| | | EDTAVYKCNTVEDILAWGQGTLVTVEP |
| 172 | hu1D7v158-8 VHH | EVQLLESGGGEVQPGGSLRLSCVASRGKISQYRMGWYRQAPGKQ |
| | | RELVAHISNTGDAAEYAESVKGRFTISRDNAKNALYLQMNSLRA |
| | | EDTAVYKCNTVEDILAWGQGTLVTVEP |
| 173 | hu1D7v158-9 VHH | EVQLLESGGGEVQPGGSLRLSCVASRGKISQYRMGWYRQAPGKE |
| | | REFVAHISNTGDAAEYVAPVKGRFTISRDNAKNALYLQMNSLRA |
| | | EDTAVYKCNTVEDILAWGQGTLVTVEP |
| 174 | hu1D7v158-10 VHH | EVQLLESGGGEVQPGGSLRLSCVASRGKISQYRMGWYRQAPGKQ |
| | | RELVAHISNTGDAAEYVAPVKGRFTISRDNAKNALYLQMNSLRA |
| | | EDTAVYYCNTVEDILAWGQGTLVTVEP |
| 180 | Consensus humanized | EVQLLESGGGEVQPGGSLRLSCX₁ASX₂GX₃X₄SQYRMGWYRQ |
| | 1D7 (c1D7) | APGKX₅X₆EX₇VX₈X₉ISNX₁₀GX₁₁X₁₂X₁₃X₁₄YX₁₅X₁₆X₁₇VK |
| | | X₁₈RFTISRDX₁₉X₂₀KNX₂₁X₂₂YLQMX₂₃SLRAEDTAVYX₂₄C |
| | | X₂₅X₂₆VEDILAWGQGTLVTVX₂₇P |
| | | wherein: X₁ is V or A; X₂ is R or G; X₃ is K or |
| | | T; X₄ is I or F; X₅ is Q, G or E; |
| | | X₆ is R or L; X₇ is L, W or F; X₈ is A or |
| | | S; X₉ is H or A; X₁₀ is T or S; X₁₁ is D or G; |
| | | X₁₂ is A or S; X₁₃ is A or T; X₁₄ is E or Y; |
| | | X₁₅ is V or A; X₁₆ is D, E, A, G, V, S, Y, L or Q; |
| | | X₁₇ is S, P, T, A, V, L, I, or G; X₁₈ is G |
| | | or D; X₁₉ is S or N; X₂₀ is T or A; X₂₁ is A |
| | | or T; X₂₂ is V or L; X₂₃ is N or S; X₂₄ |
| | | is K or Y; X₂₅ is N, S, E, Y, A, S, G, |
| | | Q; X₂₆ is S, T, A, L, V, N or G; and |
| | | X₂₇ is K, R, E, or D |
| 32 | human IgG1 Fc region | DKTHTCPPCPAPELLGGPSVFLFPPKPKDTLMISRTPEVTCVVV |
| | | DVSHEDPEVKFNWYVDGVEVHNAKTKPREEQYNSTYRVVSVLTV |
| | | LHQDWLNGKEYKCKVSNKALPAPIEKTISKAKGQPREPQVYTLP |
| | | PSRDELTKNQVSLTCLVKGFYPSDIAVEWESNGQPENNYKTTPP |
| | | VLDSDGSFFLYSKLTVDKSRWQQGNVFSCSVMHEALHNHYTQKS |
| | | LSLSPGK |
| 33 | human IgG1 Fc xELL | DKTHTCPPCPAPGGPSVFLFPPKPKDTLMISRTPEVTCVVVDVS |
| | | HEDPEVKFNWYVDGVEVHNAKTKPREEQYNSTYRVVSVLTVLHQ |
| | | DWLNGKEYKCKVSNKALPAPIEKTISKAKGQPREPQVYTLPPSR |
| | | DELTKNQVSLTCLVKGFYPSDIAVEWESNGQPENNYKTTPPVLD |
| | | SDGSFFLYSKLTVDKSRWQQGNVFSCSVMHEALHNHYTQKSLSL |
| | | SPGK |
| 181 | human IgG1 Fc-NNT | GGPSVFLFPPKPKDTLMISRTPEVTCVVVDVSHEDPEVKFNWYV |
| | | DGVEVHNAKTKPREEQYNSTYRVVSVLTVLHQDWLNGKEYKCKV |
| | | SNKALPAPIEKTISKAKGQPREPQVYTLPPSRDELTKNQVNLTC |
| | | LVKGFYPSDIAVEWESNGQPENNYKTTPPVLDSDGSFFLNSTLT |
| | | VDKSRWQQGNVFSCSVMHEALHNHYTQKSLSLSPGK |
| 34 | Fc region M252Y and | DKTHTCPPCPAPELLGGPSVFLFPPKPKDTLYISRTPEVTCVVV |
| | M428V (YV) S354C | DVSHEDPEVKFNWYVDGVEVHNAKTKPREEQYNSTYRVVSVLTV |
| | T366W knob | LHQDWLNGKEYKCKVSNKALPAPIEKTISKAKGQPREPQVYTLP |
| | | PCRDELTKNQVSLWCLVKGFYPSDIAVEWESNGQPENNYKTTPP |
| | | VLDSDGSFFLYSKLTVDKSRWQQGNVFSCSVVHEALHNHYTQKS |
| | | LSLSPGK |
| 35 | Fc region M252Y, | DKTHTCPPCPAPELLGGPSVFLFPPKPKDTLYISRTPEVTCVVV |
| | M428V, H435R (YVR) | DVSHEDPEVKFNWYVDGVEVHNAKTKPREEQYNSTYRVVSVLTV |
| | T366S, L368A, Y407V | LHQDWLNGKEYKCKVSNKALPAPIEKTISKAKGQPREPQVCTLP |
| | hole | PSRDELTKNQVSLSCAVKGFYPSDIAVEWESNGQPENNYKTTPP |
| | | VLDSDGSFFLVSKLTVDKSRWQQGNVFSCSVVHEALHNRYTQKS |
| | | LSLSPGK |
| 36 | Fc region xELL H435R | DKTHTCPPCPAPGGPSVFLFPPKPKDTLMISRTPEVTCVVVDVS |
| | | HEDPEVKFNWYVDGVEVHNAKTKPREEQYNSTYRVVSVLTVLHQ |
| | | DWLNGKEYKCKVSNKALPAPIEKTISKAKGQPREPQVYTLPPSR |
| | | DELTKNQVSLTCLVKGFYPSDIAVEWESNGQPENNYKTTPPVLD |
| | | SDGSFFLYSKLTVDKSRWQQGNVFSCSVMHEALHNRYTQKSLSL |
| | | SPGK |
| 37 | Fc region xELL | DKTHTCPPCPAPGGPSVFLFPPKPKDTLYISRTPEVTCVVVDVS |
| | M252Y and M428V | HEDPEVKFNWYVDGVEVHNAKTKPREEQYNSTYRVVSVLTVLHQ |
| | (YV) | DWLNGKEYKCKVSNKALPAPIEKTISKAKGQPREPQVYTLPPSR |
| | | DELTKNQVSLTCLVKGFYPSDIAVEWESNGQPENNYKTTPPVLD |
| | | SDGSFFLYSKLTVDKSRWQQGNVFSCSVVHEALHNHYTQKSLSL |
| | | SPGK |
| 38 | Fc region xELL | DKTHTCPPCPAPGGPSVFLFPPKPKDTLYISRTPEVTCVVVDVS |
| | M252Y and M428L | HEDPEVKFNWYVDGVEVHNAKTKPREEQYNSTYRVVSVLTVLHQ |
| | (YL) | DWLNGKEYKCKVSNKALPAPIEKTISKAKGQPREPQVYTLPPSR |
| | | DELTKNQVSLTCLVKGFYPSDIAVEWESNGQPENNYKTTPPVLD |
| | | SDGSFFLYSKLTVDKSRWQQGNVFSCSVLHEALHNHYTQKSLSL |
| | | SPGK |
| 39 | Fc region xELL | DKTHTCPPCPAPGGPSVFLFPPKPKDTLYISRTPEVTCVVVDVS |
| | M252Y, M428L, | HEDPEVKFNWYVDGVEVHNAKTKPREEQYNSTYRVVSVLTVLHQ |
| | H435R (YLR) | DWLNGKEYKCKVSNKALPAPIEKTISKAKGQPREPQVYTLPPSR |
| | | DELTKNQVSLTCLVKGFYPSDIAVEWESNGQPENNYKTTPPVLD |
| | | SDGSFFLYSKLTVDKSRWQQGNVFSCSVLHEALHNRYTQKSLSL |
| | | SPGK |
| 40 | Fc region xELL | DKTHTCPPCPAPGGPSVFLFPPKPKDTLYISRTPEVTCVVVDVS |
| | M252Y, M428V, | HEDPEVKFNWYVDGVEVHNAKTKPREEQYNSTYRVVSVLTVLHQ |
| | H435R (YVR) | DWLNGKEYKCKVSNKALPAPIEKTISKAKGQPREPQVYTLPPSR |
| | | DELTKNQVSLTCLVKGFYPSDIAVEWESNGQPENNYKTTPPVLD |
| | | SDGSFFLYSKLTVDKSRWQQGNVFSCSVVHEALHNRYTQKSLSL |
| | | SPGK |
| 41 | Fc region xELL S354C | DKTHTCPPCPAPGGPSVFLFPPKPKDTLMISRTPEVTCVVVDVS |
| | T366W knob | HEDPEVKFNWYVDGVEVHNAKTKPREEQYNSTYRVVSVLTVLHQ |
| | | DWLNGKEYKCKVSNKALPAPIEKTISKAKGQPREPQVYTLPPCR |
| | | DELTKNQVSLWCLVKGFYPSDIAVEWESNGQPENNYKTTPPVLD |
| | | SDGSFFLYSKLTVDKSRWQQGNVFSCSVMHEALHNHYTQKSLSL |
| | | SPGK |
| 42 | Fc region xELL H435R | DKTHTCPPCPAPGGPSVFLFPPKPKDTLMISRTPEVTCVVVDVS |
| | S354C T366W knob | HEDPEVKFNWYVDGVEVHNAKTKPREEQYNSTYRVVSVLTVLHQ |
| | | DWLNGKEYKCKVSNKALPAPIEKTISKAKGQPREPQVYTLPPCR |
| | | DELTKNQVSLWCLVKGFYPSDIAVEWESNGQPENNYKTTPPVLD |
| | | SDGSFFLYSKLTVDKSRWQQGNVFSCSVMHEALHNRYTQKSLSL |
| | | SPGK |
| 43 | Fc region xELL | DKTHTCPPCPAPGGPSVFLFPPKPKDTLYISRTPEVTCVVVDVS |
| | M252Y and M428V | HEDPEVKFNWYVDGVEVHNAKTKPREEQYNSTYRVVSVLTVLHQ |
| | (YV) S354C T366W | DWLNGKEYKCKVSNKALPAPIEKTISKAKGQPREPQVYTLPPCR |
| | knob | DELTKNQVSLWCLVKGFYPSDIAVEWESNGQPENNYKTTPPVLD |
| | | SDGSFFLYSKLTVDKSRWQQGNVFSCSVVHEALHNHYTQKSLSL |
| | | SPGK |
| 44 | Fc region xELL | DKTHTCPPCPAPGGPSVFLFPPKPKDTLYISRTPEVTCVVVDVS |
| | M252Y and M428L | HEDPEVKFNWYVDGVEVHNAKTKPREEQYNSTYRVVSVLTVLHQ |
| | (YL) S354C T366W | DWLNGKEYKCKVSNKALPAPIEKTISKAKGQPREPQVYTLPPCR |
| | knob | DELTKNQVSLWCLVKGFYPSDIAVEWESNGQPENNYKTTPPVLD |
| | | SDGSFFLYSKLTVDKSRWQQGNVFSCSVLHEALHNHYTQKSLSL |
| | | SPGK |
| 45 | Fc region xELL | DKTHTCPPCPAPGGPSVFLFPPKPKDTLYISRTPEVTCVVVDVS |
| | M252Y, M428L, | HEDPEVKFNWYVDGVEVHNAKTKPREEQYNSTYRVVSVLTVLHQ |
| | H435R (YLR) S354C | DWLNGKEYKCKVSNKALPAPIEKTISKAKGQPREPQVYTLPPCR |
| | T366W knob | DELTKNQVSLWCLVKGFYPSDIAVEWESNGQPENNYKTTPPVLD |
| | | SDGSFFLYSKLTVDKSRWQQGNVFSCSVLHEALHNRYTQKSLSL |
| | | SPGK |
| 46 | Fc region xELL | DKTHTCPPCPAPGGPSVFLFPPKPKDTLYISRTPEVTCVVVDVS |
| | M252Y, M428V, | HEDPEVKFNWYVDGVEVHNAKTKPREEQYNSTYRVVSVLTVLHQ |
| | H435R (YVR) S354C | DWLNGKEYKCKVSNKALPAPIEKTISKAKGQPREPQVYTLPPCR |
| | T366W knob | DELTKNQVSLWCLVKGFYPSDIAVEWESNGQPENNYKTTPPVLD |
| | | SDGSFFLYSKLTVDKSRWQQGNVFSCSVVHEALHNRYTQKSLSL |
| | | SPGK |
| 47 | Fc region xELL T366S, | DKTHTCPPCPAPGGPSVFLFPPKPKDTLMISRTPEVTCVVVDVS |
| | L368A, Y407V hole | HEDPEVKFNWYVDGVEVHNAKTKPREEQYNSTYRVVSVLTVLHQ |
| | | DWLNGKEYKCKVSNKALPAPIEKTISKAKGQPREPQVCTLPPSR |
| | | DELTKNQVSLSCAVKGFYPSDIAVEWESNGQPENNYKTTPPVLD |
| | | SDGSFFLVSKLTVDKSRWQQGNVFSCSVMHEALHNHYTQKSLSL |
| | | SPGK |
| 48 | Fc region xELL | DKTHTCPPCPAPGGPSVFLFPPKPKDTLMISRTPEVTCVVVDVS |
| | H435R, T366S, | HEDPEVKFNWYVDGVEVHNAKTKPREEQYNSTYRVVSVLTVLHQ |
| | L368A, Y407V hole | DWLNGKEYKCKVSNKALPAPIEKTISKAKGQPREPQVCTLPPSR |
| | | DELTKNQVSLSCAVKGFYPSDIAVEWESNGQPENNYKTTPPVLD |
| | | SDGSFFLVSKLTVDKSRWQQGNVFSCSVMHEALHNRYTQKSLSL |
| | | SPGK |
| 49 | Fc region xELL | DKTHTCPPCPAPGGPSVFLFPPKPKDTLYISRTPEVTCVVVDVS |
| | M252Y and M428V | HEDPEVKFNWYVDGVEVHNAKTKPREEQYNSTYRVVSVLTVLHQ |
| | (YV) T366S, L368A, | DWLNGKEYKCKVSNKALPAPIEKTISKAKGQPREPQVCTLPPSR |
| | Y407V hole | DELTKNQVSLSCAVKGFYPSDIAVEWESNGQPENNYKTTPPVLD |
| | | SDGSFFLVSKLTVDKSRWQQGNVFSCSVVHEALHNHYTQKSLSL |
| | | SPGK |
| 50 | Fc region xELL | DKTHTCPPCPAPGGPSVFLFPPKPKDTLYISRTPEVTCVVVDVS |
| | M252Y and M428L | HEDPEVKFNWYVDGVEVHNAKTKPREEQYNSTYRVVSVLTVLHQ |
| | (YL) T366S, L368A, | DWLNGKEYKCKVSNKALPAPIEKTISKAKGQPREPQVCTLPPSR |
| | Y407V hole | DELTKNQVSLSCAVKGFYPSDIAVEWESNGQPENNYKTTPPVLD |
| | | SDGSFFLVSKLTVDKSRWQQGNVFSCSVLHEALHNHYTQKSLSL |
| | | SPGK |
| 51 | Fc region xELL | DKTHTCPPCPAPGGPSVFLFPPKPKDTLYISRTPEVTCVVVDVS |
| | M252Y, M428L, | HEDPEVKFNWYVDGVEVHNAKTKPREEQYNSTYRVVSVLTVLHQ |
| | H435R (YLR) T366S, | DWLNGKEYKCKVSNKALPAPIEKTISKAKGQPREPQVCTLPPSR |
| | L368A, Y407V hole | DELTKNQVSLSCAVKGFYPSDIAVEWESNGQPENNYKTTPPVLD |
| | | SDGSFFLVSKLTVDKSRWQQGNVFSCSVLHEALHNRYTQKSLSL |
| | | SPGK |
| 52 | Fc region xELL | DKTHTCPPCPAPGGPSVFLFPPKPKDTLYISRTPEVTCVVVDVS |
| | M252Y, M428V, | HEDPEVKFNWYVDGVEVHNAKTKPREEQYNSTYRVVSVLTVLHQ |
| | H435R (YVR) T366S, | DWLNGKEYKCKVSNKALPAPIEKTISKAKGQPREPQVCTLPPSR |
| | L368A, Y407V hole | DELTKNQVSLSCAVKGFYPSDIAVEWESNGQPENNYKTTPPVLD |
| | | SDGSFFLVSKLTVDKSRWQQGNVFSCSVVHEALHNRYTQKSLSL |
| | | SPGK |
| 53 | Fc region H435R | DKTHTCPPCPAPELLGGPSVFLFPPKPKDTLMISRTPEVTCVVV |
| | | DVSHEDPEVKFNWYVDGVEVHNAKTKPREEQYNSTYRVVSVLTV |
| | | LHQDWLNGKEYKCKVSNKALPAPIEKTISKAKGQPREPQVYTLP |
| | | PSRDELTKNQVSLTCLVKGFYPSDIAVEWESNGQPENNYKTTPP |
| | | VLDSDGSFFLYSKLTVDKSRWQQGNVFSCSVMHEALHNRYTQKS |
| | | LSLSPGK |
| 54 | Fc region M252Y and | DKTHTCPPCPAPELLGGPSVFLFPPKPKDTLYISRTPEVTCVVV |
| | M428V (YV) | DVSHEDPEVKFNWYVDGVEVHNAKTKPREEQYNSTYRVVSVLTV |
| | | LHQDWLNGKEYKCKVSNKALPAPIEKTISKAKGQPREPQVYTLP |
| | | PSRDELTKNQVSLTCLVKGFYPSDIAVEWESNGQPENNYKTTPP |
| | | VLDSDGSFFLYSKLTVDKSRWQQGNVFSCSVVHEALHNHYTQKS |
| | | LSLSPGK |
| 55 | Fc region M252Y and | DKTHTCPPCPAPELLGGPSVFLFPPKPKDTLYISRTPEVTCVVV |
| | M428L (YL) | DVSHEDPEVKFNWYVDGVEVHNAKTKPREEQYNSTYRVVSVLTV |
| | | LHQDWLNGKEYKCKVSNKALPAPIEKTISKAKGQPREPQVYTLP |
| | | PSRDELTKNQVSLTCLVKGFYPSDIAVEWESNGQPENNYKTTPP |
| | | VLDSDGSFFLYSKLTVDKSRWQQGNVFSCSVLHEALHNHYTQKS |
| | | LSLSPGK |
| 56 | Fc region M252Y, | DKTHTCPPCPAPELLGGPSVFLFPPKPKDTLYISRTPEVTCVVV |
| | M428L, H435R (YLR) | DVSHEDPEVKFNWYVDGVEVHNAKTKPREEQYNSTYRVVSVLTV |
| | | LHQDWLNGKEYKCKVSNKALPAPIEKTISKAKGQPREPQVYTLP |
| | | PSRDELTKNQVSLTCLVKGFYPSDIAVEWESNGQPENNYKTTPP |
| | | VLDSDGSFFLYSKLTVDKSRWQQGNVFSCSVLHEALHNRYTQKS |
| | | LSLSPGK |
| 57 | Fc region M252Y, | DKTHTCPPCPAPELLGGPSVFLFPPKPKDTLYISRTPEVTCVVV |
| | M428V, H435R (YVR) | DVSHEDPEVKFNWYVDGVEVHNAKTKPREEQYNSTYRVVSVLTV |
| | | LHQDWLNGKEYKCKVSNKALPAPIEKTISKAKGQPREPQVYTLP |
| | | PSRDELTKNQVSLTCLVKGFYPSDIAVEWESNGQPENNYKTTPP |
| | | VLDSDGSFFLYSKLTVDKSRWQQGNVFSCSVVHEALHNRYTQKS |
| | | LSLSPGK |
| 58 | Fc region S354C | DKTHTCPPCPAPELLGGPSVFLFPPKPKDTLMISRTPEVTCVVV |
| | T366W knob | DVSHEDPEVKFNWYVDGVEVHNAKTKPREEQYNSTYRVVSVLTV |
| | | LHQDWLNGKEYKCKVSNKALPAPIEKTISKAKGQPREPQVYTLP |
| | | PCRDELTKNQVSLWCLVKGFYPSDIAVEWESNGQPENNYKTTPP |
| | | VLDSDGSFFLYSKLTVDKSRWQQGNVFSCSVMHEALHNHYTQKS |
| | | LSLSPGK |
| 59 | Fc region H435R | DKTHTCPPCPAPELLGGPSVFLFPPKPKDTLMISRTPEVTCVVV |
| | S354C T366W knob | DVSHEDPEVKFNWYVDGVEVHNAKTKPREEQYNSTYRVVSVLTV |
| | | LHQDWLNGKEYKCKVSNKALPAPIEKTISKAKGQPREPQVYTLP |
| | | PCRDELTKNQVSLWCLVKGFYPSDIAVEWESNGQPENNYKTTPP |
| | | VLDSDGSFFLYSKLTVDKSRWQQGNVFSCSVMHEALHNRYTQKS |
| | | LSLSPGK |
| 60 | Fc region M252Y and | DKTHTCPPCPAPELLGGPSVFLFPPKPKDTLYISRTPEVTCVVV |
| | M428L (YL) S354C | DVSHEDPEVKFNWYVDGVEVHNAKTKPREEQYNSTYRVVSVLTV |
| | T366W knob | LHQDWLNGKEYKCKVSNKALPAPIEKTISKAKGQPREPQVYTLP |
| | | PCRDELTKNQVSLWCLVKGFYPSDIAVEWESNGQPENNYKTTPP |
| | | VLDSDGSFFLYSKLTVDKSRWQQGNVFSCSVLHEALHNHYTQKS |
| | | LSLSPGK |
| 61 | Fc region M252Y, | DKTHTCPPCPAPELLGGPSVFLFPPKPKDTLYISRTPEVTCVVV |
| | M428L, H435R (YLR) | DVSHEDPEVKFNWYVDGVEVHNAKTKPREEQYNSTYRVVSVLTV |
| | S354C T366W knob | LHQDWLNGKEYKCKVSNKALPAPIEKTISKAKGQPREPQVYTLP |
| | | PCRDELTKNQVSLWCLVKGFYPSDIAVEWESNGQPENNYKTTPP |
| | | VLDSDGSFFLYSKLTVDKSRWQQGNVFSCSVLHEALHNRYTQKS |
| | | LSLSPGK |
| 62 | Fc region M252Y, | DKTHTCPPCPAPELLGGPSVFLFPPKPKDTLYISRTPEVTCVVV |
| | M428V, H435R (YVR) | DVSHEDPEVKFNWYVDGVEVHNAKTKPREEQYNSTYRVVSVLTV |
| | S354C T366W knob | LHQDWLNGKEYKCKVSNKALPAPIEKTISKAKGQPREPQVYTLP |
| | | PCRDELTKNQVSLWCLVKGFYPSDIAVEWESNGQPENNYKTTPP |
| | | VLDSDGSFFLYSKLTVDKSRWQQGNVFSCSVVHEALHNRYTQKS |
| | | LSLSPGK |
| 63 | Fc region T366S, | DKTHTCPPCPAPELLGGPSVFLFPPKPKDTLMISRTPEVTCVVV |
| | L368A, Y407V hole | DVSHEDPEVKFNWYVDGVEVHNAKTKPREEQYNSTYRVVSVLTV |
| | | LHQDWLNGKEYKCKVSNKALPAPIEKTISKAKGQPREPQVCTLP |
| | | PSRDELTKNQVSLSCAVKGFYPSDIAVEWESNGQPENNYKTTPP |
| | | VLDSDGSFFLVSKLTVDKSRWQQGNVFSCSVMHEALHNHYTQKS |
| | | LSLSPGK |
| 64 | Fc region H435R, | DKTHTCPPCPAPELLGGPSVFLFPPKPKDTLMISRTPEVTCVVV |
| | T366S, L368A, Y407V | DVSHEDPEVKFNWYVDGVEVHNAKTKPREEQYNSTYRVVSVLTV |
| | hole | LHQDWLNGKEYKCKVSNKALPAPIEKTISKAKGQPREPQVCTLP |
| | | PSRDELTKNQVSLSCAVKGFYPSDIAVEWESNGQPENNYKTTPP |
| | | VLDSDGSFFLVSKLTVDKSRWQQGNVFSCSVMHEALHNRYTQKS |
| | | LSLSPGK |
| 65 | Fc region M252Y and | DKTHTCPPCPAPELLGGPSVFLFPPKPKDTLYISRTPEVTCVVV |
| | M428V (YV) T366S, | DVSHEDPEVKFNWYVDGVEVHNAKTKPREEQYNSTYRVVSVLTV |
| | L368A, Y407V hole | LHQDWLNGKEYKCKVSNKALPAPIEKTISKAKGQPREPQVCTLP |
| | | PSRDELTKNQVSLSCAVKGFYPSDIAVEWESNGQPENNYKTTPP |
| | | VLDSDGSFFLVSKLTVDKSRWQQGNVFSCSVVHEALHNHYTQKS |
| | | LSLSPGK |
| 66 | Fc region M252Y and | DKTHTCPPCPAPELLGGPSVFLFPPKPKDTLYISRTPEVTCVVV |
| | M428L (YL) T366S, | DVSHEDPEVKFNWYVDGVEVHNAKTKPREEQYNSTYRVVSVLTV |
| | L368A, Y407V hole | LHQDWLNGKEYKCKVSNKALPAPIEKTISKAKGQPREPQVCTLP |
| | | PSRDELTKNQVSLSCAVKGFYPSDIAVEWESNGQPENNYKTTPP |
| | | VLDSDGSFFLVSKLTVDKSRWQQGNVFSCSVLHEALHNHYTQKS |
| | | LSLSPGK |
| 67 | Fc region M252Y, | DKTHTCPPCPAPELLGGPSVFLFPPKPKDTLYISRTPEVTCVVV |
| | M428L, H435R (YLR) | DVSHEDPEVKFNWYVDGVEVHNAKTKPREEQYNSTYRVVSVLTV |
| | T366S, L368A, Y407V | LHQDWLNGKEYKCKVSNKALPAPIEKTISKAKGQPREPQVCTLP |
| | hole | PSRDELTKNQVSLSCAVKGFYPSDIAVEWESNGQPENNYKTTPP |
| | | VLDSDGSFFLVSKLTVDKSRWQQGNVFSCSVLHEALHNRYTQKS |
| | | LSLSPGK |
| 68 | Fc region xELL | DKTHTCPPCPAPGGPSVFLFPPKPKDTLMISRTPEVTCVVVDVS |
| | P329G, H435R, T366S, | HEDPEVKFNWYVDGVEVHNAKTKPREEQYNSTYRVVSVLTVLHQ |
| | L368A, Y407V hole | DWLNGKEYKCKVSNKALGAPIEKTISKAKGQPREPQVCTLPPSR |
| | (lacking C-terminal | DELTKNQVSLSCAVKGFYPSDIAVEWESNGQPENNYKTTPPVLD |
| | lysine) | SDGSFFLVSKLTVDKSRWQQGNVFSCSVMHEALHNRYTQKSLSL |
| | | SPG |
| 69 | Fc region xELL P329G | DKTHTCPPCPAPGGPSVFLFPPKPKDTLMISRTPEVTCVVVDVS |
| | knob (lacking C- | HEDPEVKFNWYVDGVEVHNAKTKPREEQYNSTYRVVSVLTVLHQ |
| | terminal lysine) | DWLNGKEYKCKVSNKALGAPIEKTISKAKGQPREPQVYTLPPCR |
| | | DELTKNQVSLWCLVKGFYPSDIAVEWESNGQPENNYKTTPPVLD |
| | | SDGSFFLYSKLTVDKSRWQQGNVFSCSVMHEALHNHYTQKSLSL |
| | | SPG |
| 70 | Fc region 2 IgG1 xELL | DKTHTCPPCPAPGGPSVFLFPPKPKDTLMISRTPEVTCVVVDVS |
| | knob (lacking C- | HEDPEVKFNWYVDGVEVHNAKTKPREEQYNSTYRVVSVLTVLHQ |
| | terminal lysine) | DWLNGKEYKCKVSNKALPAPIEKTISKAKGQPREPQVYTLPPCR |
| | | DELTKNQVSLWCLVKGFYPSDIAVEWESNGQPENNYKTTPPVLD |
| | | SDGSFFLYSKLTVDKSRWQQGNVFSCSVMHEALHNHYTQKSLSL |
| | | SPG |
| 71 | Wild type human IL-2 | APTSSSTKKTQLQLEHLLLDLQMILNGINNYKNPKLTRMLTFKF |
| | | YMPKKATELKHLQCLEEELKPLEEVLNLAQSKNFHLRPRDLISN |
| | | INVIVLELKGSETTFMCEYADETATIVEFLNRWITFCQSIISTL |
| | | T |
| 9 | Fc region 2 IgG1 xELL | DKTHTCPPCPAPGGPSVFLFPPKPKDTLMISRTPEVTCVVVDVS |
| | knob-IL-2-T3G-H16A- | HEDPEVKFNWYVDGVEVHNAKTKPREEQYNSTYRVVSVLTVLHQ |
| | P65R-D84S-C125S | DWLNGKEYKCKVSNKALPAPIEKTISKAKGQPREPQVYTLPPCR |
| | | DELTKNQVSLWCLVKGFYPSDIAVEWESNGQPENNYKTTPPVLD |
| | | SDGSFFLYSKLTVDKSRWQQGNVFSCSVMHEALHNHYTQKSLSL |
| | | SPGGSGGSAPGSSSTKKTQLQLEALLLDLQMILNGINNYKNPKL |
| | | TRMLTFKFYMPKKATELKHLQCLEEELKRLEEVLNLAQSKNFHL |
| | | RPRSLISNINVIVLELKGSETTFMCEYADETATIVEFLNRWITF |
| | | SQSIISTLT |
| 10 | 5C8 VHH-Fc region | EVQLVESGGGLVQAGGSLRLSCAASGRPFSNYAMGWFRQAPGKE |
| | xELL H435R, T366S, | REFVAAISWSGGSTSYADSVKGRFTISRDNAKNTVYLQMNSPKP |
| | L368A, Y407V hole | EDTAIYYCAAQFSGADYGFGRLGIRGYEYDYWGQGTQVTVKPGG |
| | | GGDKTHTCPPCPAPGGPSVFLFPPKPKDTLMISRTPEVTCVVVD |
| | | VSHEDPEVKFNWYVDGVEVHNAKTKPREEQYNSTYRVVSVLTVL |
| | | HQDWLNGKEYKCKVSNKALPAPIEKTISKAKGQPREPQVCTLPP |
| | | SRDELTKNQVSLSCAVKGFYPSDIAVEWESNGQPENNYKTTPPV |
| | | LDSDGSFFLVSKLTVDKSRWQQGNVFSCSVMHEALHNRYTQKSL |
| | | SLSPGK |
| 11 | 5C8 VHH-Fc region 2 | EVQLVESGGGLVQAGGSLRLSCAASGRPFSNYAMGWFRQAPGKE |
| | IgG1 xELL knob-IL-2- | REFVAAISWSGGSTSYADSVKGRFTISRDNAKNTVYLQMNSPKP |
| | T3A-H16A-E61R- | EDTAIYYCAAQFSGADYGFGRLGIRGYEYDYWGQGTQVTVKPGG |
| | P65R-D84Y-C125S | GGDKTHTCPPCPAPGGPSVFLFPPKPKDTLMISRTPEVTCVVVD |
| | | VSHEDPEVKFNWYVDGVEVHNAKTKPREEQYNSTYRVVSVLTVL |
| | | HQDWLNGKEYKCKVSNKALPAPIEKTISKAKGQPREPQVYTLPP |
| | | CRDELTKNQVSLWCLVKGFYPSDIAVEWESNGQPENNYKTTPPV |
| | | LDSDGSFFLYSKLTVDKSRWQQGNVFSCSVMHEALHNHYTQKSL |
| | | SLSPGGSGGSAPASSSTKKTQLQLEALLLDLQMILNGINNYKNP |
| | | KLTRMLTFKFYMPKKATELKHLQCLERELKRLEEVLNLAQSKNF |
| | | HLRPRYLISNINVIVLELKGSETTFMCEYADETATIVEFLNRWI |
| | | TFSQSIISTLT |
| 12 | 6C4 VHH-Fc region 2 | EVQLVESGGGLVQAGGSLRLSCAVSVRTFSDYRMGWFRQAPGKE |
| | IgG1 xELL knob-IL-2- | REFVSTISWSGGLTYYADSVKGRFTISRDNSKNTLYLQMNSLKP |
| | T3A-H16A-E61R- | EDTAVYYCAAGGGYAGGTYYHPEEWGQGTQVTVKPGGGGDKTHT |
| | P65R-D84Y-C125S | CPPCPAPGGPSVFLFPPKPKDTLMISRTPEVTCVVVDVSHEDPE |
| | | VKFNWYVDGVEVHNAKTKPREEQYNSTYRVVSVLTVLHQDWLNG |
| | | KEYKCKVSNKALPAPIEKTISKAKGQPREPQVYTLPPCRDELTK |
| | | NQVSLWCLVKGFYPSDIAVEWESNGQPENNYKTTPPVLDSDGSF |
| | | FLYSKLTVDKSRWQQGNVFSCSVMHEALHNHYTQKSLSLSPGGS |
| | | GGSAPASSSTKKTQLQLEALLLDLQMILNGINNYKNPKLTRMLT |
| | | FKFYMPKKATELKHLQCLERELKRLEEVLNLAQSKNFHLRPRYL |
| | | ISNINVIVLELKGSETTFMCEYADETATIVEFLNRWITFSQSII |
| | | STLT |
| 13 | 6C4 VHH-Fc region | EVQLVESGGGLVQAGGSLRLSCAVSVRTFSDYRMGWFRQAPGKE |
| | xELL H435R, T366S, | REFVSTISWSGGLTYYADSVKGRFTISRDNSKNTLYLQMNSLKP |
| | L368A, Y407V hole | EDTAVYYCAAGGGYAGGTYYHPEEWGQGTQVTVKPGGGGDKTHT |
| | | CPPCPAPGGPSVFLFPPKPKDTLMISRTPEVTCVVVDVSHEDPE |
| | | VKFNWYVDGVEVHNAKTKPREEQYNSTYRVVSVLTVLHQDWLNG |
| | | KEYKCKVSNKALPAPIEKTISKAKGQPREPQVCTLPPSRDELTK |
| | | NQVSLSCAVKGFYPSDIAVEWESNGQPENNYKTTPPVLDSDGSF |
| | | FLVSKLTVDKSRWQQGNVFSCSVMHEALHNRYTQKSLSLSPGK |
| 14 | 401A9v1-VHH Fc | EVQLVESGGGEVQPGGSLRLSCAASGSINSINVMEWYRQAPGKE |
| | region xELL H435R, | RDLVAGITSDGDTNYAESVKGRFTISRDNAKNTLYLQMSSLRAE |
| | T366S, L368A, Y407V | DTAVYYCRARDWGSLTDYWGQGTLVTVKPGGGGDKTHTCPPCPA |
| | hole | PGGPSVFLFPPKPKDTLMISRTPEVTCVVVDVSHEDPEVKFNWY |
| | | VDGVEVHNAKTKPREEQYNSTYRVVSVLTVLHQDWLNGKEYKCK |
| | | VSNKALPAPIEKTISKAKGQPREPQVCTLPPSRDELTKNQVSLS |
| | | CAVKGFYPSDIAVEWESNGQPENNYKTTPPVLDSDGSFFLVSKL |
| | | TVDKSRWQQGNVFSCSVMHEALHNRYTQKSLSLSPGK |
| 15 | 401A9v1 VHH-Fc | EVQLVESGGGEVQPGGSLRLSCAASGSINSINVMEWYRQAPGKE |
| | region 2 IgG1 xELL | RDLVAGITSDGDTNYAESVKGRFTISRDNAKNTLYLQMSSLRAE |
| | knob-IL-2-T3A- | DTAVYYCRARDWGSLTDYWGQGTLVTVKPGGGGDKTHTCPPCPA |
| | H16A-E61R-P65R- | PGGPSVFLFPPKPKDTLMISRTPEVTCVVVDVSHEDPEVKFNWY |
| | D84Y-C125S | VDGVEVHNAKTKPREEQYNSTYRVVSVLTVLHQDWLNGKEYKCK |
| | | VSNKALPAPIEKTISKAKGQPREPQVYTLPPCRDELTKNQVSLW |
| | | CLVKGFYPSDIAVEWESNGQPENNYKTTPPVLDSDGSFFLYSKL |
| | | TVDKSRWQQGNVFSCSVMHEALHNHYTQKSLSLSPGGSGGSAPA |
| | | SSSTKKTQLQLEALLLDLQMILNGINNYKNPKLTRMLTFKFYMP |
| | | KKATELKHLQCLERELKRLEEVLNLAQSKNFHLRPRYLISNINV |
| | | IVLELKGSETTFMCEYADETATIVEFLNRWITFSQSIISTLT |
| 16 | 1D7 VHH-Fc region 2 | QVQLVQSGGGLVQPGGSLRLSCVASRGKISQYRMGWYRQAPGKQ |
| | IgG1 xELL knob-IL-2- | RELVAHISNTGDAAEYVDSVKGRFTISRDSTKNAVYLQMNSLKP |
| | T3A-H16A-E61R- | EDTAVYKCNSVEDILAWGQGTQVTVKPGGGGDKTHTCPPCPAPG |
| | P65R-D84Y-C125S | GPSVFLFPPKPKDTLMISRTPEVTCVVVDVSHEDPEVKFNWYVD |
| | | GVEVHNAKTKPREEQYNSTYRVVSVLTVLHQDWLNGKEYKCKVS |
| | | NKALPAPIEKTISKAKGQPREPQVYTLPPCRDELTKNQVSLWCL |
| | | VKGFYPSDIAVEWESNGQPENNYKTTPPVLDSDGSFFLYSKLTV |
| | | DKSRWQQGNVFSCSVMHEALHNHYTQKSLSLSPGGSGGSAPASS |
| | | STKKTQLQLEALLLDLQMILNGINNYKNPKLTRMLTFKFYMPKK |
| | | ATELKHLQCLERELKRLEEVLNLAQSKNFHLRPRYLISNINVIV |
| | | LELKGSETTFMCEYADETATIVEFLNRWITFSQSIISTLT |
| 78 | IL-2-T3A-H16A- E61R-P65R-D84Y- C125S (IL-2_X) | 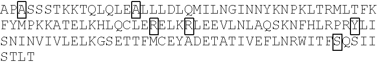 |
| 79 | IL-2-T3A-H16A- P65R-D84S-C125S (IL-2_Z) | 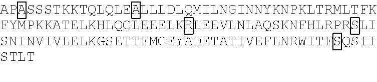 |
| 157 | IL-2-T3G-H16A- P65R-D84S-C125S (IL-2_Y) | 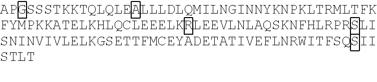 |
| 160 | OprI18v13 VHH-Fc | EVQLVESGGGEVQPGGSLRLSCAASETIFRFNTMAWYRQAPGKR |
| | region 2 IgG1 xELL | RELVGYITWAGRTGYAESVEGRFTISRDNAKNTVYLQMSSLRAE |
| | knob-IL-2-T3G- | DTAVYYCNKHGSSFTQDYWGQGTLVTVKPGGGGDKTHTCPPCPA |
| | H16A-P65R-D84S- | PGGPSVFLFPPKPKDTLMISRTPEVTCVVVDVSHEDPEVKFNWY |
| | C125S | VDGVEVHNAKTKPREEQYNSTYRVVSVLTVLHQDWLNGKEYKCK |
| | | VSNKALPAPIEKTISKAKGQPREPQVYTLPPCRDELTKNQVSLW |
| | | CLVKGFYPSDIAVEWESNGQPENNYKTTPPVLDSDGSFFLYSKL |
| | | TVDKSRWQQGNVFSCSVMHEALHNHYTQKSLSLSPGGSGGSAPG |
| | | SSSTKKTQLQLEALLLDLQMILNGINNYKNPKLTRMLTFKFYMP |
| | | KKATELKHLQCLEEELKRLEEVLNLAQSKNFHLRPRSLISNINV |
| | | IVLELKGSETTFMCEYADETATIVEFLNRWITFSQSIISTLT |
| 161 | OprI18v13 VHH-Fc | EVQLVESGGGEVQPGGSLRLSCAASETIFRFNTMAWYRQAPGKR |
| | region xELL H435R, | RELVGYITWAGRTGYAESVEGRFTISRDNAKNTVYLQMSSLRAE |
| | T366S, L368A, Y407V | DTAVYYCNKHGSSFTQDYWGQGTLVTVKPGGGGDKTHTCPPCPA |
| | hole | PGGPSVFLFPPKPKDTLMRSRTPEVTCVVVDVSHEDPEVKFNWY |
| | | VDGVEVHNAKTKPREEQYNSTYRVVSVLTVLHQDWLNGKEYKCK |
| | | VSNKALPAPIEKTISKAKGQPREPQVCTLPPSRDELTKNQVSLS |
| | | CAVKGFYPSDIAVEWESNGQPENNYKTTPPVLDSDGSFFLVSKL |
| | | TVDKSRWQQGNVFSCSVMHEALHNHYTQKSLSLSPGK |
| 162 | 5T4-hz16G10v11 | EVQLVESGGGEVQPGGSLRLSCAASGRPFSSYAMGWFRQAPGKE |
| | VHH-Fc region 2 | RETVAAVSRNAGSSYYAESVKGRFTISRDNAKNTVYLQMSSLRA |
| | IgG1 xELL-knob-IL2- | EDTAVYYCAARSAAYSRSSETYTEKHDYTYWGQGTLVTVKPGGG |
| | T3G-H16A-P65R- | GDKTHTCPPCPAPGGPSVFLFPPKPKDTLMISRTPEVTCVVVDV |
| | D84S-C125S | SHEDPEVKENWYVDGVEVHNAKTKPREEQYNSTYRVVSVLTVLH |
| | | QDWLNGKEYKCKVSNKALPAPIEKTISKAKGQPREPQVYTLPPC |
| | | RDELTKNQVSLWCLVKGFYPSDIAVEWESNGQPENNYKTTPPVL |
| | | DSDGSFFLYSKLTVDKSRWQQGNVFSCSVMHEALHNHYTQKSLS |
| | | LSPGGSGGSAPGSSSTKKTQLQLEALLLDLQMILNGINNYKNPK |
| | | LTRMLTFKFYMPKKATELKHLQCLEEELKRLEEVLNLAQSKNFH |
| | | LRPRSLISNINVIVLELKGSETTFMCEYADETATIVEFLNRWIT |
| | | FSQSIISTLT |
| 163 | 5D7 VHH-Fc region | EVQLVESGGGLVQAGGSLRLSCIASGRTFSNYAMGWFRQAPGKE |
| | xELL H435R, T366S, | REFVAAISWSGGMTDHADSVKGRFTISRDNAKNTVYLQMNSLKP |
| | L368A, Y407V hole | EDTAVYYCAAAFAGDIPYGSSWYGDPTTYDYWGQGTQVTVKPGG |
| | | GGDKTHTCPPCPAPGGPSVFLFPPKPKDTLMISRTPEVTCVVVD |
| | | VSHEDPEVKFNWYVDGVEVHNAKTKPREEQYNSTYRVVSVLTVL |
| | | HQDWLNGKEYKCKVSNKALPAPIEKTISKAKGQPREPQVCTLPP |
| | | SRDELTKNQVSLSCAVKGFYPSDIAVEWESNGQPENNYKTTPPV |
| | | LDSDGSFFLVSKLTVDKSRWQQGNVFSCSVMHEALHNRYTQKSL |
| | | SLSPGK |
| 164 | 1D7v158 VHH-Fc | EVQLLESGGGEVQPGGSLRLSCVASRGKISQYRMGWYRQAPGKQ |
| | region 2 IgG1 xELL | RELVAHISNTGDAAEYVAPVKGRFTISRDNAKNALYLQMNSLRA |
| | knob-IL-2-T3A- | EDTAVYKCNTVEDILAWGQGTLVTVEPGGGGDKTHTCPPCPAPG |
| | H16A-E61R-P65R- | GPSVFLFPPKPKDTLMISRTPEVTCVVVDVSHEDPEVKFNWYVD |
| | D84Y-C125S | GVEVHNAKTKPREEQYNSTYRVVSVLTVLHQDWLNGKEYKCKVS |
| | | NKALPAPIEKTISKAKGQPREPQVYTLPPCRDELTKNQVSLWCL |
| | | VKGFYPSDIAVEWESNGQPENNYKTTPPVLDSDGSFFLYSKLTV |
| | | DKSRWQQGNVFSCSVMHEALHNHYTQKSLSLSPGGSGGSAPASS |
| | | STKKTQLQLEALLLDLQMILNGINNYKNPKLTRMLTFKFYMPKK |
| | | ATELKHLQCLERELKRLEEVLNLAQSKNFHLRPRYLISNINVIV |
| | | LELKGSETTFMCEYADETATIVEFLNRWITFSQSIISTLT |

What is claimed is:

1. A polypeptide comprising at least one VHH domain that binds γδ TCR and a modified IL-2, wherein at least one VHH domain that binds γδ TCR comprises a CDR1 comprising the amino acid sequence of SEQ ID NO: 3, 144, 146, 147, or 148; a CDR2 comprising the amino acid sequence of SEQ ID NO: 4, 150, 151, 152, 153, 154, 155, or 156; and a CDR3 comprising the amino acid sequence of SEQ ID NO: 5, and wherein the modified IL-2 comprises T3A or T3G, H16A, P65R, C125S, and D84S or D84Y mutations relative to a wild type human IL-2 comprising the amino acid sequence of SEQ ID NO: 71.

2. The polypeptide of claim 1, wherein the CDR1 comprises the amino acid sequence of SEQ ID NO: 3, the CDR2 comprises the amino acid sequence of SEQ ID NO: 4, and the CDR3 comprises the amino acid sequence of SEQ ID NO: 5.

3. The polypeptide of claim 1 or 2, wherein the modified IL-2 comprises T3A, H16A, P65R, C125S, and D84S mutations.

4. The polypeptide of claim 1 or 2, wherein the modified IL-2 comprises T3A, H16A, E61R, P65R, C125S, and D84Y mutations.

5. The polypeptide of any one of claims 1-4, wherein the modified IL-2 comprises an amino acid sequence that is at least 90%, at least 95%, at least 97%, at least 98%, at least 99%, or 100% identical to the amino acid sequence of SEQ ID NO: 78 or 79 or 157.

6. The polypeptide of any one of claims 1, 2, or 4, wherein the modified IL-2 comprises the amino acid sequence of SEQ ID NO: 78.

7. The polypeptide of any one of claims 1-6, wherein at least one VHH domain, or each VHH domain, is humanized.

8. The polypeptide of any one of claim 1-7, wherein at least one VHH domain comprises SEQ ID NO: 180, wherein X₁, X₂, X₃, X₄, X₅, X₆, X₇, X₈, X₉, X₁₀, X₁₁, X₁₂, X₁₃, X₁₄, X₁₅, X₁₆, X₁₇, X₁₈, X₁₉, X₂₀, X₂₁, X₂₂, X₂₃, X₂₄, X₂₅, and X₂₆ are independently selected, and wherein X₁ is V or A; X₂ is R or G; X₃ is I or F; X₄ is Q, G or E; X₅ is R or L; X₆ is L, W or F; X₇ is A or S; X₈ is H or A; X₉ is T or S; X₁₀ is D or G; X₁₁ is A or S; X₁₂ is A or T; X₁₃ is E or Y; X₁₄ is V or A; X₁₅ is D, E, A or Q; X₁₆ is S, P, T, or G; X₁₇ is G or D; X₁₈ is S or N; X₁₉ is T or A; X₂₀ is A or T; X₂₁ is V or L; X₂₂ is N or S; X₂₃ is K or Y; X₂₄ is N or S; X₂₅ is S, T or G; and X₂₆ is K, R, E, or D.

9. The polypeptide of any one of claims 1-8, wherein at least one VHH domain comprises an amino acid sequence:
a) at least 85%, at least 90%, at least 95%, or at least 99% identical to an amino acid sequence selected from SEQ ID NOs: 2, 17-19, 21-23, 25-31, 72-77, 80, 81, 84-86, 88-93, 95, 97-107, 109, 111-129, 131-133, 135-137, 139-143, 158-159, and 166-179; or
b) selected from SEQ ID NO: 17-19, 21-23, 25-31, 72-77, 80, 81, 84-86, 88-93, 95, 97-107, 109, 111-129, 131-133, 135-137, 139-143, 158-159, and 166-179.

10. The polypeptide of any one of claims 1-9, wherein at least one VHH domain comprises the amino acid sequence of SEQ ID NO: 99, 143, or 158.

11. The polypeptide of any one of claims 1-10, comprising two VHH domains.

12. The polypeptide of any one of claims 1-10, comprising three VHH domains.

13. The polypeptide of any one of claims 1-12, wherein the polypeptide comprises at least one antigen-binding domain that binds an antigen other than γδ TCR.

14. The polypeptide of claim 13, wherein the at least one antigen-binding domain is a VHH domain.

15. The polypeptide of claim 13 or claim 14, wherein the polypeptide comprises at least one VHH domain that binds human serum albumin.

16. The polypeptide of claim 15, wherein the polypeptide comprises one VHH domain that binds γδ TCR, one VHH domain that binds human serum albumin, and the modified IL-2, optionally wherein the modified IL-2 is fused to the C-terminus of the VHH domain that binds human serum albumin.

17. The polypeptide of any one of claims 1-16, wherein the polypeptide does not comprise an Fc region.

18. The polypeptide of any one of claims 1-16, wherein the polypeptide comprises an Fc region.

19. The polypeptide of claim 18, wherein the Fc region comprises an amino acid sequence selected from SEQ ID NOs: 32-70, optionally wherein the Fc region lacks the C-terminal lysine residue.

20. The polypeptide of claim 18 or claim 19, wherein the modified IL-2 is fused to the C-terminus of the Fc region.

21. The polypeptide of any one of claims 1-10, and 18-20, wherein the polypeptide comprises one VHH domain that binds γδ TCR, an Fc region, and a modified IL-2.

22. The polypeptide of any one of claims 1-21, wherein the polypeptide comprises at least one antigen-binding domain that binds an antigen other than γδ TCR, optionally wherein at least one antigen-binding domain that binds an antigen other than γδ TCR binds a tumor cell antigen.

23. The polypeptide of claim 22, comprising at least one antigen-binding domain that binds Lag3, TGFBR1, TGFBR2, Fas, TNFR2, 1-92-LFA-3, 5T4, Alpha-4 integrin, Alpha-V integrin, alpha4beta1 integrin, alpha4beta7 integrin, AGR2, Anti-Lewis-Y, Apelin J receptor, APRIL, B7-H3, B7-H4, B7-H6, BAFF, BCMA, BTLA, C5 complement, C-242, CA9, CA19-9, (Lewis a), Carbonic anhydrase 9, CD2, CD3, CD6, CD9, CD11a, CD19, CD20, CD22, CD24, CD25, CD27, CD28, CD30, CD33, CD38, CD39, CD40, CD40L, CD41, CD44, CD44v6, CD47, CD51, CD52, CD56, CD64, CD70, CD71, CD73, CD74, CD80, CD81, CD86, CD95, CD117, CD123, CD125, CD132, (IL-2RG), CD133, CD137, CD138, CD166, CD172A, CD248, CDH6, CEACAM5 (CEA), CEACAM6 (NCA-90), CLAUDIN-3, CLAUDIN-4, cMet, Collagen, Cripto, CSFR, CSFR-1, CTLA4, CTGF, CXCL10, CXCL13, CXCR1, CXCR2, CXCR4, CYR61, DL44, DLK1, DLL3, DLL4, DPP-4, DSG1, EDA, EDB, EGFR, EGFRviii, Endothelin B receptor (ETBR), ENPP3, EpCAM, EPHA2, EPHB2, ERBB3, F protein of RSV, FAP, FcRH5, FGF-2, FGF8, FGFR1, FGFR2, FGFR3, FGFR4, FLT-3, Folate receptor alpha (FRα), GAL3ST1, G-CSF, G-CSFR, GD2, GITR, GLUT1, GLUT4, GM-CSF, GM-CSFR, GP IIb/IIIa receptors, Gp130, GPIIB/IIIA, GPNMB, GPRC5D, GRP78, HAVCAR1, HER2/neu, HER3, HER4, HGF, hGH, HVEM, Hyaluronidase, ICOS, IFNalpha, IFNbeta, IFNgamma, IgE, IgE Receptor (FceRI), IGF, IGF1R, IL1B, IL1R, IL2, IL11, IL12, IL12p40, IL-12R, IL-12Rbetal, IL13, IL13R, IL15, IL17, IL18, IL21, IL23, IL23R, IL27/IL27R (wsx1), IL29, IL-31R, IL31/IL31R, IL2R, IL4, IL4R, IL6, IL6R, Insulin Receptor, Jagged Ligands, Jagged 1, Jagged 2, KISS1-R, LAG-3, LIF-R, Lewis X, LIGHT, LRP4, LRRC26, Ly6G6D, LyPD1, MCSP, Mesothelin, MICA, MICB, MRP4, MUC1, Mucin-16 (MUC16, CA-125), Na/K ATPase, NGF, Nicastrin, Notch Receptors, Notch 1, Notch 2, Notch 3, Notch 4, NOV, OSM-R, OX-40, PAR2, PDGF-AA, PDGF-BB, PDGFRalpha, PDGFRbeta, PD-1, PD-L1, PD-L2, Phosphatidyl-serine, P1GF, PSCA, PSMA, PSGR, RAAG12, RAGE, SLC44A4, Sphingosine 1 Phosphate, STEAP1, STEAP2, TAG-72, TAPA1, TEM-8, TGFbeta, TIGIT, TIM-3, TLR2, TLR4, TLR6, TLR7, TLR8, TLR9, TMEM31, TNFalpha, TNFR, TNFRS12A, TRAIL-R1, TRAIL-R2, Transferrin, Transferrin receptor, TRK-A, TRK-B, TROP-2 uPAR, VAP1, VCAM-1, VEGF, VEGF-A, VEGF-B, VEGF-C, VEGF-D, VEGFR1, VEGFR2, VEGFR3, VISTA, WISP-1, WISP-2, or WISP-3.

24. The polypeptide of claim 22 or 23, wherein at least one antigen-binding domain that binds an antigen other than γδ TCR is a VHH domain.

25. The polypeptide of claim 24, where each antigen-binding domain that binds an antigen other than γδ TCR is a VHH domain.

26. The polypeptide of any one of claims 21-23, wherein at least one antigen-binding domain that binds an antigen other than γδ TCR comprises a heavy chain variable region and a light chain variable region.

27. The polypeptide of claim 26, wherein each antigen-binding domain that binds an antigen other than γδ TCR comprises a heavy chain variable region and a light chain variable region.

28. A complex comprising a first polypeptide and a second polypeptide, wherein the first polypeptide is the polypeptide of any one of claims 18-27, wherein the first polypeptide comprises a first Fc region, and wherein the second polypeptide comprises a second Fc region, wherein the first and second Fc regions are the same or different.

29. A complex comprising a first polypeptide and a second polypeptide, wherein the first polypeptide comprises at least one VHH domain that binds γδ TCR and a first Fc region and the second polypeptide comprises a second Fc region and a modified IL-2, wherein at least one VHH domain that binds γδ TCR comprises a CDR1 comprising the amino acid sequence of SEQ ID NO: 3, 144, 146, 147, or 148; a CDR2 comprising the amino acid sequence of SEQ ID NO: 4, 150, 151, 152, 153, 154, 155, or 156; and a CDR3 comprising the amino acid sequence of SEQ ID NO: 5, and wherein the modified IL-2 comprises T3A or T3G, H16A, P65R, C125S, and D84S or D84Y mutations relative to a wild type human IL-2 comprising the amino acid sequence of SEQ ID NO: 71.

30. The complex of claim 29, wherein the CDR1 comprises the amino acid sequence of SEQ ID NO: 3, the CDR2 comprises the amino acid sequence of SEQ ID NO: 4, and the CDR3 comprises the amino acid sequence of SEQ ID NO: 5.

31. The complex of claim 29 or claim 30, wherein the modified IL-2 comprises T3A, H16A, P65R, C125S, and D84S mutations.

32. The complex of claim 29 or claim 30, wherein the modified IL-2 comprises T3A, H16A, E61R, P65R, C125S, and D84Y mutations.

33. The complex of any one of claims 29-32, wherein the modified IL-2 comprises an amino acid sequence that is at least 90%, at least 95%, at least 97%, at least 98%, at least 99%, or 100% identical to the amino acid sequence of SEQ ID NO: 78 or 79 or 157.

34. The complex of any one of claims 29, 30, and 32, wherein the modified IL-2 comprises the amino acid sequence of SEQ ID NO: 78.

35. The complex of any one of claims 29-34, wherein at least one VHH domain, or each VHH domain, is humanized.

36. The complex of any one of claim 29-35, wherein at least one VHH domain comprises SEQ ID NO: 180, wherein X₁, X₂, X₃, X₄, X₅, X₆, X₇, X₈, X₉, X₁₀, X₁₁, X₁₂, X₁₃, X₁₄, X₁₅, X₁₆, X₁₇, X₁₈, X₁₉, X₂₀, X₂₁, X₂₂, X₂₃, X₂₄, X₂₅, X₂₆ and X₂₇ are independently selected, and wherein X₁ is V or A; X₂ is R or G; X₃ is K or T; X₄ is I or F; X₅ is Q, G or E; X₆ is R or L; X₇ is L, W or F; X₈ is A or S; X₉ is H or A; X₁₀ is T or S; X₁₁ is D or G; X₁₂ is A or S; X₁₃ is A or T; X₁₄ is E or Y; X₁₅ is V or A; X₁₆ is D, E, A, G, V, S, Y, L or Q; X₁₇ is S, P, T, A, V, L, I, or G; X₁₈ is G or D; X₁₉ is S or N; X₂₀ is T or A; X₂₁ is A or T; X₂₂ is V or L; X₂₃ is N or S; X₂₄ is K or Y; X₂₅ is N, S, E, Y, A, S, G, Q; X₂₆ is S, T, A, L, V, N or G; and X₂₇ is K, R, E, or D.

37. The complex of any one of claims 29-36, wherein at least one VHH domain comprises:
a) an amino acid sequence at least 85%, at least 90%, at least 95%, or at least 99% identical to an amino acid sequence selected from SEQ ID NOs: 2, 17-19, 21-23, 25-31, 72-77, 80, 81, 84-86, 88-93, 95, 97-107, 109, 111-129, 131-133, 135-137, 139-143, 158-159, and 166-179; or
b) an amino acid sequence selected from SEQ ID NO: 17-19, 21-23, 25-31, 72-77, 80, 81, 84-86, 88-93, 95, 97-107, 109, 111-129, 131-133, 135-137, 139-143, 158-159, and 166-179.

38. The complex of any one of claims 29-37, wherein at least one VHH domain comprises the amino acid sequence of SEQ ID NO: 99, 143, or 158.

39. The complex of any one of claims 28-38, wherein the second polypeptide comprises at least one antigen-binding domain.

40. The complex of claim 39, wherein the second polypeptide comprises at least one antigen-binding domain that binds an antigen other than γδ TCR, optionally wherein if the antigen-binding domain that binds an antigen other than a γδ TCR comprises a heavy chain variable region and a light chain variable region, then the heavy chain variable region is fused to a heavy chain constant region comprising the second Fc region.

41. The complex of claim 40, wherein at least one antigen-binding domain that binds an antigen other than γδ TCR binds a tumor cell antigen.

42. The complex of claim 40 or 41, wherein at least one antigen-binding domain that binds an antigen other than γδ TCR binds Lag3, TGFBR1, TGFBR2, Fas, TNFR2, 1-92-LFA-3, 5T4, Alpha-4 integrin, Alpha-V integrin, alpha4beta1 integrin, alpha4beta7 integrin, AGR2, Anti-Lewis-Y, Apelin J receptor, APRIL, B7-H3, B7-H4, B7-H6, BAFF, BCMA, BTLA, C5 complement, C-242, CA9, CA19-9, (Lewis a), Carbonic anhydrase 9, CD2, CD3, CD6, CD9, CD11a, CD19, CD20, CD22, CD24, CD25, CD27, CD28, CD30, CD33, CD38, CD39, CD40, CD40L, CD41, CD44, CD44v6, CD47, CD51, CD52, CD56, CD64, CD70, CD71, CD73, CD74, CD80, CD81, CD86, CD95, CD117, CD123, CD125, CD132, (IL-2RG), CD133, CD137, CD138, CD166, CD172A, CD248, CDH6, CEACAM5 (CEA), CEACAM6 (NCA-90), CLAUDIN-3, CLAUDIN-4, cMet, Collagen, Cripto, CSFR, CSFR-1, CTLA4, CTGF, CXCL10, CXCL13, CXCR1, CXCR2, CXCR4, CYR61, DL44, DLK1, DLL3, DLL4, DPP-4, DSG1, EDA, EDB, EGFR, EGFRviii, Endothelin B receptor (ETBR), ENPP3, EpCAM, EPHA2, EPHB2, ERBB3, F protein of RSV, FAP, FcRH5, FGF-2, FGF8, FGFR1, FGFR2, FGFR3, FGFR4, FLT-3, Folate receptor alpha (FRα), GAL3ST1, G-CSF, G-CSFR, GD2, GITR, GLUT1, GLUT4, GM-CSF, GM-CSFR, GP IIb/IIIa receptors, Gp130, GPIIB/IIIA, GPNMB, GPRC5D, GRP78, HAVCAR1, HER2/neu, HER3, HER4, HGF, hGH, HVEM, Hyaluronidase, ICOS, IFNalpha, IFNbeta, IFNgamma, IgE, IgE Receptor (FceRI), IGF, IGF1R, IL1B, IL1R, IL2, IL11, IL12, IL12p40, IL-12R, IL-12Rbetal, IL13, IL13R, IL15, IL17, IL18, IL21, IL23, IL23R, IL27/IL27R (wsx1), IL29, IL-31R, IL31/IL31R, IL2R, IL4, IL4R, IL6, IL6R, Insulin Receptor, Jagged Ligands, Jagged 1, Jagged 2, KISS1-R, LAG-3, LIF-R, Lewis X, LIGHT, LRP4, LRRC26, Ly6G6D, LyPD1, MCSP, Mesothelin, MICA, MICB, MRP4, MUC1, Mucin-16 (MUC16, CA-125), Na/K ATPase, NGF, Nicastrin, Notch Receptors, Notch 1, Notch 2, Notch 3, Notch 4, NOV, OSM-R, OX-40, PAR2, PDGF-AA, PDGF-BB, PDGFRalpha, PDGFRbeta, PD-1, PD-L1, PD-L2, Phosphatidyl-serine, P1GF, PSCA, PSMA, PSGR, RAAG12, RAGE, SLC44A4, Sphingosine 1 Phosphate, STEAP1, STEAP2, TAG-72, TAPA1, TEM-8, TGFbeta, TIGIT, TIM-3, TLR2, TLR4, TLR6, TLR7, TLR8, TLR9, TMEM31, TNFalpha, TNFR, TNFRS12A, TRAIL-R1, TRAIL-R2, Transferrin, Transferrin receptor, TRK-A, TRK-B, TROP-2 uPAR, VAP1, VCAM-1, VEGF, VEGF-A, VEGF-B, VEGF-C, VEGF-D, VEGFR1, VEGFR2, VEGFR3, VISTA, WISP-1, WISP-2, or WISP-3.

43. The complex of any one of claims 39-42, wherein at least one antigen-binding domain of the second polypeptide is a VHH domain.

44. The complex of claim 43, wherein the second polypeptide comprises at least one VHH domain that binds γδ TCR.

45. The complex of claim 44, wherein the second polypeptide comprises at least one VHH domain that binds γδ TCR comprising a CDR1 comprising the amino acid sequence of SEQ ID NO: 3, 144, 146, 147, or 148; a CDR2 comprising the amino acid sequence of SEQ ID NO: 4, 150, 151, 152, 153, 154, 155, or 156; and a CDR3 comprising the amino acid sequence of SEQ ID NO: 5.

46. The complex of claim 44 or 45, wherein the second polypeptide comprises at least one VHH domain that binds γδ TCR comprising a CDR1 comprising the amino acid sequence of SEQ ID NO: 3; a CDR2 comprising the amino acid sequence of SEQ ID NO: 4; and a CDR3 comprising the amino acid sequence of SEQ ID NO: 5.

47. The complex of any one of claims 44-46, wherein at least one VHH domain, or each VHH domain, of the second polypeptide is humanized.

48. The complex of any one of claims 44-47, wherein at least one VHH domain of the second polypeptide comprises SEQ ID NO: 180, wherein X₁, X₂, X₃, X₄, X₅, X₆, X₇, X₈, X₉, X₁₀, X₁₁, X₁₂, X₁₃, X₁₄, X₁₅, X₁₆, X₁₇, X₁₈, X₁₉, X₂₀, X₂₁, X₂₂, X₂₃, X₂₄, X₂₅, and X₂₆ are independently selected, and wherein X₁ is V or A; X₂ is R or G; X₃ is I or F; X₄ is Q, G or E; X₅ is R or L; X₆ is L, W or F; X₇ is A or S; X₈ is H or A; X₉ is T or S; X₁₀ is D or G; X₁₁ is A or S; X₁₂ is A or T; X₁₃ is E or Y; X₁₄ is V or A; X₁₅ is D, E, A or Q; X₁₆ is S, P, T, or G; X₁₇ is G or D; X₁₈ is S or N; X₁₉ is T or A; X₂₀ is A or T; X₂₁ is V or L; X₂₂ is N or S; X₂₃ is K or Y; X₂₄ is N or S; X₂₅ is S, T or G; and X₂₆ is K, R, E, or D.

49. The complex of any one of claims 44-48, wherein at least one VHH domain of the second polypeptide comprises:
a) an amino acid sequence at least 85%, 90%, 95%, or at least 99% identical to an amino acid sequence selected from SEQ ID NOs: 2, 17-19, 21-23, 25-31, 72-77, 80, 81, 84-86, 88-93, 95, 97-107, 109, 111-129, 131-133, 135-137, 139-143, 158-159, and 166-179; or
b) an amino acid sequence selected from SEQ ID NO: 17-19, 21-23, 25-31, 72-77, 80, 81, 84-86, 88-93, 95, 97-107, 109, 111-129, 131-133, 135-137, 139-143, 158-159, and 166-179.

50. The complex of any one of claims 44-49, wherein at least one VHH domain of the second polypeptide comprises the amino acid sequence of SEQ ID NO: 99, 143, or 158.

51. The complex of any one of claims 44-50, wherein the second polypeptide comprises one VHH domain that binds γδ TCR.

52. The complex of any one of claims 28-51, wherein the first Fc region comprises at least one knob mutation and the second Fc region comprises at least one hole mutation; or wherein the first Fc region comprises at least one hole mutation and the second Fc region comprises at least one knob mutation.

53. The complex of claim 52, wherein the first or second Fc region comprises a T366W mutation and the other of the first or second Fc region comprises T366S, L368A, and Y407V mutations.

54. The complex of claim 53, wherein the Fc region comprising the T366S, L368A, and Y407V mutations further comprises a H435R or H435K mutation.

55. The complex of any one of claims 28-54, wherein the complex forms under physiological conditions.

56. The polypeptide or complex of any one of claims 1-55, wherein the γδ TCR is human γδ TCR.

57. The polypeptide or complex of any one of claims 1-56, wherein the VHH domain binds to a human γδ TCR comprising human gamma9 and human delta2.

58. A pharmaceutical composition comprising the polypeptide or complex of any one of claims 1-57, and a pharmaceutically acceptable carrier.

59. An isolated nucleic acid that encodes the polypeptide or complex of any one of claims 1-58.

60. A vector comprising the nucleic acid of claim 59.

61. A host cell comprising the nucleic acid of claim 59 or the vector of claim 60.

62. A host cell that expresses the polypeptide or complex of any one of claims 1-57.

63. A method of producing the polypeptide or complex of any one of claims 1-57, comprising incubating the host cell of claim 61 or claim 62 under conditions suitable for expression of the polypeptide or complex.

64. The method of claim 63, further comprising isolating the polypeptide or complex.

65. A method of increasing γδ T cell proliferation comprising contacting T cells with the polypeptide or complex of any one of claims 1-57.

66. The method of claim 65, wherein the T cells are in vitro.

67. The method of claim 65, wherein the T cells are in vivo.

68. A method of treating cancer comprising administering to a subject with cancer a pharmaceutically effective amount of the polypeptide or complex of any one of claims 1-57, or the pharmaceutical composition of claim 58.

69. The method of claim 68, wherein the cancer is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer; gastrointestinal cancer; glioblastoma; hepatic carcinoma; hepatoma; intra-epithelial neoplasm; kidney or renal cancer; larynx cancer; liver cancer; lung cancer; small-cell lung cancer; non-small cell lung cancer; adenocarcinoma of the lung; squamous carcinoma of the lung; melanoma; myeloma; neuroblastoma; oral cavity cancer; ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma; Hodgkin's lymphoma; non-Hodgkin's lymphoma; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; chronic lymphocytic leukemia (CLL); acute lymphoblastic leukemia (ALL); Hairy cell leukemia; and chronic myeloblastic leukemia.

70. The method of claim 68 or 69, further comprising administering an additional therapeutic agent.

71. The method of claim 70, wherein the additional therapeutic agent is an anti-cancer agent.

72. The method of claim 71, wherein the anti-cancer agent is selected from a chemotherapeutic agent, an anti-cancer biologic, radiation therapy, CAR-T therapy, and an oncolytic virus.

73. The method of claim 72, wherein the additional therapeutic agent is an anti-cancer biologic.

74. The method of claim 73, wherein the anti-cancer biologic is an agent that inhibits PD-1 and/or PD-L1.

75. The method of claim 73, wherein the anti-cancer biologic is an agent that inhibits VISTA, gpNMB, B7H3, B7H4, HHLA2, CTLA4, or TIGIT.

76. The method of any one of claims 73-75, wherein the anti-cancer agent is an antibody.

77. The method of claim 73, wherein the anti-cancer biologic is a cytokine.

78. The method of claim 72, wherein the anti-cancer agent is CAR-T therapy.

79. The method of claim 72, wherein the anti-cancer agent is an oncolytic virus.

80. The method of any one of claims 68-79, further comprising tumor resection and/or radiation therapy.
